(19) United States
(12) Patent Application Publication
(10) Pub. No.: US 2026/0263485 A1
(43) Pub. Date: Sep. 10, 2026

(54) AMINOSTEROL COMPOUNDS, AMINOSTEROL-CYCLODEXTRIN FORMULATIONS, AND METHODS OF USING THE SAME

(71) Applicant: Enterin, Inc., Philadelphia, PA (US)

(72) Inventors: Denise BARBUT, Philadelphia, PA (US); Michael ZASLOFF, Philadelphia, PA (US); William A. KINNEY, Philadelphia, PA (US)

(73) Assignee: Enterin, Inc., Philadelphia, PA (US)

(21) Appl. No.: 18/877,724

(22) PCT Filed: Jun. 27, 2023
(86) PCT No.: PCT/US2023/026357

§ 371 Date: Dec. 20, 2024

Related U.S. Application Data
(60) Provisional application No. 63/356,910, filed on Jun. 29, 2022.
(60) Provisional application No. 63/459,107, filed on Apr. 13, 2023.
(60) Provisional application No. 63/461,875, filed on Apr. 25, 2023.

(51) Int. Cl. A61K 31/58; A61K 9/00; A61K 31/575; A61K 47/69; C07J 41/00; C07J 43/00

(52) U.S. Cl. CPC A61K 31/58; A61K 9/0019; A61K 31/575; A61K 47/6951; C07J 41/0005; C07J 43/003

(57) ABSTRACT

Described are novel aminosterol compounds and additionally aminosterol-cyclodextrin formulations suitable for injectable administration, and methods of using the same.

### CROSS-REFERENCE TO RELATED APPLICATIONS

The present application claims the benefit of priority of U.S. Provisional Application No. 63/356,910, filed on Jun. 29, 2022: U.S. Provisional Application No. 63/459,107, filed on Apr. 13, 2023; and U.S. Provisional Application No. 63/461,875, filed on Apr. 25, 2023, the contents of which are specifically incorporated by reference.

### FIELD OF THE INVENTION

The present application relates generally to novel aminosterol compounds, aminosterol-cyclodextrin compositions, and methods of treatment using such compounds and compositions.

### BACKGROUND OF THE INVENTION

Aminosterols are amino derivatives of a sterol. Exemplary aminosterols include squalamine (ENOT-0l) and aminosterol 1436 (ENT-02) (also known as trodusqluernine and MSI-1436). These aminosterols were discovered by Michael Zasloffin the spiny dogfish shark Squalus acanthias (U.S. Pat. No. 5,192,756) and they exhibit diverse pharmacological activity in mammalian systems. Aminosterol 1436 exhibits pharmacology in vertebrates causing weight loss and adipose tissue mobilization (Zasloff et al., 2001). Squalamine has antiviral, antibiotic, antifungal, and anticancer activity, and inhibits aggregation of the α-synuclein protein characteristic in Parkinson's disease.

Other recently described aminosterols include (1) ENT-03 and ENT-05 (US-2023/0123701), (2) ENT-04, ENT-07, ENT-08, ENT-09, ENT-10, and [ENT-11 (US 2023/0125585), and (3) ENT-06 (U S 2022/0372066).

It is well understood that stereochemical purity of drugs is important in some therapeutics, single-enantiomer or compounds having stereodefinition at key carbon centers can provide greater selectivities for their biological targets, improved therapeutic indices, and/or better pharmacokinetics than a mixture of isomers.

There is a need in the art for new aminosterol compounds, as such compounds are useful in treating a variety of human disease. There is also a need in the art for new pharmaceutical formulations of aminosterols, as the aminosteroid compounds are useful in treating a variety of human disease. The present invention satisfies these needs.

### SUMMARY OF THE INVENTION

In one aspect, disclosed are aminosterol compounds designated ENT-12, ENT-13, ENT-14, ENT-14S, ENT-14R, ENT-15, ENT-16, ENT-17 (also known as M-PU), ENT-18, ENT-18S, ENT-18R, ENT-19 (ENT-19 is the racemic version of M10-PU), ENT-19S, ENT-20, ENT-21, ENT-21 S, ENT-2IR, ENT-21 mixed isomers, ENT-22, ENT-22S, ENT-22R, ENT-22 mixed isomers, ENT-23, ENT-24, ENT-25, ENT-25R, ENT-25S (also known as M11-RB), ENT-25 mixed isomers, ENT-26, ENT-27, ENT-28, ENT-29, ENT-30, ENT-31, ENT-32, ENT-33, M10-PU/M14-RB, M2-RB, M3-RB, MS-RB, M5-RB, M16-RB, Aminosterol Compound A, Aminosterol Compound B, and Amninosterol Compound C, or a pharmaceutically acceptable salt, solvate, prodrug, enantiomer, or derivative thereof, with the structure of each compound shown below:

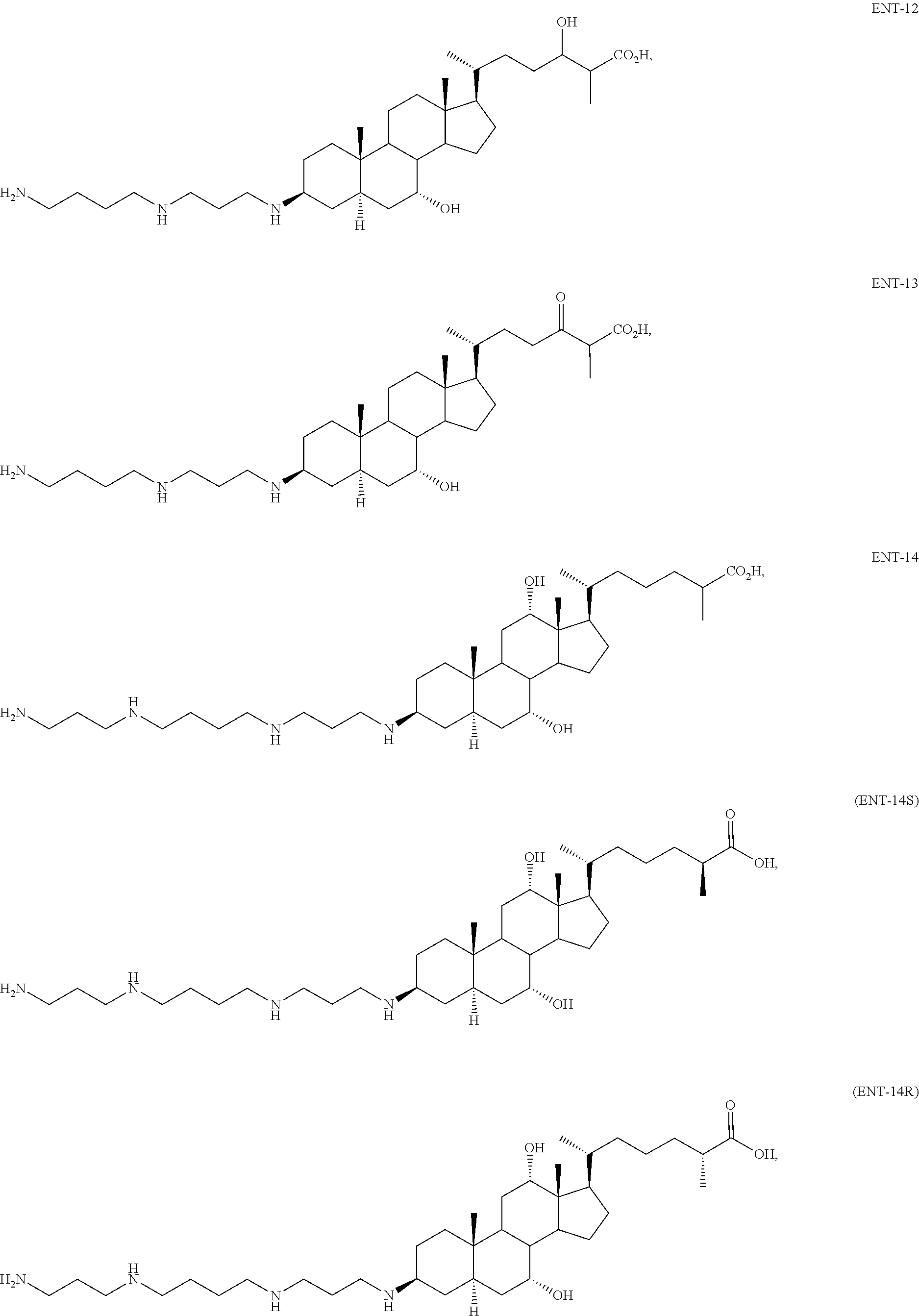

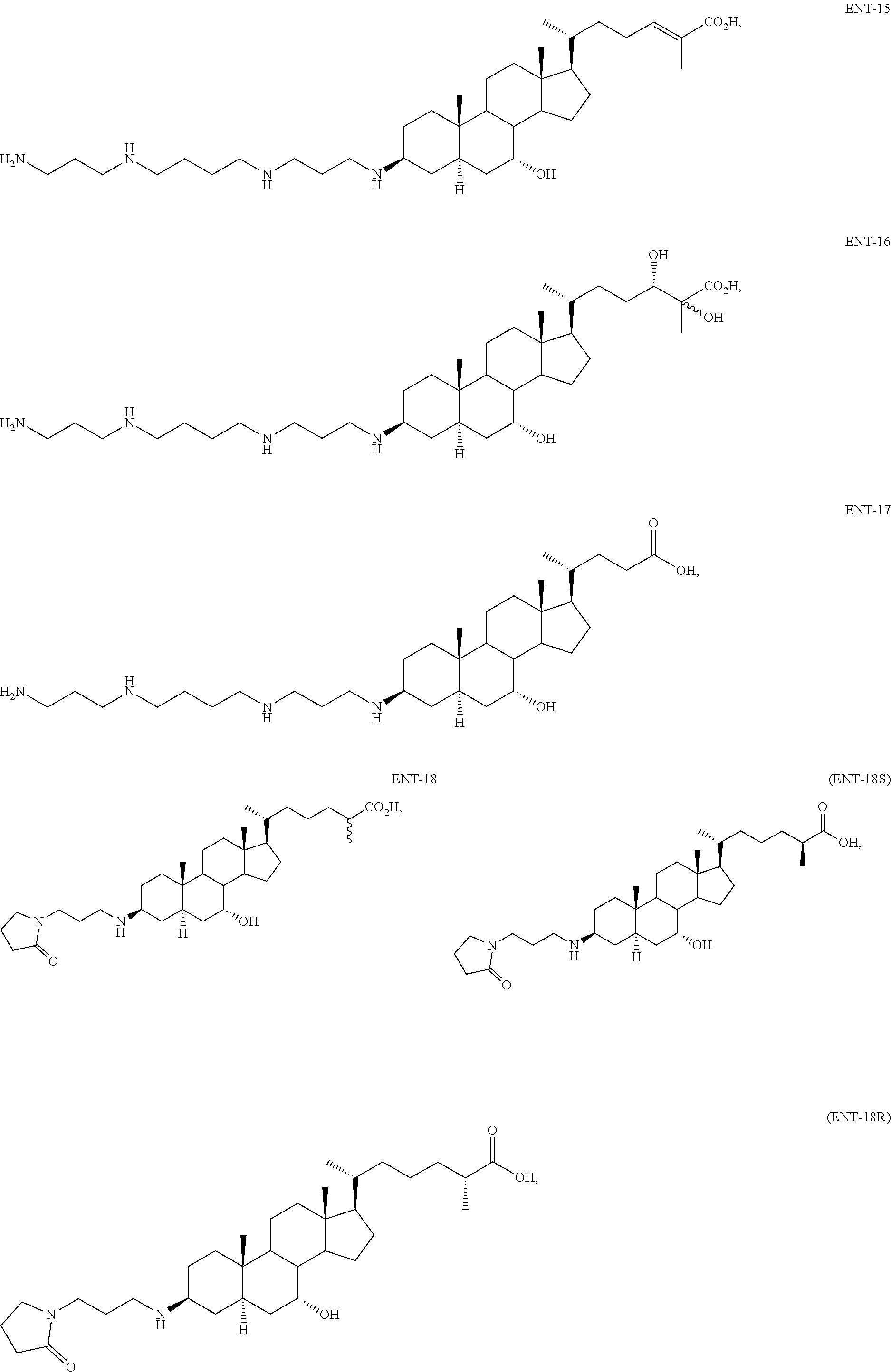

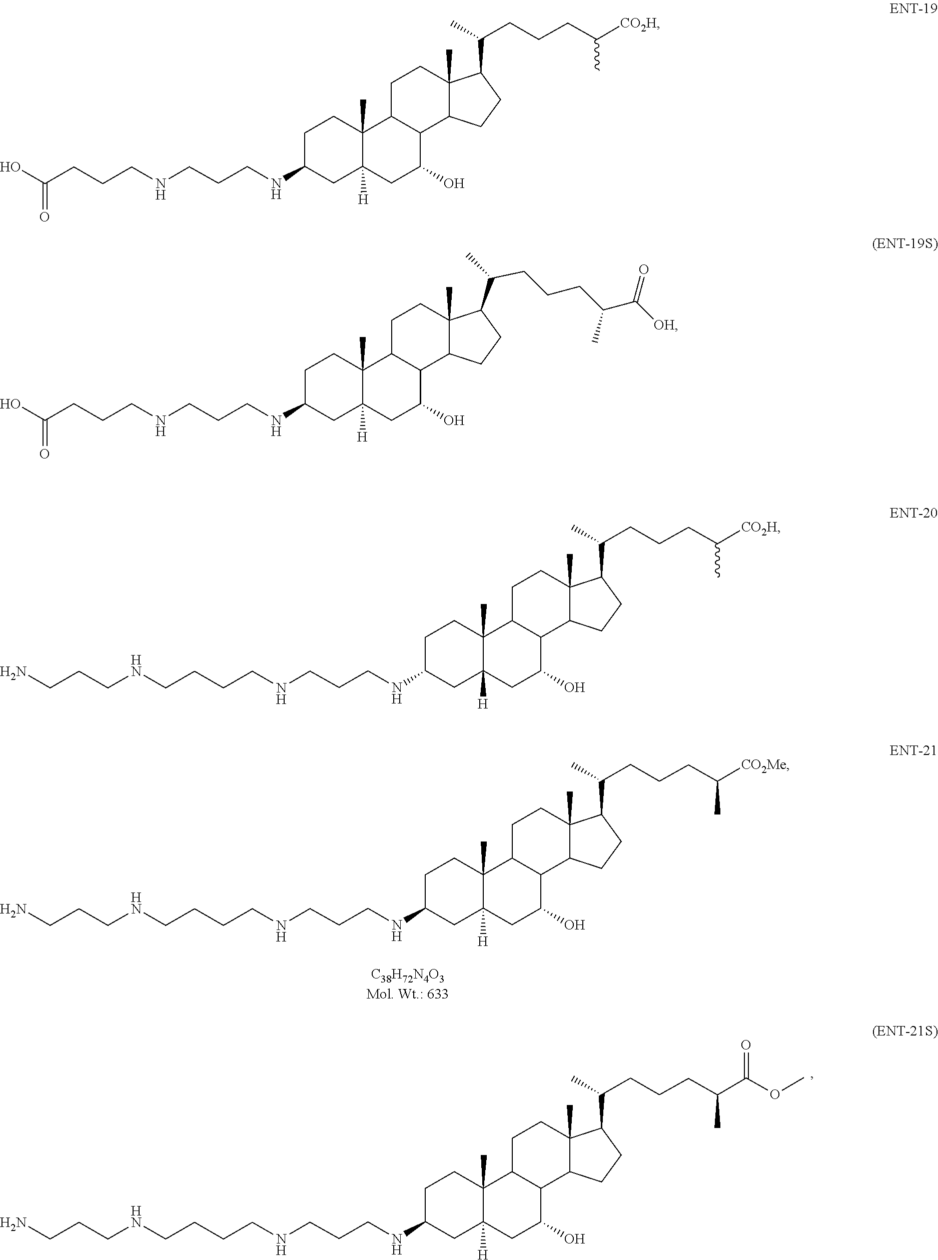

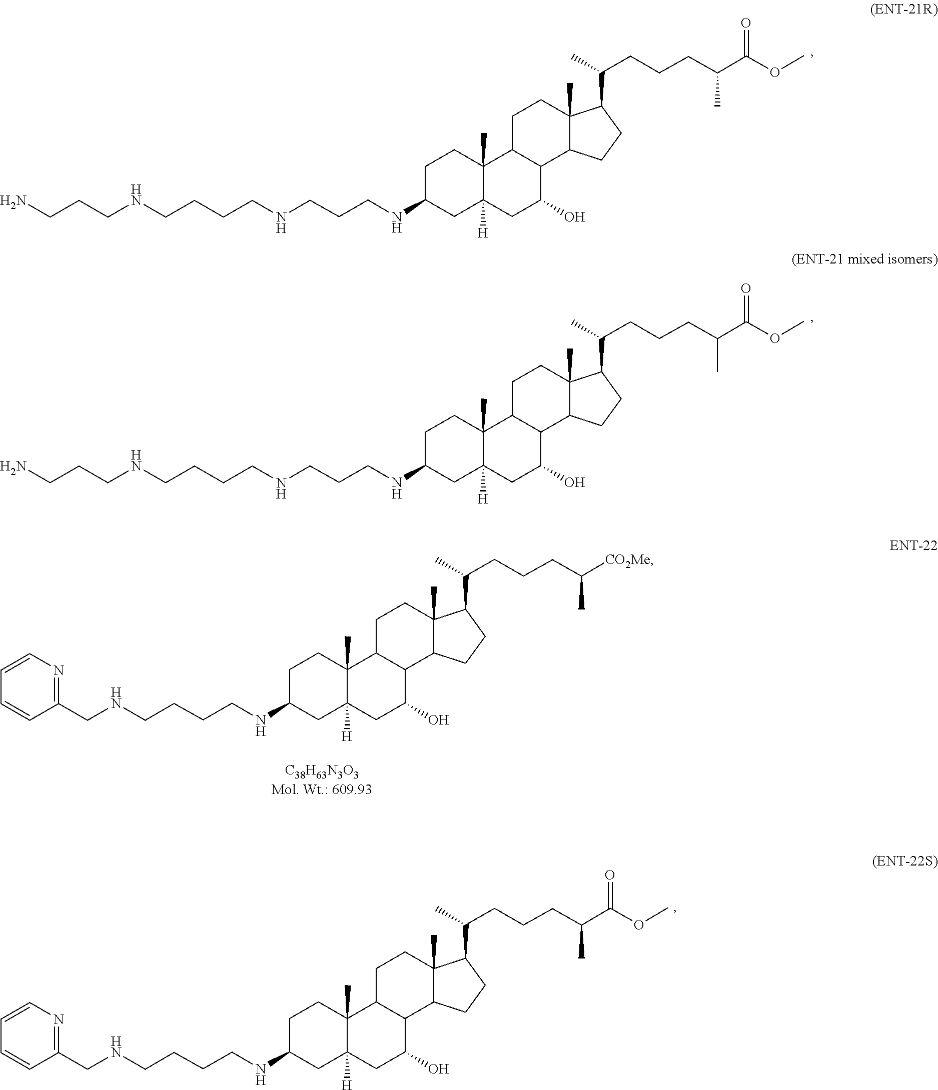 [SMILES: COC(=O)[C@H](C)CCC[C@@H](C)[C@H]1CCC2C3C(CC[C@@]21C)[C@@]1(C)CC[C@H](NCCCNCCCCNCCCN)C[C@@H]1C[C@H]3O ; COC(=O)C(C)CCC[C@@H](C)[C@H]1CCC2C3C(CC[C@@]21C)[C@@]1(C)CC[C@H](NCCCNCCCCNCCCN)C[C@@H]1C[C@H]3O ; COC(=O)[C@@H](C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)[C@@H](C)CCC[C@@H](C)[C@H]1CCC2C3C(CC[C@@]21C)[C@@]1(C)CC[C@H](NCCCCNCc2ccccn2)C[C@@H]1C[C@H]3O]

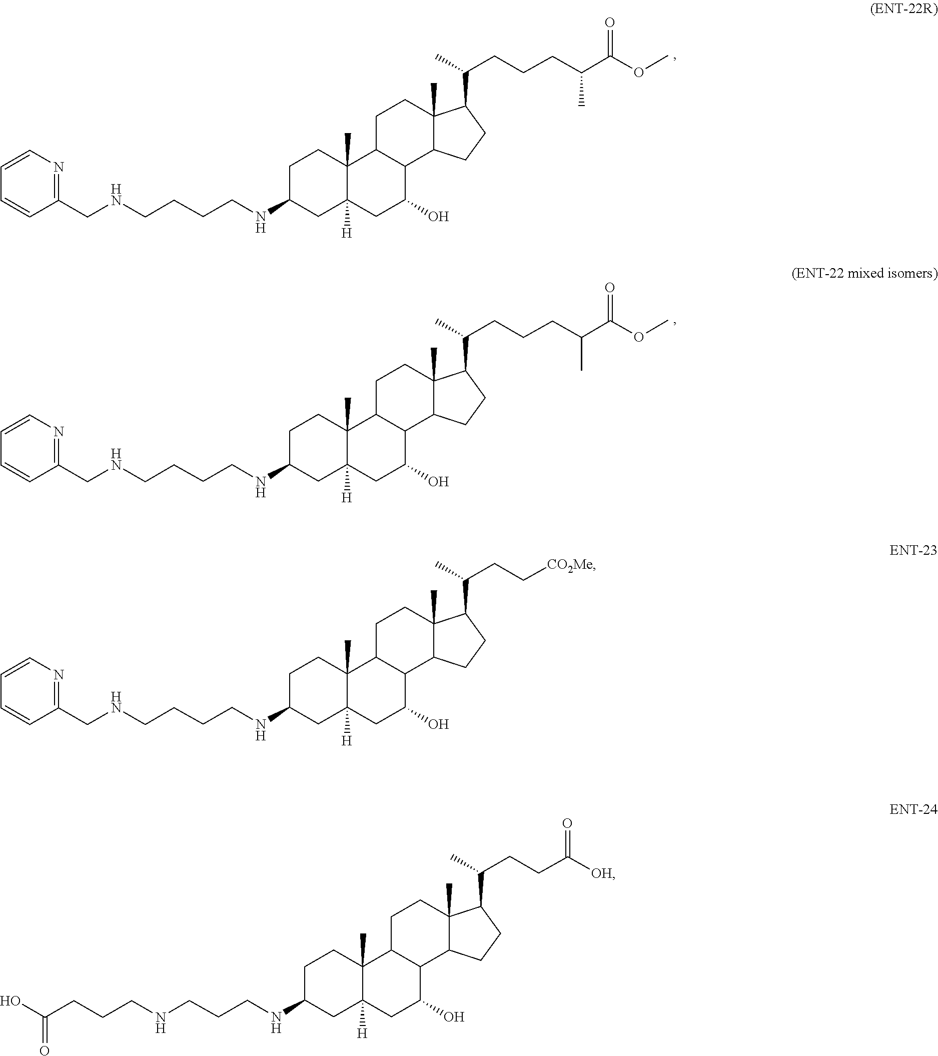 [SMILES: COC(=O)[C@H](C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)C(C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)CC[C@@H](C)[C@H]1CCC2C3C(CC[C@@]21C)[C@@]1(C)CC[C@H](NCCCCNCc2ccccn2)C[C@@H]1C[C@H]3O ; C[C@H](CCC(=O)O)[C@H]1CCC2C3C(CC[C@@]21C)[C@@]1(C)CC[C@H](NCCCNCCCC(=O)O)C[C@@H]1C[C@H]3O]

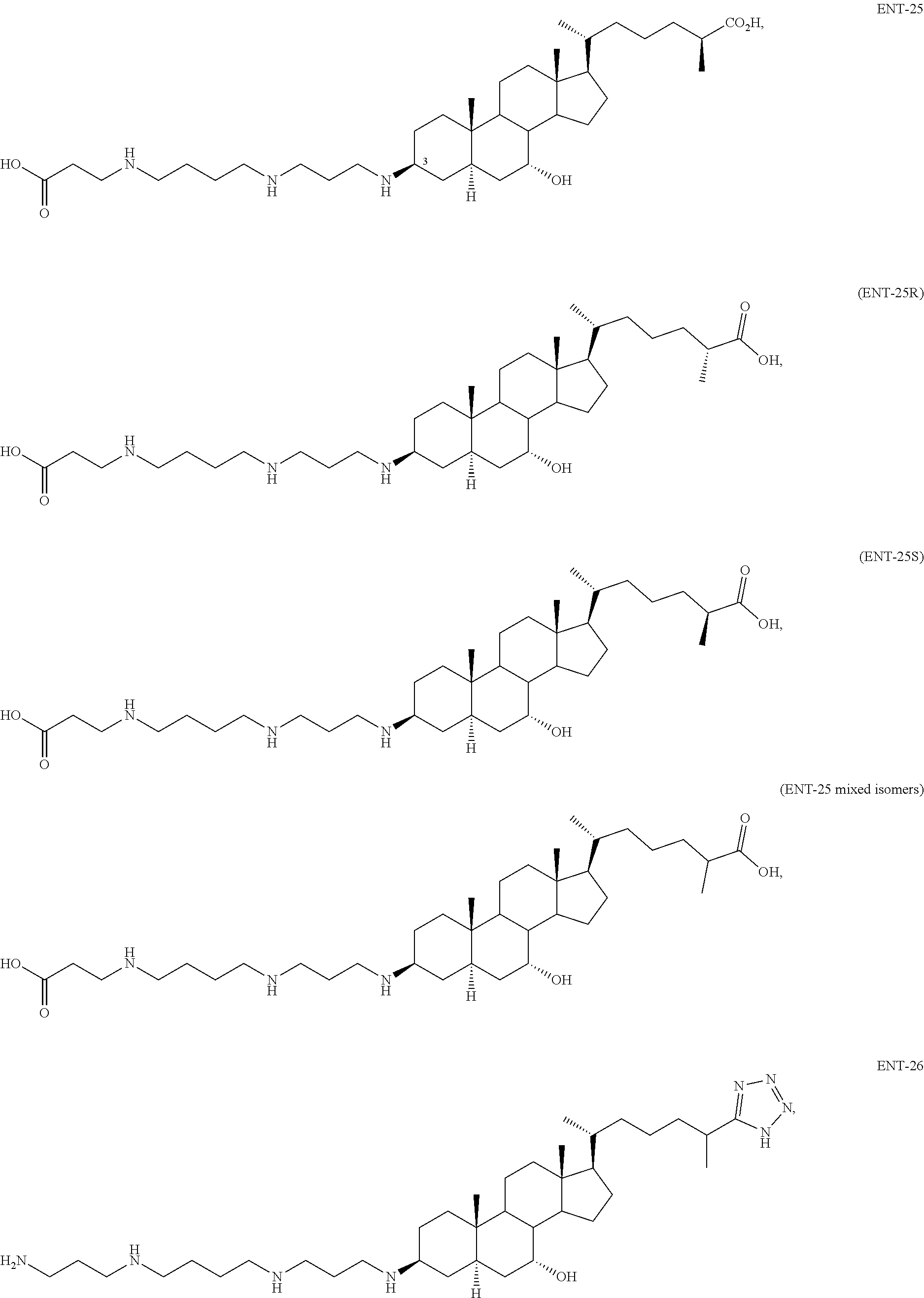

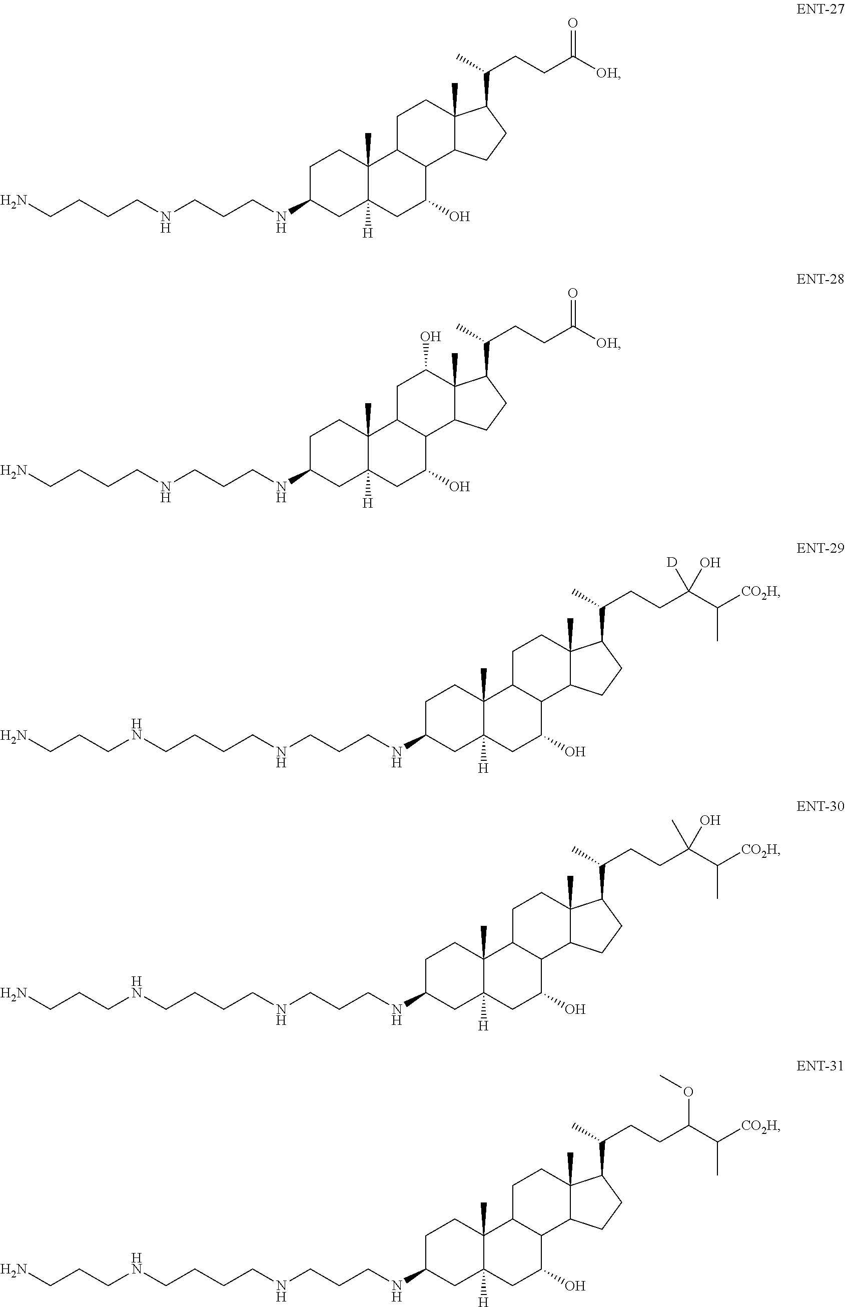 [SMILES: C[C@H](CCC(=O)O)[C@H]1CCC2C3C(CC[C@@]21C)[C@@]1(C)CC[C@H](NCCCNCCCCN)C[C@@H]1C[C@H]3O ; C[C@H](CCC(=O)O)[C@H]1CCC2C3C(C[C@H](O)[C@@]21C)[C@@]1(C)CC[C@H](NCCCNCCCCN)C[C@@H]1C[C@H]3O ; [2H]C(O)(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(C(=O)O)C(C)(O)CC[C@@H](C)[C@H]1CCC2C3C(CC[C@@]21C)[C@@]1(C)CC[C@H](NCCCNCCCCNCCCN)C[C@@H]1C[C@H]3O ; COC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O]

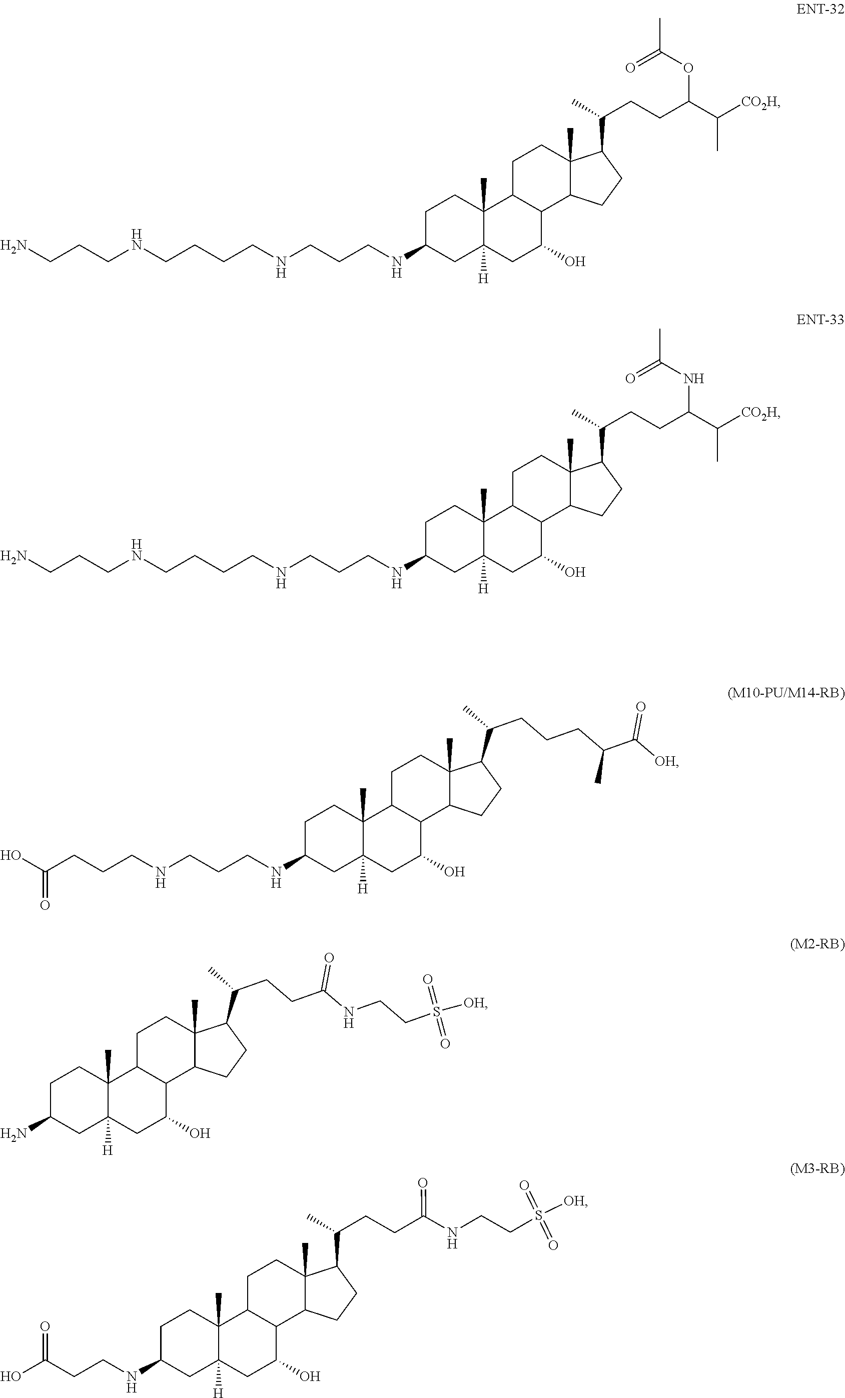

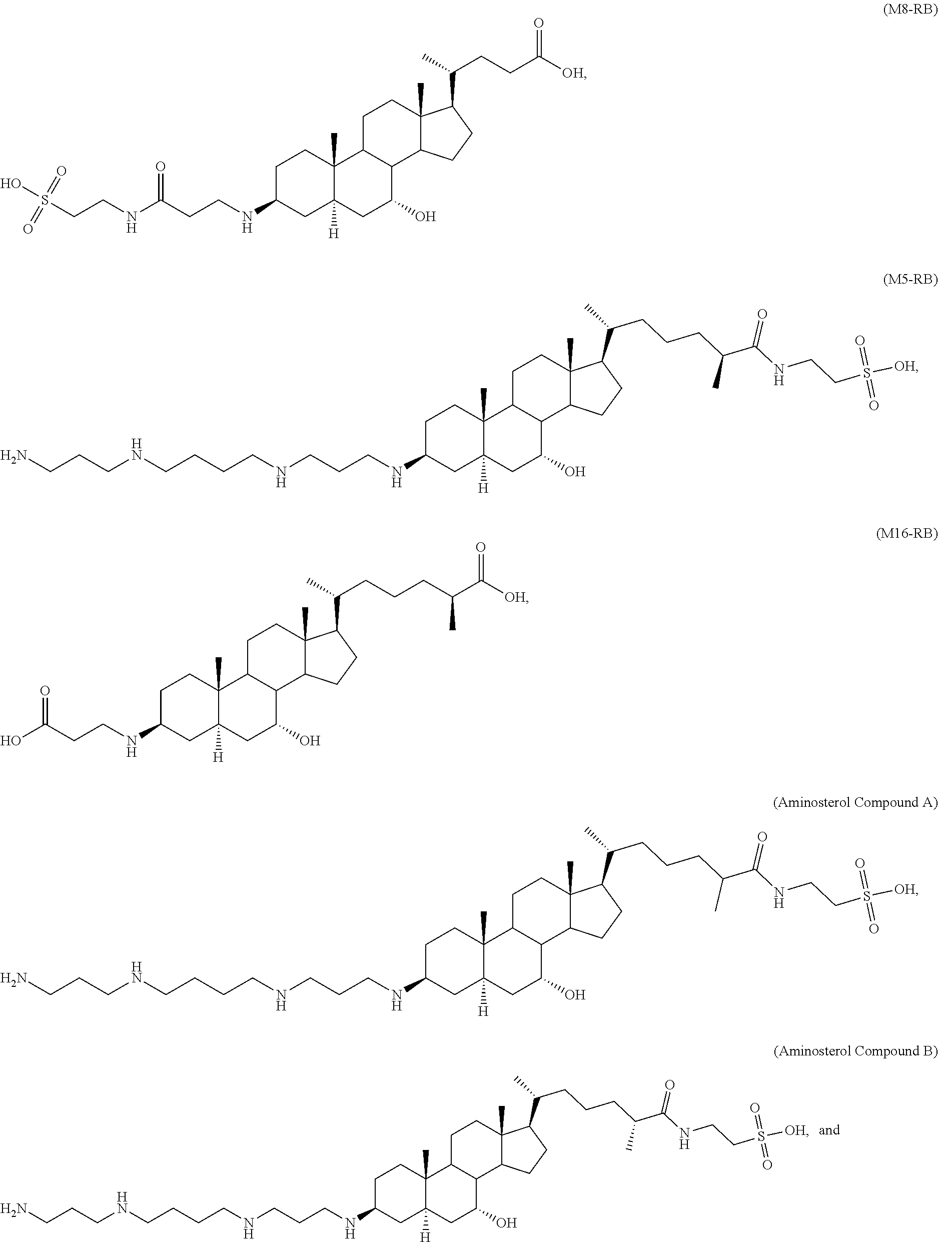

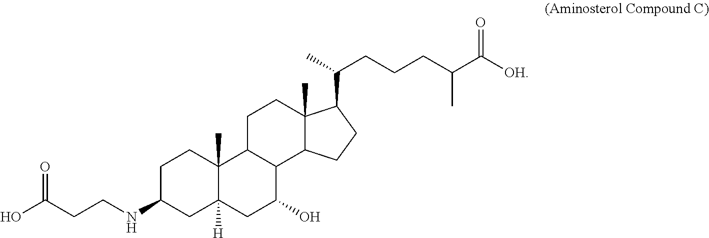 [SMILES: CC(CCC[C@@H](C)[C@H]1CCC2C3C(CC[C@@]21C)[C@@]1(C)CC[C@H](NCCC(=O)O)C[C@@H]1C[C@H]3O)C(=O)O]

In another aspect, disclosed is the aminosterol compound having the following chemical structure (Formula (I)).

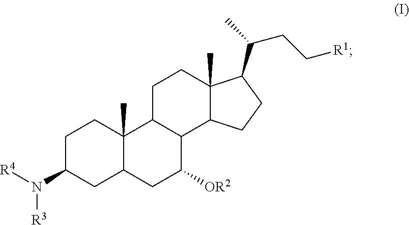

wherein:
R¹ is an optionally substituted C₁-C₈ alkyl, optionally substituted 2 to 8-membered heteroalkyl, optionally substituted heterocyclyl, or optionally substituted C₃-C₈ cycloalkyl;
R² is H or —C(O)R⁵, wherein R⁵ is an optionally substituted aryl, optionally substituted heteroaryl, optionally substituted C₁-C₆ alkyl, optionally substituted heterocyclyl, or optionally substituted C₁-C₈ cycloalkyl; and
R³ and R⁴ are each independently H or an optionally substituted 2 to 1l-membered heteroalkyl;
or a pharmaceutically acceptable salt, solvate, prodrug, or derivative thereof; wherein the compound is not:
(1) (24R)-3β-({3-[(3-Aminopropyl)amino]propyl)amino)-7α-hydroxycholestan-24-yl hydrogen sulfate (squalamine);
(2) (3R,6R)-6-((3S,5R,7R,8R,9S,10S,13R,14S,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptan-3-yl hydrogen sulfate (aminosterol 1436);
(3) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid (ENT-03);
(4) (2R,6R)-6-((3S,5R,7R,10S,13R,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2-methylheptanoic acid (C₂₅ (R) ENT-03);
(5) (6R)-6-((3S,5R,7R,10S,13R,17R)-3-((3-((4-((3-aminopropyl)amino)butyl)amino)propyl)amino)-7-hydroxy-10,13-dimethylhexadecahydro-11-1-cyclopenta[a]phenanthren-17-yl)-2-methylheptanoic-2,3,3-d3 acid (ENT-03 d3);
(6) (6R)-6-((3S,5R,7R,10S,13,17R)-3-((3-((4-((3-aminopropyl)amino)butyl)amino)propyl)amino)-7-(benzoyloxy)-10,13-dimethylhexadecahydro-1H-cyclopenta[a]phenanthren-17-yl)-2-methylheptanoic acid;
(7) (6R)-6-((3R,5S,7R,10S,13R,17R)-3-((3-((4-aminobutyl)amino)propyl)amino)-7-hydroxy-10,13-dimethylhexadecahydro-11H-cyclopenta[a]phenanthren-17-yl)-2-methylheptanoic acid;
(8) (6R)-6-((3R,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid;
(9) (6R)-6-((3R,5S,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylheptanoic acid;
(10) (6R)-6-((3R,5S,7R,10S,13R,17R)-3-((3-((4-((3-aminopropyl)amino)butyl)amino)propyl)amino)-7,12-dihydroxy-10,13-dimethylhexadecahydro-1H-cylopenta[a]phenanthren-17-yl)-2-methylheptanoic acid;
(11) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic-2-d acid;
(12) (6R)-6-((3S,5R,7R,10S,13R,17R)-3-(3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic-2-d acid;
(13) (6R)-6-((3S,5R,7R,10S,13R,1 7R)-3-((3-((4-aminobutyl)amino)propyl)amino)-7,12-dihydroxy-10,13-dimethylhexadecahydro-1 I-cyclopenta[a]phenanthren-17-yl)-2-methylheptanoic-2-d acid;
(14) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methylheptanoic-2-d acid;
(15) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-(methyl-d3)heptanoic acid;
(16) (6R)-6-((3S,5R,7R,10S,13R,17R)-3-((3-((4-((3-aminopropyl)amino)butyl)amino)propyl)amino)-7-hydroxy-10,13-dimethylhexadecahydro-1i-cyclopenta[a]phenanthren-17-yl)-2-(methyl-d3)heptanoic acid;
(17) (6R)-6-((3S,5R,7R,10S,13R,17R)-3-((3-((4-aminobutyl)amino)propyl)amino)-7,12-dihydroxy-10,13-dimethylhexadecahydro-1-1cyclopenta[a]phenanthren-1 7-yl)-2-(methyl-d3)heptanoic acid;
(18) (6R)-6-((3S,5R,7R,10S,13R,17,R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-(methyl-d3)heptanoic acid;
(19) (6R,E)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methyhept-2-enoic acid;
(20) (6R,E)-6-((3S,5R,7R,10S, 13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylhept-2-enoic acid;
(21) (6R,E)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylhept-2-enoic acid;
(22) (6R,E)-6-((3S,R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-1 7-yl)-2-methylhept-2-enoic acid;
(23) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-3-hydroxy-2-methylheptanoic acid;
(24) (6R)-6-((3S,5R,7R,10S,1 3R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-3-hydroxy-2-methylheptanoic acid;
(25) (6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-3-hydroxy-2-methylheptanoic acid;
(26) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-3-hydroxy-2-methylheptanoic acid;
(27) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)heptan-3-one;
(28) (6R)-6-((3S,5R,7R,1S,13R17R)-3-((3-((4-((3-aminopropyl)amino)butyl)amino)propyl)amino)-7-hydroxy-10,13-dimethylhexadecahydro-1H-cyclopenta[a]phenanthren-17-yl)heptan-3-one;
(29) (6R)-6-((3S,5R,7R,10S,13R,17R)-3-(3-((4-aminobutyl)amino)propyl)amino)-7,12-dihydroxy-10,13-dimethylhexadecahydro-1HF-cyclopenta[a]phenanthren-1 7-yl)heptan-3-one;
(30) (6R)-6-((3S,5R,7R,10S,13R,17R)-3-((3-((4-((3-aminopropyl)amino)butyl)amino)propyl)amino)-7, 12-dihydroxy-10,13-dimethylhexadecahydro-1H-cyclopenta[a]phenanthren-17-yl)heptan-3-one;
(31) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methyl-3-oxoheptanoic acid;
(32) (6R)-6-((3S,5R,7R,10S,13R,17R)-3-((3-((4-((3-aminopropyl)amino)butyl)amino)propyl)amino)-7-hydroxy-10,13-dimethylhexadecahydro-11-1-cyclopenta[a]phenanthren-17-yl)-2-methyl-3-oxoheptanoic acid;
(33) (6R)-6-((3S,5R,7R,10S,13R,17R)-3-((3-((4-aminobutyl)amino)propyl)amino)-7,12-dihydroxy-10,13-dimethylhexadecahydro-1 H-cyclopenta[a]phenanthren-17-yl)-2-methyl-3-oxoheptanoic acid,
(34) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoic acid;
(35) methyl(6R)-6-((3S,5R,7R,10S,13R,17R)-3-((3-((4-aminobutyl)amino)propyl)amino)-7-hydroxy-10,13-dimethylhexadecahydro-11-1-cyclopenta[a]phenanthren-17-yl)-2-methyl-3-oxoheptanoate;
(36) methyl(6R)-6-((3S,5R,7R,10S,13R,17R)-3-((3-((4-((3-aminopropyl)amino)butyl)amino)propyl)amino)-7-hydroxy-10,13-dimethylhexadecahydro-11H-cyclopenta[a]phenanthren-17-yl)-2-methyl-3-oxoheptanoate;
(37) methyl(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoate;
(38) methyl(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoate;
(39) isopropyl(6R)-6-((3S,5R,7R,10S,13R,17R)-3-((3-((4-aminobutyl)amino)propyl)amino)-7-hydroxy-1Q,13-dimethylhexadecahydro-1H-cyclopenta[a]phenanthren-17-yl)-2-methyl-3-oxoheptanoate;
(40) isopropyl(6R)-6-((35,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoate;
(41) isopropyl(6R)-6-((3S,5R,7R,10S,13R,17R)-3-((3-((4-aminobutyl)amino)propyl)amino)-7,1 2-dihydroxy-10,13-dimethylhexadecahydro-1H-cyclopenta[a]phenanthren-17-yl)-2-methyl-3-oxoheptanoate;
(42) isopropyl(6R)-6-((3S,5R,7R,10S,13R,1-7R)-3-((3-((4-((3-aminopropyl)amino)butyl)amino)propyl)amino)-7,12-dihydroxy-10,13-dimethylhexadecahydro-1 H-cyclopenta[a]phenanthren-17-yl)-2-methyl-3-oxoheptanoate;
(43) (6R)-6-((35,5R,7R,105,13R,17R)-3-(3-((4-aminobutyl)amino)propyl)amino)-7-hydroxy-10,13-dimethylhexadecahydro-1H-cyclopenta[a]phenantrene-17-yl)-2,3-dihydroxy-2-methylheptanoic acid;
(44) (6R)-6-((3S,5R,7R,10S,13R,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2,3-dihydroxy-2-methylheptanoic acid;
(45) (6R)-6-((3S,5R,7R,10S,13R,17R)-3-((3-((4-aminobutyl)amino)propyl)amino)-7,12-dihydroxy-10, 13-dimethylhexadecahydro-1H-cyclopenta[a]phenanthren-17-yl)-2,3-dihydroxy-2-methylheptanoic acid;
(46) (6R)-6-((3S,5R,7R, 10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2,3-dihydroxy-2-methylheptanoic acid;
(47) (6R)-6-((3S,5R,7R,10S,13R,17R)-3-((3-((4-aminobutyl)amino)propyl)amino)-7-hydroxy-10,13-dimethylhexadecahydro-11-1-cyclopenta[a]phenanthren-17-yl)-2-methylheptane-2,3-diol;
(48) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptane-2,3-diol;
(49) (3S,5R,7R,10S,13R,17R)-3-((3-((4-aminobutyl)amino)propyl)amino)-17-((2R)-5,6-dihydroxy-6-methylheptan-2-yl)-10,13-dimethylhexadecahydro-1H-cyclopenta[a]phenanthrene-7,12-diol;
(50) (3S,5R,7R,10S,13R,17R)-3-((3-((4-((3-aminopropyl)amino)butyl)amino)propyl)amino)-17-((2R)-5,6-dihydroxy-6-methylheptan-2-yl)-10,13-dimethylhexadecahydro-1H-cyclopenta[a]phenanthrene-7,12-diol;
(51) (6R)-6-((3S,5R,7R,10S,13R,17[)-3-(3-((4-ammoniobutyl)ammonio)propyl)ammonio)-7,12-dihydroxy-10,13-dimethylhexadecahydro-1H-cyclopenta[a]phenanthren-17-yl)-2-methylheptanoate (ENP-05);
(52) (6R)-6-((3S,5R,7R,10S,13R,17R)-3-((3-((4-ammoniobutyl)ammonio)propyl)ammonio)-7-hydroxy-10,1 3-dimethylhexadecahydro-1H-cyclopenta[a]phenanthren-17-yl)-2-methylheptanoate (ENT-06)
(53) (6R)-6-((3S,5R,R,10S,13R,17R)-3-((3-((4-((3-ammonio propyl)ammonio)butyl)ammonio)propyl)ammonio)-7-hydroxy-10,13-dimethylhexadecahydro-1H-cyclopenta[a]phenanthren-17-yl)-3-hydroxy-2-methylheptanoate-2,3-d2;
(54) (2R,6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid (C₂₅(S) ENT-03);
(55) (2R,6R)-6-((3R,5S,7R,101S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
(56) (2S,6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
(57) (2R,6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid (C₂₅ (R) ENT-06);
(59) (2S,6R)-6-((3S,5R,7R,10S,1 3R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid (C₂₅ (S) ENT-06);
(60) (6R)-6-((3S,5R,7R,10S,13R)-3-((3-((4-aminobutyl)amino)propylamino)-7-hydroxy-10,13-dimethylhexadecahydro-1H-cyclopenta[a]phenanthren-17-yl)-2-(methyl-d3)heptan-3-yl-1,1,1,2-d4 hydrogen sulfate (D2-squalamine);
(61) (3R,6R)-6-((3S,5RR,10S,13R,17R)-3-((3-((4-aminobutyl)amino)propyl)amino)-7-hydroxy-10,13-dimethylhexadecahydro-1H-cyclopenta[a]phenanthren-1 7-yl)-2-methylheptan-3-yl-3-d hydrogen sulfate (24-D₁-squalamine); or
(62) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-(methyl-d3)heptan-3-yl-1,1,1,2-d4 hydrogen sulfate (D₇-1436).

In another aspect, the aminosterol has the structure of Formula (I), wherein R¹ is selected from

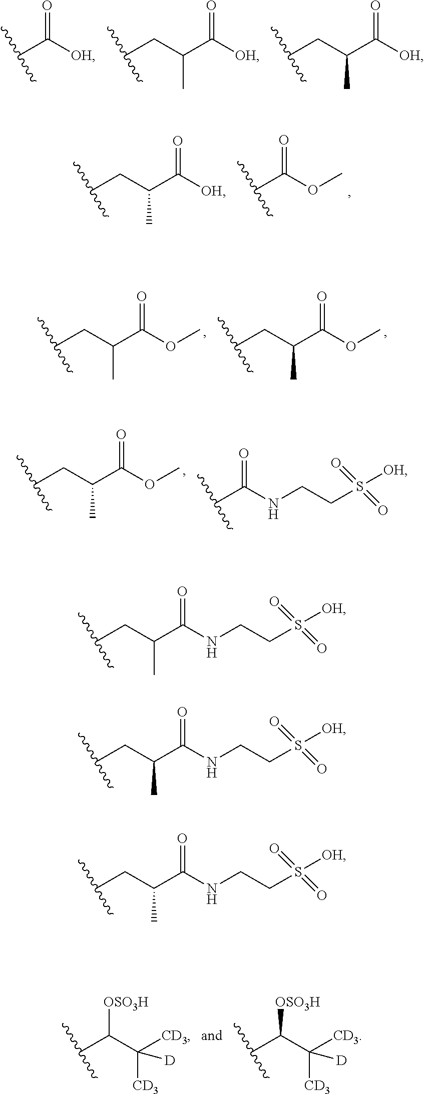

In another aspect, the aminosterol has the structure of Formula (I), wherein R² is H.

In another aspect, the aminosterol has the structure ofFormula (I), wherein R³ and R⁴ are independently selected from H,

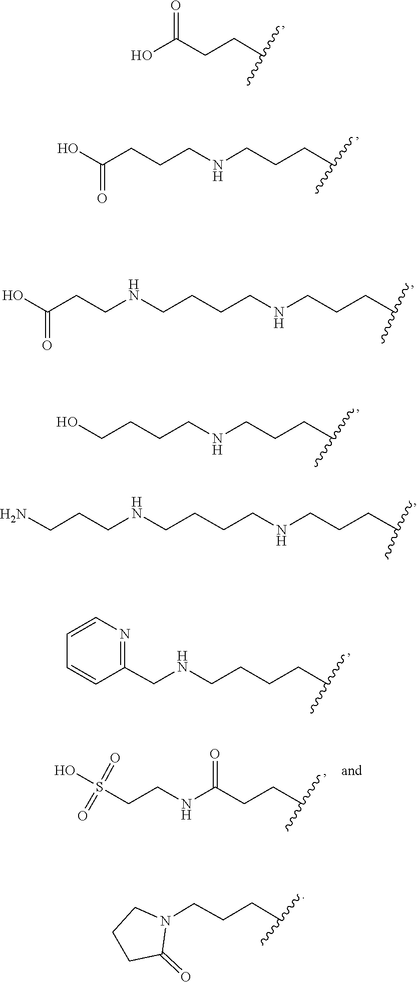

In another aspect, the aminosterol has the structure of Formula (I), wherein R³ is H and R⁴ is independently selected from

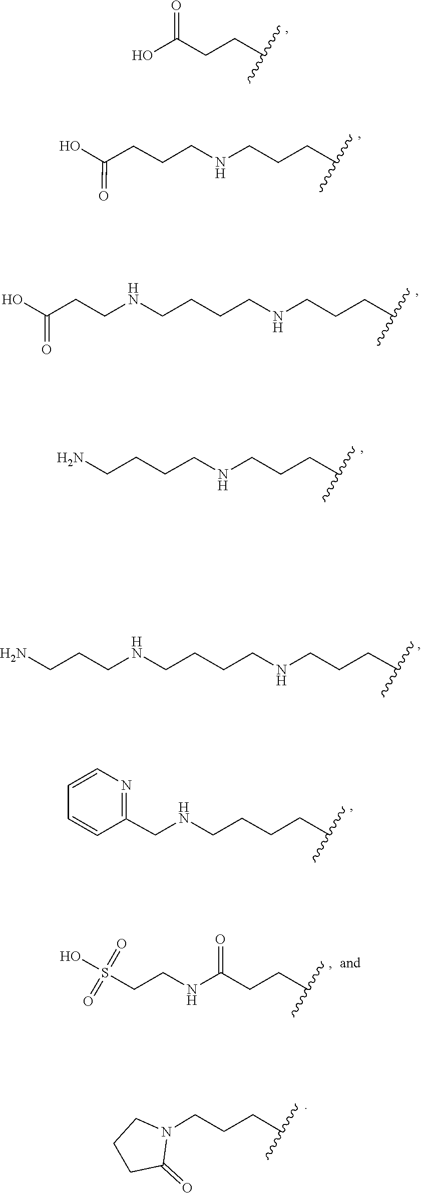

In some embodiment, the compound as disclosed herein has a diasteromeric excess (de), with respect to the C₂₅ carbon is about 80% to about 90%, about 90% to about 95%, about 95% to about 99%, about 99% to about 99.9%, or about 100%.

In a further aspect, described are deuterated aminosterol compounds, where an aminosterol compound described herein has at least one hydrogen of the aminosterol compound replaced with deuterium.

In one aspect, a composition is provided comprising an aminosterol compound disclosed herein, or a pharmaceutically acceptable salt, solvate, prodrug, or derivative thereof, and at least one pharmaceutically acceptable carrier or excipient. In some embodiments, the composition comprises one or more of the following: an aqueous carrier; a buffer, a sugar; and/or a polyol compound. In some embodiments, the composition comprises at least one additional active agent.

In some embodiments, the aminosterol, or a pharmaceutically acceptable salt, solvate, prodrug, or derivative thereof, is of pharmaceutically acceptable grade.

In some embodiments, the aminosterol, or a pharmaceutically acceptable salt, solvate, prodrug, or derivative thereof, is in the form of a phosphate salt, hydrochloride salt, or sodium salt.

In some embodiments, the composition is formulated: (a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and/or (c).

In some embodiments, the composition is formulated for oral administration. In some embodiments, the composition is formulated as an oral tablet or capsule. In some embodiments, the composition is formulated for intranasal administration.

In another aspect of the disclosure, encompassed is an aqueous aminosterol formulation comprising: (a) an aminosterol, or a pharmaceutically acceptable salt, solvate, prodrug, enantiomer, or derivative thereof; and (b) a cyclodextrin, wherein the formulation is buffered to a pH of about 4 to 9, and wherein the cyclodextrin is reversibly bound to a molecule of the aminosterol, or a pharmaceutically acceptable salt, solvate, prodrug, or derivative thereof, to form a cyclodextrin-aminosterol complex. The cyclodextrin can be, for example, a beta cyclodextrin (such as an unmodified beta cyclodextrin) or a gamma cyclodextrin (such as an unmodified gamma cyclodextrin). In other aspects, the cyclodextrin can be Sulfobutylether-β-Cyclodextrin (SBE-β-CD).

In another aspect of the disclosure, the pH can be from about 5 to about 8, or about 7 to about 8. In one embodiment, the pH can be from about 73 to about 7.5, such as 7.4.

In one embodiment of the disclosure, the buffer can be selected from the group consisting of an acetate buffer, phosphate buffer, citrate buffer, histidine buffer, tris(hydroxymethyl)aminomethane (TRIS), sodium phosphate, citric acid, acetic acid, KH₂PO₄, CHES, Borate, tromethanine, gluconic acid, lactic acid, tartaric acid, aspartic acid, glutamic acid, citric acid cycle intermediates, [tris(hydroxymethyl)methylamino]propanesulfonic acid (TAPS), 2-(bis(2-hydroxyethyl)amino)acetic acid (Bicine), N-[tris(hydroxymethly)methyl]glycine (Tricine), 3-[N-tris(hydroxymethyl)methylamino]-2-hydroxypropanesulfonic acid (TABSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), piperazine-N,N′-bis(2-ethanesulfonic acid) (PIPES), dimethylarsenic acid (Cacadylate), and 2-(N-morpholino)ethanesulfonic acid (MES). In one aspect, the aqueous aminosterol formulation is buffered with phosphate. In a further aspect, the aqueous aminosterol formulation can be buffered with tris(hydroxymethyl)aminomethane (Tris).

In another embodiment of the disclosure, the cyclodextrin can be present in an amount of from about 10% up to about 40%, about 15% up to about 30%, about 18% to about 25%, or about 20% up to about 25%. In addition, the cyclodextrin can be present in an amount of about 20% up to about 25%.

In one embodiment of the disclosure, the aminosterol can be present in an amount of from about 20 mg/ml up to about 75 mg/ml. In another aspect, the aminosterol can be present in an amount of from about 30 mg/ml up to about 65 mg/ml, in an amount of from about 40 mg/ml up to about 55 mg/ml, or in an amount of about 50 mg/ml.

In another aspect of the disclosure, the ratio of the concentrations (in mg/ml) of the aminosterol to the cyclodextrin can be about 0.1 to about 0.75. In addition, the ratio of the concentrations (in mg/ml) of the aminosterol to the cyclodextrin can be about 0.15 to about 0.5, or the ratio of the concentrations (in mg/ml) of the aminosterol to the cyclodextrin can be about 020.

In a further aspect of the disclosure, the aqueous aminosterol formulation is for administration intravenously, subcutaneously, or intramuscularly.

In one embodiment of the disclosure, the aminosterol is any aminosterol described herein including but not limited to, one of ENT-01, squalamine phosphate, ENT-02 (aminosterol 1436), ENT-03, ENT-03S (C₂₅ (S) ENT-03), ENT-03R (C25 (R) ENT-03), ENT-03 mixed isomers, ENT-04, ENT-05, ENT-06, ENT-07, ENT-08, ENT-09, ENT-10, ENT-111, ENT-12, ENT-13, ENT-14, ENT-14S, ENT-14R, ENT-15, ENT-16, ENT-17 (also known as M1-PU), ENT-18, ENT-18S, ENT-18R, NT-19 (ENT-19 is the racemic version of M10-PU), ENT-1DS, ENT-20, ENT-21, ENT-21S, ENT-21R, ENT-21 mixed isomers, ENT-22 ENT-22S, ENT-22R, ENT-22 mixed isomers, ENT-23, FENT-24, ENT-25, ENT-25R, ENT-25S (also known as M11-RB), ENT-25 mixed isomers, ENT-26, ENT-27, ENT-28, ENT-29, ENT-30, ENT-31, ENT-32, ENT-33, MID-PU/M1 14-RB, M2-RB, M3-RB, M8-RB, M5-RB, M16-RB, Aminosterol Compound A, Aminosterol Compound B, and Amninosterol Compound C, or a pharmaceutically acceptable salt, solvate, prodrug, enantiomer, or derivative thereof, with the structure of each compound shown below:

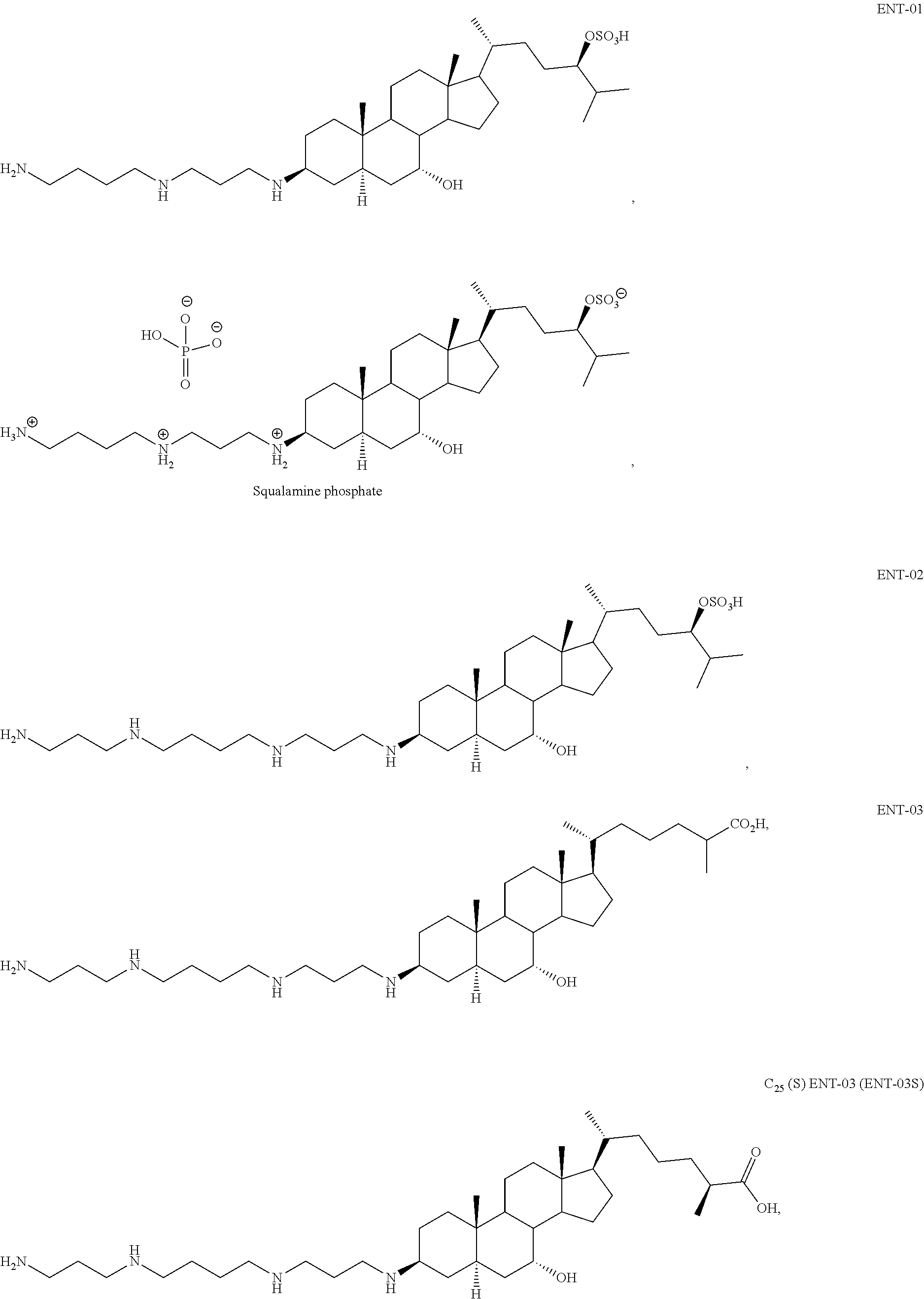

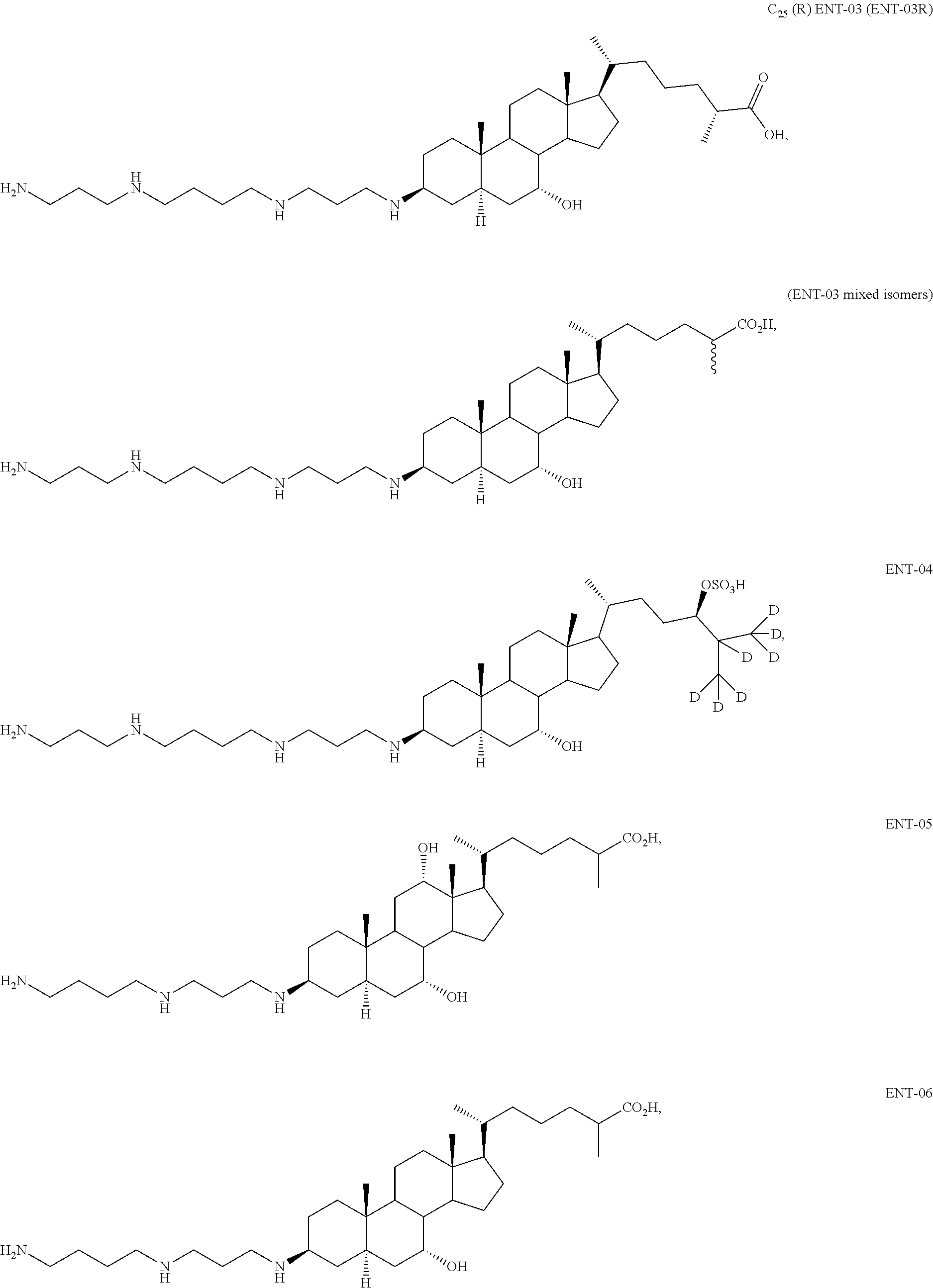

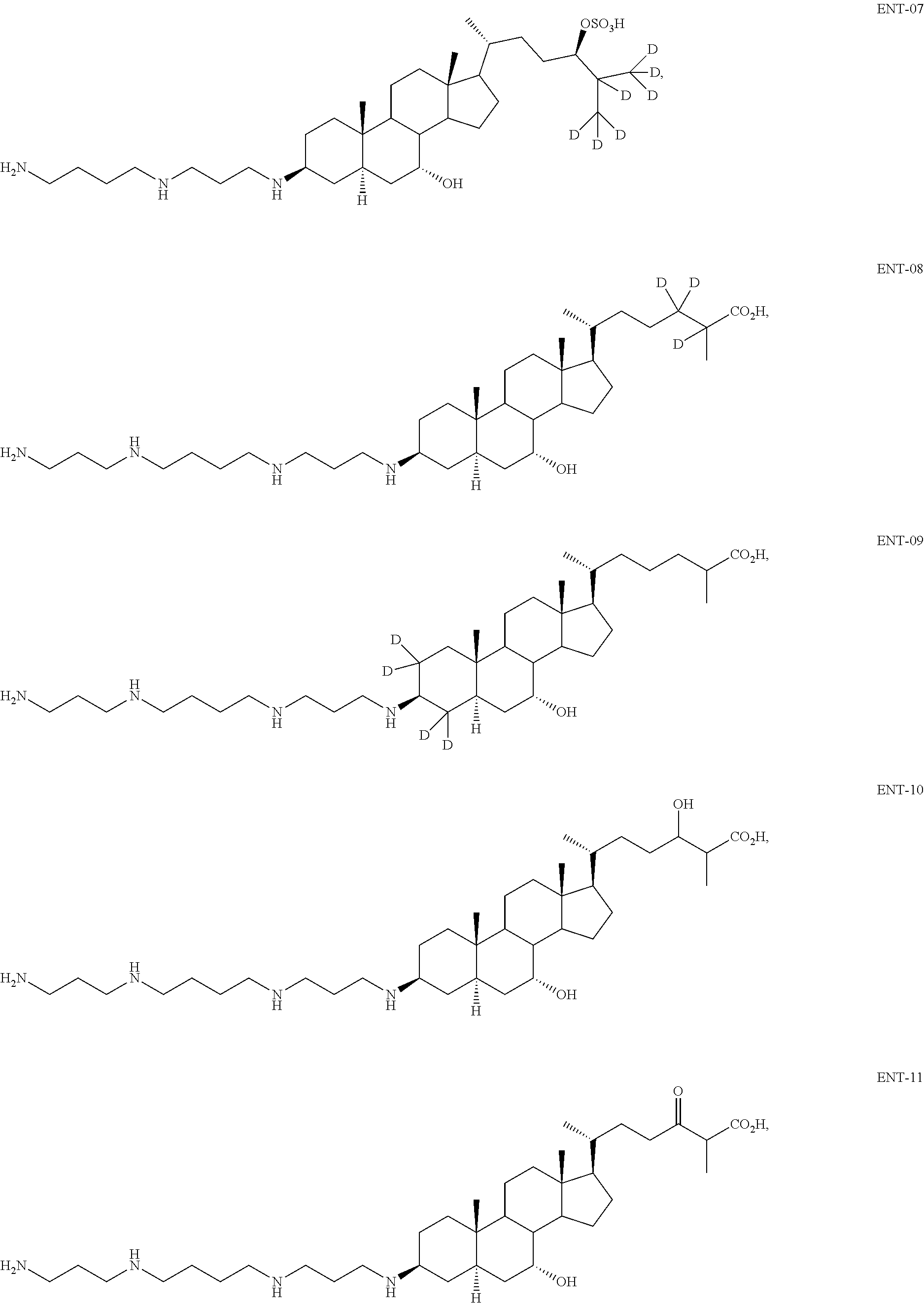

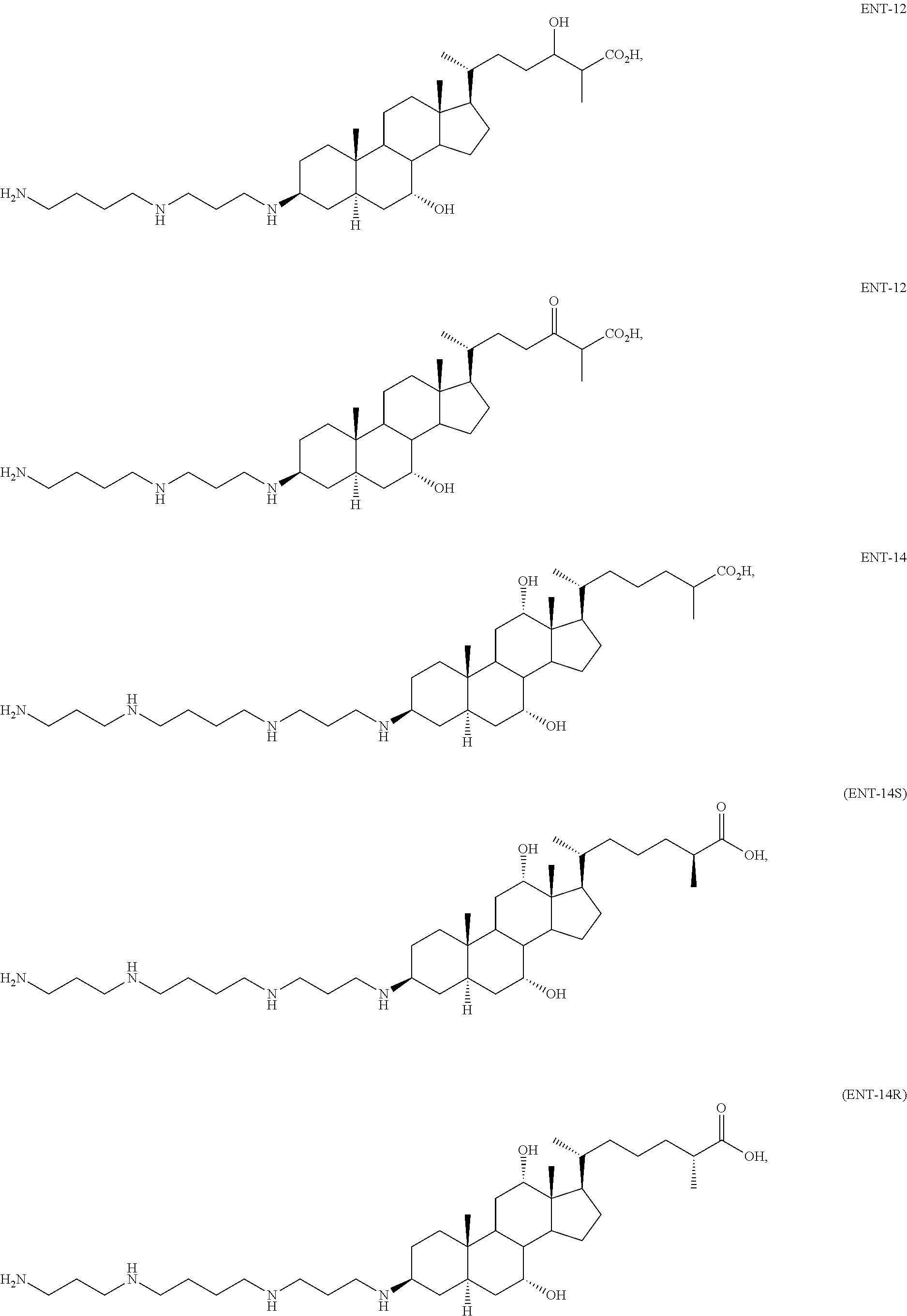

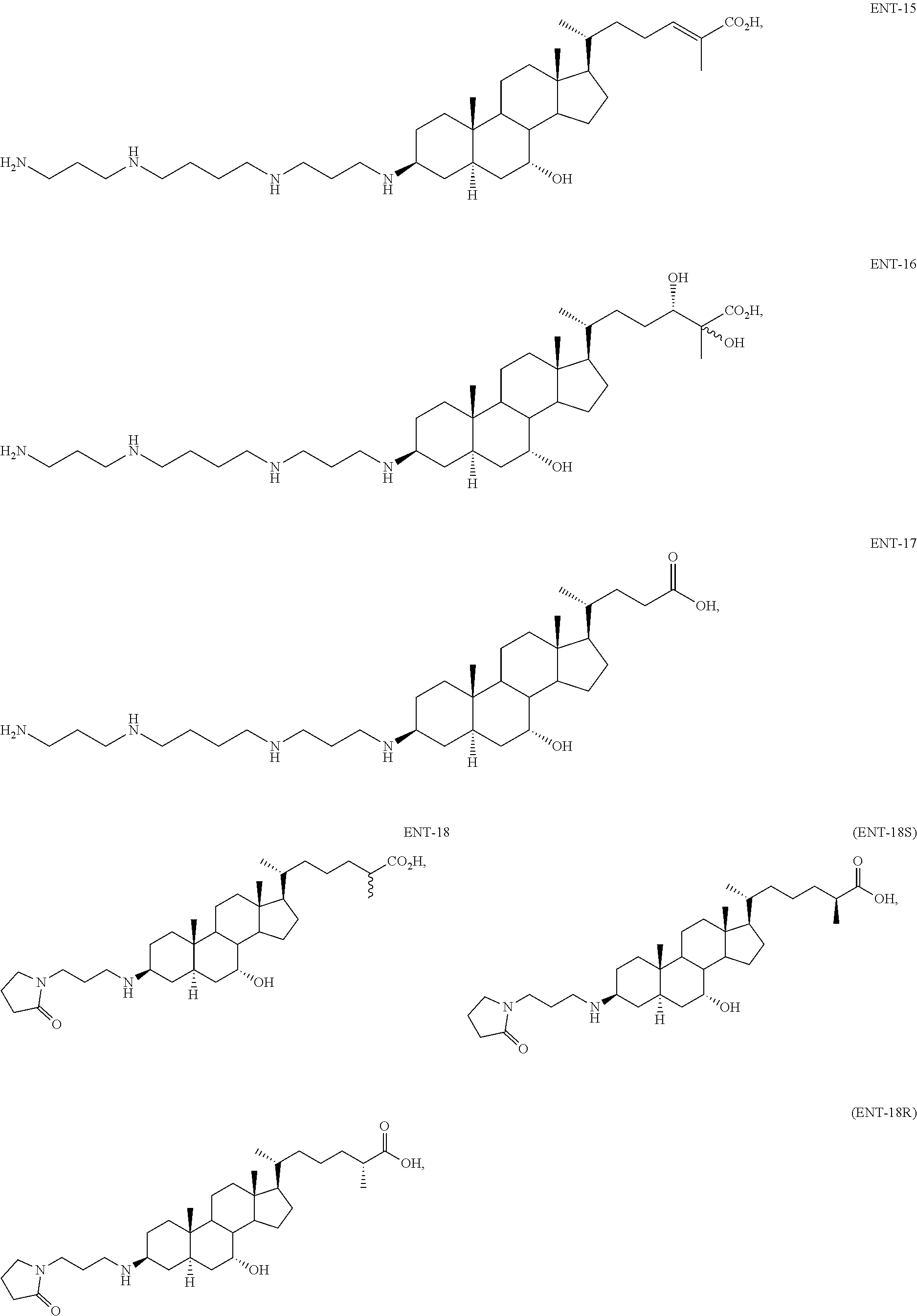

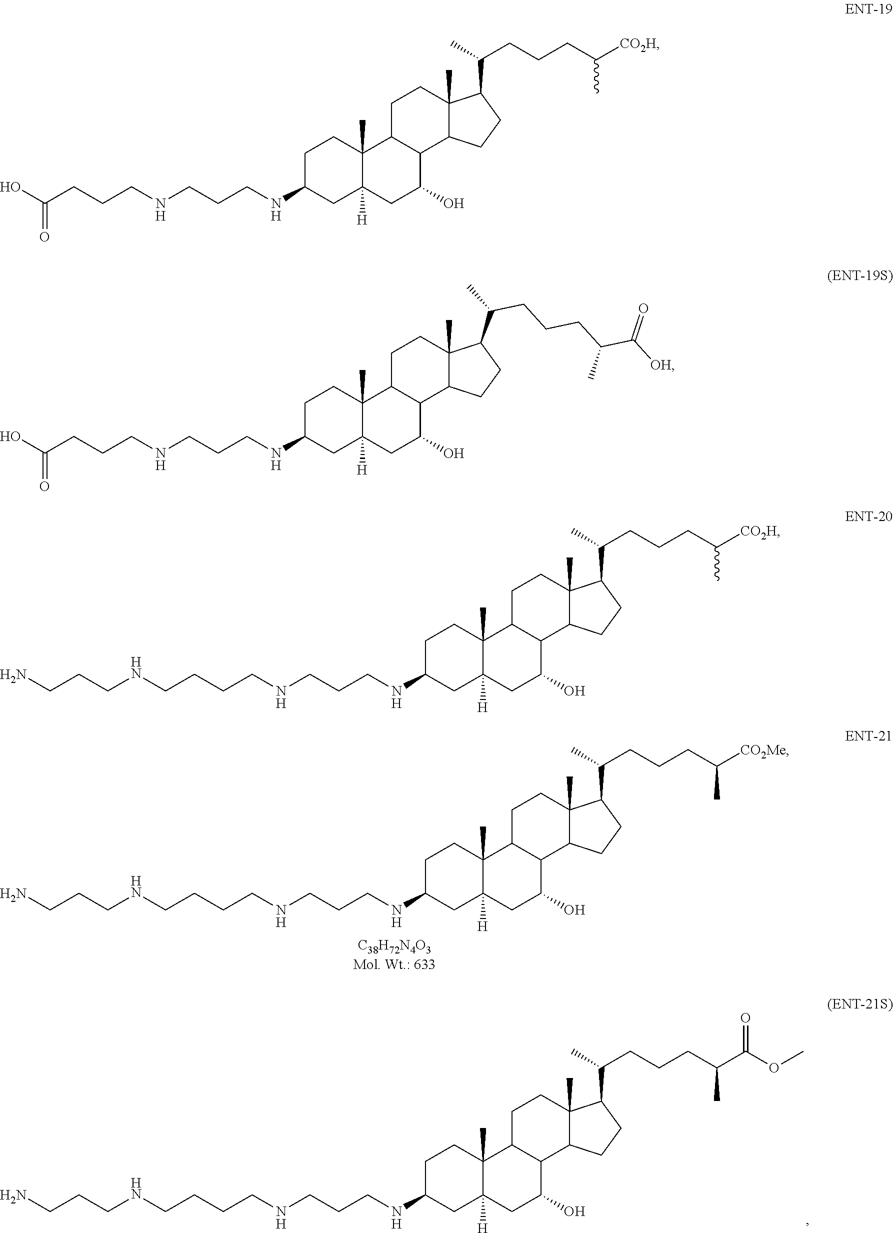

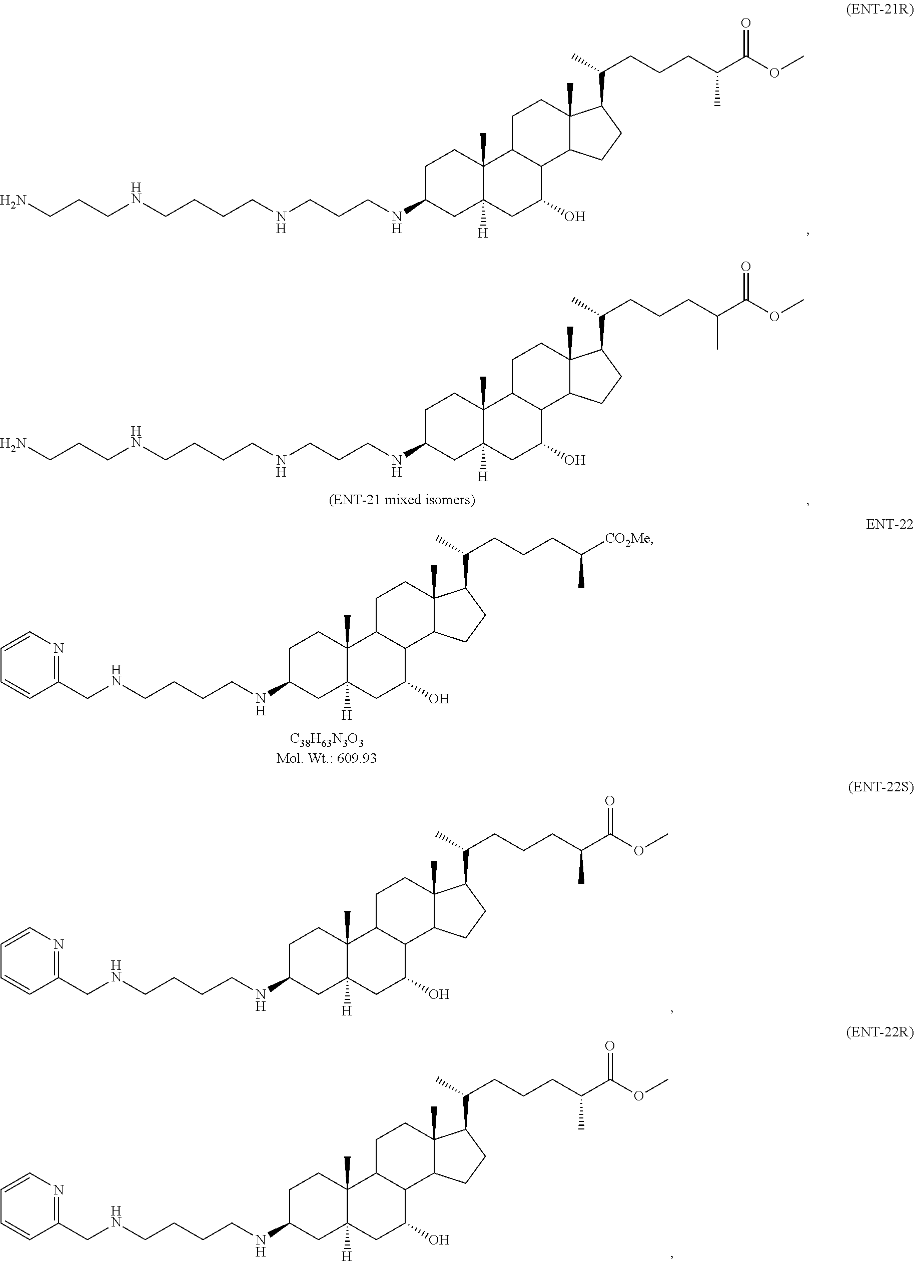

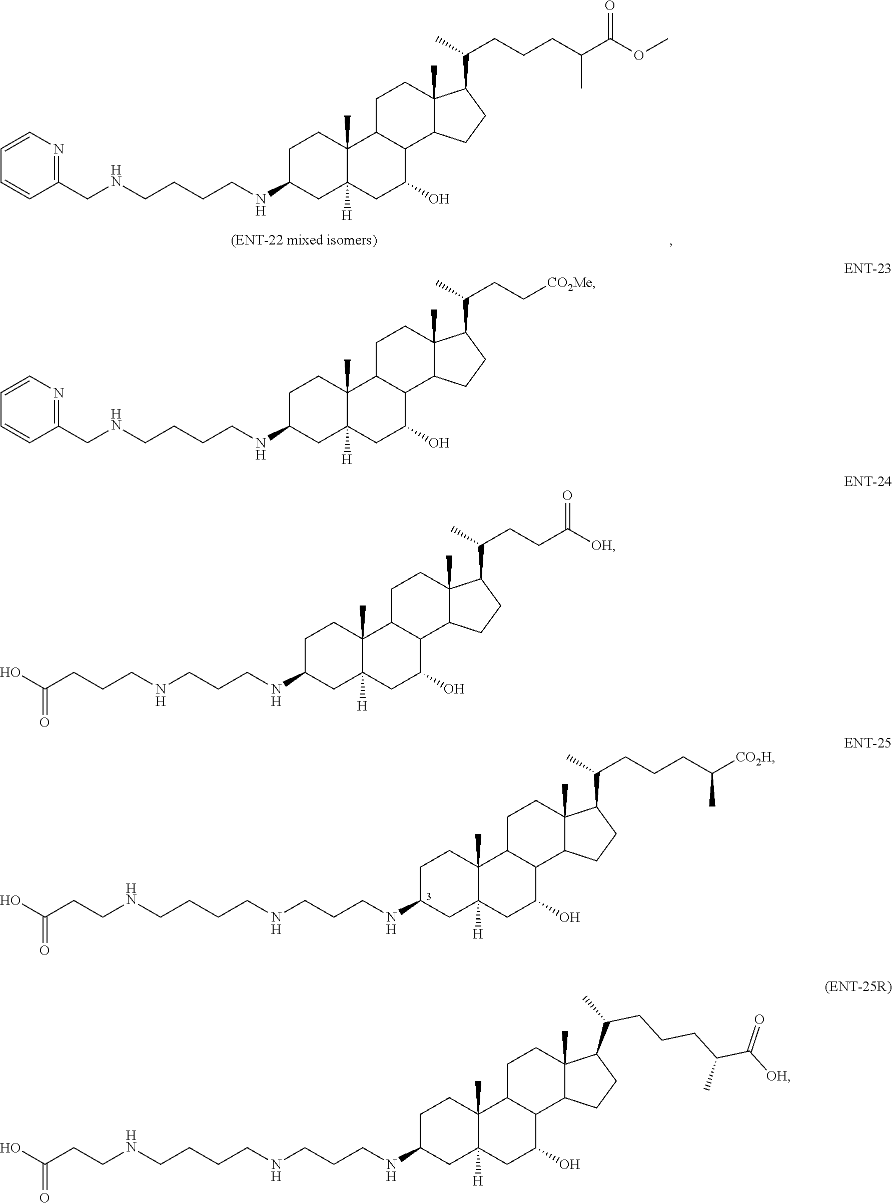 [SMILES: COC(=O)C(C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)CC[C@@H](C)[C@H]1CCC2C3C(CC[C@@]21C)[C@@]1(C)CC[C@H](NCCCCNCc2ccccn2)C[C@@H]1C[C@H]3O ; C[C@H](CCC(=O)O)[C@H]1CCC2C3C(CC[C@@]21C)[C@@]1(C)CC[C@H](NCCCNCCCC(=O)O)C[C@@H]1C[C@H]3O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; C[C@H](CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O]

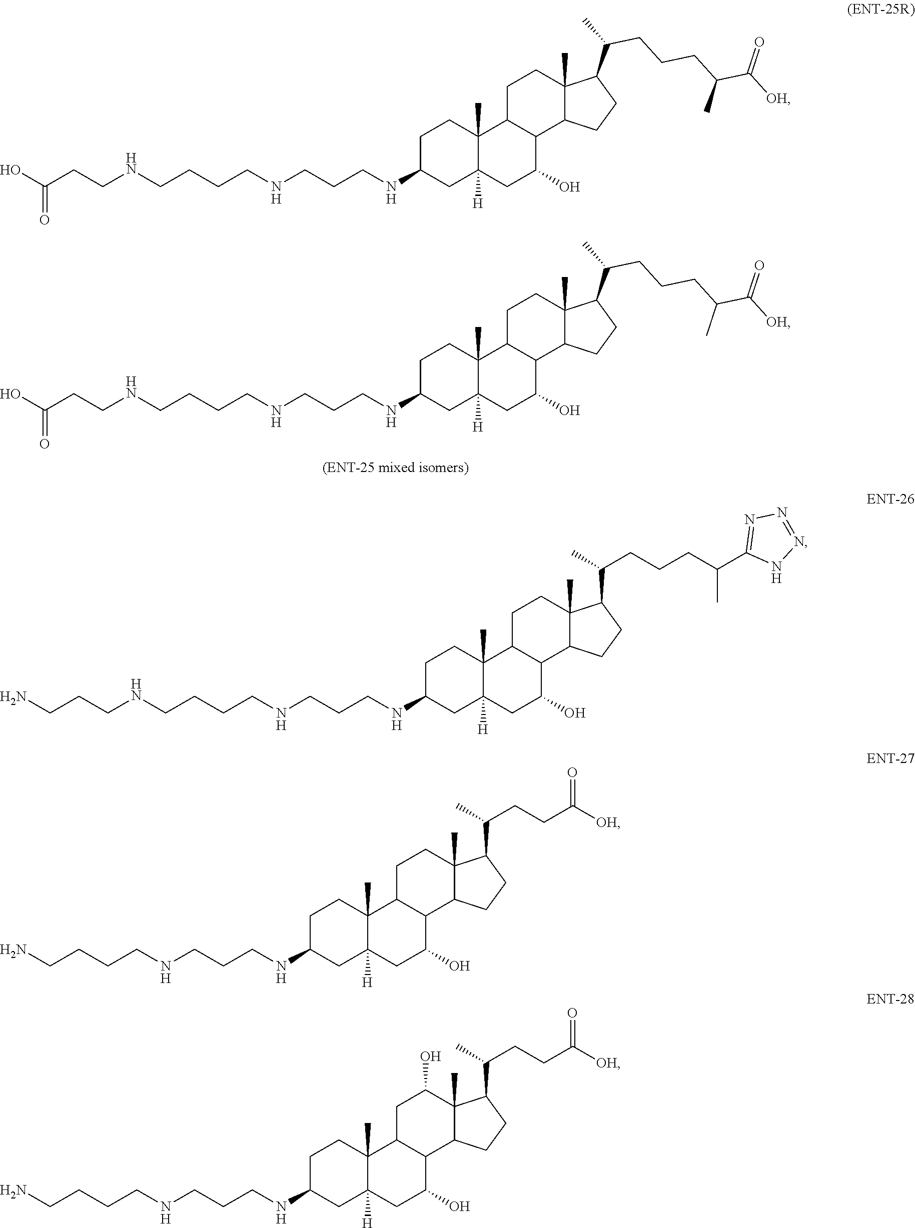 [SMILES: C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; CC(CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O ; CC(CCC[C@@H](C)[C@H]1CCC2C3C(CC[C@@]21C)[C@@]1(C)CC[C@H](NCCCNCCCCNCCCN)C[C@@H]1C[C@H]3O)c1nnn[nH]1 ; C[C@H](CCC(=O)O)[C@H]1CCC2C3C(CC[C@@]21C)[C@@]1(C)CC[C@H](NCCCNCCCCN)C[C@@H]1C[C@H]3O ; C[C@H](CCC(=O)O)[C@H]1CCC2C3C(C[C@H](O)[C@@]21C)[C@@]1(C)CC[C@H](NCCCNCCCCN)C[C@@H]1C[C@H]3O]

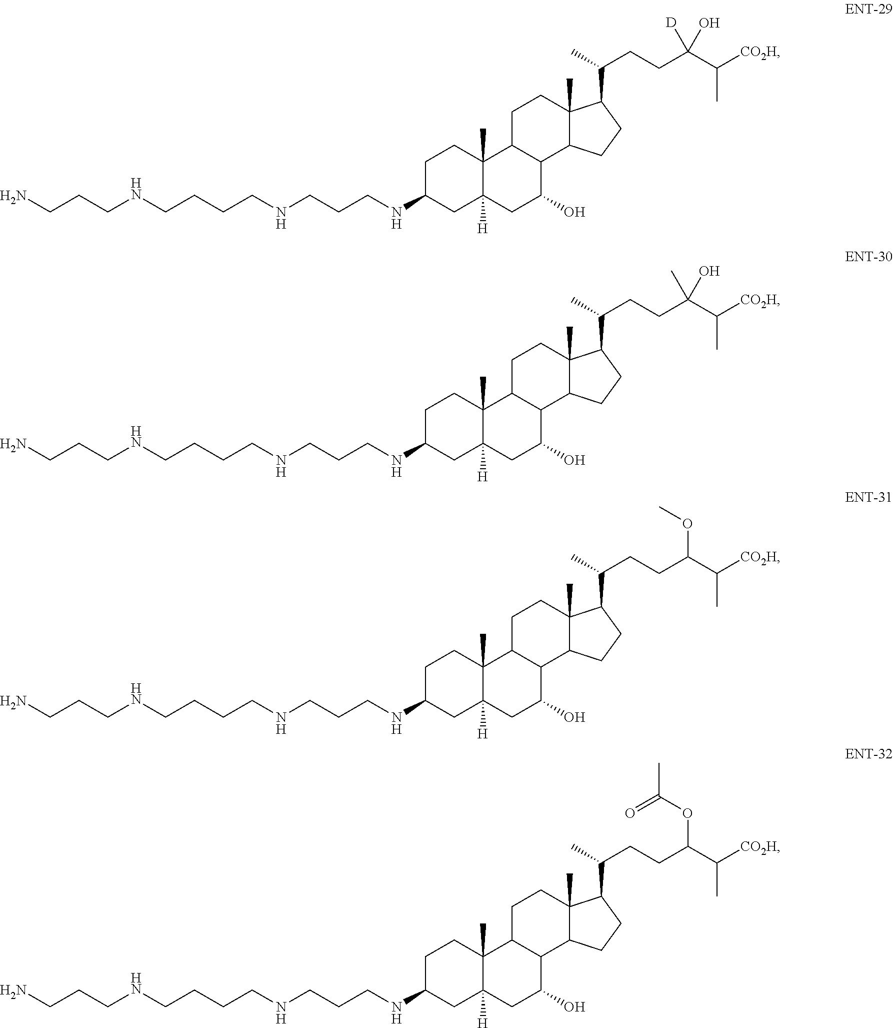 [SMILES: [2H]C(O)(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(C(=O)O)C(C)(O)CC[C@@H](C)[C@H]1CCC2C3C(CC[C@@]21C)[C@@]1(C)CC[C@H](NCCCNCCCCNCCCN)C[C@@H]1C[C@H]3O ; COC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(=O)OC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O]

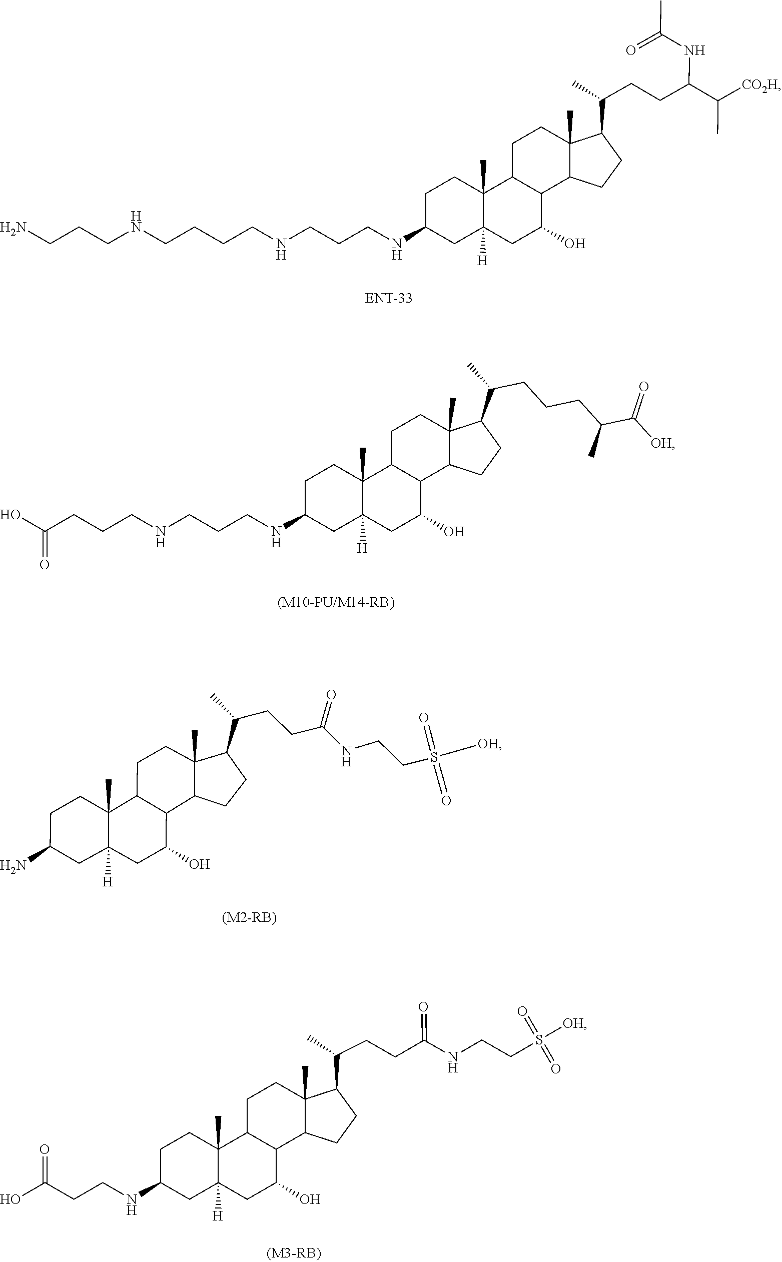 [SMILES: CC(=O)NC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; C[C@H](CCC[C@H](C)C(=O)O)[C@H]1CCC2C3C(CC[C@@]21C)[C@@]1(C)CC[C@H](NCCCNCCCC(=O)O)C[C@@H]1C[C@H]3O ; C[C@H](CCC(=O)NCCS(=O)(=O)O)[C@H]1CCC2C3C(CC[C@@]21C)[C@@]1(C)CC[C@H](N)C[C@@H]1C[C@H]3O ; C[C@H](CCC(=O)NCCS(=O)(=O)O)[C@H]1CCC2C3C(CC[C@@]21C)[C@@]1(C)CC[C@H](NCCC(=O)O)C[C@@H]1C[C@H]3O]

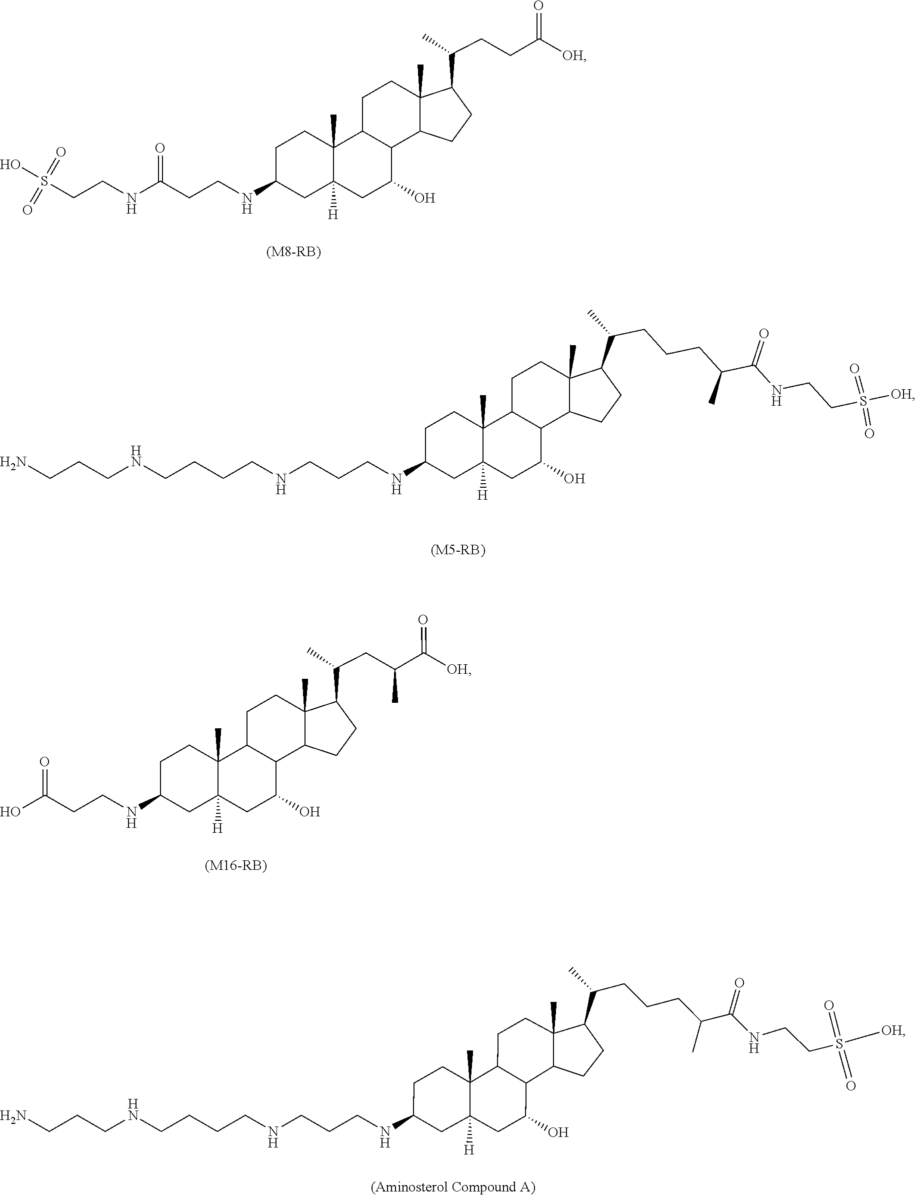 [SMILES: C[C@H](CCC(=O)O)[C@H]1CCC2C3C(CC[C@@]21C)[C@@]1(C)CC[C@H](NCCC(=O)NCCS(=O)(=O)O)C[C@@H]1C[C@H]3O ; C[C@H](CCC[C@H](C)C(=O)NCCS(=O)(=O)O)[C@H]1CCC2C3C(CC[C@@]21C)[C@@]1(C)CC[C@H](NCCCNCCCCNCCCN)C[C@@H]1C[C@H]3O ; C[C@H](C[C@H](C)C(=O)O)[C@H]1CCC2C3C(CC[C@@]21C)[C@@]1(C)CC[C@H](NCCC(=O)O)C[C@@H]1C[C@H]3O ; CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O]

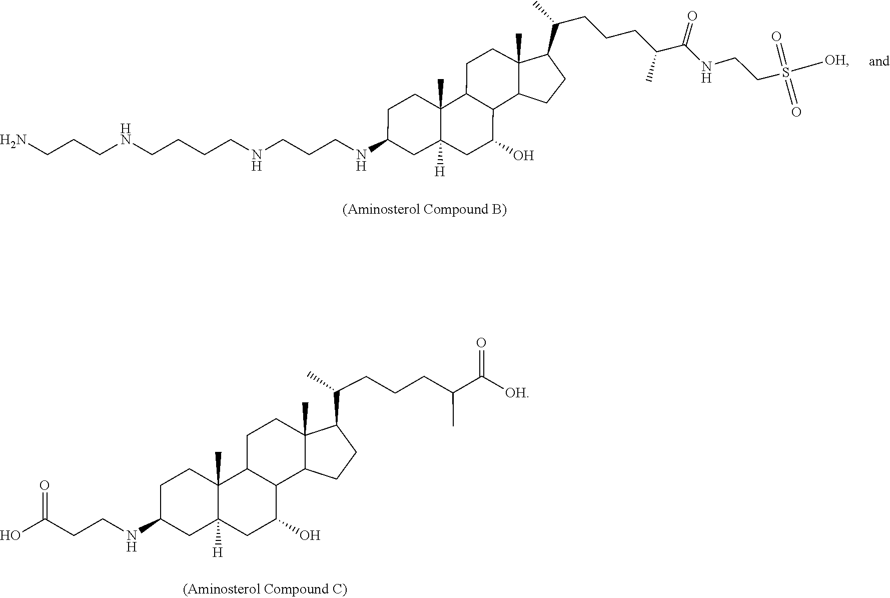 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; CC(CCC[C@@H](C)[C@H]1CCC2C3C(CC[C@@]21C)[C@@]1(C)CC[C@H](NCCC(=O)O)C[C@@H]1C[C@H]3O)C(=O)O]

In one embodiment of the disclosure, the aminosterol is ENT-03S. In another aspect, the aminosterol can be ENT-03R. In yet another aspect, the aminosterol is racemic ENT-03.

In another aspect of the disclosure, the aqueous aminosterol formulation comprises 50 mg/ml of any aminosterol compound described herein, or a pharmaceutically acceptable salt, solvate, prodrug, enantiomer, or derivative thereof, and about 20 to about 23% SBE-β-CD, buffered to pH 7.3-7.5 with Tris.

In a further aspect of the disclosure, the aqueous aminosterol formulation comprises 50 mg/ml of ENT-03R and about 20 to about 25% 2HPβ-CD, buffered to pH 70.3-7.5 with phosphate. In another aspect of the disclosure, the aqueous aminosterol formulation comprises 50 mg/ml of ENT-03S and about 20 to about 25% 2HP-β-CD buffered to pH 7 3-7.5 with phosphate.

In yet another aspect of the disclosure, the aqueous aminosterol formulation comprises 50 mg/ml of a racemic aminosterol compound described herein, or a pharmaceutically acceptable salt, solvate, prodrug, enantiomer, or derivative thereof, and about 20 to about 25% 2HP-β-CD, buffered to pH 7.3-7.5 with phosphate.

In yet another aspect, the aqueous aminosterol formulation comprises 50 mg/ml of racemic LNT-03 and about 20 to about 25% 2HP-β-CD, buffered to pH 7.3-7.5 with phosphate.

The cyclodextrin formulation can also comprise a deuterated aminosterol compound, where an aminosterol compound described herein has at least one hydrogen of the aminosterol compound replaced with deuterium.

In one aspect, a method of treating a subject in need having a condition susceptible to treatment with an aminosterol is provided, the method comprising administering to the subject a therapeutically effective amount of an aminosterol compound, or a pharmaceutically acceptable salt, solvate, prodrug, or derivative thereof, or a composition disclosed herein, including but not limited to an aminosterol-cyclodextrin composition described herein.

In some embodiments, the condition is correlated with abnormal α-synuclein pathology and/or dopaminergic dysfunction. In a further aspect, the condition is correlated with protein misfolding. For example, the misfolded protein can be α-synuclein, tatu, and/or amyloid-$.

In one aspect, provided is a method of treating, preventing, and/or slowing the onset or progression of a condition or disorder, or a related symptom, correlated with abnormal α-synuclein pathology, dopaminergic dysfunction, and/or protein misfolding, in a subject in need.

The method comprises administering a therapeutically effective amount of an aminosterol compound or a composition disclosed herein, including but not limited to an aminosterol-cyclodextrin composition. In some embodiments, the related symptom: (a) is selected from the group consisting of constipation, hallucinations, cognitive impairment, and inflammation; and/or (b) is correlated with a synucleopathy, a neurodegenerative disease, a neurological disease or disorder, a psychological and/or behavior disorder, or a cerebral or general ischemic disorder or condition; and/or (c) the condition or disorder can be a synucleopathy, a neurodegenerative disease, a neurological disease or disorder, a psychological and/or behavior disorder, or a cerebral or general ischemic disorder or condition.

In some embodiments, (a) the synucleopathy, neurodegenerative disease, or neurological disease or disorder is selected from the group consisting of Parkinson's disease, Alzheimer's disease, schizophrenia, multiple system atrophy, Lewy body dementia, dementia with Lewy bodies, Huntington's Disease, Multiple Sclerosis, Amyotrophic Lateral Sclerosis, Friedreich's ataxia, vascular dementia, spinal muscular atrophy, supranuclear palsy, progressive nuclear palsy, frontotemporal dementia, progressive nuclear palsy, Guadeloupian Parkinsonism, spinocerebellar ataxia, parkinsonism, traumatic brain injury, degenerative processes associated with aging, and dementia of aging; (b) the psychological or behavior disorder is selected from the group consisting of depression, autism, autism spectrum disorder, down syndrome, Gaucher's disease, Krabbe's disease, lysosomal conditions affecting glycosphingolipid metabolism, ADHD, ADD, agitation, anxiety, delirium, irritability, illusion and delusions, amnesia, apathy, bipolar disorder, disinhibition, aberrant motor and obsessive--compulsive behaviors, addiction, cerebral palsy, epilepsy, major depressive disorder, and sleep disorders such as REM sleep behavior disorder (RED), sleep fragmentation, REM behavior disorder, circadian rhythm dysfunction, sleep apnea, and cognitive impairment; or (c) the cerebral or general ischemic disorder or condition is selected from the group consisting of microangiopathy, intrapartum, cerebral ischemia, cerebral ischemia during/after cardiac arrest or resuscitation, cerebral ischernia due to intraoperative problems, cerebral ischemia during carotid surgery, chronic cerebral ischernia due to stenosis of blood-supplying arteries to the brain, sinus thrombosis or thrombosis of cerebral veins, cerebral vessel malformations, diabetic retinopathy, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, cardiac conduction defects, high blood pressure, low blood pressure, and pulmonary edema.

In one aspect, a method of treating, preventing, and/or slowing the onset or progression a cerebral or general ischemic disorder and/or a related symptom, correlated with abnormal α-synuclein pathology, dopaminergic dysfunction, and/or protein misfolding in a subject in need is provided, the method comprising administering a therapeutically effective amount of an aminosterol compound or a composition disclosed herein, including but not limited to an aminosterol-cyclodextrin composition.

In one aspect, a method of inhibiting one or more regulatory phosphatases in a subject is provided, the method comprising administering to the subject a therapeutically effective amount of an aminosterol compound or a composition disclosed herein. In some embodiments, the regulatory phosphatase comprises one or more of protein Ser/Thr phosphatases including type 1 (PP1) and type 2 (PP2, i.e., PP2A, PP2C and PP2B) such as PPPICA, PPPICB, PPP1CC, PPP2CA, PPP2CB, PPP3CA, PPP3CB, PPP3CC, PPP4C PPP5C, and PPP6C; Class I Cys-based protein tyrosine phosphatases (PTPs); Class 11 Cys-based PTPs; Class III Cys-based PTPs; Class IV Cys-based DSPs (dual-specificity phosphatases); PTPs such as PTP1B, CDCll4s (CDCll4A, CDC14B, CDC14C, CDKN3); phosphatase and tensin homologs such as PTEN; slingshots such as SSH1, SSH2, and SSH3; dual specificity phosphatases such as DUSP1, DUSP2, DUSP3, DUSP4, DUSP5, DUSP6, DSP7, DISP8, DUSP9, DUSP10, DUSP1 1, IUSP12, DUSP13, DUSP14, DUSP15, DUSP16, DUSP18, DUSP19, DUSP21, DUSP22, DUSP23, DUSP26, DUSP27, and DUSP28; and other phosphatases such as CTDPI, CTDSP1, CTDSP2, CTDSPL, DULLARD, EPM2A, 1LKAP, MDSP, PGAM5, PHLPP1, PHPLPP2, PPEF1, PPEF2, PPM1A, PPM1B, PPM1D, PPM1E, PPM1F, PPM1G, PPM1H, PPM1H, PPM1K, PPM1L, PPM1M, PPM1N, PPTC7. PTPMT1, SSU72, UBLCP1, PP1B, PP1A, PP2Aalpha/PP2R1A complex, PTPN6/SHP1, PTPRC/CD45, DUSP22/MKPX, PTPN2/TC-PTP, PTPN7/LC-PTP, PTPN12/PTP-PEST, PTPN11/PTP1B-CD, PTPN11/SHP2, PTPN11/SHP2-FL, and/or PTPN11/SHlP2-FL(E76K). In some embodiments, the regulatory phosphatase comprises protein tyrosine phosphatase 1B (PTP1B).

In one aspect, a method of suppressing, preventing and/or slowing the onset or progression of appetite or weight gain, including but not limited to obesity, and/or one or more related symptoms, in a subject in need thereof is provided, the method comprising administering to the subject a therapeutically effective amount of an aminosterol compound or a composition disclosed herein, including but not limited to an aminosterol-cyclodextrin composition.

In another aspect, a method of improving or resolving impaired reproductive function, impaired fertility and/or impaired reproductive behavior in a subject in need thereof, is provided. The method comprises administering to the subject a therapeutically effective amount of an aminosterol compound or a composition disclosed herein, including but not limited to an aminosterol-cyclodextrin composition.

In a further aspect, a nm method of treating, preventing, and/or improving diabetes, which can be either type 1 or type 2 diabetes, in a subject in need thereof, is provided. The method comprises administering to the subject a therapeutically effective amount of an aminosterol compound or a composition disclosed herein, including but not limited to an aminosterol-cyclodextrin composition.

In some embodiments (a) the method of administration comprises oral, nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof; and/or (b) the method of administration is nasal administration, oral administration, or a combination thereof.

In some embodiments, the therapeutically effective amount of the aminosterol compound, or a pharmaceutically acceptable salt, solvate, prodrug, or derivative thereof, comprises: (a) about 0 1 to about 20 mg/kg body weight of the subject; (b) about 0.1 to about 15 mg/kg body weight of the subject; (c) about 0.1 to about 10 mg/kg body weight of the subject; (d) about 0.1 to about 5 mg/kg body weight of the subject; or (e) about 0.1 to about 2.5 mg/kg body weight of the subject.

In some embodiments, the therapeutically effective amount of the aminosterol compound, or a pharmaceutically acceptable salt, solvate, prodrug, or derivative thereof, comprises: (a) about 0.001 to about 500 mg/day; (b) about 0.001 to about 250 ng/day; (c) about 0.001 to about 125 mg/day; (d) about 0.001 to about 50 mg/day; (e) about 0.001 to about 25 mg/day; (f) about 0.001 to about 10 mg/day; (g) about 0.001 to about 6 mg/day; (h) about 0.001 to about 4 mg/day; or (i) about 0.001 to about 2 mg/day.

In some embodiments, the method of administration comprises oral administration and wherein the therapeutically effective amount of the aminosterol compound, or a pharmaceutically acceptable salt, solvate, prodrug, or derivative thereof, comprises: about 1 to about 300 mg/day; or about 25 to about 500 mg/day.

In some embodiments, the aminosterol compound, or a pharmaceutically acceptable salt, solvate, prodrug, or derivative thereof, is administered in combination with at least one additional active or therapeutic agent to achieve either an additive or synergistic effect. In some embodiments, the additional active or therapeutic agent is administered via a method selected from the group consisting of (a) concomitantly: (b) as an admixture; (c) separately and simultaneously or concurrently; and (d) separately and sequentially. In some embodiments, the additional active or therapeutic agent is a second aminosterol having a different structure from the aminosterol administered as described in the above paragraphs.

In some embodiments administration of the composition comprises administration on an empty stomach, optionally within two hours of the subject waking; and/or no food is consumed by the subject after about 60 to about 90 minutes from administration of the composition.

In some embodiments, the method further comprises (a) determining a dosage of the aminosterol, or a pharmaceutically acceptable salt, solvate, prodrug, or derivative thereof, for the subject, wherein the aminosterol dosage is determined based on the effectiveness of the aminosterol dosage in improving or resolving a symptom being evaluated, (b) followed by administering a composition comprising the dosage of the aminosterol to the subject for a period of time, wherein the method comprises: (i) identifying a symptom to be evaluated, wherein the symptom is susceptible to treatment with an aminosterol; (ii) identifying a starting dosage of an aminosterol thereof for the subject; (iii) administering an escalating dosage of the aminosterol to the subject over a period of time until an effective dosage for the symptom being evaluated is identified, wherein the effective dosage is the aminosterol dosage where improvement or resolution of the symptom is observed, and fixing the aminosterol dosage at that level for that particular symptom in that particular subject. In some embodiments, improvement or resolution of the symptom is measured using a clinically recognized scale or tool.

In some embodiments: (a) the composition is administered orally and: (i) the starting aminosterol dosage ranges from about 10 mug up to about 150 mg/day; (ii) the dosage of the aminosterol for the subject following escalation is fixed at a range of from about 2.5 mg up to about 500 tug/day; and/or (iii) the dosage of the aminosterol or a salt or derivative thereof is escalated in about 25 mg increments; or (b) the composition is administered intranasally and: (i) the starting aminosterol dosage ranges from about 0.001 mg to about 3 mg/day; (ii) the dosage of the aminosterol for the subject following escalation is fixed at a range of from about 0.001 mg up to about 6 mg/day; (iii) the dosage of the aminosterol for the subject following escalation is a dosage which is subtherapeutic when given orally or by injection; and/or (iv) the dosage of the aminosterol is escalated in increments of about 0 1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 6, about 1.7, about 1.8, about 1.9, or about 2 mg.

In some embodiments: (a) the dosage of the aminosterol, or a pharmaceutically acceptable salt, solvate, prodrug, or derivative thereof, is escalated every about 3 to about 5 days; and/or (b) the starting aminosterol dosage is higher if the symptom being evaluated is severe; and/or (c) the symptom is correlated with abnormal α-synuclein pathology and/or dopaminergic dysfunction.

In some embodiments, the symptom to be evaluated is selected from the group consisting of (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part IL of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part V of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (1) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature: (t) improvement in breathing or apnea; (u) correction of cardiac conduction defect; (v) amelioration of pain; (w) restoration of bladder sensation and urination; (x) urinary incontinence; and/or (y) control of nocturia.

In some embodiments, the symptom to be evaluated is constipation, and wherein: (a) the fixed escalated aminosterol dosage for constipation is defined as the aminosterol dosage that results in a complete spontaneous bowel movement (CSB3M) within 24 hours of dosing on at least 2 of 3 days at a given dosage; (b) if average complete spontaneous bowel movement (CSBM) or average spontaneous bowel movement (SBM) is greater than or equal to 1 per week, then the starting aminosterol dosage prior to escalation is 75 mg/day; and/or (c) if average CSBM or SBM is less than 1 per week, then the starting aminosterol dosage prior to escalation is 150 mg/day.

In one aspect, a method of increasing gene transcription in the gut of a subject is provided, the method comprising administering to the subject a therapeutically effective amount of an aminosterol compound or composition disclosed herein. In some embodiments, the increase in gene transcription is for one or more genes selected from the group consisting of caspase 14, collagen type XVII alpha 1, corneodesmosin, cornifelin, cystatin E/M, dermokine, desmocollin 1, desmnoglein I beta, filaggrin, gap junction protein beta 4, gap junction protein beta 6,1419 imprinted maternally expressed transcript, hornerin, kallikrein related-peptidase 7 chymotryptic stratum, keratin 1, keratin 10, keraminocyte differentiation associated protein, keraminocyte expressed proline-rich, late cornified envelope IA I, late cornified envelope 1A2, late cornified envelope 1B, late cornified envelope 1C, late cornified envelope IE, late cornified envelope 1F, late cornified envelope 1G, late cornified envelope 1H, late cornified envelope 1I, late cornified envelope 1J, late cornified envelope 1L, late cornified envelope 1M, late cornified envelope 3C, late cornified envelope 3E, late cornified envelope 3F, lectin galactose binding soluble 7, loricrin, sciellin, myoglobin, myosin binding protein C slow-type, myosin heavy polypeptide 1 skeletal muscle, myosin heavy polypeptide 8 skeletal muscle, myosin light chain phosphorylatable fast ske, myosin light polypeptide 3, myozenin 1, myozenin 2, and titin-cap.

In some embodiments, the increase in gene transcription is selected from about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 125%, about 1²5% to about 150%, about 150% to about 175%, about 175% to about 200%, about 200% to about 250%, about 250% to about 300%, about 300% to about 350%, about 350% to about 400%, about 400% to about 450%, about 500% to about 600%, about 600% to about 700%, about 700% to about 800%, about 800% to about 900%, about 900% to about 1000%, or about 1000% to about 1500%.

In one aspect, a method of inhibiting one or more regulatory phosphatases to achieve a therapeutic or prophylactic benefit is provided, the method comprising to a subject administering a therapeutically effective amount of an aminosterol compound, or a pharmaceutically acceptable salt, solvate, prodrug, or derivative thereof, or a composition disclosed herein.

In all of the methods described herein, the subject can be a human.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE FIGURES

FIG. 1 shows XIC chromotagraphs from full MS/ddMS2 scans of ENT-03S in rat plasma samples.

FIG. 2 shows XIC chromatograms from full MS/ddMS2 scans of ENT-03S in rat urine samples.

FIG. 3 shows the MS/IS fragmentation spectrum of ENT-03S.

FIG. 4 shows the MS/MS fragmentation spectrum of an ENT-03S derivative designated as M1-PU.

FIG. 5 shows the MS/NIS fragmentation spectrum of an ENT-03S derivative designated as M2-PU.

FIG. 6 shows the MS/MS fragmentation spectrum of an ENT-03S derivative designated as M4-PU.

FIG. 7 shows the MS/MS fragmentation spectrum of an ENT-03S derivative designated as M5-PU.

FIG. 8 shows the MS/MS fragmentation spectrum of an ENT-03S derivative designated as M6-PU.

FIG. 9 shows the MS/MS fragmentation spectrum of an ENT-03S derivative designated as M7-PU.

FIG. 10 shows the MS/MS fragmentation spectrum of an ENT-03S derivative designated as M8-PU.

FIG. 11 shows the MS/MS fragmentation spectrum of an ENT-03S derivative designated as M9-PU.

FIG. 12 shows the MS/MS fragmentation spectrum of an ENT-03S derivative designated as M10-PLU

FIG. 13 shows proposed metabolic pathways for ENT-03S from rat plasma and urine samples.

FIG. 14 shows a chromatogram for ENT-19.

FIG. 15 shows XIC chromotagraphs from full MS/ddMS2 scans of ENT-03S rat bile samples.

FIG. 16 shows the MS/MS fragmentation spectrum of ENT-03S.

FIG. 17 shows the MS/MS fragmentation spectrum of an ENT-03S derivative designated as M1-RB.

FIG. 18 shows the MS/MS fragmentation spectrum of an ENT-03S derivative designated as M2-RB.

FIG. 19 shows the MS/MS fragmentation spectrum of an ENT-03S derivative designated as M3-RB.

FIG. 20 shows the MS/MS fragmentation spectrum of an ENT-03S derivative designated as M4-RB.

FIG. 21 shows the MS/MS fragmentation spectra of an ENT-03S derivative designated as M5-RB.

FIG. 22 shows the MS/MS fragmentation spectrum of an ENT-03S derivative designated as M6-RB.

FIG. 23 shows the MS/MS fragmentation spectrum of an ENT-03S derivative designated as MS-RB.

FIG. 24 shows the MS/MS fragmentation spectrum of an ENT-03S derivative designated as M9-RB.

FIG. 25 shows the MS/MS fragmentation spectrum of an ENT-03S derivative designated as M10-RB.

FIG. 26 shows the MS/MS fragmentation spectrum of an ENT-03S derivative designated as ENT-25S (M11-1).

FIG. 27 shows the MS/MS fragmentation spectrum of an ENT-03S derivative designated as M12-RB.

FIG. 28 shows the MS/MS fragmentation spectrum of an ENT-03S derivative designated as M13-RB.

FIG. 29 shows the MS/MS fragmentation spectrum of an ENT-03S derivative designated as M14-RB.

FIG. 30 shows the MS/MS fragmentation spectrum of an ENT-03S derivative designated as M15-RB.

FIG. 31 shows the MS/MS fragmentation spectrum of an ENT-03S derivative designated as M16-R13.

FIG. 32 shows the MS/IS fragmentation spectrum of an ENT-03S derivative designated as M17-RB.

FIG. 33 shows proposed metabolic pathways for ENT-03S from rat bile samples.

FIG. 34 shows the mean plasma concentration vs time profile for the aminosterol ENT-03S formulated with a cyclodextrin following 10 mg/kg subcutaneous injection in Male SD rats.

FIG. 35 shows the mean plasma concentration vs time profile for the aminosterol ENT-03S in a formulation lacking a cyclodextrin following 10 mg/kg subcutaneous injection in Male SD rats.

### DETAILED DESCRIPTION

### 1 Overview of the Invention

This invention relates to aminosteroid compositions useful in methods of treating human disease. The specific compounds disclosed include those designated ENT-12, ENT-13, ENT-14, ENT-14S, ENT-14R, ENT-15, ENT-16, ENT-17 (also known as M1-PU), ENT-18, ENT-18S, ENT-18R, ENT-19 (ENT-19 is the racemic version of M10-PU), ENT-19S, ENT-20, ENT-2, ENT-21S, ENT-21R, ENT-21 mixed isomers, ENT-22, ENT-22S. ENT-22R, ENT-22 mixed isomers, ENT-23, ENT-24, ENT-25, ENT-25R, ENT-25S (also known as M11-RB), ENT-25 mixed isomers, ENT-26, ENT-27, ENT-28, ENT-29, ENT-30, ENT-31, ENT-32, ENT-33, M10-PU/M14-RB, M2-RB, M3-RB, M8-RB, M5-RB, M16-RB, Aminosterol Compound A, Aminosterol Compound B, and Aminosterol Compound C, or a pharmaceutically acceptable salt, solvate, prodrug, enantiomer, or derivative thereof.

Aminosterol 1436 was discovered to inhibit Protein Tyrosine Phosphatase I B (PTP1B) both in vitro and in vivo. When administered to obese mice, aminosterol 1436 reduced food intake, adiposity and body weight, corrected insulin resistance and reversed fatty liver disease. In early stage human clinical trials, it exhibited dose dependent weight reduction and increased insulin sensitivity.

Since aminosterol 1436 exhibited pharmacological activity in numerous vertebrate species, it was theorized that a functional analogue was present in higher vertebrates. Aminosterol 1436 is comprised of a sulfated planar (“flat”) bile alcohol linked to spermine. Although no similar compound has been isolated from any mammalian source to date, planar C-26 bile acids, such as 7-alpha-hydroxy-3-oxo-4 cholestenoic acid (7-HOCA), are amongst the most abundant bile acids present in mouse and human cerebrospinal fluid. Based on this information and additional research, ENT-03 was identified and described in WO 2021/025973.

ENT-03 was detected in brain, liver, kidney, and GI tract of neonatal mice. Highest concentrations were detected in brain. ENT-03 isolated from brain was represented by both R and S diastereomers at the chiral C25 center, in about a 60% to 40% ratio, respectively. The concentration in brain was low at birth, reached peak concentrations over the first postnatal week, and declined rapidly as the animals aged. The low concentrations of ENT-03 in the GI tract suggested that milk was not the source of the compound.

INT-03 was assayed in vitro against a bank of commercially available phosphatases. PTP1B was the most potently inhibited of the phosphatases assayed. The inhibitory “fingerprint” of ENT-03 resembled that of aminosterol 1436. Properties of ENT-03 include increasing insulin sensitivity as a centrally active PTP1B inhibitor, as well as inducing prolonged reduction in food intake and body weight after both peripheral and central administration to lean rodents. In sum, ENT-03 causes reduced food consumption and resultant weight loss. Further, ENT-03 reduces hepatic steatosis, and acutely increases insulin sensitivity. Further, ENT-03 maintains its effects on metabolism after chronic intranasal dosing.

Based on the ENT-03 properties identified, novel metabolites of ENT-3 were identified. Throughout this application, the nomenclature “PU” refers to compounds isolated from rat plasma and urine, and “RB” refers to compounds isolated from rat bile.

In addition, this disclosure relates to the formulation of aminosteroid compounds to be delivered to subjects for the treatment of disease. The specific compounds considered are aminosteroids related to the class of mammalian polyamine-bile acids, including pharmaceutically acceptable salts, solvates, prodrugs, or derivatives thereof. This aspect of the disclosure encompasses the novel aminosterols detailed herein as well as aminosterols described in the prior art, such as squalamine (ENT-01), squalamine phosphate, aminosterol 1436 (ENT-02) (also known as trodusquemine and MSI-1436), ENT-03, ENT-05 (US-2023/0123701), ENT-04, ENT-07, ENT-08, ENT-09, ENT-10, ENT-11 (US 2023/0125585), and ENT-06 (US 2022/0372066).

Aminosterols described herein share biophysical properties that are incompatible with subcutaneous administration. In particular, the chemical structure of aminosterols predicts that, at physiological pH, the molecules will be highly water soluble, anphipathic, and cationic. When injected into animal tissues, solutions of membrane-active or membrane-disruptive aminosterols damage tissues through lytic action. The extent of tissue damage is directly proportional to the aminosterol concentration. The tissue damage correlates with severe pain when administered, making the aminosterol compounds unsuitable and undesirable for injectable therapeutic use (subcutaneously, intramuscularly, intravenously, etc.). In addition, as a consequence of both the positive charge and the tissue damage incurred, bioavailability of the subcutaneously administered aminosteroid is compromised. In sum, as a consequence of detergency, aminosterols cannot be administered subcutaneously (or IM, IV, etc.) at concentrations that are required to achieve a therapeutic effect.

The present disclosure details a method of preparing a parenteral formulation of an aminosterol, or a pharmaceutically acceptable salt, solvate, prodrug, or derivative thereof, using cyclodextrins. The novel formulations surprisingly result in pain-free subcutaneous administration, and further exhibit acceptable bioavailability.

Based on pharmacological studies in rodents, the probable dose of an aminosterol that must be administered to a 75 kg person is between about 10 to about 50 mg. Since the maximal volume of a subcutaneous dose is about 1 nil, an aminosterol concentration of between 10 to 50 mg/ml would be administered. At these concentrations, administration of an aminosterol would cause intense burning pain, similar to a bee sting, and cause tissue necrosis at the injection site.

The present disclosure is directed to the discovery of an ideal formulation of an aminosterol, or a pharmaceutically acceptable salt, solvate, prodrug, or derivative thereof, which is painless on injection and permits rapid diffusion of the drug from the injection site into the blood stream.

In another aspect of the disclosure, formulations of an aminosterol, or a pharmaceutically acceptable salt, solvate, prodrug, or derivative thereof, contain at least about 50 mg/ml of aminosterol, or a pharmaceutically acceptable salt, solvate, prodrug, or derivative thereof. In another aspect, the formulation is an aqueous homogeneous solution that can be filter sterilized.

In a further aspect of the disclosure, the aminosterol formulations comprise a minimum of excipients and exhibit the necessary chemical and physical stability required of the drug product.

Because the aminosterol-cyclodextrin formulations described herein are for injectable administration, e.g. intravenous, subcutaneous, or intramuscular, it is preferable that the formulations be buffered to an appropriate pH-. An appropriate pH for intravenous and intramuscular injection is 2-11, and an appropriate pH for subcutaneous injection is 4-9. In one aspect, the formulation is for subcutaneous administration and the pH is about 7-8. In other aspects, the pH of the buffered aqueous aminosterol cyclodextrin formulation is between about 7 to 8, or about 7.2 to 7.5, or about 7.4.

The pH can be controlled by a buffer, such as acetate, phosphate, citrate, histidine, tris(hydroxymethyl)aminomethane (TRIS), and others, to an ideal range of pH 5-8. Commonly used buffers include, for example, sodium phosphate, citric acid, acetic acid, KH₂PO₄, CHES, Borate, tromethamine, histidine, gluconic acid, lactic acid, tartaric acid, aspartic acid, glutamic acid, citric acid cycle intermediates, [tris(hydroxymethyl)methylamino]propanesulfonic acid (TAPS), 2-(bis(2-hydroxyethyl)amino)acetic acid (Bicine), N-[tris(hydroxymethyl)methyl]glycine (Tricine), 3-[N-tris(hydroxymethyl)methylamino]-2-hydroxypropanesulfonic acid (TABSO), 4-(2-hydroxyethyl)-1-piperazineethanesulfonic acid (HEPES), 2-[[1, 3-dihydroxy-2-(hydroxymethyl)propan-2-yl]amino]ethanesulfonic acid (TES), 3-(N-morpholino)propanesulfonic acid (MOPS), piperazine-N,N′-bis(2-ethanesulfonic acid) (PIPES), dimethylarsenic acid (Cacadylate), and 2-(N-morpholino)ethanesulfonic acid (MES).

In one aspect of the disclosure, the aminosterol cyclodextrin formulation comprises the following, at a pH of 7.4: 50 mg/ml ENT-03 (active), 25% 2-hydroxypropyl-beta-cyclodextrin, and 80 mM phosphate.

In another aspect of the disclosure, the aminosterol cyclodextrin formulation comprises the following, at a pH of 7.3:

TABLE 1

| Component | Amount | |
|---|---|---|
| ENT-03S Na Powder (~82.68% | 23 g (active Free | |
| purity) | Base, 20.125 g) | |
| (2-Hydroxypropyl)-β-cyclodextrin | 100.7 | g |
| Phosphoric acid | ~30.84 | mmol |

| | |
|---|---|
| Milli Q Water (Particle free) | Up to 402.5 mL |

i. Novel Aminosterols

Novel aminosterol compounds disclosed herein are designated ENT-12, ENT-13, ENT-14, ENT-145, ENT-14R, ENT-15, ENT-16, ENT-17 (also known as M1-PU), ENT-18, ENT-18S, ENT-_8R, ENT-19 ([ENT-19 is the racemic version of 10-PU), ENT-9S, ENT-20, ENT-2, ENT-215, ENT-21R, ENT-21 mixed isomers, ENT-22, ENT-22S, ENT-22R, ENT-22 mixed isomers, ENT-23, ENT-24, ENT-25, ENT-25R, ENT-255 (also known as M11-RB), ENT-25 mixed isomers, ENT-26, ENT-27, ENT-28, ENT-29, ENT-30, ENT-31, ENT-32, ENT-33, M10-PU/M14-RB, M2-RB, M3-RB, M8-RB, M5-RB, M16-RB, Amninosterol Compound A, Aminosterol Compound 13, and Aminosterol Compound C, or a pharmaceutically acceptable salt, solvate, prodrug, enantiomer, or derivative thereof, with the structure of each compound shown below:

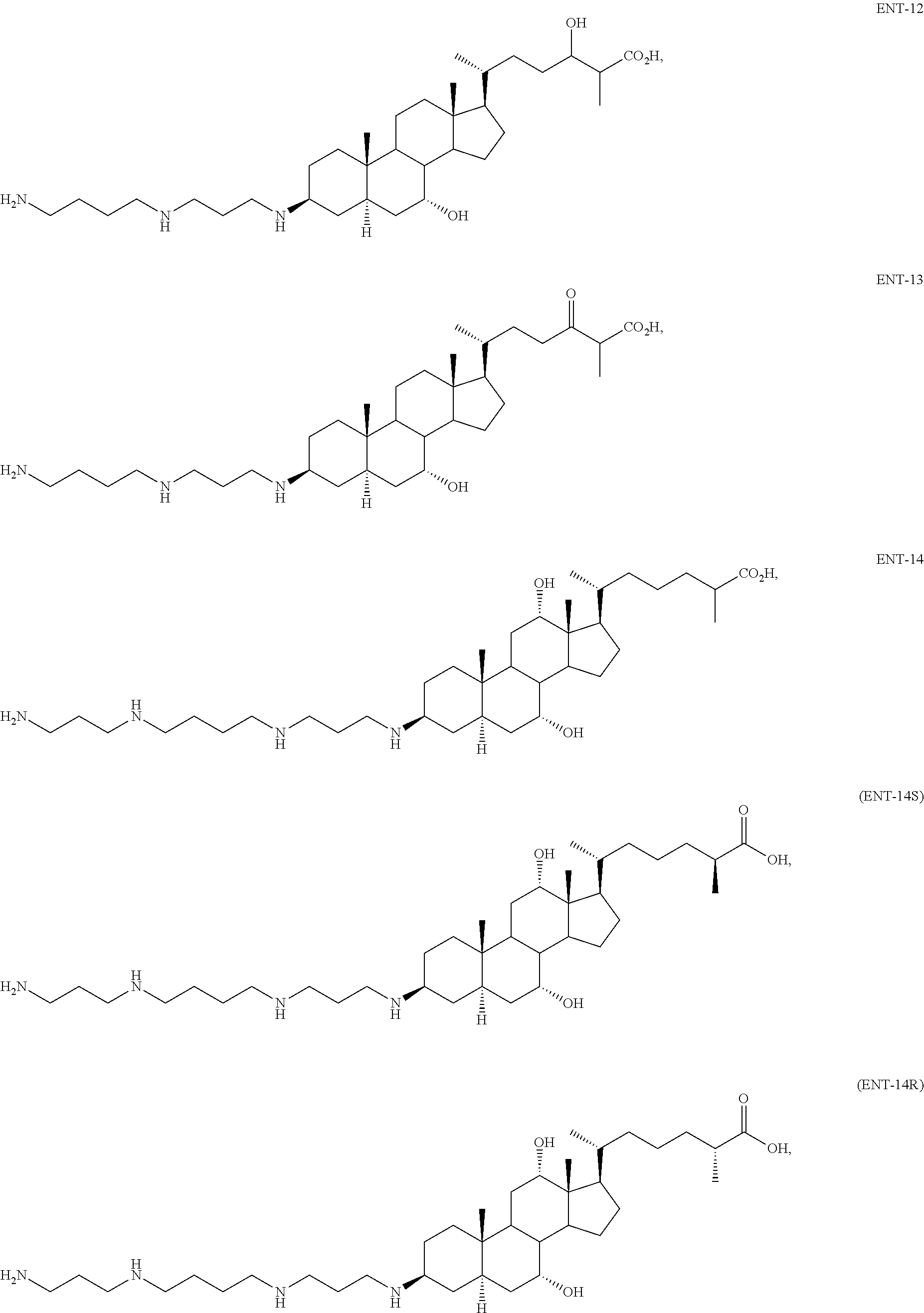

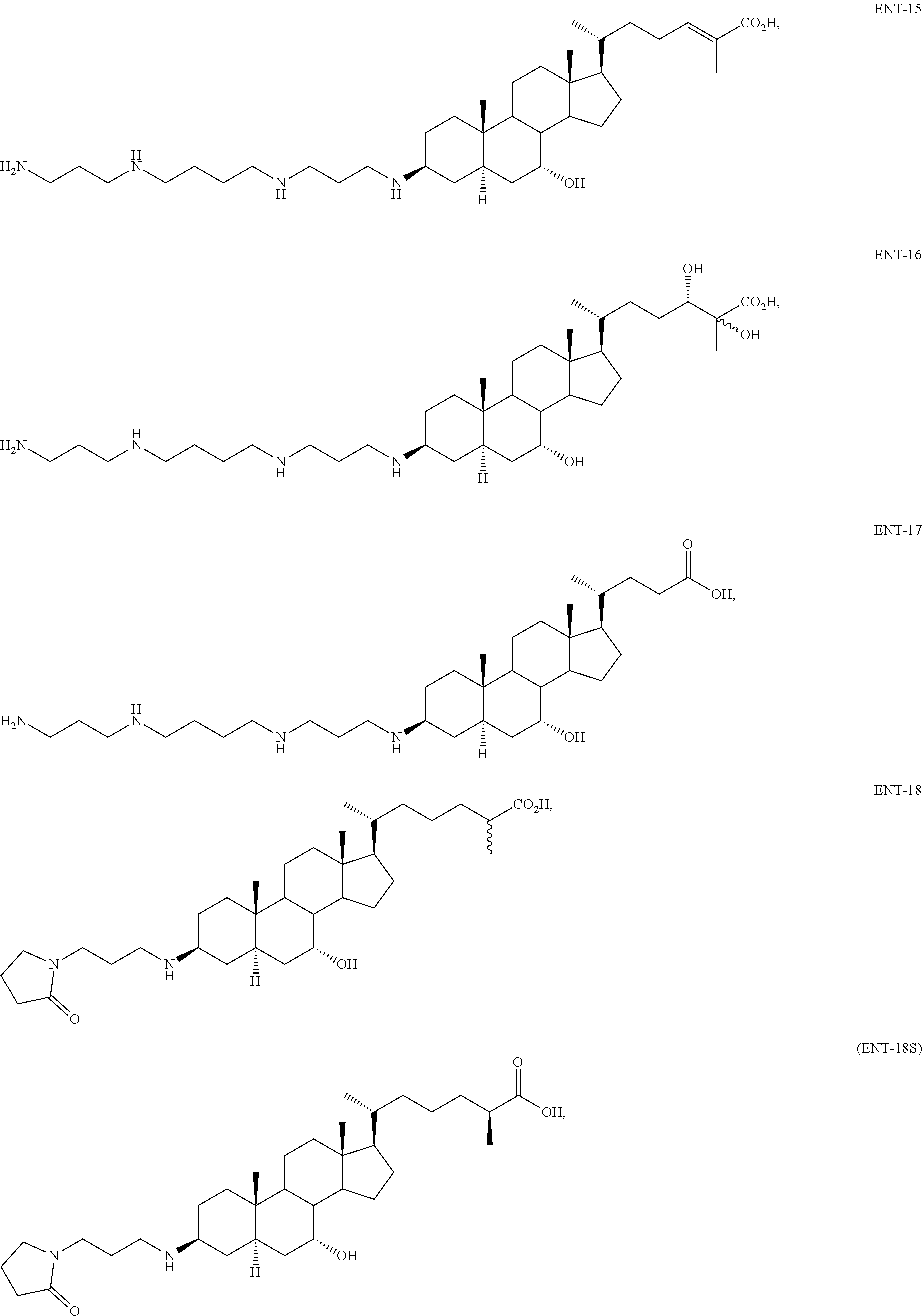 [SMILES: C/C(=C\CC[C@@H](C)[C@H]1CCC2C3C(CC[C@@]21C)[C@@]1(C)CC[C@H](NCCCNCCCCNCCCN)C[C@@H]1C[C@H]3O)C(=O)O ; C[C@H](CC[C@H](O)C(C)(O)C(=O)O)[C@H]1CCC2C3C(CC[C@@]21C)[C@@]1(C)CC[C@H](NCCCNCCCCNCCCN)C[C@@H]1C[C@H]3O ; C[C@H](CCC(=O)O)[C@H]1CCC2C3C(CC[C@@]21C)[C@@]1(C)CC[C@H](NCCCNCCCCNCCCN)C[C@@H]1C[C@H]3O ; CC(CCC[C@@H](C)[C@H]1CCC2C3C(CC[C@@]21C)[C@@]1(C)CC[C@H](NCCCN2CCCC2=O)C[C@@H]1C[C@H]3O)C(=O)O ; C[C@H](CCC[C@H](C)C(=O)O)[C@H]1CCC2C3C(CC[C@@]21C)[C@@]1(C)CC[C@H](NCCCN2CCCC2=O)C[C@@H]1C[C@H]3O]

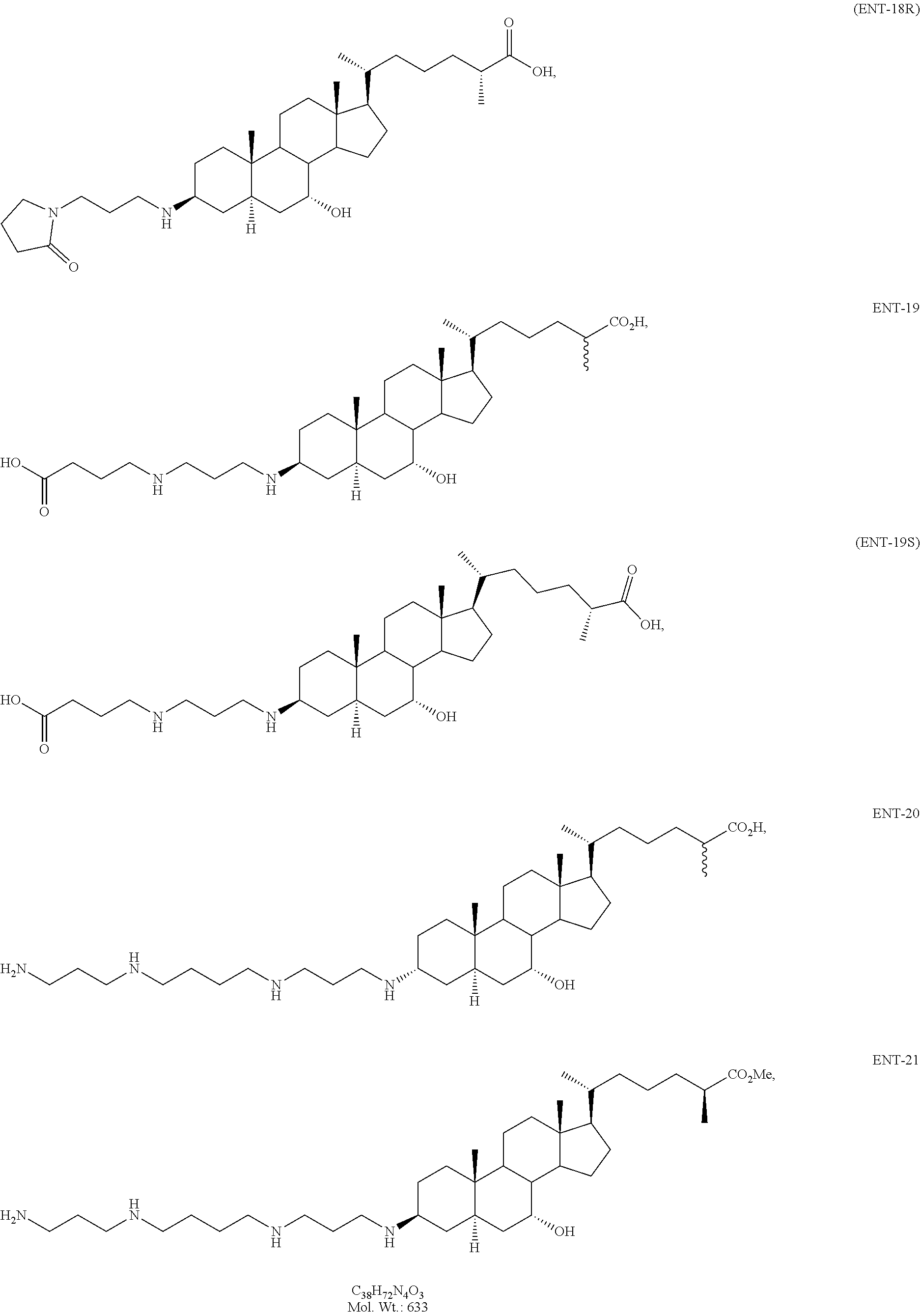

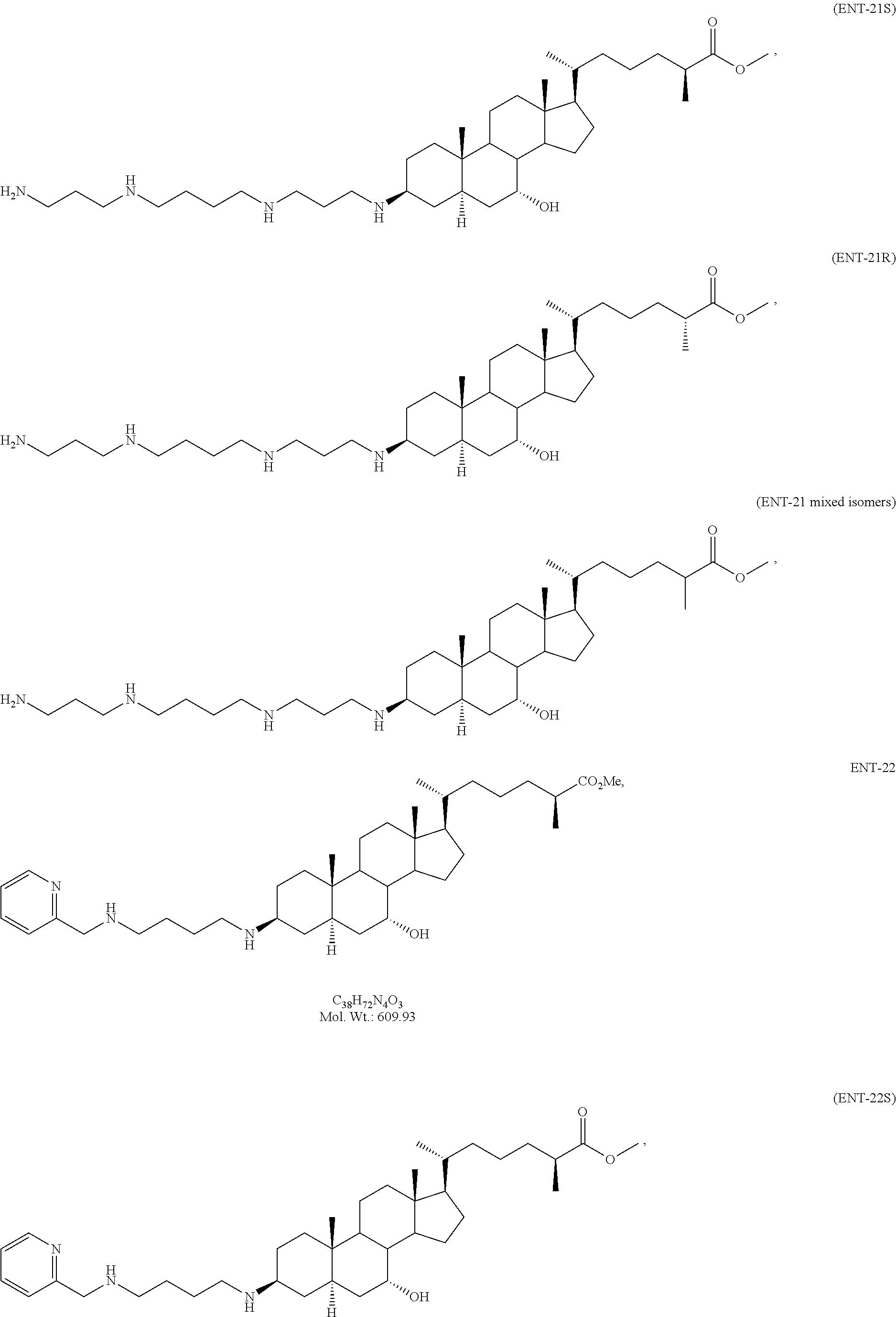

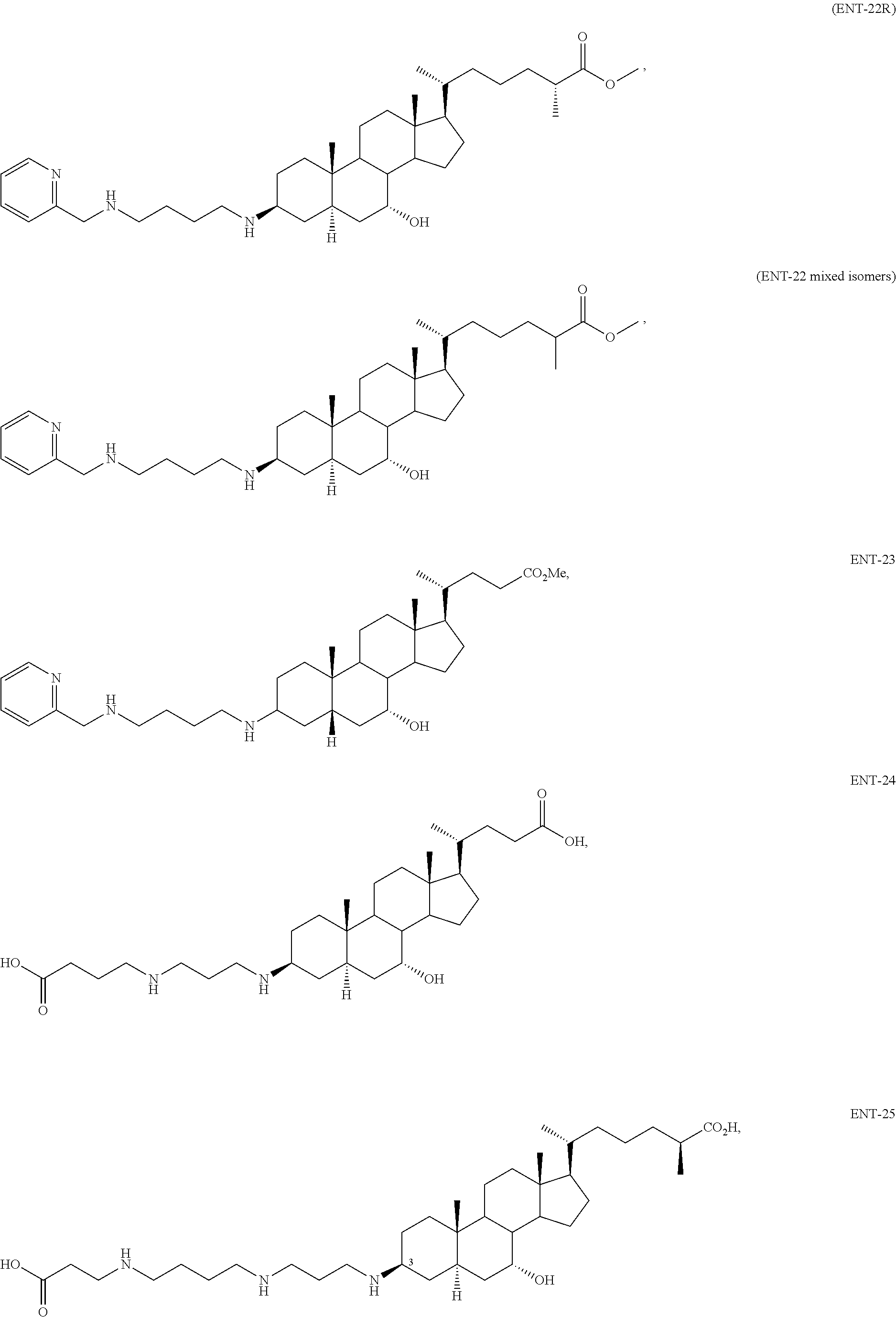 [SMILES: COC(=O)[C@H](C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)C(C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)CC[C@@H](C)[C@H]1CCC2C3C(CC[C@@]21C)[C@@]1(C)CCC(NCCCCNCc2ccccn2)C[C@H]1C[C@H]3O ; C[C@H](CCC(=O)O)[C@H]1CCC2C3C(CC[C@@]21C)[C@@]1(C)CC[C@H](NCCCNCCCC(=O)O)C[C@@H]1C[C@H]3O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O]

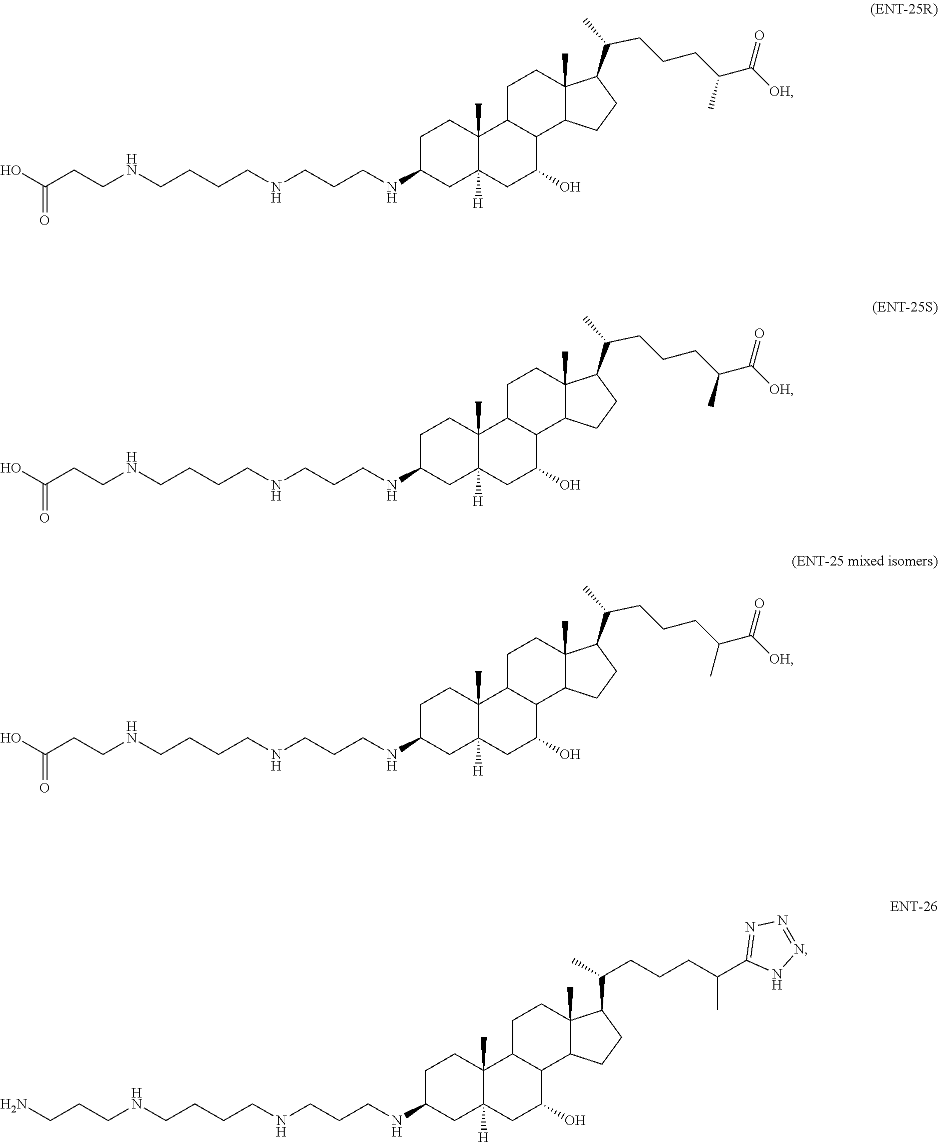 [SMILES: C[C@H](CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; CC(CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O ; CC(CCC[C@@H](C)[C@H]1CCC2C3C(CC[C@@]21C)[C@@]1(C)CC[C@H](NCCCNCCCCNCCCN)C[C@@H]1C[C@H]3O)c1nnn[nH]1]

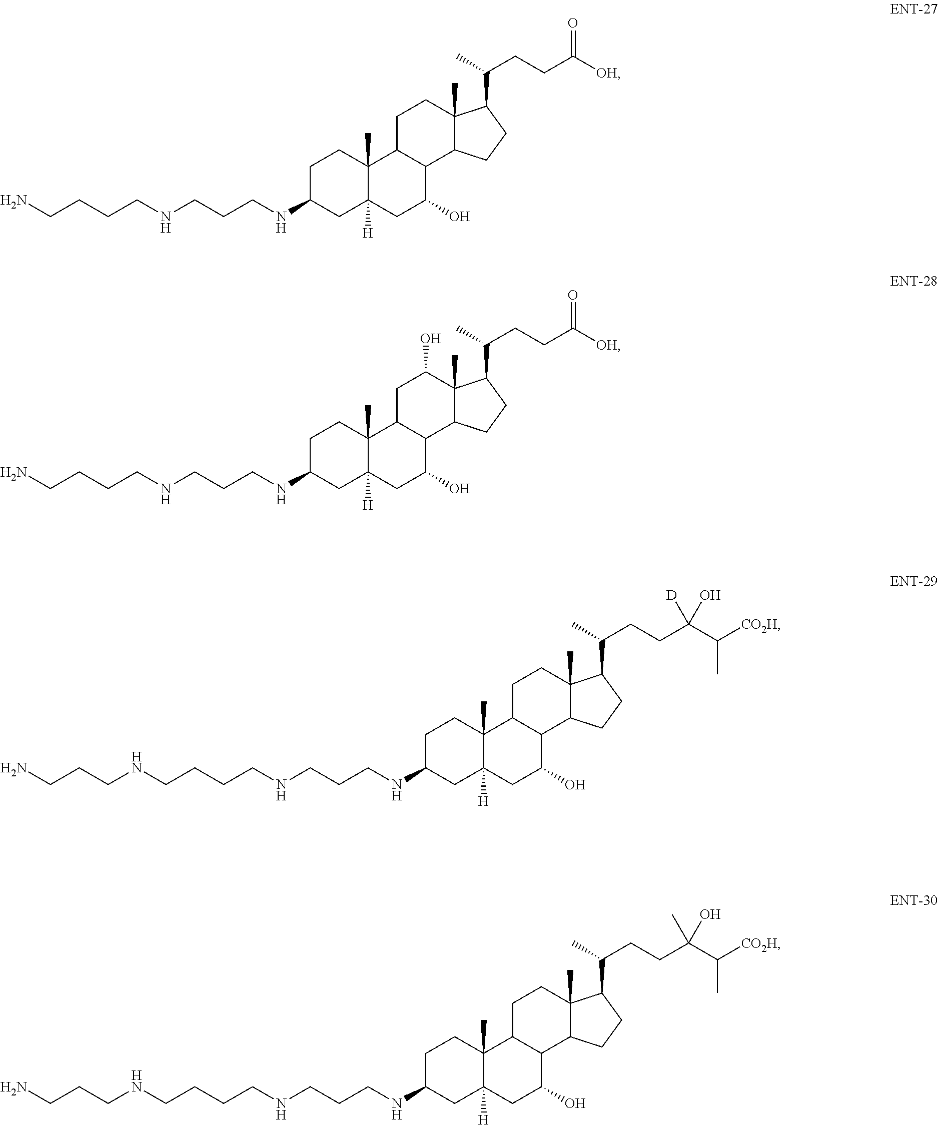 [SMILES: C[C@H](CCC(=O)O)[C@H]1CCC2C3C(CC[C@@]21C)[C@@]1(C)CC[C@H](NCCCNCCCCN)C[C@@H]1C[C@H]3O ; C[C@H](CCC(=O)O)[C@H]1CCC2C3C(C[C@H](O)[C@@]21C)[C@@]1(C)CC[C@H](NCCCNCCCCN)C[C@@H]1C[C@H]3O ; [2H]C(O)(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(C(=O)O)C(C)(O)CC[C@@H](C)[C@H]1CCC2C3C(CC[C@@]21C)[C@@]1(C)CC[C@H](NCCCNCCCCNCCCN)C[C@@H]1C[C@H]3O]

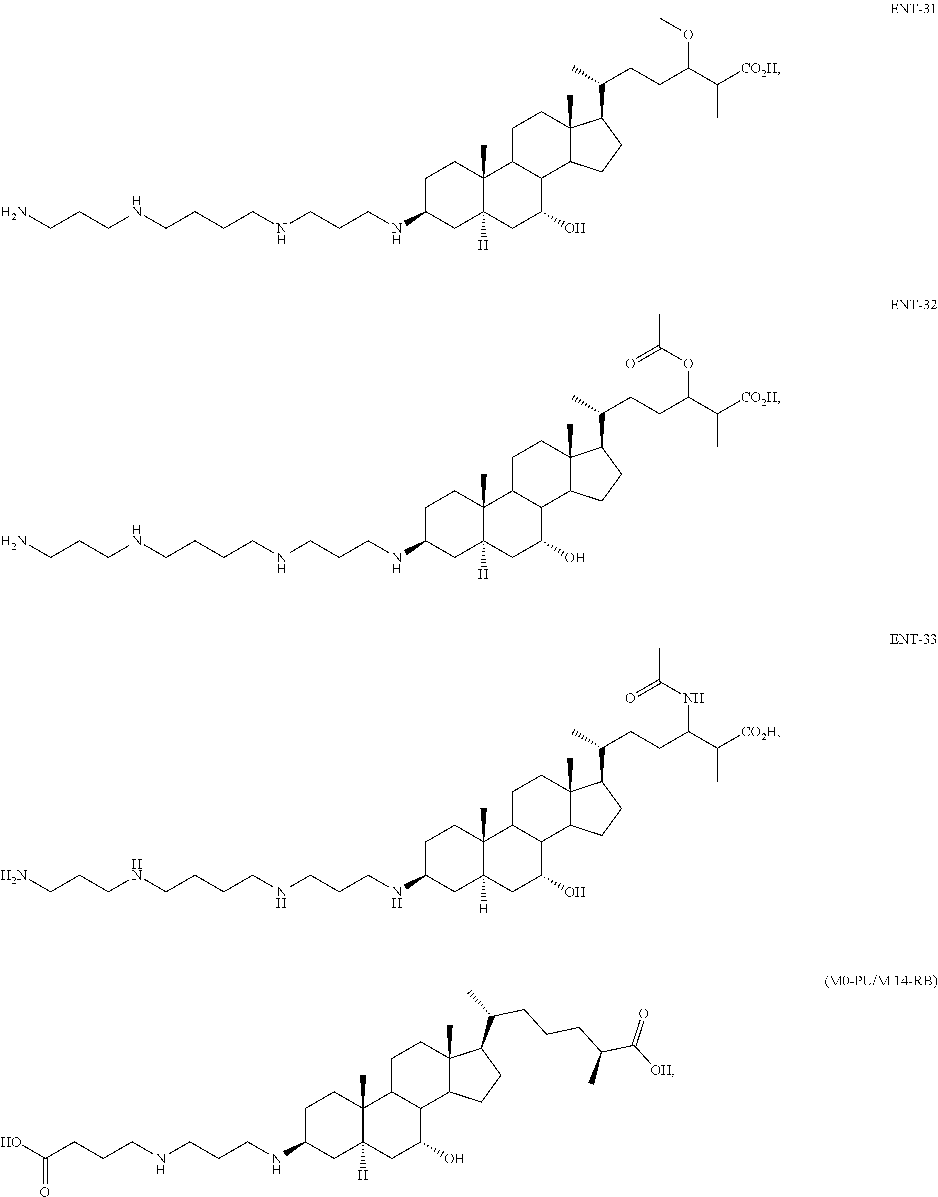 [SMILES: COC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(=O)OC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(=O)NC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; C[C@H](CCC[C@H](C)C(=O)O)[C@H]1CCC2C3C(CC[C@@]21C)[C@@]1(C)CC[C@H](NCCCNCCCC(=O)O)C[C@@H]1C[C@H]3O]

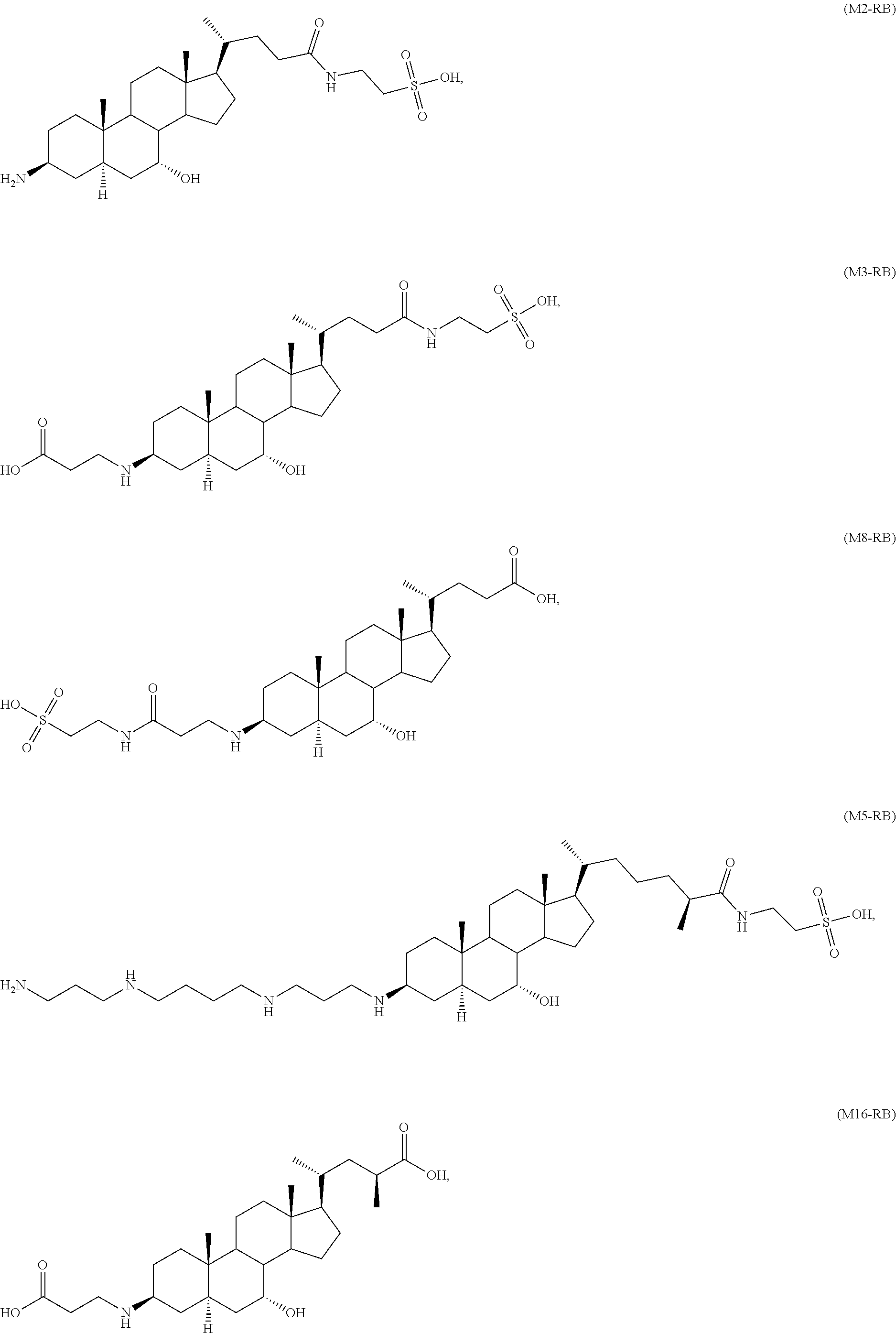 [SMILES: C[C@H](CCC(=O)NCCS(=O)(=O)O)[C@H]1CCC2C3C(CC[C@@]21C)[C@@]1(C)CC[C@H](N)C[C@@H]1C[C@H]3O ; C[C@H](CCC(=O)NCCS(=O)(=O)O)[C@H]1CCC2C3C(CC[C@@]21C)[C@@]1(C)CC[C@H](NCCC(=O)O)C[C@@H]1C[C@H]3O ; C[C@H](CCC(=O)O)[C@H]1CCC2C3C(CC[C@@]21C)[C@@]1(C)CC[C@H](NCCC(=O)NCCS(=O)(=O)O)C[C@@H]1C[C@H]3O ; C[C@H](CCC[C@H](C)C(=O)NCCS(=O)(=O)O)[C@H]1CCC2C3C(CC[C@@]21C)[C@@]1(C)CC[C@H](NCCCNCCCCNCCCN)C[C@@H]1C[C@H]3O ; C[C@H](C[C@H](C)C(=O)O)[C@H]1CCC2C3C(CC[C@@]21C)[C@@]1(C)CC[C@H](NCCC(=O)O)C[C@@H]1C[C@H]3O]

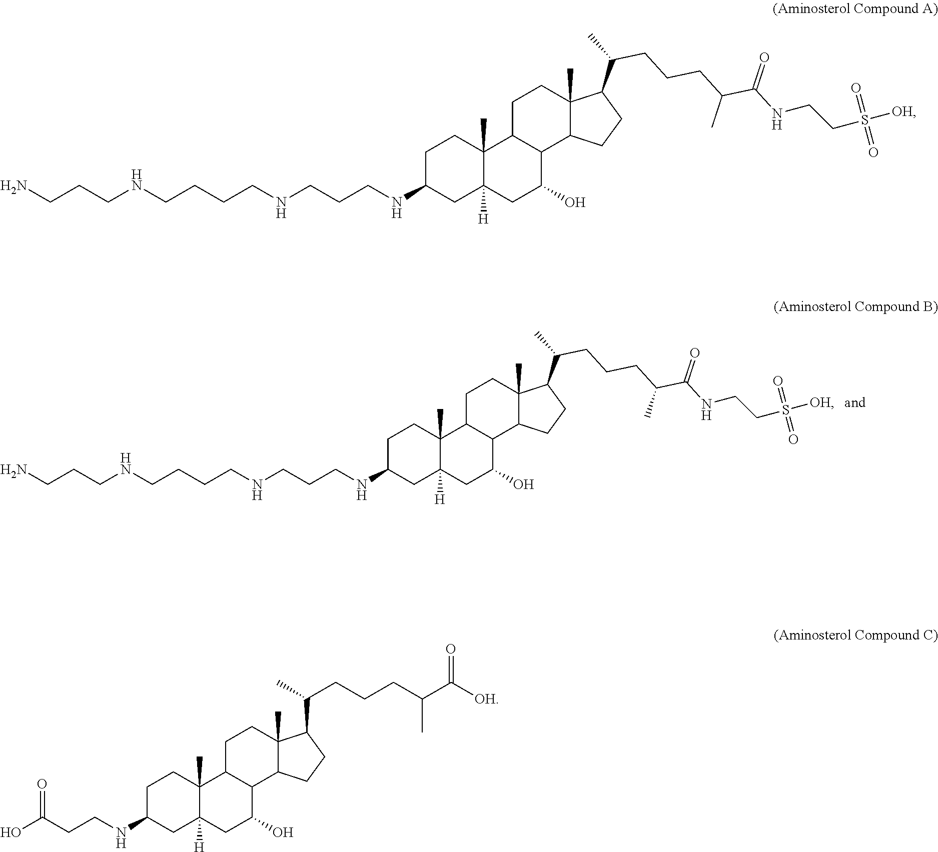 [SMILES: CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; "and" ; CC(CCC[C@@H](C)[C@H]1CCC2C3C(CC[C@@]21C)[C@@]1(C)CC[C@H](NCCC(=O)O)C[C@@H]1C[C@H]3O)C(=O)O]

Enantiomers (R and S), and racemic mixtures, of all aminosterol compounds disclosed herein are encompassed by the present invention.

In another aspect, disclosed is the aminosterol compound having the following chemical structure (Formula (I))

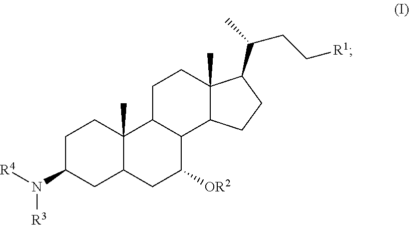

wherein:
R¹ is an optionally substituted C₁-C₈ alkyl, optionally substituted 2 to 8-membered heteroalkyl, optionally substituted heterocyclyl, or optionally substituted C₃-C₈ cycloalkyl;
R¹ is H or —C(O)R⁵, wherein R⁵ is an optionally substituted aryl, optionally substituted heteroaryl, optionally substituted C-C-alkyl, optionally substituted heterocyclyl, or optionally substituted C₃-C₈ cycloalkyl; and
R³ and R⁴ are each independently H or an optionally substituted 2 to 14-membered heteroalkyl, or a pharmaceutically acceptable salt, solvate, prodrug, or derivative thereof;
wherein the compound is not:
   (1) (24R)-3β-((3-[(3-Aminopropyl)amino]propyl}amino)-7α-hydroxycholestan-24-yl hydrogen sulfate (squalamine);
   (2) (3R,6R)-6-((3S,5R,7R,8R,9S,10S,13R,14S,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2-methylheptan-3-yl hydrogen sulfate (aminosterol 1436);
   (3) (6R)-6-((3S,5R,7R,10S,13R,17R)-3-((3-((4((3-aminopropyl)amino)butyl)amino)propyl)amino)-7-hydroxy-10,13-dimethylhexadecahydro-1H-cyclopenta[a]phenanthren-17-yl)-2-methylheptanoic acid (ENT-03);
   (4) (2R,6R)-6-((3S,5R,7R,10S,13R, 17R)-3((3-(4-((3-aminopropyl)amino)butyl)amino)propyl)amino)-7-hydroxy-10,13-dimethylhexadecahydro-1-1-cyclopenta[a]phenanthren-17-yl-2-methylheptanoic acid (C₂₅ (R) ENT-03);
   (5) (6R)-6-((3S,5R,7R, 10S,13R,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2-methylheptanoic-2,3,3-d3 acid (ENT-03 d3):
   (6) (6R)-6-((3S,5R,7R,10S,13R,17R)-3-((3-((4-((3-aminopropyl)amino)butyl)amino)propyl)amino)-7-(benzoyloxy)-10,13-dimethylhexadecahydro-1H-cyclopenta[a]phenanthren-17-yl)-2-methylheptanoic acid;
   (7) (6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
   (8) (6R)-6-((3R,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid:
   (9) (6R)-6-((3R,5S,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylheptanoic acid;
   (10) (6R)-6-((3R,5S,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methylheptanoic acid;
   (11) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic-2-d acid;
   (12) (6R)-6-((3S,5R,7R,10S,13R,17R)-3-((3-((4-((3-aminopropyl)amino)butyl)amino)propyl)amino)-7-hydroxy-10,]3-dimethylhexadecahydro-1 H-cyclopenta[a]phenanthren-17-yl)-2-methylheptanoic-2-.d acid;
   (13) (6R)-6-((3S,5R,7R,10S,13R,17R)-3-((3-((4-aminobutyl)amino)propyl)amino)-7,12-dihydroxy-10,13-dimethylhexadecahydro-11H-cyclopenta[a]phenanthren-17-yl)-2-methylheptanoic-2-d acid;
   (14) (6R)-6-((3S,5R,7R,10S,131,17R)-3-((3-((4-((3-aminopropyl)amino)butyl)amino)propyl)amino)-7,12-dihydroxy-10,13-dimethylhexadecahydro-1H-cyclopenta[a]phenanthren-1 7-yl)-2-methylheptanoic-2-d acid;
   (15) (6R)-6-((3S,5R,7R,10S,13R,1 7K)-3-((3-((4-aminobutyl)amino)propyl)amino)-7-hydroxy-10,13-dimethylhexadecahydro-1H-cyclopenta[a]phenanthren-17-yl)-2-(methyl-d3)heptanoic acid;
   (16) (6R)-6-((3S,5R,7R,10S,13R,17R)-3-((3-((4-((3-aminopropyl)amino)butyl)amino)propyl)amino)-7-hydroxy-10,13-dimethylhexadecahydro-11-1-cyclopenta[a]phenanthren-17-yl)-2-(methyl-d3)heptanoic acid;
   (17) (6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-(methyl-d3)heptanoic acid;
   (18) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-(methyl-d3)heptanoic acid;
   (19) (6R,E)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylhept-2-enoic acid;
   (20) (6R,E)-6-((3S,5R,7R,10S,13R17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylhept-2-enoic acid;
   (21) (6R,E)-6-((3S,5R,7R,10S,13R,17R)-3-((3-((4-aminobutyl)amino)propyl)amino)-7,12-dihydroxy-10,13-dimethylhexadecahydro-1-1-cyclopenta[a]phenanthren-1 7-yl)-2-methylhept-2-enoic acid;
   (22) (6R,E)-6-((3S,5R, 7R,10S,13R,17R)-3-((3-((4-((3-aminopropyl)amino)butyl)amino)propyl)amino)-7, 12-dihydroxy-10, 13-dimethylhexadecahydro-1H-cyclopenta[a]phenanthren-17-yl)-2-methylhept-2-enoic acid;
   (23) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-3-hydroxy-2-methylheptanoic acid;
   (24) (6R)-6-((3S,5R,R,10S,13R,17R)-3-((3-((4-((3-aminopropyl)amino)butyl)amino)propyl)amino)-7-hydroxy-10,13-dimethylhexadecahydro-11H-cyclopenta[a]phenanthren-17-y)-3-hydroxy-2-methylheptanoic acid;
   (25) (6R)-6-((3S,5R,7R,1 0S,13R,17R)-3-((3-((4-aminobutyl)amino)propyl)amino)-7,12-dihydroxy-10,13-dimethylhexadecahydro-1H-cyclopenta[a]phenanthren-17-yl)-3-hydroxy-2-methylheptanoic acid;
   (26) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-3-hydroxy-2-methylheptanoic acid;
   (27) (6R)-6-((3S,5R,7R,10S,13R,17R)-3-((3-((4-aminobutyl)amino)propyl)amino)-7-hydroxy-10,13-dimethylhexadecahydro-HII-cyclopenta[a]phenanthren-17-yl)heptan-3-one;
   (28) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)heptan-3-one;
   (29) (6R)-6-((3S,5R,7R,10S,13R,17R)-3-((3-((4-aminobutyl)amino)propyl)amino)-7,12-dihydroxy-10,13-dimethylhexadecahydro-H-cyclopenta[a]phenanthren-17-yl)heptan-3-one;
   (30) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)heptan-3-one;
   (31) (6R)-6-((3S,5R,7R,10S,13R,17R)-3-((3-((4-aminobutyl)amino)propyl)amino)-7-hydroxy-10,13-dimethylhexadecahydro-1I-cyclopenta[a]phenanthren-17-yl)-2-methyl-3-oxoheptanoic acid;
   (32) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoic acid;
   (33) (6R)-6-((3S,5R,7R,10S,13R,1 7R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoic acid;
   (34) (6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-1 7-yl)-2-methyl-3-oxoheptanoic acid;
   (35) methyl(6R)-6-((3S,5R,7R,10S,13R,17R)-3-((3-((4-aminobutyl)amino)propyl)amino)-7-hydroxy-10,13-dimethylhexadecahydro-1 H-cyclopenta[a]phenanthren-17-yl)-2-methyl-3-oxoheptanoate;
   (36) methyl(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoate;
   (37) methyl(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoate;
   (38) methyl(6R)-6-((3 S,5R,7R,10S, 13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoate;
   (39) isopropyl(6R)-6-((3S,5R,7R,10S,13R,17R)-3-((3-((4-aminobutyl)amino)propyl)amino)-7-hydroxy-10,13-dimethylhexadecahydro-11H-cyclopenta[a]phenanthren-17-yl)-2-methyl-3-oxoheptanoate;
   (40) isopropyl(6R)-6-((35,5R,7R,10S,13R,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2-methyl-3-oxoheptanoate;
   (41) isopropyl(6R)-6-((3S,5R,7R,10S,13R,17R)-3-((3((4-aminobutyl)amino)propyl)amino-7,12-dihydroxy-10,13-dimethylhexadecahydro-1 H-cyclopenta[a]phenanthren-17-yl)-2-methyl-3-oxoheptanoate;
   (42) isopropyl(61)-6((3 S,5R,7R,10S,13R,17R)-3-((3-((4-(3-aminopropyl)amino)butyl)amino)propyl)amino)-7,12-dihydroxy-10,13-dimethylhexadecahydro-114-cyclopenta[a]phenanthren-1 7-yl)-2-methyl-3-oxoheptanoate;
   (43) (6R)-6-((3S,5R,7R,10S,13R,17R)-3-((3-((4-aminobutyl)amino)propyl)amino)-7-hydroxy-10,13-dimethylhexadecahydro-1-cyclopenta[a]phenanthren-17-yl)-2,3-dihydroxy-2-methylheptanoic acid;
   (44) (6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2,3-dihydroxy-2-methylheptanoic acid;
   (45) (6R)-6-((35,5R7R,10S,13R,17R)-3-((3-((4-aminobutyl)amino)propyl)amino)-7,12-dihydroxy-10,13-dimethylhexadecahydro-11-1-cyclopenta[a]phenanthren-1 7-yl)-2,3-dihydroxy-2-methylheptanoic acid;
   (46) (6R)-6-((3S,5R,7R,10S,13R,17R)-3-((3-((4-((3-aminopropylamino)butyl)amino)propyl)amino)-7,12-dihydroxy-10,13-dimethylhexadecahydro-1H-cylopenta[a]phenanthren-17-yl)-2,3-dihydroxy-2-methylheptanoic acid;
   (47) (6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptane-2,3-diol;
   (48) (6R)-6-((3S,5R,7R,10S,13R,17R)-3-((3-((4-((3-aminopropyl)amino)butyl)amino)propyl)amino)-7-hydroxy-10,13-dimethylhexadecahydro-1 H-cyclopenta[a]phenanthren-1′7-yl)-2-methylheptane-2,3-diol;
   (49) (3S,5R,7R,10S,13R,17R)-3-((3-((4-aminobutyl)amino)propyl)amino)-17-((2R)-5,6-dihydroxy-6-methylheptan-2-yl)-10,13-dimethylhexadecahydro-1H-cyclopenta[a]phenanthrene-7,12-diol;
   (50) (3S,5,7R,10 S,13R,17R)-3-((3-((4-((3-aminopropyl)amino)butyl)amino)propyl)amino)-17-((2R)-5, 6-dihydroxy-6-methylheptan-2-yl)-10,13-dimethylhexadecahydro-1H-cyclopenta[a]phenanthrene-7,12-diol;
   (51) (6R)-6-((3S,5R,7R,10S,13R,17R)-3-((3-((4-aminobutyl)amino)propyl)ammonio)-7,12-dihydroxy-10,13-dimethylhexadecahydro-1-cyclopenta[a]phenanthren-17-yl)-2-methylheptanoate (ENT-05);
   (52) (6R)-6-((3S,5R,7R,10S,13R,17R)-3-((3-((4-aminobutyl)ammonio)propyl)ammonio)-7-hydroxy-10,13-dimethylhexadecahydro-11-cyclopenta[a]phenanthren-17-yl)-2-methylheptanoate (ENT-06);
   (53) (6R)-6-((3S,5R,7R,10S,13R,17R)-3-((3-((4-((3-ammonio propyl)amrinone)butyl)amrinone)propyl)ammonio)-7-hydroxy-10,13-dimethylhexadecahydro-11H-cyclopenta[a]phenanthren-17-yl)-3-hydroxy-2-methylheptanoate-2,3-d2;
   (54) (2S,6R)-6-((3S,5R,7R,10S,13R,17R)-3-((3-((4-((3-aminopropyl)amino)butyl)amino)propyl)amino)-7-hydroxy-10,13-dimethylhexadecahydro-1 H-cyclopenta[a]phenanthren-17-yl)-2-methylheptanoic acid (C₂₅ (S) ENT-03);
   (55) (2R,6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
   (56) (2S,6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
   (57) (2R,6R)-6-((3S,5R,7R10S,13R,17R)-3-((3-((4-aminobutyl)amino)propyl)amino)-7-hydroxy-10,13-dimethylhexadecahydro-1H-cyclopenta[a]phenanthren-1 7-yl)-2-methylheptanoic acid (C₂₅ (R) ENT-06),
   (59) (2S,6R)-6-((3S,5R,7R,10S,13R,17R)-3-((3-((4-aminobutyl)amino)propyl)amino)-7-hydroxy-10,13-dimethylhexadecahydro-1-cyclopenta[a]phenanthren-17-yl)-2 methylheptanoic acid (C₂₅ (S) ENT-06);
   (60) (6R)-6-((3S,5R,7R,105,13R)-3-((3-((4-aminobutyl)amino)propyl)amino)-7-hydroxy-10,13-dimethylhexadecahydro-11-H-cyclopentane[a]phenanthren-17-yl)-2-(methyl-d3)heptan-3-yl-1,1,1,2-d4 hydrogen sulfate (D7-squalamine);
   (61) (3R,6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptan-3-yl-3-d hydrogen sulfate (24-D₁-squalamine); or
   (62) (6R)-6-((3S,5R,7R,10S,13R,17R-3-((3-((4-((3-aminopropyl)amino)butyl)amino)propyl)amino)-7-hydroxy-10,13-dimethylhexadecahydro-1H-cyclopenta[a]phenanthren-17-yl)-2-(methyl-d3)heptan-3-yl-1,1,1 ,2-d4 hydrogen sulfate (D-1436).

In another aspect, the aminosterol has the structure of Formula (I), wherein R¹ is selected from

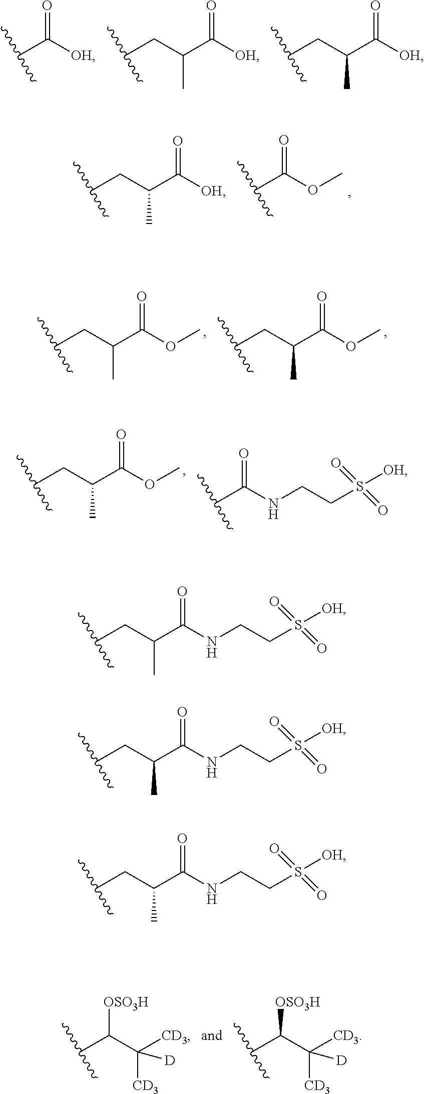

In another aspect, the aminosterol has the structure of Formula (I) wherein R² is H.

In another aspect, the aminosterol has the structure of Formula (I), wherein R³ and R⁴ are independently selected from H,

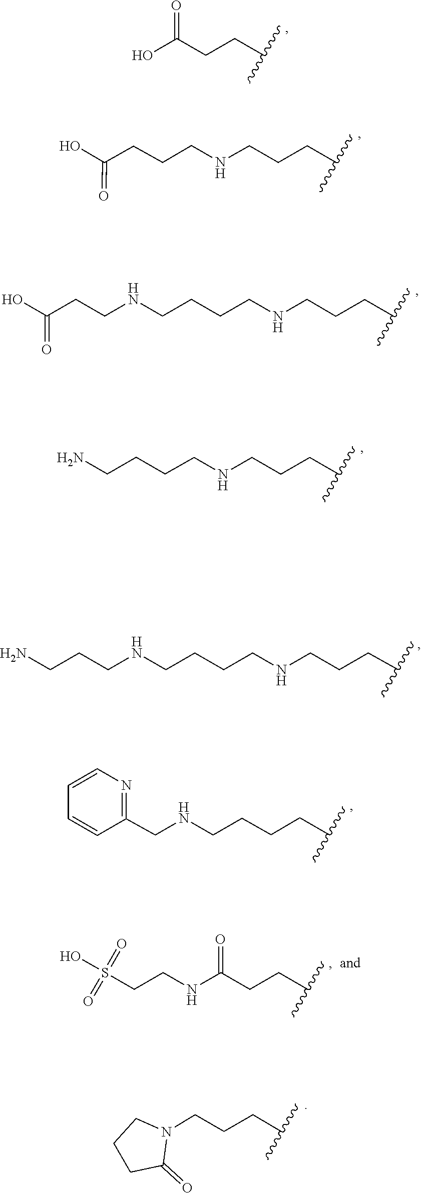

In another aspect, the aminosterol has the structure of, Formula (I), wherein R³ is H and R⁴ is independently selected from

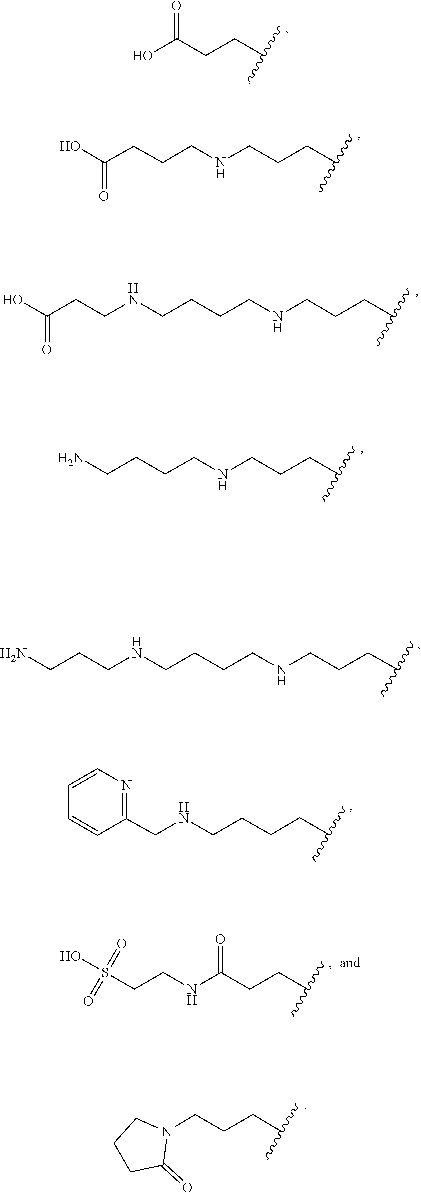

In some embodiment, the compound as disclosed herein has a diastereomeric excess (de), with respect to the C₂₅ carbon is about 80% to about 90%, about 90% to about 95%, about 95% to about 99%, about 99% to about 99.9%, or about 100%.

Given the chemical structure, the aminosterol compounds described herein can be made using standard chemical synthesis techniques. For example, coupling of polyamines to 3-ketosteroids is described in US-2023/0123701, as well as control of the C25 stereochemistry. Preparation of the described aminosterol molecules can also be accomplished by synthesis of the required polyamine and 3-keto steroids by methods standard in the art.

### III. Deuterated Aminosterol Compounds

In another aspect of the disclosure, described are deuterated aminosterol compounds, where at least one hydrogen of an aminosterol compound described herein is replaced with deuterium. A deuterated form of an aminosterol may have improved safety, better tolerability and/or enhanced efficacy. Deuterium is a naturally-occurring, stable, non-radioactive isotope of hydrogen. The aminosterol can be any compound described herein, including but not limited to ENT-01, squalamine phosphate, ENT-02 (aminosterol 1436), ENT-03, ENT-03S (C₂₅ (S) ENT-03), ENT-03R (C₂₅ (R) ENT-03), ENT-03 mixed isomers, ENT-04, ENT-05, ENT-06, ENT-07, ENT-08, ENT-09, ENT-10, ENT-11, ENT-12, ENT-13, ENT-14, ENT-14S, ENT-14R, ENT-15, ENT-16, ENT-17 (also known as M1-PU), ENT-1S, ENT-18S, ENT-18R, ENT-19 (ENT-19 is the racemic version of M10-PU), ENT-19S, ENT-20, ENT-21, ENT-21S, ENT-2IR, ENT-21 mixed isomers, ENT-22, ENT-22S, ENT-22R, ENT-22 mixed isomers, ENT-23, ENT-24, ENT-25, ENT-25R, ENT-25S (also known as M1I-RB), ENT-25 mixed isomers, ENT-2 ENT-27, ENT-28, ENT-29, ENT-30, ENT-31, ENT-32, ENT-33, M10-PU/M14-RB, M-2-RB, M3-RB, M8-RB, MS-RB, M16-RB, Aminosterol Compound A, Aminosterol Compound B, and Aminosterol Compound C, or a pharmaceutically acceptable salt, solvate, prodrug, enantiomer, or derivative thereof.

Deuterated aminosterols, where one or more of the hydrogen atoms contained in the aminosterol molecule have been replaced by its heavier stable isotope deuterium, are anticipated to have a lower rate of metabolism and therefore a longer half-life and/or reduced or less frequent dosing to obtain a desired therapeutic effect. Deuterium-carbon bonds are generally about six to 10 times more stable than the corresponding hydrogen-carbon bond, These stronger bonds are more difficult to break, which can slow the rate of bond cleavage. This effect upon rate is called the kinetic isotope effect (KIE).

Deuteration of aminosterols can produce compounds having improved pharmacokinetic and/or toxicological properties, such as improved safety, efficacy and/or tolerability, as compared to unmodified aminosterols, due the stronger deuterium-carbon bond modifying the metabolism of the aminosterols.

In another aspect, for the deuterated aminosterol compounds described herein, any atom not designated as deuterium is present at its natural isotopic abundance. In another embodiment, the deuterium incorporation at each designated deuterium atom is at least about 90%, at least about 95%, or at least about 97%.

In yet another embodiment, the deuterated aminosterol compounds of the invention have an isotopic enrichment factor selected from the group consisting of at least about 3500, at least about 4000, at least about 4500, at least about 5000, at least about 5500, at least about 6000, at least about 6333.3, at least about 6466.7, at least about 6600, and at least about 6633.3. In one embodiment, the compounds of the invention have an isotopic enrichment factor of at least 3500 for one deuterium at a single position of the compound.

In one embodiment, the deuterated aminosterol compounds of the invention are a pharmaceutically acceptable salt, such as a phosphate salt.

In one embodiment, the deuterated aminosterol compounds of the invention have a longer half-life as compared to an undeuterated form of the same aminosterol. For example, the half-life can be increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 902”, about 95%, or about 100%.

In one aspect in the methods of the invention, the deuterated aminosterol compound has an improved safety profile, as measured by a decrease in incidence of one or more adverse events evaluated using a clinically recognized scale or tool, as compared to the same aminosterol compound which has not been deuterated. For example, the safety profile can be improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect in the methods of the invention, the deuterated aminosterol compound has an improved efficacy as compared to the same aminosterol compound which has not been deuterated. For example, the improved efficacy can be measured by improvement of one or more disease symptoms evaluated using a clinically recognized scale or tool. For example, the efficacy improvement can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%/0, about 95%, or about 100%.

In another aspect in the methods of the invention, the deuterated aminosterol compound has an improved tolerability, measured using a clinically recognized scale or tool, as compared to the same compound which has not been deuterated. For example, the tolerability can be improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another aspect in the methods of the invention, the deuterated aminosterol compound has a reduced required dosage amount and/or dosing frequency, as compared to the same aminosterol compound which has not been deuterated, to obtain the same or improved desired therapeutic effect as measured using a clinically recognized scale or tool. For example, a deuterated aminosterol composition according to the invention, can have a dosage about 5% less than the same but unmodified aminosterol to obtain the same therapeutic effect. In other embodiments, the dosage is about 10%, about 15%, about 20%, about 25%, about 30%, about 35%, about 40%, about 45%, or about 50% less than that of the same but unmodified aminosterol to obtain the same or improved therapeutic effect.

### IV. Aminosterol-Cyclodextrin Formulations

In one aspect, the invention disclosed describes a method of “masking” the aminosteroid in such a way as to eliminate its membrane disruptive property during its residence at the site of injection, thereby eliminating local pain, tissue damage, and improving bioavailability. The “masking” of an aminosterol is accomplished by mixing an aqueous solution of an aminosterol, or a pharmaceutically acceptable salt, solvate, prodrug, or derivative thereof, with an aqueous solution of a cyclodextrin of the appropriate configuration. Upon mixing, a molecule of the cyclodextrin will reversibly bind to a molecule of the aminosterol, or a pharmaceutically acceptable salt, solvate, prodrug, or derivative thereof, to form a cyclodextrin-aminosterol complex.

As is well known in the field, cyclodextrins have a 3-dimensional cup-like structure that permits them to encase (capture) smaller organic compounds. The biophysical properties of the resulting complex is usually dominated by the biophysical characteristics of the cyclodextrin.

Cyclodextrins are comprised of glucose molecules linked end-to end to form circular structures, As such, they exhibit the properties expected for oligosaccharides, namely high water solubility and excellent tissue compatibility. For this reason, cyclodextrins are widely used in the pharmaceutical industry to solubilize compounds that have poor water solubility.

In the present disclosure, cyclodextrins serve to “mask” the detergency of the aminosterol, or a pharmaceutically acceptable salt, solvate, prodrug, or derivative thereof, at the site of injection. To achieve this result, the concentration of the cyclodextrin in the formulation must be increased to drive the equilibrium towards full complexation of the aminosterol, or a pharmaceutically acceptable salt, solvate, prodrug, or derivative thereof. Since the complex between the cyclodextrin and aminosterol, or a pharmaceutically acceptable salt, solvate, prodrug, or derivative thereof, is non-covalent, the components exist as free molecules and complex in an equilibrium determined by the stability of the complex, and the concentrations of the free molecules. As the complex diffuses from the site of injection and enters the systemic circulation, the concentration of the cyclodextrin decreases and the complex dissociates, liberating the aminosterol, or a pharmaceutically acceptable salt, solvate, prodrug, or derivative thereof.

Cyclodextrins exist in three size classes based on their number of glucose units: Alpha, 6; Beta, 7; and Gamma, 8. The “cup” size of the cyclodextrin increases as the number of glucose units increase. In addition, the cyclodextrin can be chemically decorated with substituents such as alkyl hydroxy groups, anionic and cationic groups, which can further influence complex formation with an intended ligand.

Numerous methods have been described to determine the extent of complex formation using procedures such as isothermal calorimetry and NMR. In the present disclosure, the optimal cyclodextrin concentration required to fully mask an aminosterol, or a pharmaceutically acceptable salt, solvate, prodrug, or derivative thereof, can be approximated by titrating a solution of cyclodextrin to an aqueous solution of the aminosterol to a concentration where the solution no longer bubbles when agitated. Since “bubbling” reflects a reduction in surface tension, and aminosterols have detergent properties, when the concentration of free aminosterol falls below a few μg/ml, then robust “bubbling” disappears. Further adjustment of the cyclodextrin concentration is then based on the clinical response to an injection of the aminosterol formulation to a subject. If a subcutaneous injection is painful, then the concentration of the cyclodextrin can be increased.

The high concentration of aminosteroid that must be formulated requires that the cyclodextrin have high water solubility. In addition, at these high concentrations the cyclodextrin selected must not exhibit aggregation in solution, a property commonly observed with natural cyclodextrins. Only very specific chemically modified cyclodextrins will serve as satisfactory carriers.

An exemplary cyclodextrin is 2HydroxyPropylβ-Cyclodextrin (2HIP-β-CD). 2HIP-β-CD is a β-CD) chemically decorated with hydroxypropyl moieties. The “cup size” is sufficiently large to accommodate the ENT-03 molecule, or a pharmaceutically acceptable salt, solvate, prodrug, or derivative thereof. The stability provided by the hydrophobic interactions permits complex formation at concentrations of the cyclodextrin. Upon dilution into tissue fluids, the hydrophobic bonds are sufficiently weak to permit dissociation, which is further promoted by the binding of the aminosteroid to plasma proteins. 2HP-β-CID is water soluble to concentrations in excess of 60%, permitting the formulation of high concentrations of the aminosteroid.

Other exemplary cyclodextrins include, but are not limited to, chemically modified or unmodified beta and gamma cyclodextrins. Alpha cyclodextrins are unlikely to be suitable due to their small “cup” size. Exemplary chemically modified or unmodified beta and gamma cyclodextrins include, for example, beta cyclodextrin, gamma cyclodextrin, Hydroxypropyl-β-cyclodextrin, Sulfobutyl ether-β-cyclodextrin, Randomly methylated-β-cyclodextrin, Hydroxypropyl-γ-cyclodextrin, as well as polymerized cyclodextrins such as Epichlorohydrin-β-cyclodextrin and Carboxy methyl epichlorohydrin beta cyclodextrin. In one aspect of the disclosure the cyclodextrin utilized in the aminosterol formulation is cited in the FDA's list of Inactive Pharmaceutical Ingredients and/or categorized on the generally regarded as safe (GRAS) list of the FDA.

Any aminosterol described in the present disclosure, as well as aminosterols disclosed in WO 2021/025973 and US 2023/0123701 (both for “Human Aminosterol ENT-03 Compounds, Related Compositions Comprising The Same, And Methods Of Using The Same”), WO 2021/025974 and US 2022/0372066 (both for “Human Squalamine Derivatives, Related Compositions Comprising The Same, And Methods Of Using The Same”), and US 2023/0125585 (for “Dosing Protocols and Regimens for Aminosterol Treatment), all of which are specifically incorporated by reference, as well as pharmaceutically acceptable salts, solvates, prodrugs, enantiomers, or derivatives thereof, can be utilized in the described cyclodextrin formulations. The aminosterol can also be present in a racemic mixture.

Exemplary aminosterols that can be utilized in the formulations herein also include, but are not limited to, any one of ENT-01, squalamine phosphate, ENT-02 (aminosterol 1436), ENT-03, ENT-03S (C₂₅ (S) ENT-03), ENT-03R (C₂₅ (R) ENT-03), ENT-03 mixed isomers, ENT-04, ENT-05, ENT-06, ENT-07, ENT-08, ENT-09, ENT-10, ENT-11, ENT-12, ENT-13, ENT-14, ENT-14S, ENT-14R, ENT-15, ENT-16, ENT-17 (also known as M1-PU), ENT-18, ENT-18S, ENT-18R, ENT-19 (ENT-19 is the racemic version of M10-PU), ENT-19S, ENT-20, ENT-21, ENT-21S, ENT-21R, ENT-21 mixed isomers, ENT-22, ENT-22S, ENT-22R, ENT-22 mixed isomers, ENT-23, ENT-24, ENT-25, ENT-25R, ENT-25S (also known as M1 1-RB), ENT-25 mixed isomers, ENT-26, ENT-27, ENT-28, ENT-29, ENT-30, ENT-31, ENT-32, ENT-33, M10-PU/M14-RB, M2-RB, M3-RB, M8-RB, M5-RB, M16-RB, Aminosterol Compound A, Aminosterol Compound B, and Aminosterol Compound C, or a pharmaceutically acceptable salt, solvate, prodrug, enantiomer, or derivative thereof, with the structure of each compound shown below:

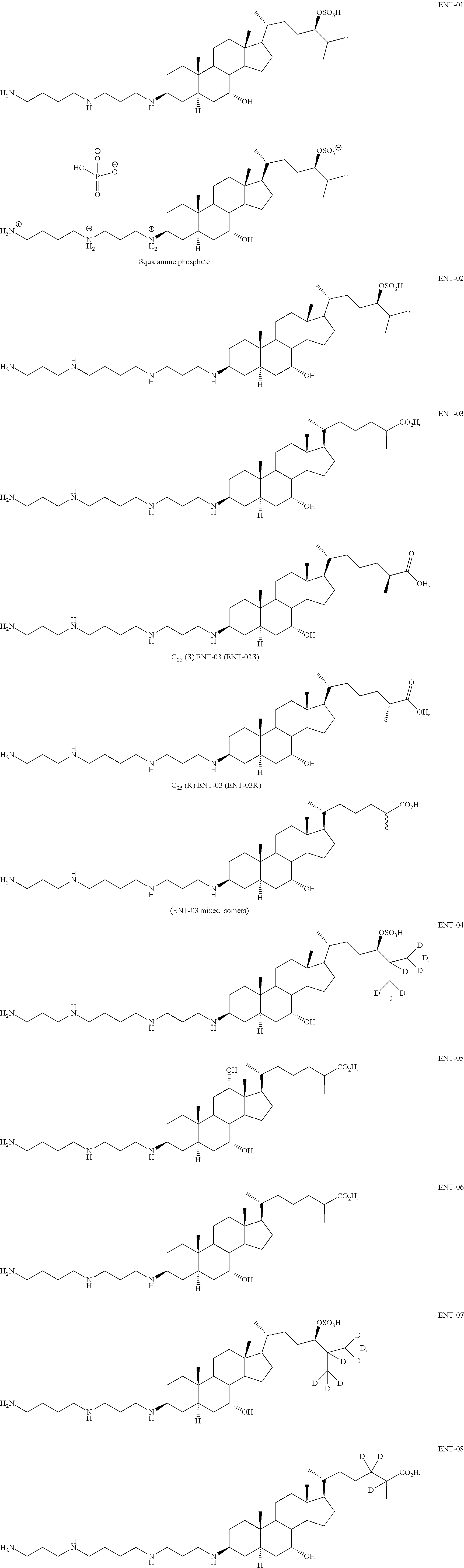

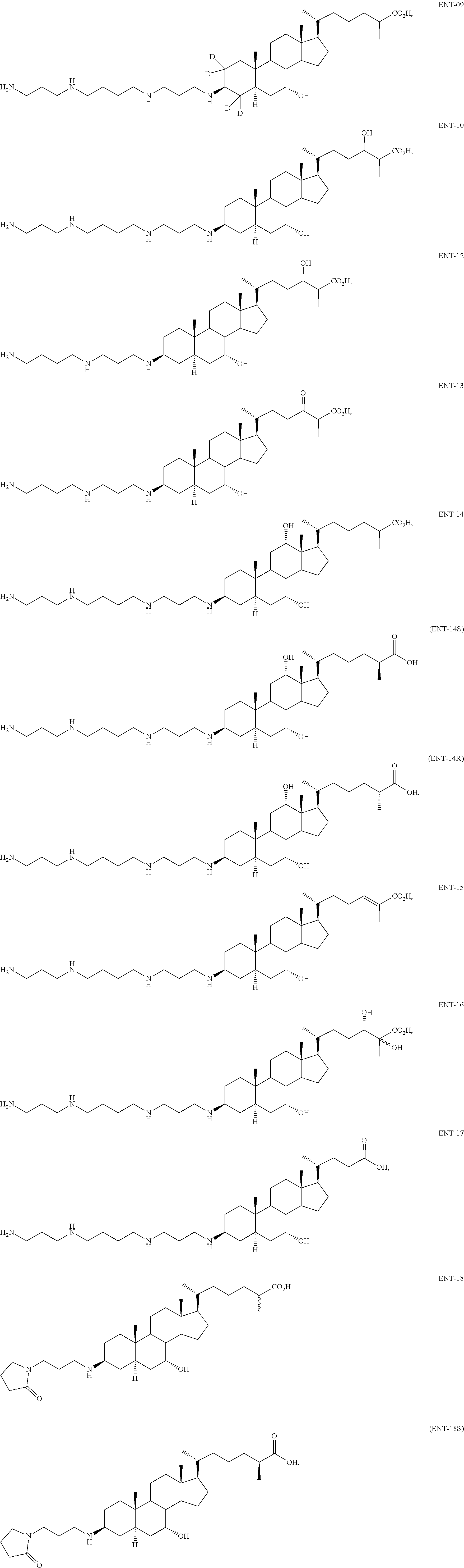

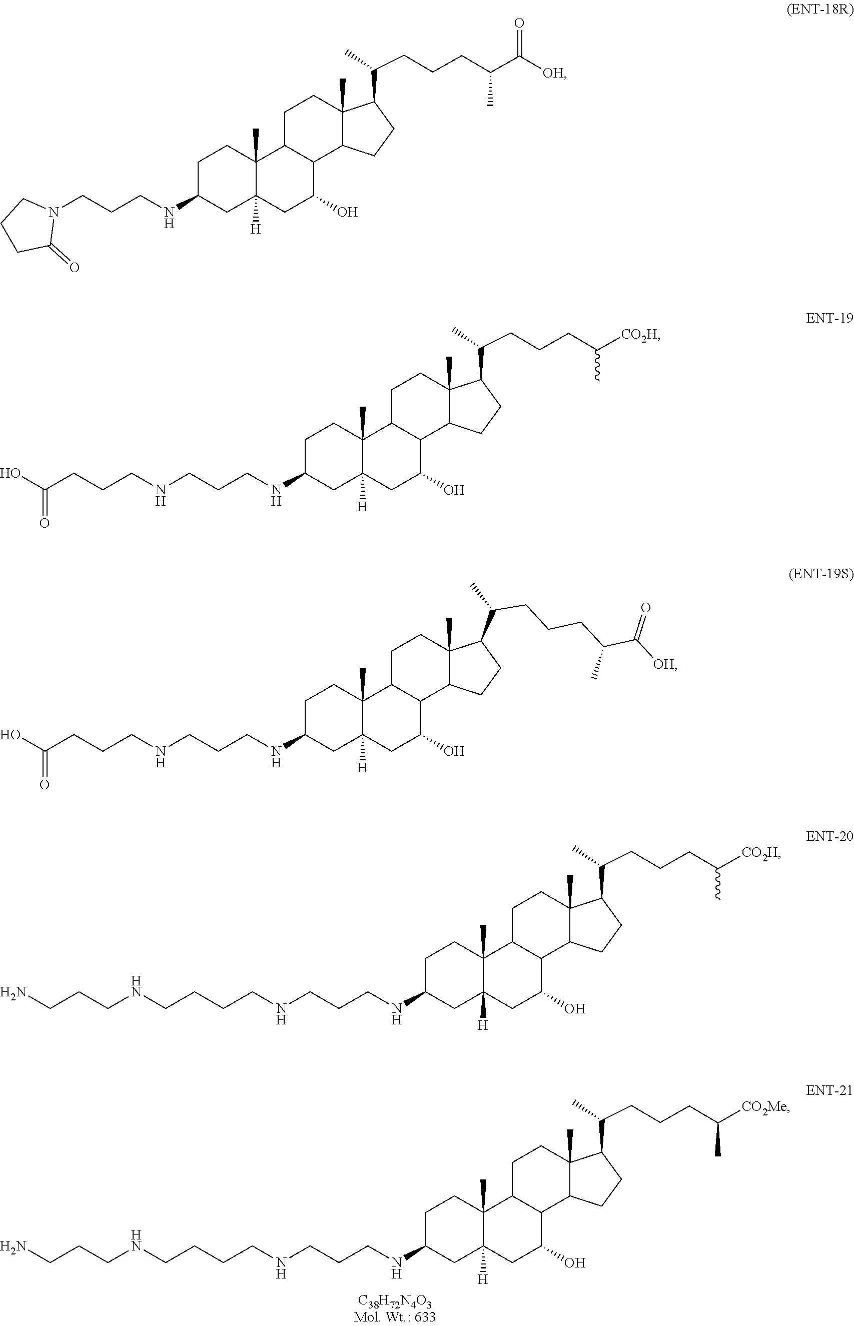

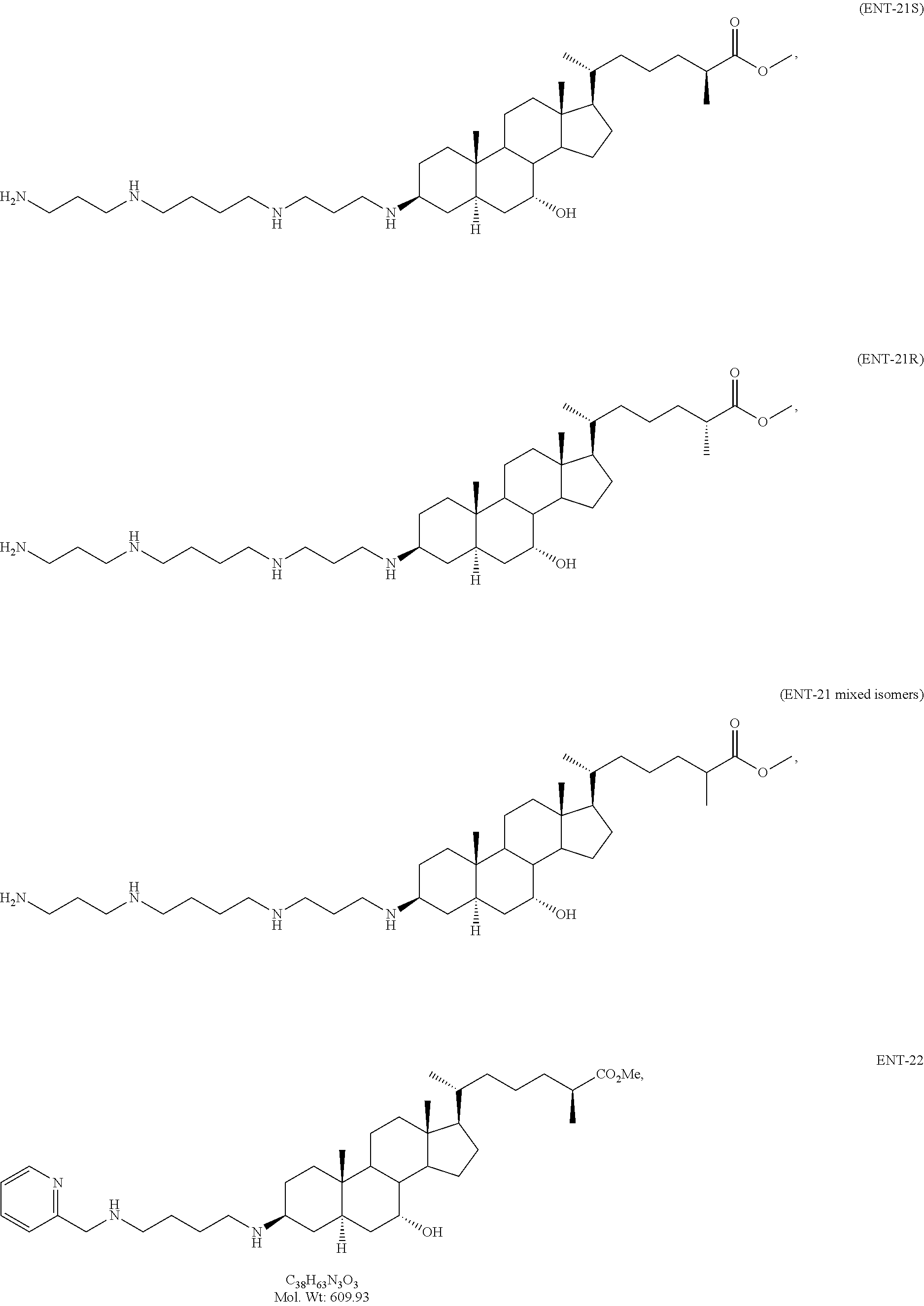 [SMILES: COC(=O)[C@@H](C)CCC[C@@H](C)[C@H]1CCC2C3C(CC[C@@]21C)[C@@]1(C)CC[C@H](NCCCNCCCCNCCCN)C[C@@H]1C[C@H]3O ; COC(=O)[C@H](C)CCC[C@@H](C)[C@H]1CCC2C3C(CC[C@@]21C)[C@@]1(C)CC[C@H](NCCCNCCCCNCCCN)C[C@@H]1C[C@H]3O ; COC(=O)C(C)CCC[C@@H](C)[C@H]1CCC2C3C(CC[C@@]21C)[C@@]1(C)CC[C@H](NCCCNCCCCNCCCN)C[C@@H]1C[C@H]3O ; COC(=O)[C@@H](C)CCC[C@@H](C)[C@H]1CCC2C3C(CC[C@@]21C)[C@@]1(C)CC[C@H](NCCCCNCc2ccccn2)C[C@@H]1C[C@H]3O]

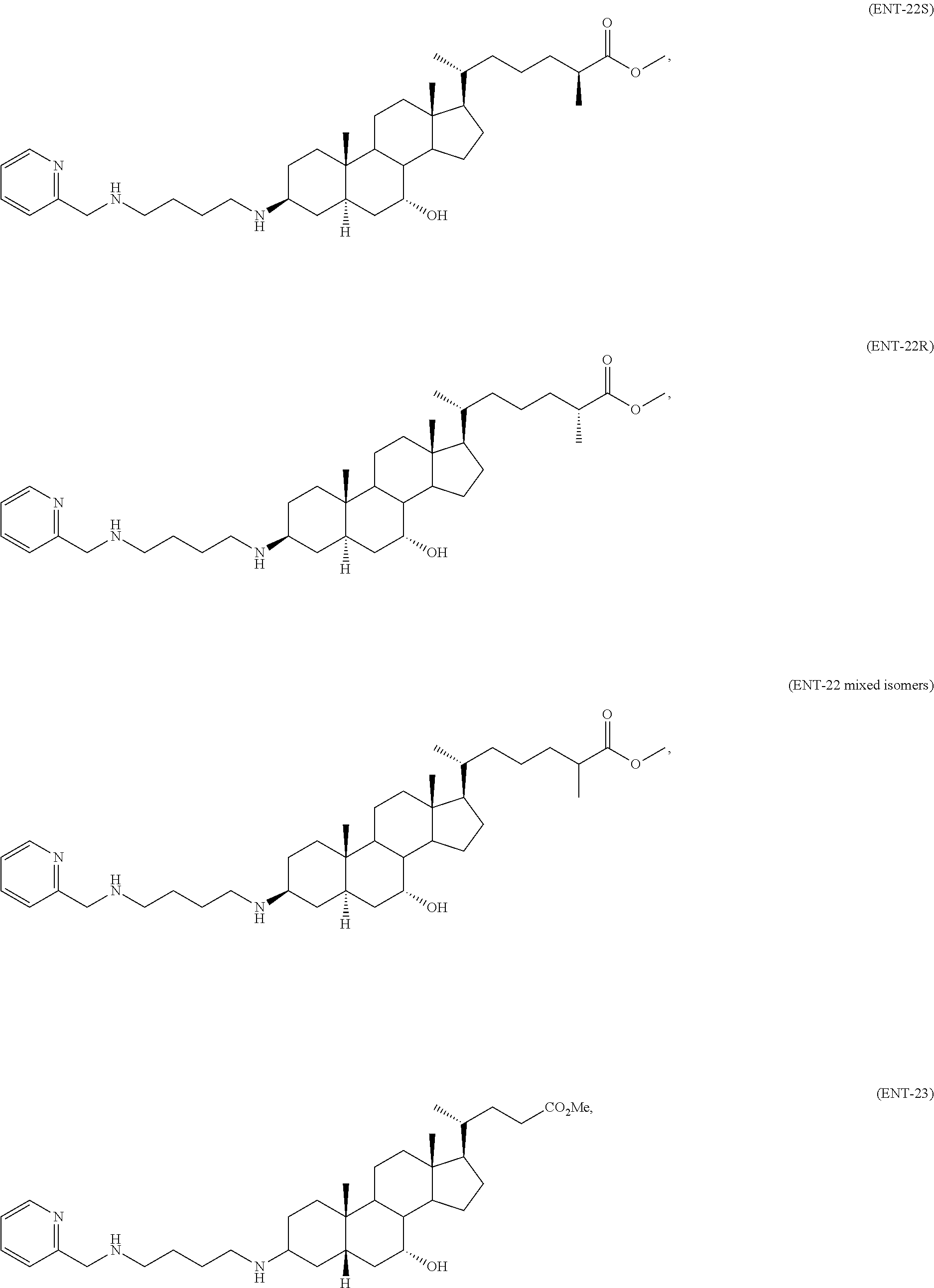 [SMILES: COC(=O)[C@@H](C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)[C@H](C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)C(C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)CC[C@@H](C)[C@H]1CCC2C3C(CC[C@@]21C)[C@@]1(C)CCC(NCCCCNCc2ccccn2)C[C@H]1C[C@H]3O]

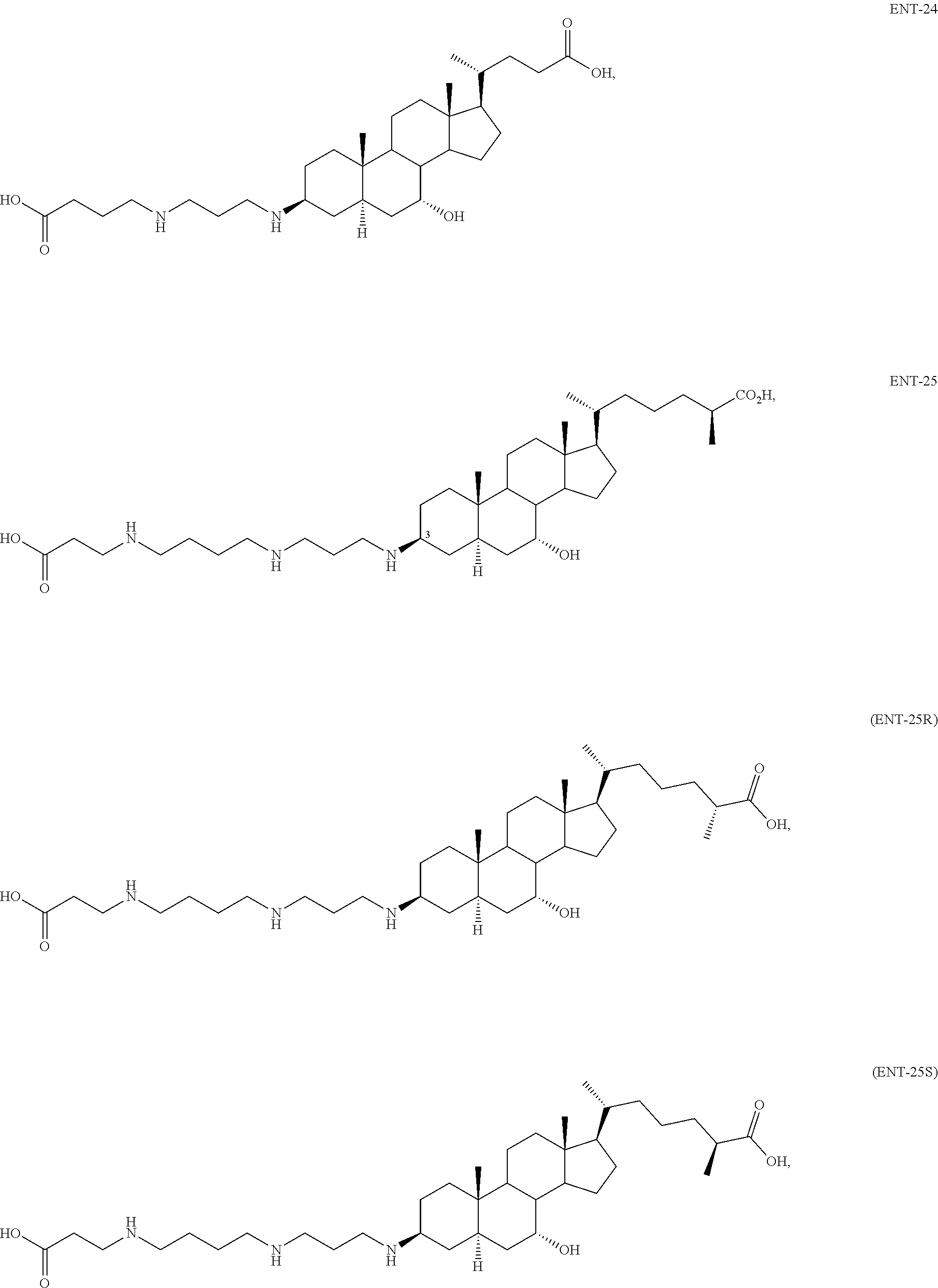 [SMILES: C[C@H](CCC(=O)O)[C@H]1CCC2C3C(CC[C@@]21C)[C@@]1(C)CC[C@H](NCCCNCCCC(=O)O)C[C@@H]1C[C@H]3O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; C[C@H](CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O]

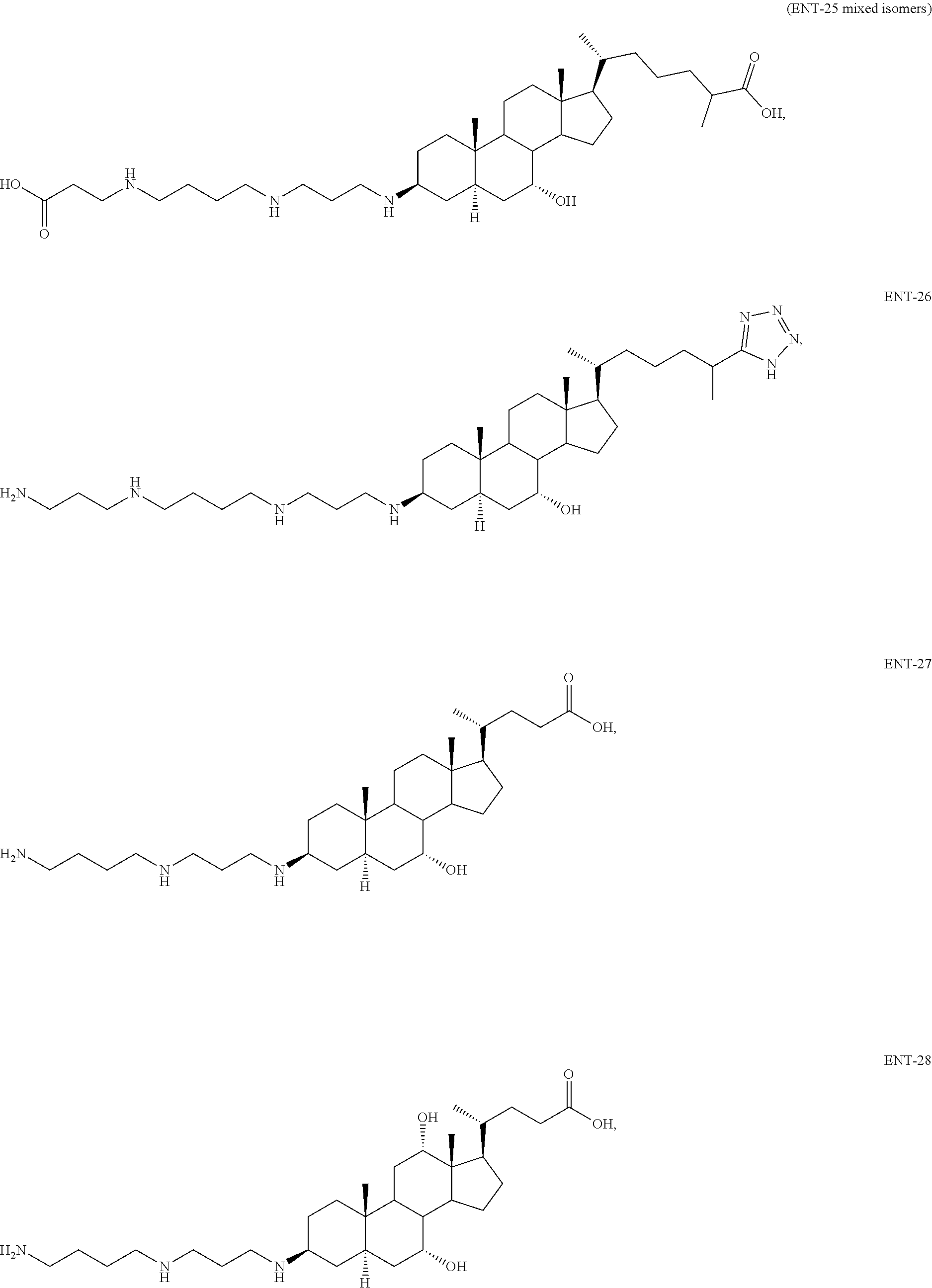 [SMILES: CC(CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O ; CC(CCC[C@@H](C)[C@H]1CCC2C3C(CC[C@@]21C)[C@@]1(C)CC[C@H](NCCCNCCCCNCCCN)C[C@@H]1C[C@H]3O)c1nnn[nH]1 ; C[C@H](CCC(=O)O)[C@H]1CCC2C3C(CC[C@@]21C)[C@@]1(C)CC[C@H](NCCCNCCCCN)C[C@@H]1C[C@H]3O ; C[C@H](CCC(=O)O)[C@H]1CCC2C3C(C[C@H](O)[C@@]21C)[C@@]1(C)CC[C@H](NCCCNCCCCN)C[C@@H]1C[C@H]3O]

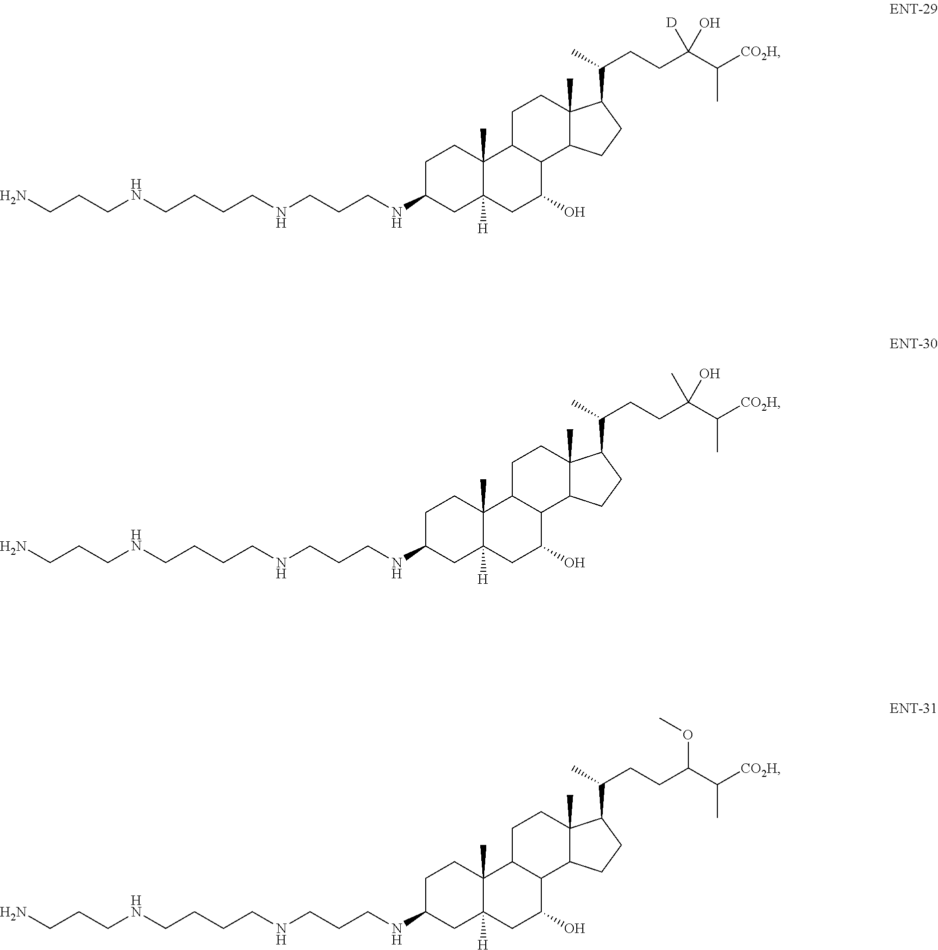 [SMILES: [2H]C(O)(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(C(=O)O)C(C)(O)CC[C@@H](C)[C@H]1CCC2C3C(CC[C@@]21C)[C@@]1(C)CC[C@H](NCCCNCCCCNCCCN)C[C@@H]1C[C@H]3O ; COC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O]

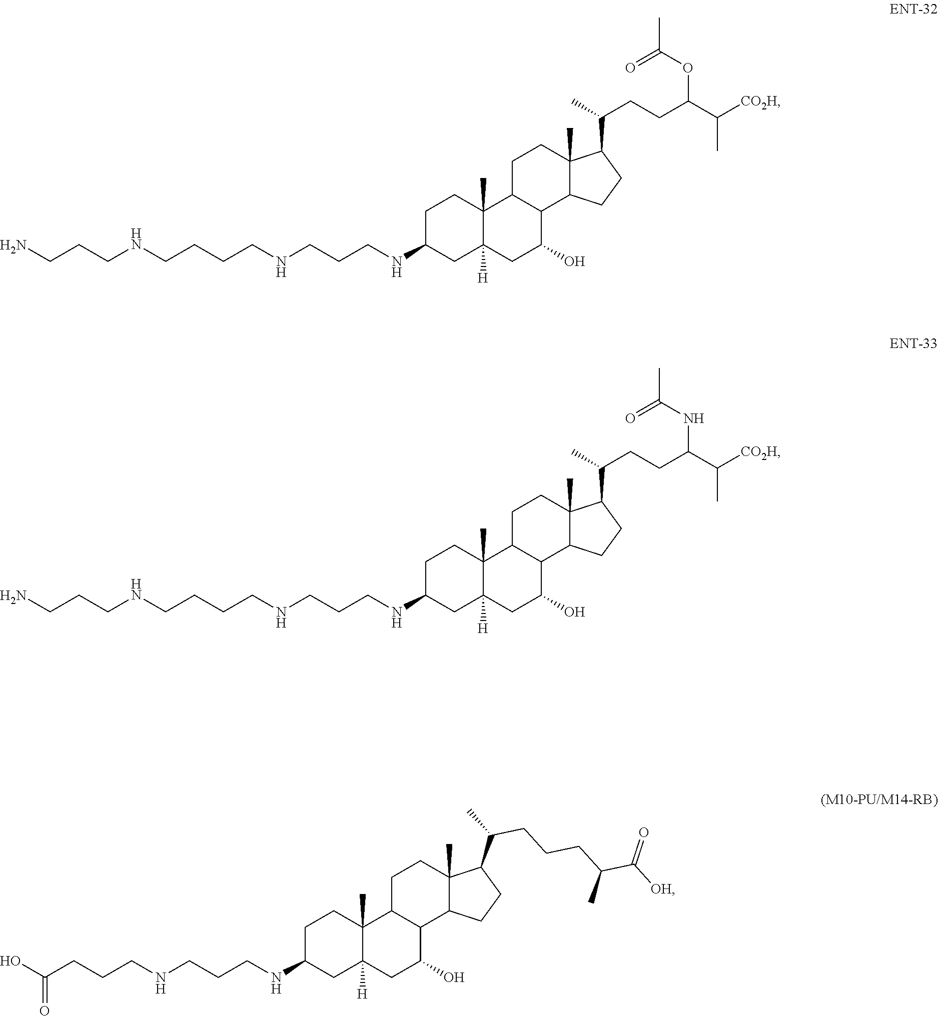 [SMILES: CC(=O)OC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(=O)NC(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; C[C@H](CCC[C@H](C)C(=O)O)[C@H]1CCC2C3C(CC[C@@]21C)[C@@]1(C)CC[C@H](NCCCNCCCC(=O)O)C[C@@H]1C[C@H]3O]

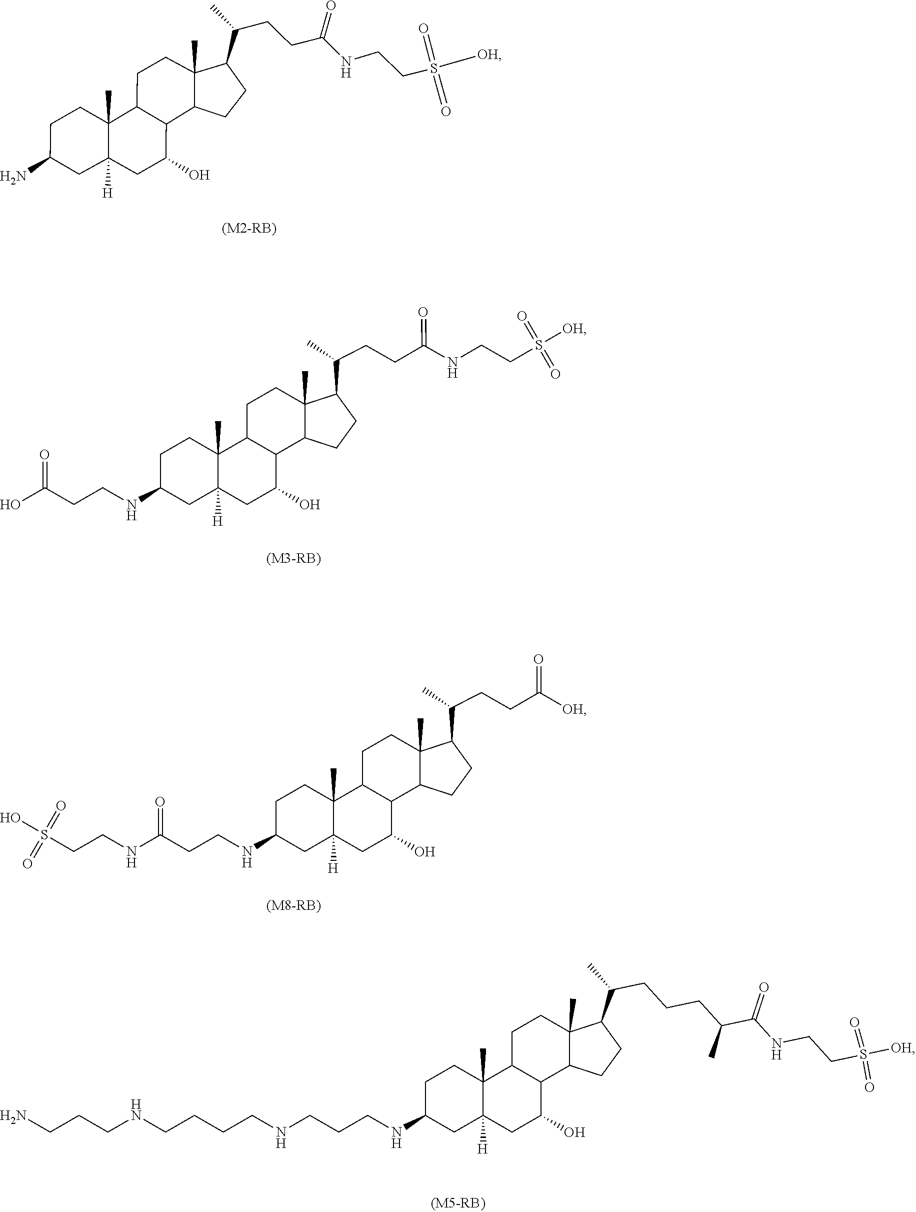 [SMILES: C[C@H](CCC(=O)NCCS(=O)(=O)O)[C@H]1CCC2C3C(CC[C@@]21C)[C@@]1(C)CC[C@H](N)C[C@@H]1C[C@H]3O ; C[C@H](CCC(=O)NCCS(=O)(=O)O)[C@H]1CCC2C3C(CC[C@@]21C)[C@@]1(C)CC[C@H](NCCC(=O)O)C[C@@H]1C[C@H]3O ; C[C@H](CCC(=O)O)[C@H]1CCC2C3C(CC[C@@]21C)[C@@]1(C)CC[C@H](NCCC(=O)NCCS(=O)(=O)O)C[C@@H]1C[C@H]3O ; C[C@H](CCC[C@H](C)C(=O)NCCS(=O)(=O)O)[C@H]1CCC2C3C(CC[C@@]21C)[C@@]1(C)CC[C@H](NCCCNCCCCNCCCN)C[C@@H]1C[C@H]3O]

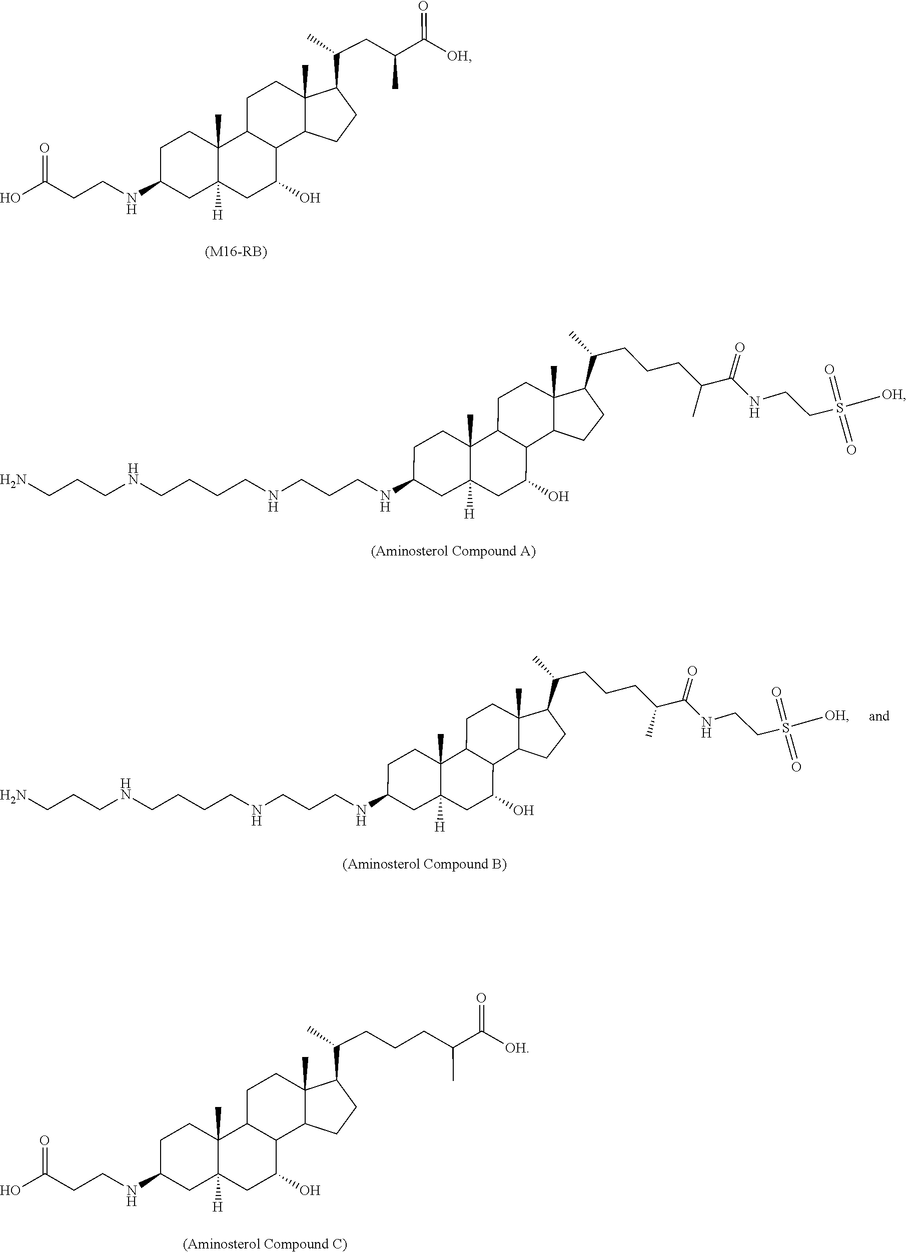 [SMILES: C[C@H](C[C@H](C)C(=O)O)[C@H]1CCC2C3C(CC[C@@]21C)[C@@]1(C)CC[C@H](NCCC(=O)O)C[C@@H]1C[C@H]3O ; CC(CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; CC(CCC[C@@H](C)[C@H]1CCC2C3C(CC[C@@]21C)[C@@]1(C)CC[C@H](NCCC(=O)O)C[C@@H]1C[C@H]3O)C(=O)O]

### V. Compositions

### A. Pharmaceutical Carriers

In another aspect, provided herein are compositions comprising an aminosterol compound disclosed herein, or a pharmaceutically acceptable salt, solvate, prodrug, or derivative thereof, and one or more pharmaceutically acceptable carriers and/or excipients. The composition does not require the presence of a cyclodextrin. Administration of an aminosterol disclosed herein, or a pharmaceutically acceptable salt, solvate, or prodrug thereof, may comprise administration of the composition. While it is possible for an aminosterol, or a pharmaceutically acceptable salt, solvate or prodrug thereof, to be administered alone, it is preferable to administer it as a pharmaceutical formulation, together with one or more pharmaceutically acceptable carriers. The carrier(s) must be “acceptable” in the sense of being compatible with the aminosterol, or a pharmaceutically acceptable salt, solvate or prodrug thereof, and not deleterious to the recipients thereof.

In instances where aerosol administration is appropriate, an aminosterol described herein, or a pharmaceutically acceptable salt, solvate or prodrug thereof, can be formulated as an aerosol using standard procedures. The term “aerosol” includes any gas-borne suspended phase of a compound described herein which is capable of being inhaled into the bronchioles or nasal passages, and includes dry powder and aqueous aerosol, and pulmonary and nasal aerosols. Specifically, aerosol includes a gas-born suspension of droplets of a compound described herein, as may be produced in a metered dose inhaler or nebulizer, or in a mist sprayer. Aerosol also includes a dry powder composition of a composition of the present technology suspended in air or other carrier gas, which may be delivered by insufflation from an inhaler device, for example.

See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al. Pharmacol Toxicol Methods, 27:143-159 (1992).

### B. Dosage Forms

The aminosterol compositions may conveniently be presented in unit dosage form and may be prepared by any of the methods well known in the art of pharmacy. Exemplary aminosterol dosage forms include, but are not limited to, oral, intranasal, and injectable (IP, IV, or IM). Preferably, the aminosterol formulation is administered orally, intranasally, or a combination thereof. In yet another embodiment, administration comprises non-oral administration.

Formulations or compositions of the present technology may be packaged together with, or included in a kit with, instructions or a package insert. “Pharmaceutically acceptable carrier” refers to a non-toxic solid, semisolid or liquid filler, diluent, encapsulating material or formulation auxiliary of any type.

Pharmaceutical compositions according to the present technology may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel” PH101 and Avicel P1102, microcrystallite cellulose, and silicified microcrystalline cellulose (ProSolv SMCC™). Suitable lubricants, including agents that act on the flowability of the powder to be compressed, may include colloidal silicon dioxide, such as Aerosil 200, tale, stearic acid, magnesium stearate, calcium stearate, and silica gel Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel” PH101 and Avicel PH102; lactose such as lactose monohydrate, lactose anhydrous, and Pharmatose DCL21 dibasic calcium phosphate such as Emcompress”; mannitol; starch; sorbitol; sucrose; and glucose. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

### C. Dosages & Dosing Period

Dosage of an aminosterol described herein can range from about 1 to about 500 mg/day, or any amount in-between these two values. In some embodiments, a subject is administered a therapeutically effective dose of an aminosterol described herein. The therapeutically effect amount of the at least one aminosterol or a salt or derivative thereof in the methods of the disclosure can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the disclosure can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

Oral dosage of an aminosterol described herein can range from about 1 to about 500 mg/day, or any amount in-between these two values. In one embodiment, the method of administration comprises oral administration and the therapeutically effective amount of the aminosterol comprises (i) about 1 to about 300 mg/day; (ii) about 25 to about 300 mg/day; (iii) about 50 to about 300 mg/ay; or (iv) about 75 to about 300 mg/day. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 3 10, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of the aminosterol. Examples of such intranasal aminosterol low dosages include, but are not limited to, about 0 001 to about 6 mg/day, or any amount in-between these two values. In some embodiments, the method of administration comprises nasal administration and the therapeutically effective amount of the aminosterol comprises (i) about 0.001 to about 6 mg/day; (ii) about 0.001 to about 4 mg/day; or (iii) about 0.001 to about 2 mg/day. For example, the low dosage of an intranasally administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 11, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3 5, about 3.6, about 3.7, about 3.8, about 3.9, about 4, about 4.1, about 4.2, about 4.3, about/1.4 about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that the same dosage would not provide any pharmacological effect if administered by any other route—e.g. a “subtherapeutic” dosage, and, in addition, does not result in negative effects. For example, as described herein, Compound III (ENT-03) has the pharmacological effects of a reduction in food intake and weight loss. Therefore, in certain embodiments of the IN methods of the disclosure, if the aminosterol may be Compound III (ENT-03) or a salt, solvate, prodrug, or derivative thereof, then if the same IN Compound III dosage is administered via another route, such as oral, IIP, or IV, then the Compound III dosage will not result in a noticeable reduction in food intake or noticeable weight loss Similarly, some aminosterols are known to produce the pharmacological effects of nausea, vomiting and/or reduced blood pressure. Thus, in certain embodiments of the IN methods of the disclosure, if the aminosterol has this effect when given IN, then if the same IN aminosterol dosage is administered via another route, such as oral, IP, or IV, then the aminosterol dosage will not result in noticeable nausea, vomiting, and/or a reduction in blood pressure. In some embodiments, intranasal administration comprises delivery of the aminosterol to the brain. Suitable exemplary aminosterol dosages are described above.

Aminosterol doses can be de-escalated (reduced) if any given aminosterol dose induces a persistent undesirable side effect, such as diarrhea, vomiting, or nausea. In another embodiment, a dose of an aminosterol can be varied plus or minus a defined amount to enable a modest reduction in a dose to eliminate adverse events, or a modest increase in a dose if clinical results suggest this is desirable—e.g. no or minimal adverse events and potential increased efficacy with a modest increase in dose. For example, in one embodiment an aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 1 7%, about 18%, about 19%, or about 20%.

The pharmaceutical composition comprising an aminosterol or a derivative, salt, solvate, or prodrug thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g. twice daily). In an exemplary embodiment, dosing is 1×/day.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8 5, about 9, about 9 5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for I or more years, etc. Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 ruin, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 min, about 60 ruin, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the disclosure, the aminosterol dosage is taken within about 15 min, about 30 min, about 45 min, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the disclosure, following the aminosterol dosage the subject has a period of about 15 min, about 30 min, about 45 min, about 1 hours, about 1.25 hrs, about 1.5 hrs, about 1.75 hrs, about 2 hrs, about 2.25 hrs, about 2.5 hrs, about 2.75 hrs, or about 3 hours without food.

### D. “Fixed Aminosterol Dose”

In one aspect, the present application relates to the discovery of a method to determine a “fixed dose” of an aminosterol described herein, that is not age, size, or weight dependent but rather is individually calibrated. The “fixed dose” obtained through this method yields highly effective results in treating the symptom(s) based on which the “fixed dose” was determined, related symptoms along the “brain-gut” axis, and the underlying disorder. Further, contemplated herein are methods of leveraging this same “fixed dose” method for methods of prevention of the underlying disorder. The present disclosure is not limited to methods whereby a fixed aminosterol dosage is determined for a specific patient.

A “fixed aminosterol dose,” also referred to herein as a “fixed escalated aminosterol dose,” which will be therapeutically effective is determined for each patient by establishing a starting dose of an aminosterol composition and a threshold for improvement of a particular symptom which is used as a tool or marker for evaluating the effectiveness of the aminosterol dosage. Following determining a starting aminosterol dosage for a particular patient, the aminosterol dose is then progressively escalated by a consistent amount over consistent time intervals until the desired improvement is achieved; this aminosterol dosage is the “fixed escalated aminosterol dosage” for that particular patient for that particular symptom. In exemplary embodiments, an orally administered aminosterol dose is escalated every about 3 to about 5 days by about 25 mg until the desired improvement is reached, Symptoms evaluated, along with tools for measuring symptom improvement, may be specifically described below, including but not limited to constipation, hallucinations, sleep disturbances (e.g. REM disturbed sleep or circadian rhythm dysfunction), cognitive impairment, depression, or alpha-synuclein aggregation.

This therapeutically effective “fixed dose” is then maintained throughout treatment and/or prevention. Thus, even if the patient goes “off drug” and ceases taking the aminosterol composition, the same “fixed dose” is taken with no ramp up period following re-initiation of aminosterol treatment. Not to be bound by theory, it is believed that the aminosterol dose is dependent on the severity of nerve damage relating to the symptom establishing the “fixed dose” threshold—e.g. for constipation, the dose may be related to the extent of nervous system damage in the patient's gut.

Dose escalation: When determining a “fixed aminosterol dosage” for a particular patient, a patient is started at a lower dose and then the dose is escalated until a positive result is observed for the symptom being evaluated. An exemplary symptom to be evaluated can be constipation, but any symptom associated with the disease or disorder to be treated can be used as a marker for evaluating aminosterol dosage. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a patient experiencing severe constipation, defined as less than one spontaneous bowel movement (SBM) a week, the starting oral aminosterol dose can be about 150 mg/day or greater. In contrast, for a patient having moderate constipation, e.g. defined as having more than one SBM a week, the starting oral aminosterol dose can be about 75 mg/day. Thus, as an example, a patient experiencing moderate constipation can be started at an oral aminosterol dosage of about 75 mg/day, whereas a patient experiencing severe constipation can be started at an oral aminosterol dosage of about 150 mg/day.

In other embodiments, a patient experiencing moderate symptoms (for the symptom being used to calculate a fixed escalated aminosterol dose) can be started at an oral aminosterol dosage of from about 10 mg/day to about 75 mg/day, or any amount in-between these values. For example, the starting oral aminosterol dosage for a moderate symptom can be about 10, about 15, about 20, about 25, about 30, about 35, about 40, about 45, about 60, about 65, about 70, or about 75 mg/day.

In yet further embodiments, when the patient is experiencing severe symptoms (for the symptom being used to calculate the fixed escalated aminosterol dose), the patient can be started at an oral aminosterol dosage ranging from about 75 to about 175 mg/day, or any amount in-between these two values. For example, the starting oral aminosterol dosage for a severe symptom can be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day.

In some embodiments, the starting oral aminosterol dose may be about 125 mg or about 175 mg/day; again dependent on the severity of the symptom, such as constipation.

Starting intranasal (IN) aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0,05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In exemplary embodiments, the aminosterol dose is given periodically as needed. For example, the aminosterol dose can be given once per day. The aminosterol dose can also be given every other day, 2, 3, 4, or 5× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. For example, when the symptom being treated/measured is constipation, threshold improvement can be an increase of one SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about x/“week, about 2×/week, about every other week, or about Ix/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 05, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Other symptoms that can be used as an endpoint to determine aminosterol dosage for a patient's fixed escalated aminosterol dosage are any symptom known to be associated with the disease, disorder, or condition intended to be treated. For example, neuro disease symptoms described herein and include, but are not limited to, (a) at least one non-motor aspect of experiences of daily living as defined by Part I of the Unified Parkinson's Disease Rating Scale (UPDRS), such as for example cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the UPDRS, such as for example, speech, saliva and drooling, chewing and swallowing, eating tasks, dressing, hygiene, handwriting, turning in bed, tremors, getting out of a bed, a car, or a deep chair, walking and balance, and freezing, (c) at least one motor symptom identified in Part III of the UPDRS, such as for example, speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the UPDRS, such as for example, dyskinesias, functional impact of dyskinesias, time spent in the off state, functional impact of fluctuations, complexity of motor fluctuations, and painful off-state dystonia; (e) constipation; (f) depression; (g) cognitive impairment; (h) sleep problems or sleep disturbances; (i) circadian rhythm dysfunction; (j) hallucinations; (k) fatigue; (l) REM disturbed sleep; (m) REM behavior disorder; (n) erectile dysfunction; (o) apnea; (p) postural hypotension; (q) correction of blood pressure or orthostatic hypotension; (r) nocturnal hypertension; (s) regulation of temperature; (t) improvement in breathing or apnea; (u) correction of cardiac conduction defect; (v) amelioration of pain; (w) restoration of bladder sensation and urination; (x) urinary incontinence; and/or (v) control of nocturia.

VI. Methods of Treatment and/or Prevention

Aspects of this disclosure relate to methods of treating certain symptoms and/or methods of treating and/or preventing diseases or disorders associated with one or more of these symptoms by administration of a therapeutically effective amount of an aminosterol and/or aminosterol composition disclosed herein, or a pharmaceutically acceptable salt, solvate, prodrug, or derivative thereof, optionally present in one or more pharmaceutically acceptable carriers. In one aspect, the aminosterol composition comprises a cyclodextrin, as detailed herein.

In one embodiment, the symptoms, diseases, and/or disorders are generally correlated with abnormal αS pathology, dopaminergic dysfunction, and/or protein misfolding, including but not limited to abnormal tau pathology, abnormal amyloid β pathology, abnormal αS pathology and/or dopaminergic dysfunction, which means they are amenable to treatment with aminosterols described herein. The compositions of the present technology can be administered using any pharmaceutically acceptable method, including but not limited to oral, pulmonary, nasal, and nebulization administration In yet another embodiment, administration comprises non-oral administration. Administration of the aminosterol cyclodextrin formulations is by injection, e.g. intravenous, subcutaneous, intramuscular, etc. In one embodiment, administration comprises non-oral administration.

In some embodiments, provided herein are methods for treating a subject in need having a condition or symptom susceptible to treatment with an aminosterol, or a pharmaceutically acceptable salt, solvate, prodrug, or derivative thereof, comprising administering to the subject a therapeutically effective amount of an aminosterol or aminosterol composition described herein, or a pharmaceutically acceptable salt, solvate, or prodrug thereof. In some embodiments, provided herein are methods for treating a subject in need having a condition susceptible to treatment with an aminosterol, comprising administering to the subject a therapeutically effective amount of a composition comprising or consisting essentially of an aminosterol disclosed herein, or a pharmaceutically acceptable salt, solvate, or prodrug thereof, and one or more pharmaceutically acceptable carriers and/or excipients. The aminosterol composition can be a cyclodextrin composition.

Non-limiting examples of symptoms amenable to treatment with aminosterols, or a pharmaceutically acceptable salt, solvate, prodrug, or derivative thereof, include but are not limited to constipation, hallucinations, sleep disorders, cognitive impairment, depression, and inflammation.

Examples of diseases amenable to treatment with aminosterols, or a pharmaceutically acceptable salt, solvate, prodrug, or derivative thereof, are described herein and include but are not limited to neurological or neurodegenerative disorders or diseases. Examples of neurological or neurodegenerative disorders or diseases include, but are not limited to, Alzheimer's disease (AD), Parkinson's disease (PD), multiple system atrophy (MSA), schizophrenia, Huntington's disease (HD), Progressive supranuclear palsy, progressive nuclear palsy, supranuclear palsy, Frontotemporal dementia (FTD), vascular dementia, Amyotrophic lateral sclerosis (ALS), multiple sclerosis (MS), spinal muscular atrophy (SMA), Friedreich's ataxia, Lewy Body dementia or disease, Guadeloupian parkinsonism, spinocerebellar ataxia, and autism. In another embodiment, the aminosterols described herein and compositions comprising the same can be used in methods of treating, preventing, and/or slowing the onset or progression of psychological or behavior disorder and/or a related symptom in a subject in need is provided. In one embodiment, the psychological or behavior disorder can be, for example, depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, and cognitive impairment.

In one embodiment, provided is a method of treating, preventing, and/or slowing the onset or progression in a subject of inflammation and/or a related symptom related to αS pathology. The method comprises administering to the subject a therapeutically effective amount of at least one aminosterol disclosed herein, or a pharmaceutically acceptable salt, solvate, or prodrug thereof, and one or more pharmaceutically acceptable carriers and/or excipients. The aminosterol composition can be a cyclodextrin composition.

In another embodiment, a neurodegenerative disorder may be positively impacted by administration of a therapeutically effective amount of an aminosterol according to the disclosure. A “positive impact” includes for example slowing advancement of the condition, improving one or more symptoms, etc.

In another embodiment, a method of treating, preventing, and/or slowing the onset or progression of a cerebral or general ischemic disorder and/or a related symptom in a subject in need is provided. The cerebral or general ischemic disorder can be, for example, microangiopathy, intrapatient cerebral ischernia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, diabetic retinopathy, high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, erectile dysfunction, and pulmonary edema.

Examples of diseases amenable to treatment with aminosterols, or a pharmaceutically acceptable salt, solvate, prodrug, or derivative thereof, are described herein and include but are not limited to fertility and reproductive function and behavior. Thus, in one aspect encompassed is a method of improving or resolving impaired reproductive function, impaired fertility and/or impaired reproductive behavior in a subject in need thereof, comprising administering to the subject a therapeutically effective amount of an aminosterol compound disclosed herein, or a composition comprising such an aminosterol.

Also encompassed is a method of suppressing, preventing and/or slowing the onset or progression of appetite or weight gain, and/or one or more related symptoms, including but not limited to obesity, in a subject in need thereof, comprising administering to the subject a therapeutically effective amount of an aminosterol compound disclosed herein, or a composition comprising such an aminosterol.

In addition, encompassed is a method of suppressing, preventing and/or slowing the onset or progression of diabetes (type 1 and/or type 2), and/or one or more related symptoms, in a subject in need thereof, comprising administering to the subject a therapeutically effective amount of an aminosterol compound disclosed herein, or a composition comprising such an aminosterol.

In one embodiment, a method of inhibiting protein tyrosine phosphatase 1B (PTP1B) is provided, comprising contacting PTP1B with at least one aminosterol disclosed herein, or pharmaceutically acceptable salt, solvate, or prodrug thereof.

Exemplary symptoms correlated with abnormal αS pathology, dopaminergic dysfunction, and/or protein misfolding and amenable to aminosterol treatment include, for example, constipation, hallucinations, and other symptoms described herein. In one embodiment, provided is a method of treating, preventing, and/or slowing the onset or progression in a subject of inflammation and/or a related symptom related to αS pathology. The method comprises administering to the subject a therapeutically effective amount of at least one aminosterol disclosed herein, or pharmaceutically acceptable salt, solvate, or prodrug thereof.

Also encompassed are methods of treating and/or preventing psychological or behavior disorder, or any symptom related to such diseases or conditions. In one embodiment, the psychological or behavior disorder is depression, anxiety, delirium, irritability, illusion and delusions, amnesia, autism, apathy, bipolar disorder, disinhibition, aberrant motor and obsessive-compulsive behaviors, sleep disorders, sleep fragmentation, REM behavior disorder, circadian rhythm dysfunction, sleep apnea, or cognitive impairment

In another embodiment, psychological or behavior disorder, or any symptom related to such diseases or conditions, may be positively impacted by administration of a therapeutically effective amount of an aminosterol according to the disclosure. A “positive impact” includes for example slowing advancement of the condition, improving one or more symptoms, etc.

In one aspect, the aminosterols described herein can be administered to a subject in need to treat a microbial infection. In some embodiments, the subject in need has a condition selected from the group consisting of viral infections, microbial infections, bacterial infections, e.g. Gram-negative and/or Gram-positive bacterial infections, Mycobacteria infections, fungal infections, and/or protozoan infections. In some embodiments, the method further comprises administering one or more antiviral drugs.

In some embodiments, the viral infection is caused by a virus selected from the group consisting of Yellow Fever, Cytomegalovirus, Eastern Equine Encephalitis virus, Hepatitis B virus, Hepatitis Delta virus, Dengue virus, and Human Immunodeficiency virus. In some embodiments, the condition to be treated is a viral infection caused by a virus selected from the group consisting of “African Swine Fever Viruses,” Arbovirus, Adenoviridae, Arenaviridae, Arterivirus, Astroviridae, Baculoviridae, Bimaviridae, Birnaviridae, Bunyaviridae, Caliciviridae, Cauliinoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g.

Paramyxoviridae, Morbillivirus, Rhabdoviridae), Myoviridae, Orthomyxoviridae (e.g. Influenza A, Influenza 13, and parainfluenza), Papiloma virus, Papovaviridae, Paramyxoviridae, Prions, Parvoviridae, Phycodnaviridae, Picomaviridae (e.g. Rhinovirus, Poliovirus), Poxviridae (such as Smallpox or Vaccinia), Potyviridae, Reoviridae (e.g. Rotavirus), Retroviridae (HTLV-I, HTLV-II, Lentivirus), Rhabdoviridae, Tectiviridae, Togaviridae (e.g. Rubivirus), herpes, pox, papilloma, corona, influeina, hepatitis, sendai, sindbis, vaccinia viruses, west nile, hanta, viruses which cause the common cold, and any combination thereof. In some embodiments, the condition to be treated is selected from the group consisting of AIDS, viral meningitis, Dengue, EBV, hepatitis, a chronic disease suspected to be of viral oigin, multiple sclerosis, Type I diabetes, Type II diabetes, atherosclerosis, cardiomyopathies, Kawaski disease, aplastic anemia, and any combination thereof,

In one aspect, a method of treating or preventing an infection by a coronavirus in a subject is provided, comprising administering to the subject a therapeutically effective amount of the aminosterol compound of any embodiment herein or the composition of any embodiment herein.

In some embodiments, the coronavirus comprises a virus selected from the group consisting of an Alphacoronavinrs; a Colacovirus such as Bat coronavirus CDPHE15; a Decacovins such as Bat coronavirus HIKUI0 or Rhinolophus ferrumequinurn alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus 1HKU8; a Myotacovirus such as Myotis ricketti alphacoronavirus Sax-20 11; a nyctacovirus such as Nyctalus velutinus alphacoronavirus SC-20¹3; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus fiKU24, Human coronavirus HKU1 or Murine coronavirus; a Hibecovirus such as Bat Hp-betacoronav,irus Zhejiang2013; a Merbecovirus such as Hedgehog coronavirus 1, Middle East respiratory syndrome-related coronavirus (MERS-CoV), Pipistrellus bat coronavirus H1KU5 or Tylonvcteris bat coronavirus HKU4; a Nobecovirus such as Rousettus bat coronavirus GCCDCl or Rousettus bat coronavirus HKU9, a Sarbecovinrs such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HTKU11, Porcine coronavirus HKU1 5, Munia coronavirus HKU13 or White-eye coronavirus HKU16; a Herdecovirus such as Night heron coronavirus HKU19; a Moordecovirus such as Common moorhen coronavirus HKU21; a Garnmacoronavirus; a Cegacovirus such as Beluga whale coronavirus SW1; and an Igacovirus such as Avian coronavirus.

In some embodiments, the coronavirus is encoded by a polynucleotide comprising the sequence of SARS-CoV-2, or a polynucleotide having at least 80% sequence identity to the polynucleotide comprising the sequence of SARS-CoV-2. In some embodiments, the coronavirus comprises or is characteristic ofhuman coronavirus 229E, human coronavirus OC43, SAR S-CoV, HCoV NL63, HKU1, MERS-CoV, or SARS-CoV-2. In some embodiments, the coronavirus comprises or is characteristic of SARS-CoV-2.

### VI Patient Populations

The disclosed aminosterols and compositions comprising the same, including but not limited to aminosterol-cyclodextrin compositions, can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of any of the conditions disclosed herein. For example, genetic markers of the condition or family history may be used as signs to identify subjects likely to develop the particular condition. Thus, in some embodiments, prevention may involve first identifying a patient population at risk of developing the condition.

Alternatively, certain symptoms are considered early signs of particular disorders. Thus, in some embodiments, a patient population may be selected for being “at risk” for developing the condition based on age and experiencing symptoms associated with the condition. Further genetic or hereditary signs may be used to refine the patient population.

In some embodiments, the subject is suffering from and/or at risk of suffering from one or more conditions selected from the group consisting of obesity, fatty liver disease, type 2 diabetes, heart disease, stroke, hypertension, gallbladder disease, gout, sleep apnea, osteoarthritis, High LDL cholesterol, low HDL cholesterol, high levels of triglycerides (dyslipidemia), endometrial cancer, breast cancer, colon cancer, kidney cancer, gallbladder cancer, and liver cancer.

### VIII. Kits

Aminosterol formulations or compositions of the disclosure may be packaged together with or included in a kit along with instructions or a package insert. Such instructions or package inserts may address recommended storage conditions, such as time, temperature and light, taking into account the shelf-life of the aminosterol or derivatives or salts thereof, Such instructions or package inserts may also address the particular advantages of the aminosterol or derivatives or salts thereof, such as the ease of storage for formulations that may require use in the field, outside of controlled hospital, clinic or office conditions.

The disclosure also provides a pharmaceutical pack or kit comprising one or more containers filled with one or more aminosterol pharmaceutical compositions disclosed herein The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat-sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

### IX. Combination Therapy

In the methods of the disclosure, the aminosterol compositions may be administered alone or in combination with one or more other therapeutic agents. An example of an additional therapeutic agent is one known to treat the condition the aminosterol is being administered to treat.

Combinations may be administered either concomitantly, e.g. as an admixture, separately but simultaneously or concurrently; or sequentially. This includes presentations in which the combined agents are administered together as a therapeutic mixture, and also procedures in which the combined agents are administered separately but simultaneously, e.g. as through separate intravenous lines into the same individual. Administration “in combination” further includes the separate administration of one of the compounds or agents administered first, followed by the second. The regimen selected can be administered concurrently since activation of the aminosterol induced response does not require the systemic absorption of the aminosterol into the bloodstream and thus eliminates concern over the likelihood systemic of drug-drug interactions between the aminosterol and the administered drug.

### X. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forns “a, “an”, and “the” are used interchangeably and intended to include the plural forms as well and fall within each meaning, unless the context clearly indicates otherwise. Also, as used herein, “and/or” refers to, and encompasses, any and all possible combinations of one or more of the listed items, as well as the lack of combinations when interpreted in the alternative (“or”).

As used herein, the phrase “therapeutically effective amount” means a dose of aminosterol, or a salt or derivative thereof that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein, Those skilled in the art can adjust such amounts in accordance with standard practices as needed to treat a specific subject. The therapeutically effective amount may vary based on the route of administration and dosage form, the age and weight of the subject, and/or the severity of the subject's condition. For example one of skill in the art would understand that the therapeutically effective amount for treating a small individual may be different from the therapeutically effective amount for treating a large individual.

The term “administering” as used herein includes prescribing for administration, as well as actually administering, and includes physically administering by the subject being treated or by another.

As used herein “subject” or “patient” or “individual” refers to any subject, patient, or individual and the terms are used interchangeably herein. In this regard, the terms “subject,” “patient,” and “individual” includes mammals, and, in particular humans.

A “derivative” of an aminosterol described herein may have one or more chemical modifications which do not modify, or drastically diminish, or may improve, the activity of the aminosterol. Such “activity” may include pharmacological targets and affinity therefore, including changes in affinities for different subtypes of a particular receptor target. A “derivative” of an aminosterol in which modifications well known in the art of medicinal chemistry to “mimic” the original spatial and charge characteristics of a portion of the original structure can be introduced to improve the therapeutic characteristics of the aminosterol. In general, such modifications are introduced to influence metabolism, ease of administration, biodistribution, or any combination thereof. Examples of such variants or derivatives include, but are not limited to, (1) substitutions of the sulfate or carboxylic acid by a sulfonate, sulfate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system. Other derivatives include replacement of one or more hydrogens of the aminosterol with deuterium or the unsaturation of any one or more C-C single bonds of the aminosterol. The pharmaceutical composition can comprise one or more pharmaceutically acceptable carriers or excipients.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. When an embodiment is defined by one of these terms (e.g. “comprising”), it should be understood that this disclosure also includes alternative embodiments, such as “consisting essentially of” and “consisting of” for the embodiment.

“Substantially” or “essentially” means nearly totally or completely, for instance, 95%, 96%, 97%, 98%, 99%, or greater of some given quantity.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the disclosure.

As used herein, the term “treatment” or “treating” means any treatment of a disease or condition or associated disorder, in a patient. Inhibiting or preventing the disease or condition, that is, arresting or suppressing the development of clinical symptoms, such as neurological deficits resulting from cerebral ischemia, also included within “treatment” is a provision of neuroprotection; and/or relieving the disease or condition that is, causing the regression of clinical symptoms (e.g. increasing neurological performance or reducing neurological deficits),

In some embodiments, “treatment” encompasses “providing neuroprotection” to the subject. “Treatment” and “providing neuroprotection” may comprise the administration of the therapeutics agent(s) or compositions disclosed herein.

“Pharmaceutically acceptable salt” refers to salts of a compound, which salts are suitable for pharmaceutical use and are derived from a variety of organic and inorganic counter ions well known in the art. Pharmaceutically acceptable salts include, when the compound contains an acidic functionality, by way of example only, sodium, potassium, calcium, magnesium, ammonium, and tetraalkylammonium. When the molecule contains a basic functionality, salts of organic or inorganic acids, such as hydrochloride, hydrobromide, tartrate, mesylate, acetate, maleate, and oxalate. Stahl and Wermuth, eds, “Handbook of Pharmaceutically Acceptable Salts,” (2002), Verlag Helvetica Chimica Acta, Zorich, Switzerland), which is hereby incorporated by reference for its teachings related to pharmaceutically acceptable salts, discusses a variety of pharmaceutical salts, their selection, preparation, and use.

“Reductive amination” as used herein, refers to a synthetic procedure whereby a ketone or aldehyde is reacts with an amine to form in imine or iminium which is subsequently reduced to form an amine. Methods and conductions to affect reductive amination are known to those of ordinary skill in the art. See, for example, Dangerfield et al., J Org. Chem, 2010, 75, 5470-477; Taibakhsh et al., Synthesis, 2011, 490-496; and Abdel-Magid et al., J Org. Chem, 1996, 61, 3849-3862; the entire disclosures of which are hereby incorporated by reference.

It is to be understood, that in any aminosterol or steroid compound disclosed herein, the stereochemical configuration of the 17 and 20 carbons are equally represented as depicted in the two example steroid nuclei shown below, i.e., the configurations of the 17 and 20 carbons, as drawn below, are the same:

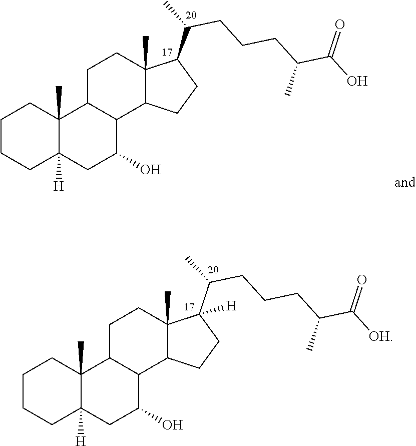 [SMILES: C[C@H](CCC[C@@H](C)[C@H]1CCC2C3C(CC[C@@]21C)[C@@]1(C)CCCC[C@@H]1C[C@H]3O)C(=O)O ; C[C@H](CCC[C@@H](C)[C@H]1CCC2C3C(CC[C@@]21C)[C@@]1(C)CCCC[C@@H]1C[C@H]3O)C(=O)O]

In any embodiment herein, wherein the ring carbons of the aminosterol (or corresponding carbons of synthetic intermediates corresponding to Compound I-R or —S) marked with exclamation points below are not stereodefined, embodiments wherein those carbons are defined as shown in Formula II are within the scope of this invention:

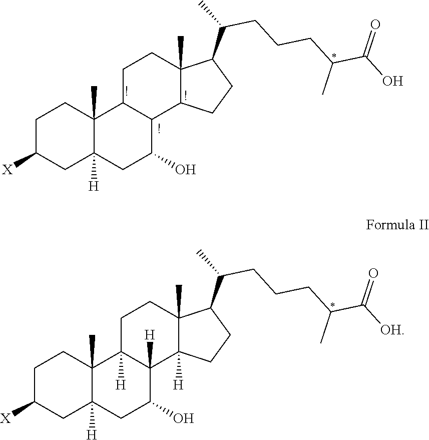

Embodiments described herein are further illustrated by, though in no way limited to, the following working examples.

### EXAMPLES

### Example 1: Identification of Metabolites of ENT-03 in Rat Plasma and Urine Samples

The purpose of this example was to identify potential metabolites of ENT-03S and to predict the possible metabolic pathways of ENT-03S in rat plasma and urine samples.

An equal number of rat urine (50 μL) were taken from different animals at various time points following administration of ENT-03S. For plasma samples, the Hamilton pooling method was used. After pooling, 50 μL of matrix was mixed with 200 μL of methanol (containing 0.1% formic acid), vortexed for 100 seconds and then centrifuged at 16,000 g for 10 minutes at 4° C. to precipitate proteins. 100 μL of the supernatant was transferred to a new tube and the supernatant was diluted with 100 μL pure water, mixed well and analysed using UPLC-MS/MS.

A total of 9 metabolites of ENT-03S were detected in rat plasma and urine samples. The metabolite information for ENT-03S in rat plasma and urine samples is listed in Table 2. All metabolites observed were less than 5% of the drug-related material in male rat plasma sample.

The peak areas of metabolites and parent drug in rat urine sample were very low and no major metabolite was observed.

TABLE 2
Metabolite information for ENT-03S

| | | | | | Metabolite peak area | |
|---|---|---|---|---|---|---|
| | | | | | percentages | |
| Peak | R.T. | Meas. m/z | | | Rat Plasma | Rat Urine |
| No. | (min) | [M + H]⁺ | Mass Shift (Da) | Biotransformation | Male | Male |
| 1 (MI) | 6.74 | 289.25586(z = 2) | −42.04695 | De-alkylation + Oxidation | 0.55% | 7.56% |
| 2 (M2) | 8.56 | 399.32544 | −220.22631 | De-alkylation + De-hydration + De-amination | 0.47% | — |
| 3 (P) | 8.56 | 310.27933(z = 2) | 0.00000 | Parent Drug | 91.29% | 71.94% |
| 4 (M4) | 9.09 | 281.75055(z = 2) | −57.05785 | Dc-alkylation | 3.07% | 5.74% |
| 5 (M5) | 9.10 | 399.32520 | −220.22631 | De-alkylation + De-hydration + De-amination | 0.14% | — |
| 6 (M6) | 9.18 | 315.27158(z = 2) | 9.98435 | De-hydrogenation + Methylation | 0.31% | — |
| 7 (M7) | 9.32 | 361.26807(z = 2) | 101.97755 | De-amination + Cysteine conjugation | 0.67% | — |
| 8 (M8) | 9.38 | 310.27930(z = 2) | — | Unknown | 2.62% | — |
| 9 (M9) | 9.75 | 281.75055(z = 2) | −57.05785 | De-alkylation | 0.47% | — |
| 10 | 10.10 | 577.45660 | −42.09457 | De-alkylation + Oxidation | 0.42% | 14.76% |
| (M10) | | | | | | |

Note:
Relative peak area determined from extracted ion chromatogram of rat plasma and urine samples.
m/z acquired from MS Full Scan spectrum of rat plasma and urine samples.
“—”: not observed.

Material and Instrument Information: ENT-03S, having a molecular weight (free base) of 618.98 g/mol, and a molecular weight of 764.82 g/mol, and HPLC purity of 97.37%, was used for the experiment. ENT-03S was stored at 2-8° C.

Plasma and urine samples were obtained from male rats at various time points following a 10 mg/kg dose of ENT-03S via subcutaneous administration in bile dicut cannulated male SD rats, as detailed in Table 3 The samples were transferred on dry ice and stored at −80° C. prior to use.

TABLE 3
Plasma Sample

| | Dose | Time (h) | | | | | | |
|---|---|---|---|---|---|---|---|---|
| Group/Sex | (mg/kg) | 0.25 | 0.5 | 1 | 2 | 4 | 8 | 24 |
| 1/Male | 10 | ✓ | ✓ | ✓ | ✓ | ✓ | ✓ | ✓ |
| | | ✓ | ✓ | ✓ | ✓ | ✓ | ✓ | ✓ |
| | | ✓ | ✓ | ✓ | ✓ | ✓ | ✓ | ✓ |

Urine Sample

| Group/ | Dose | Time (h) | |
|---|---|---|---|
| Sex | (mg/kg) | 0-8 | 8-24 |
| 1/Male | 10 | ✓ | ✓ |
| | | ✓ | ✓ |
| | | ✓ | ✓ |

Other chemicals and materials: All inorganic reagents, organic solvents and water were at least analytical laboratory reagent grade and are detailed in Table 4.

TABLE 4
Materials information

| Item | Source |
|---|---|
| Acetonitrile | Fisher Chemical |
| DMSO | Solarbio |
| Formic Acid | Fisher Chemical |
| H₂O | Prepared by Water Purification System |

Instrument information: Information on instruments used in this example is detailed in Table 5.

TABLE 5
Instrument information

| Item | Source | Model Code |
|---|---|---|
| Balance | Mettler Toledo | XS205-Du |
| Centrifuge | Eppendorf | 5424R |
| LC System | Thermo Scientific | DionexUltiMate 3000 |
| Mass Spectrometer | Thermo Scientific | Thermo Scientific |
| | | Q Exactive |
| Plate Shaker | IKA | MS3 digital |

Study design: An equal number of rat urine (50 μL) samples were taken from different animals at various time points from study No.: PH-DMPK-ETR-21-001 following administration of ENT-03S. For plasma samples, the Hamilton pooling method was used. The specific sampling volume at each time point is shown in the following Table 6:

TABLE 6

| Time (h) | Calculated volume (μL) | Sampling volume (μL) |
|---|---|---|
| 0.25 | 0.5 − 0.25 = 0.25 | 0.5 |
| 0.5 | 1 − 0.25 = 0.75 | 1.5 |
| 1 | 2 − 0.5 = 1.5 | 3 |
| 2 | 4 − 1 = 3 | 6 |
| 4 | 8 − 2 = 6 | 12 |
| 8 | 24 − 4 = 20 | 40 |
| 24 | 24 − 8 = 16 | 32 |
| Total | 40 | 80 |

Sample preparation: For urine and bile samples, an equal volume of samples (50 μL) are taken from different animals at various time points after the administration of each group of samples, and the same group of samples are mixed thoroughly. After pooling, 50 μL of matrix was mixed with 200 μL of methanol (containing 0.1% formic acid), vortexed for 100 seconds and then centrifuged at 16,000 g for 10 minutes at 4° C. to precipitate proteins. 100 μL of the supernatant was transferred to a new tube and the supernatant was diluted with 100 μL pure water and mixed well. The samples were then analysed using UPLC-MS/MS.

Data Analysis: UPLC-MS/MS was used for sample analysis, and was conducted using a DionexlUltiMate 3000/Vanquish UHPLC system. For mass spectrometry (MS), all analysis was carried out with a Q-Exactive instrument (Thermo Fisher). A full MS scan was used to trigger the data dependent acquisition (DDA) of MS/MS scan spectra. Compound dependent parameters were optimized by ENT-03S. DDA settings were used to trigger acquisition of MS² spectra for ions whose intensity is in top 5 of all, with exclusion of impurity ions (m/z 149.02332, m/z 279,15909, ma 391 28429 and in z 445 12003), after 3 occurrences for 10 s. Dynamic exclusion settings: automatic exclusion of the ions which have been taken. Bioanalytical sample analysis parameters are shown in Table 7.

TABLE 7
LC conditions

| Instrument: | Dionex UltiMate 3000 UHPLC system | | | |
|---|---|---|---|---|
| | (Thermo Fisher Scientific, USA) | | | |
| Column: | Waters XSelect HSS ™ T3, 2.5 μm, 100 × 2.1 mm | | | |
| Column | Ambient temperature | | | |
| temperature: | | | | |
| Injection volume: | 10 μL | | | |
| Mobile phase: | A: 0.1% formic acid in water | | | |
| | D: 0.1% formic acid in acetonitrile | | | |
| | | | | Elution rate |
| | Time (min) | A (%) | D (%) | (mL/min) |
| Elution | 0 | 95 | 5 | 0.5 |
| gradient: | 1.5 | 95 | 5 | 0.5 |
| | 9.0 | 65 | 35 | 0.5 |
| | 12.0 | 0 | 100 | 0.5 |
| | 14.0 | 0 | 100 | 0.5 |
| | 14.3 | 95 | 5 | 0.5 |
| | 15.0 | 95 | 5 | 0.5 |

Mass conditions

| Instrument: | Thermo Scientific Q Exactive | |
|---|---|---|
| | (Thermo Fisher Scientific, USA) | |
| Ion Source: | Ion source type | Turbo spray |
| | Ionization model: | HESI |
| | Ionisation mode: | Positive |
| | Sheath gas flow rate | 55 |
| | Aux gas flow rate | 15 |
| | Spray voltage(kV) | 4 |
| | Capillary temp.(° C.) | 350 |
| | S-lens RF level | 60 |
| | Aux gas heater temp (° C.) | 350 |
| Scan type: | Full MS/ddMS² | |
| MS Scan: | Microscans | 1 |
| | Resolution | 70,000 |
| | AGC Target | 3 × e⁶ |
| | Maximum IT | 100 ms |
| MS/MS Scan: | Microscans | 1 |
| | Resolution | 17,500 |
| | AGC Target | 1 × e⁵ |
| | Maximum IT | 50 ms |
| | Loop count | 5 |
| | MSX count | 1 |
| | NCE/stepped NCE | 30, 35, 40 |

Data Analysis: Data was acquired and processed using Xcalibur v4.1 software (Thermo Fisher Scientific) and Compound Discoverer 3.1 (Thermo Fisher Scientific) and Excel (Microsoft 2016).

Study Results: The metabolite information of ENT-03S is detailed in Table 8.

TABLE 8
The metabolite information of ENT-03S from rat plasma and urine samples

| | | | | | Metabolite peak area | |
|---|---|---|---|---|---|---|
| Peak | R.T. | Meas. m/z | | | Rat Plasma | Rat Urine |
| No. | (min) | [M + H]+ | Mass Shift (Da) | Biotransformation | Male | Male |
| 1 (MI) | 6.74 | 289.25586(z = 2) | −42.04695 | De-alkylation + Oxidation | 3.62E+06 | 1.70E+05 |
| 2 (M2) | 8.56 | 399.32544 | −220.22631 | De-alkylation + De-hydration + De-amination | 3.13E+06 | — |
| 3 (P) | 8.56 | 310.27933(z = 2) | 0.00000 | Parent Drug | 6.03E+08 | 1.62E+06 |
| 4 (M4) | 9.09 | 281.75055(z = 2) | −57.05785 | De-alkylation | 2.02E+07 | 1.29E+05 |
| 5 (M5) | 9.10 | 399.32520 | −220.22631 | De-alkylation + De-hydration + De-amination | 9.11E+05 | — |
| 6 (M6) | 9.18 | 315.27158(z = 2) | 9.98435 | De-hydrogenation + Methylation | 2.02E+06 | — |
| 7 (M7) | 9.32 | 361.26807(z = 2) | 101.97755 | De-amination + Cysteine conjugation | 4.45E+06 | — |
| 8 (M8) | 9.38 | 310.27930(z = 2) | — | Unknown | 1.73E+07 | — |
| 9 (M9) | 9.75 | 281.75055(z = 2) | −57.05785 | De-alkylation | 3.10E+06 | — |
| 10 | 10.10 | 577.45660 | −42.09457 | De-alkylation + Oxidation | 2.74E+06 | 3.32E+05 |
| (M10) | | | | | | |

Note:
Relative peak area determined from extracted ion chromatogram of rat plasma and urine samples.
“—”: not observed.
m/z acquired from MS Full Scan spectrum of rat plasma and urine samples.

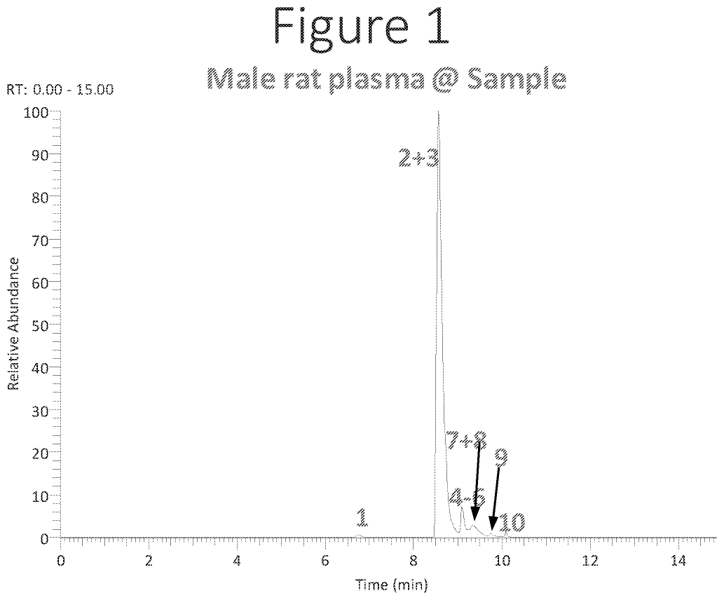

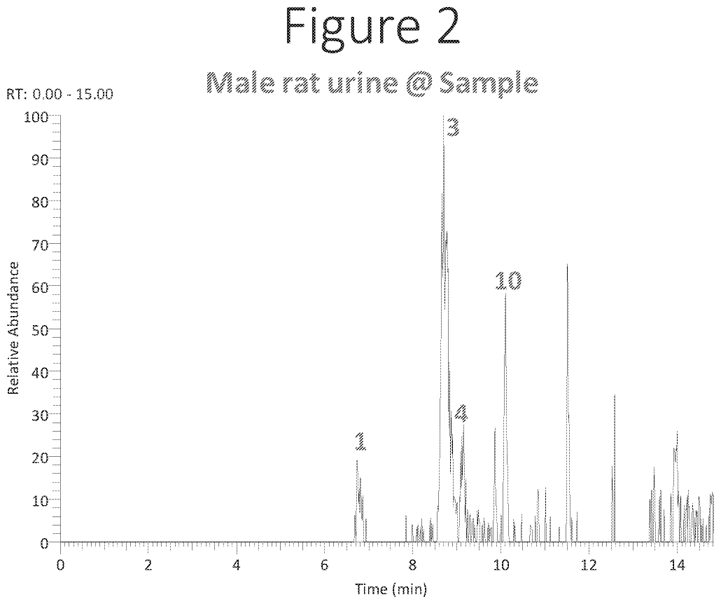

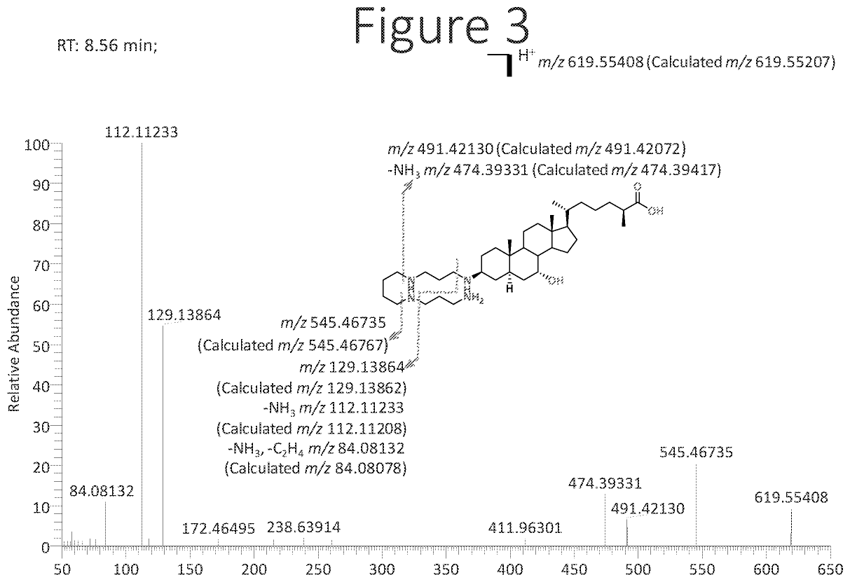

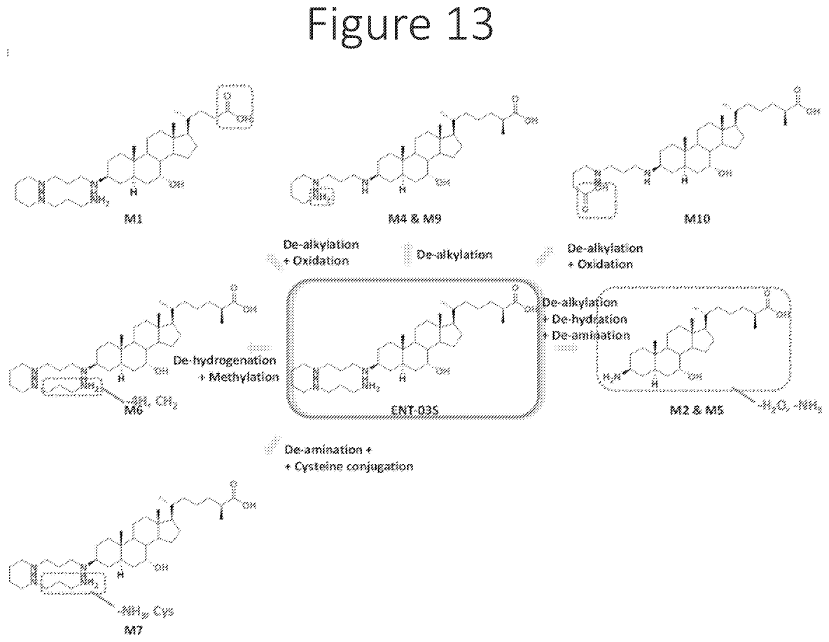

The XIC chromatograms from full MS/ddMS2 scans of ENT-03S in rat plasma and urine samples are shown in FIG. 1-FIG. 2. The MS/MS spectra of ENT-03S and metabolites are shown in FIG. 3-FIG. 12. Proposed metabolic pathways for ENT-03S from rat plasma and urine samples are shown in FIG. 13.

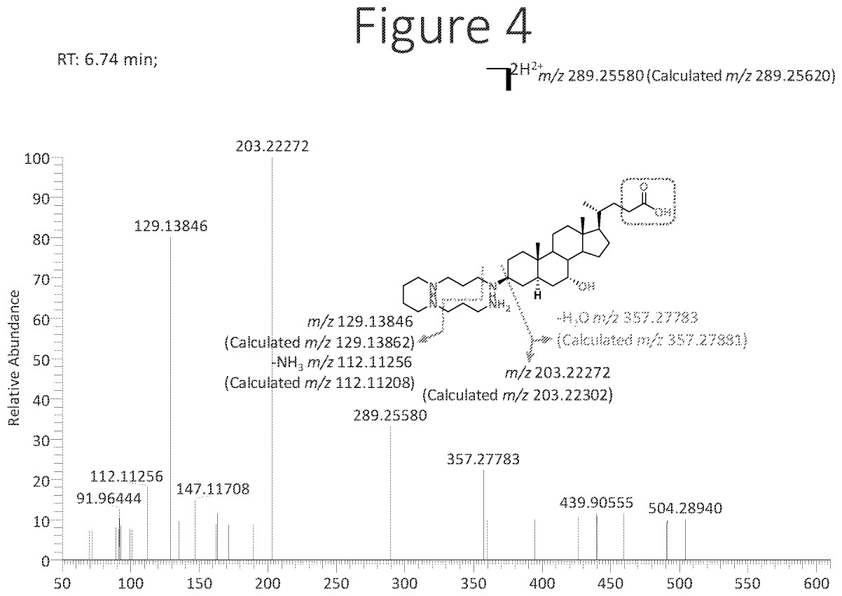

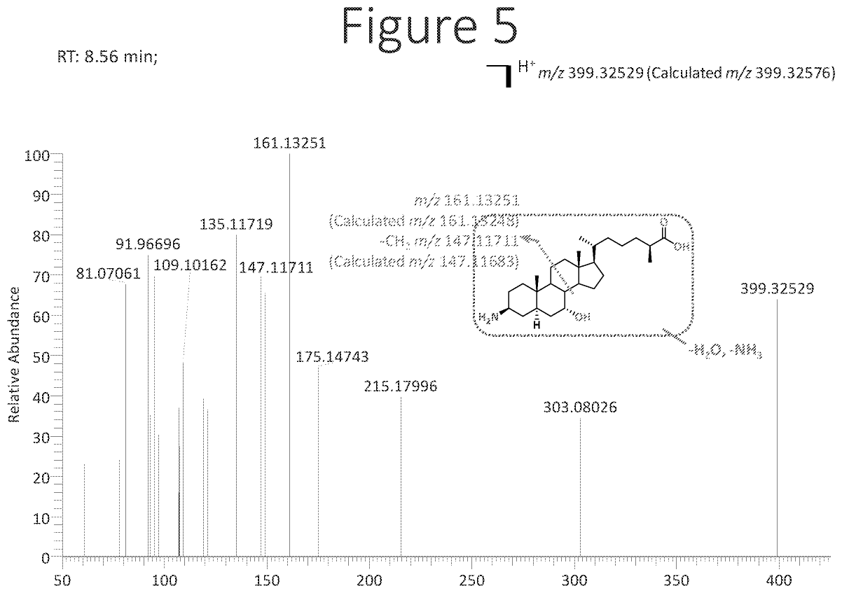

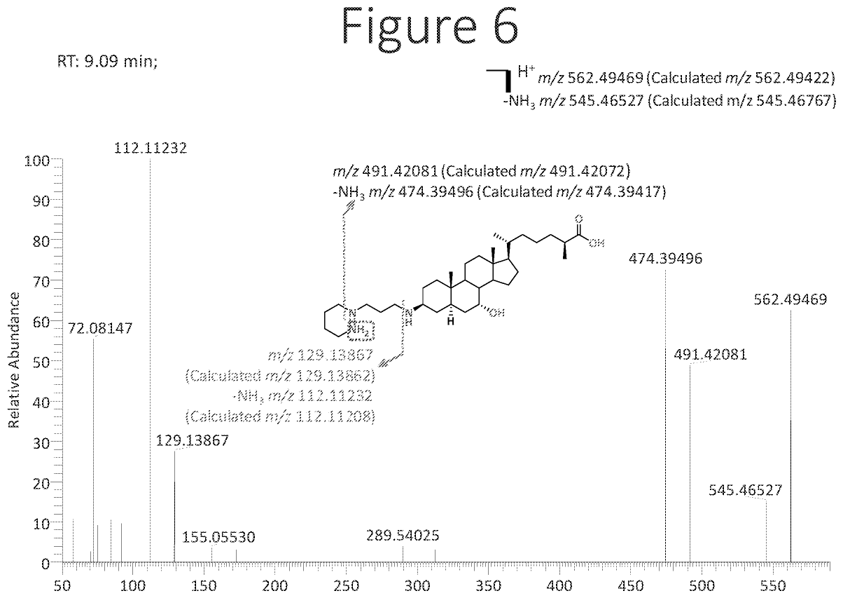

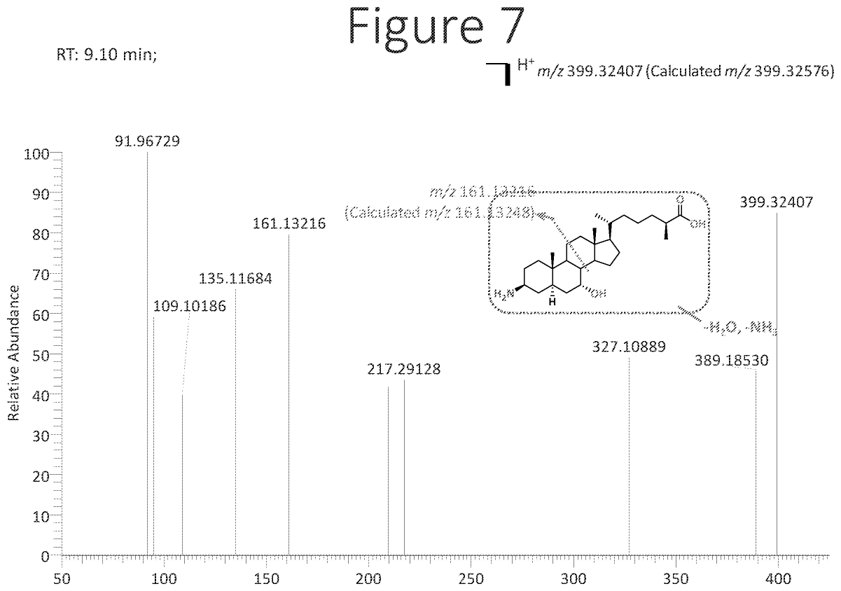

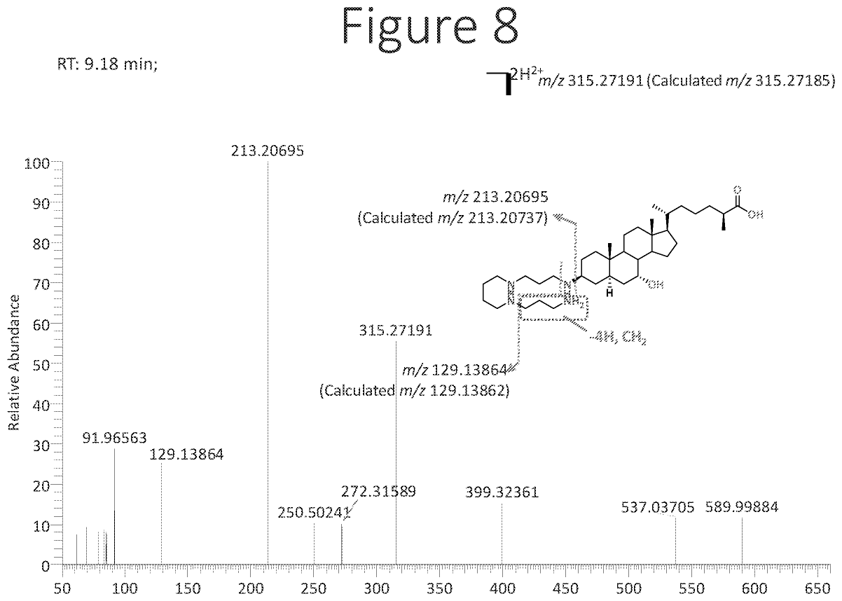

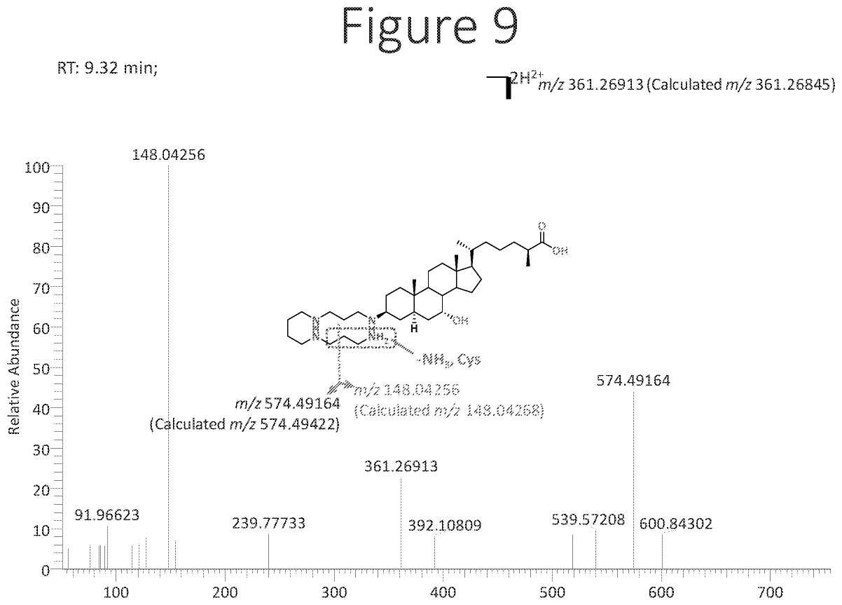

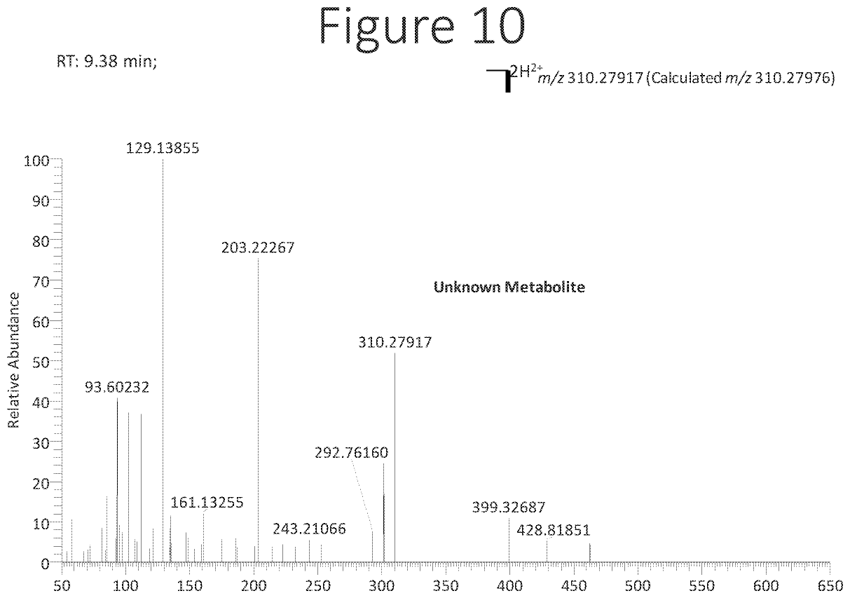

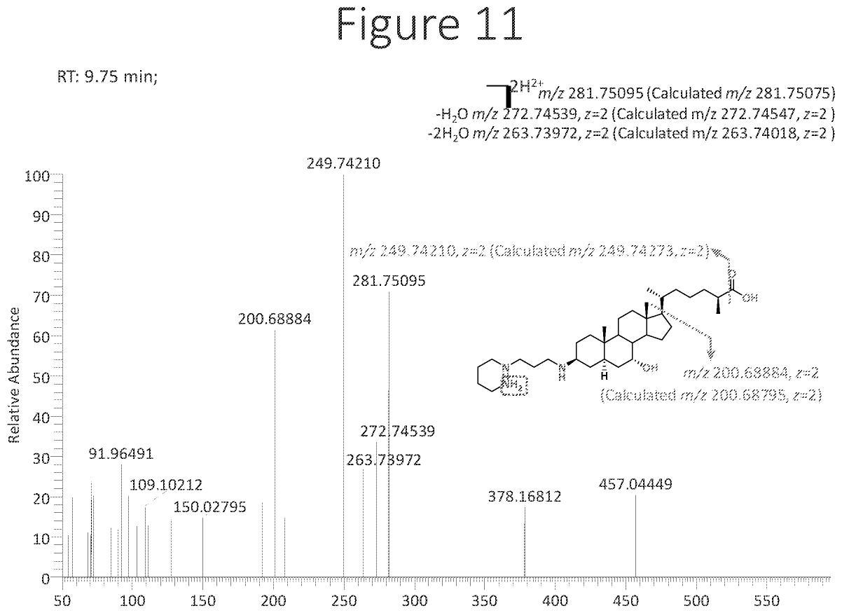

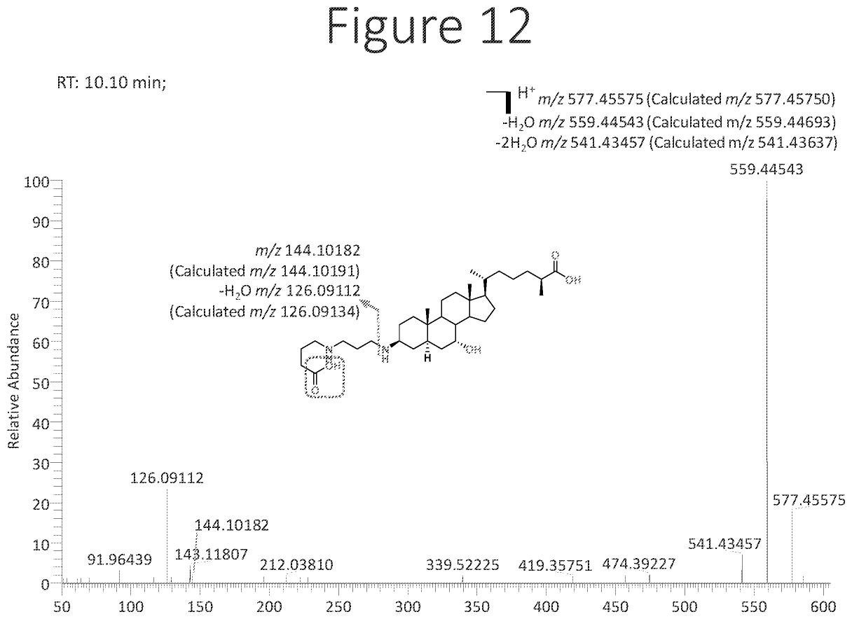

In particular, the XIC chromotagraphs from rat plasma samples are shown in FIG. 1, and the XIC chromatograms from rat urine samples are shown in FIG. 2. FIG. 3 shows the MS/MS fragmentation spectrum of ENT-03S. FIG. 4 shows the MS/MS fragmentation spectrum of an ENT-03S derivative designated as M1-PU. FIG. 5 shows the MS/MS fragmentation spectrum of an ENT-03S derivative designated as M2-PU. FIG. 6 shows the MS/MS fragmentation spectrum of an ENT-03S derivative designated as M4-PU. FIG. 7 shows the MS/MS fragmentation spectrum of an ENT-03S derivative designated as M5-PU. FIG. 8 shows the MS/MS fragmentation spectrum of an ENT-03S derivative designated as M6-PU. FIG. 9 shows the MS/MS fragmentation spectrum of an ENT-03S derivative designated as M7-PU. FIG. 10 shows the MS/MS fragmentation spectrum of an ENT-03S derivative designated as M8-PU. FIG. 11 shows the MS/MS fragmentation spectrum of an ENT-03S derivative designated as M9-PU. FIG. 12 shows the MS/MS fragmentation spectrum of an ENT-03S derivative designated as M10-PU.

### Example 2: Identification of Metabolites of ENT-03 in Rat Bile Samples

The purpose of this example was to identify potential metabolites of ENT-03S and to predict the possible metabolic pathways of ENT-03S in rat bile samples.

An equal volume of rat bile samples (50 μL) were taken from different animals at different time points following administration of ENT-03S. 50 μL of matrix was mixed with 200 μL of methanol (containing 0.1% formic acid), vortexed for 100 seconds and then centrifuged at 16,000 g for 10 minutes at 4° C. to precipitate proteins. 100 μL of the supernatant was transferred to a new tube and the supernatant diluted with 100 μt, pure water, mixed well and analyzed using UPLC-MS/MS.

A total of 16 metabolites of ENT-03S were detected in rat bile samples. The metabolite information for ENT-03S in rat bile samples is listed in Table 9. The peak area percentages of parent drug and each metabolite were determined by semi-quantitative estimation using the extracted ion chromatogram peak areas of all detected drug-related material in the sample. Metabolites with percentage of peak area>10% are proposed as major metabolites of ENT-03S. The de-alkylated and oxidised metabolite M1 and de-alkylated, oxidised and de-hydrogenated metabolite M4 were major metabolites in male rat bile samples with peak area percentages of 21.50% and 15.28%.

TABLE 9
Metabolite information for ENT-03S

| | | | | | | Metabolite peak |
|---|---|---|---|---|---|---|
| | | R.T. | Meas. m/z | Mass Shift | | area percentages |
| Peak No. | | (min) | [M + H]⁺ | (Da) | Biotransformation | Rat Bile |
| 1 | (M1) | 7.17 | 642.41388 | 22.86258 | De-alkylation + Oxidation | 21.50% |
| 2 | (M2) | 7.91 | 499.31976 | −120.23205 | De-alkylation + Oxidation + | 3.90% |
| | | | | | Taurine conjugation | |
| 3 | (M3) | 8.12 | 571.34033 | −48.21092 | De-alkylation + Oxidation + | 2.27% |
| | | | | | Taurine conjugation | |
| 4 | (M4) | 8.56 | 624.40289 | 4.85201 | De-alkylation + Oxidation + | 15.28% |
| | | | | | De-hydrogenation | |
| 5 | (M5) | 8.63 | 363.78098(z = 2) | 107.00410 | Taurine conjugation | 1.05% |
| 6 | (M6) | 9.17 | 499.31870 | −120.23205 | De-alkylation + Oxidation + | 3.71% |
| | | | | | Taurine conjugation | |
| 7 | (P) | 9.38 | 310.27917(z = 2) | 0.00000 | Parent Drug | 13.77% |
| 8 | (M8) | 9.44 | 571.34058 | −48.21092 | De-alkylation + Oxidation + | 2.46% |
| | | | | | Taurine conjugation | |
| 9 | (M9) | 9.65 | 281.75024(z = 2) | −57.05785 | De-alkylation | 7.38% |
| 10 | (M10) | 9.67 | 624.40277 | 4.85201 | De-alkylation + Oxidation + | 2.22% |
| | | | | | De-hydrogenation | |
| 11 | (M11) | 9.82 | 317.76102(z = 2) | 14.96328 | Oxidation | 9.52% |
| 12 | (M12) | 10.05 | 281.75024(z = 2) | −57.05785 | De-alkylation | 0.72% |
| 13 | (M13) | 10.12 | 317.76077(z = 2) | 14.96328 | Oxidation | 1.58% |
| 14 | (M14) | 10.16 | 577.45660 | −42.09457 | De-alkylation + Oxidation | 3.23% |
| 15 | (M15) | 10.34 | 666.44977 | — | Unknown | 4.63% |
| 16 | (M16) | 10.67 | 506.38342 | −113.16807 | De-alkylation + Oxidation | 0.69% |
| 17 | (M17) | 10.90 | 506.38336 | −113.16807 | De-alkylation + Oxidation | 2.25% |

Note:
Relative peak area determined from extracted ion chromatogram of rat bile samples.
m/z acquired from MS Full Scan spectrum of rat bile samples.
“—”: not observed.

Material and Instrument Information: ENT-03S, having a molecular weight (free base) of 618.98 g/mol, and a molecular weight of 764.82 g/mol, and HPLC purity of 97.37%, was used for the experiment. ENT-03S was stored at 2-8° C.

Bile samples were obtained from male rats at various time points following a 10 mg/kg dose of ENT-03S via subcutaneous administration in bile dicut cannulated male SD rats as detailed in Table 10. The samples were transferred on dry ice and stored at −80° C. prior to use

TABLE 10
Bile Sample

Time (h)

| Group/Sex | Dose (mg/kg) | 0-8 | 8-24 |
|---|---|---|---|
| 1/Male | 10 | ✓ | ✓ |
| | | ✓ | ✓ |
| | | ✓ | ✓ |

Other chemicals and materials: All inorganic reagents organic solvents and water were at least analytical laboratory reagent grade and are detailed in Table 11.

TABLE 11
Materials information

| Item | Source |
|---|---|
| Acetonitrile | Fisher Chemical |
| DMSO | Solarbio |
| Formic Acid | Fisher Chemical |
| H₂O | Prepared by Water Purification System |

Instrument information: Information on instruments used in this example is detailed in Table 12

TABLE 12
Instrument information

| Item | Source | Model Code |
|---|---|---|
| Balance | Mettler Toledo | XS205-Du |
| Centrifuge | Eppendorf | 5424R |
| LC System | Thermo Scientific | DionexUltiMate 3000 |
| Mass Spectrometer | Thermo Scientific | Thermo Scientific |
| | | Q Exactive |
| Plate Shaker | IKA | MS3 digital |

Study design: An equal volume of rat bile samples (50 μL) were taken from different animals at different time points following administration of ENT-03S in study No.: PII-DMPK-ETR-21-001. 50 μL of matrix was mixed with 200 μL of methanol (containing 0.1% formic acid), vortexed for 100 seconds and then centrifuged at 16,000 g for 10 minutes at 4° C. to precipitate proteins. 100 μL of the supernatant was transferred to a new tube and the supernatant diluted with 100 μL pure water, mixed well and analyzed using UPLC-MS/MS.

Sample preparation: For bile samples, an equal volume of samples (50 μL) are taken from different animals at various time points after the administration of each group of samples, and the same group of samples are mixed thoroughly.

Data Analysis: UPLC-MS/MS was used for sample analysis, and was conducted using a DionexUltiMate 3000/Vanquish UHPLC system. For mass spectrometry (MS), all analysis was carried out with a Q-Exactive instrument (Thermo Fisher). A full MS scan was used to trigger the data dependent acquisition (DDA) of MS/MS scan spectra. Compound dependent parameters were optimized by ENT-03S. DDA settings were used to trigger acquisition of MS² spectra for ions whose intensity is in top 5 of all, with exclusion of impurity ions (m/z 149.02332, m/z 279.15909, m/z 391.28429 and m/z 445.12003), after 3 occurrences for 10 s. Dynamic exclusion settings: automatic exclusion of the ions which have been taken. Bioanalytical sample analysis parameters are shown in Table 13.

TABLE 13
LC conditions

| Instrument: | Dionex UltiMate 3000 UHPLC system | | | |
|---|---|---|---|---|
| | (Thermo Fisher Scientific, USA) | | | |
| Column: | Waters XSelect HSS ™ T3, 2.5 μm, 100 × 2.1 mm | | | |
| Column | Ambient temperature | | | |
| temperature: | | | | |
| Injection volume: | 10 μL | | | |
| Mobile phase: | A: 0.1% formic acid in water | | | |
| | D: 0.1% formic acid in acetonitrile | | | |
| | | | | Elution rate |
| | Time (min) | A (%) | D (%) | (mL/min) |
| Elution | 0 | 95 | 5 | 0.5 |
| gradient: | 1.5 | 95 | 5 | 0.5 |
| | 9.0 | 65 | 35 | 0.5 |
| | 12.0 | 0 | 100 | 0.5 |
| | 14.0 | 0 | 100 | 0.5 |
| | 14.3 | 95 | 5 | 0.5 |
| | 15.0 | 95 | 5 | 0.5 |

Mass conditions

| Instrument: | Thermo Scientific Q Exactive | |
|---|---|---|
| | (Thermo Fisher Scientific, USA) | |
| Ion source: | Ion source type | Turbo spray |
| | Ionization model: | HESI |
| | Ionisation mode: | Positive |
| | Sheath gas flow rate | 55 |
| | Aux gas flow rate | 15 |
| | Spray voltage(kV) | 4 |
| | Capillary temp.(° C.) | 350 |
| | S-lens RF level | 60 |
| | Aux gas heater | 350 |
| | temp (° C.) | |
| Scan type: | Full MS/ddMS² | |
| MS Scan: | Microscans | 1 |
| | Resolution | 70,000 |
| | AGC Target | 3 × e⁶ |
| | Maximum IT | 100 ms |
| MS/MS Scan: | Microscans | 1 |
| | Resolution | 17,500 |
| | AGC Target | 1 × e⁵ |
| | Maximum IT | 50 ms |
| | Loop count | 5 |
| | MSX count | 1 |
| | NCE/stepped NCE | 30, 35, 40 |

Data Analysis: The analysis was carried out using a thermo Fisher Scientific Q Exactive instrument. Full MS scan was used to trigger the data dependent acquisition (DDA) of MS/MS scan spectra. Compound dependent parameters were optimized by ENT-03S. DDA settings were used to trigger acquisition of MS² spectra for ions whose intensity was in the top 5 of all, with exclusion of ions (m/z 149.02332, m/z 279 1 5909, m/z 391.28429 and m/z 445 12003), after 3 occurrences for 10 s. Data was acquired and processed using Xcalibur v4.1 software (Thermo Fisher Scientific) and Compound Discoverer 3.1 (Thermo Fisher Scientific) and Excel (Microsoft 2016).

Study Results: The metabolite information of ENT-03S is detailed in Table 14.

TABLE 14
The metabolite information of ENT-03S from bile samples

| | | | | | | Metabolite |
|---|---|---|---|---|---|---|
| | | R.T. | Meas. m/z | Mass | | peak area |
| Peak No. | | (min) | [M + H]⁺ | Shift (Da) | Biotransformation | Rat Bile |
| 1 | (M1) | 7.17 | 642.41388 | 22.86258 | De-alkylation + Oxidation | 2.65E+07 |
| 2 | (M2) | 7.91 | 499.31976 | −120.23205 | De-alkylation + Oxidation + | 4.80E+06 |
| | | | | | Taurine conjugation | |
| 3 | (M3) | 8.12 | 571.34033 | −48.21092 | De-alkylation + Oxidation + | 2.80E+06 |
| | | | | | Taurine conjugation | |
| 4 | (M4) | 8.56 | 624.40289 | 4.85201 | De-alkylation + Oxidation + | 1.88E+07 |
| | | | | | De-hydrogenation | |
| 5 | (M5) | 8.63 | 363.78098(z = 2) | 107.00410 | Taurine conjugation | 1.30E+06 |
| 6 | (M6) | 9.17 | 499.31870 | −120.23205 | De-alkylation + Oxidation + | 4.57E+06 |
| | | | | | Taurine conjugation | |
| 7 | (P) | 9.38 | 310.27917(z = 2) | 0.00000 | Parent Drug | 1.70E+07 |
| 8 | (M8) | 9.44 | 571.34058 | −48.21092 | De-alkylation + Oxidation + | 3.03E+06 |
| | | | | | Taurine conjugation | |
| 9 | (M9) | 9.65 | 281.75024(z = 2) | −57.05785 | De-alkylation | 9.10E+06 |
| 10 | (M10) | 9.67 | 624.40277 | 4.85201 | De-alkylation + Oxidation + | 2.73E+06 |
| | | | | | De-hydrogenation | |
| 11 | (M11) | 9.82 | 317.76102(z = 2) | 14.96328 | Oxidation | 1.17E+07 |
| 12 | (M12) | 10.05 | 281.75024(z = 2) | −57.05785 | De-alkylation | 8.90E+05 |
| 13 | (M13) | 10.12 | 317.76077(z = 2) | 14.96328 | Oxidation | 1.95E+06 |
| 14 | (M14) | 10.16 | 577.45660 | −42.09457 | De-alkylation + Oxidation | 3.98E+06 |
| 15 | (M15) | 10.34 | 666.44977 | — | Unknown | 5.71E+06 |
| 16 | (M16) | 10.67 | 506.38342 | −113.16807 | De-alkylation + Oxidation | 8.45E+05 |
| 17 | (M17) | 10.90 | 506.38336 | −113.16807 | De-alkylation + Oxidation | 2.77E+06 |

Note:
Relative peak area determined from extracted ion chromatogram of rat plasma and urine samples.
“—”: not observed.
m/z acquired from MS Full Scan spectrum of rat plasma and urine samples.

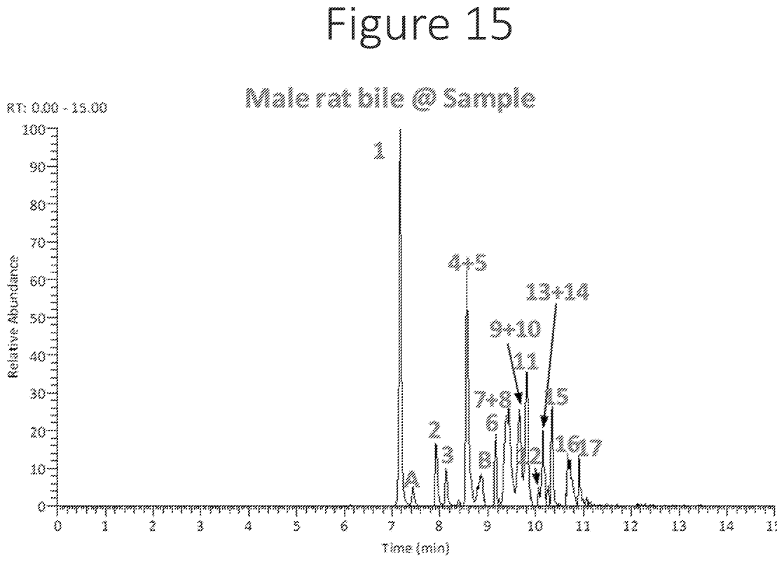

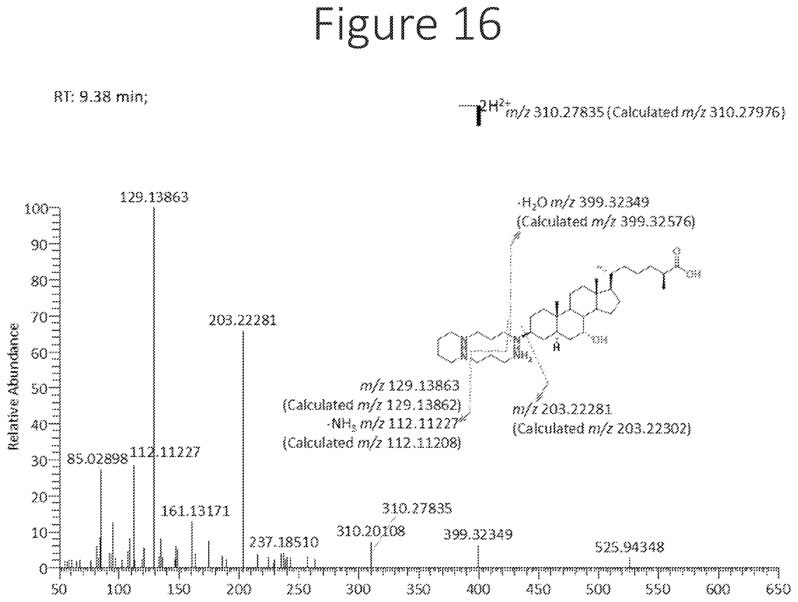

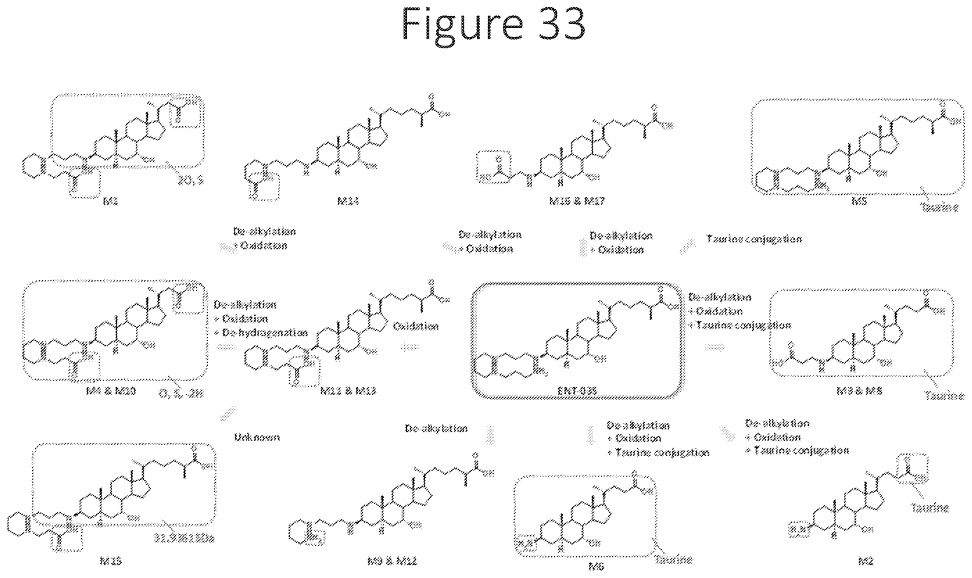

The XIC chromatograms from full MS/ddMS² scans of ENT-03S in bile samples are shown in FIG. 15. The MS/MS spectra of ENT-03S and metabolites are shown in FIG. 16-FIG. 32. Proposed metabolic pathways for ENT-03S from bile samples are shown in FIG. 33.

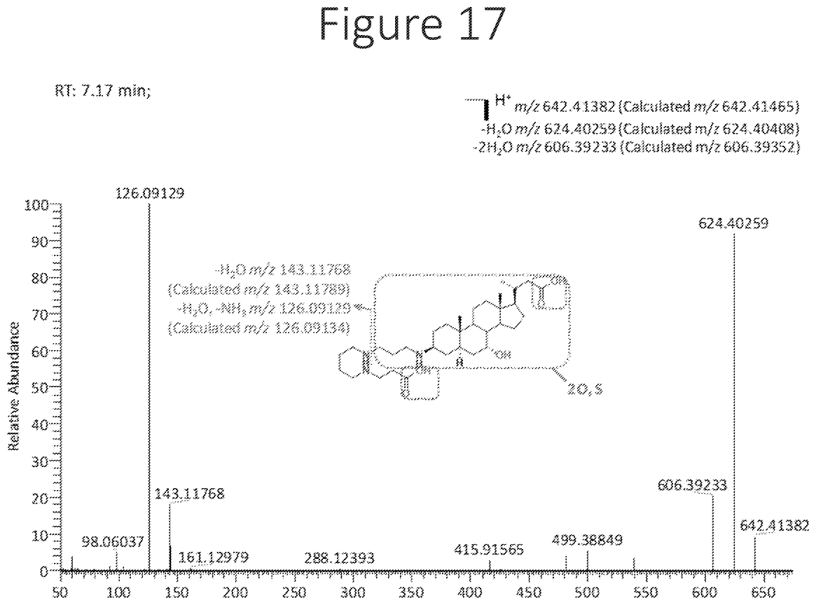

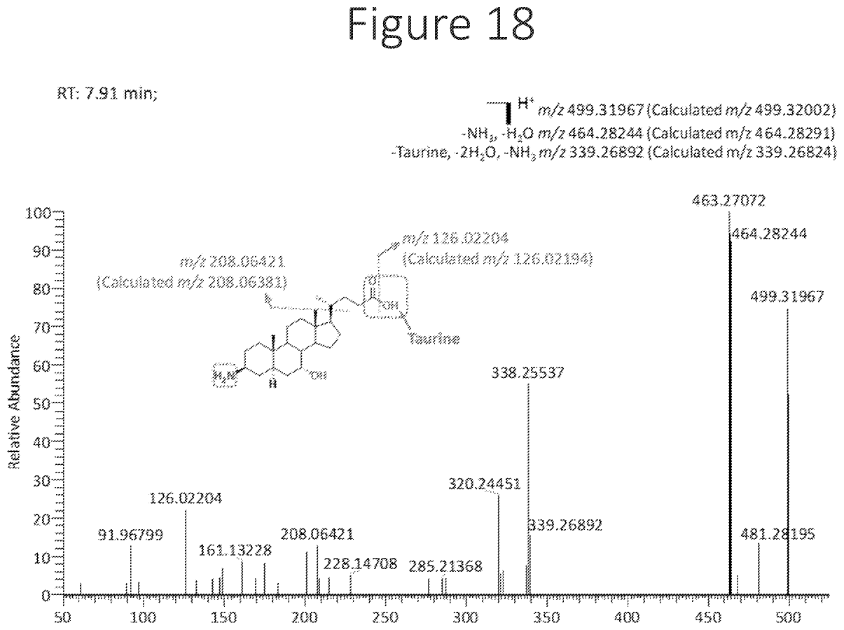

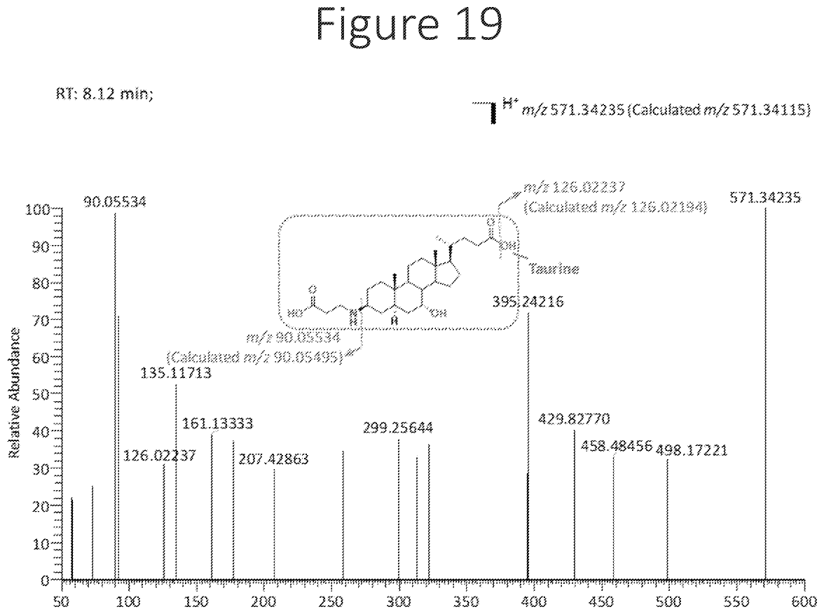

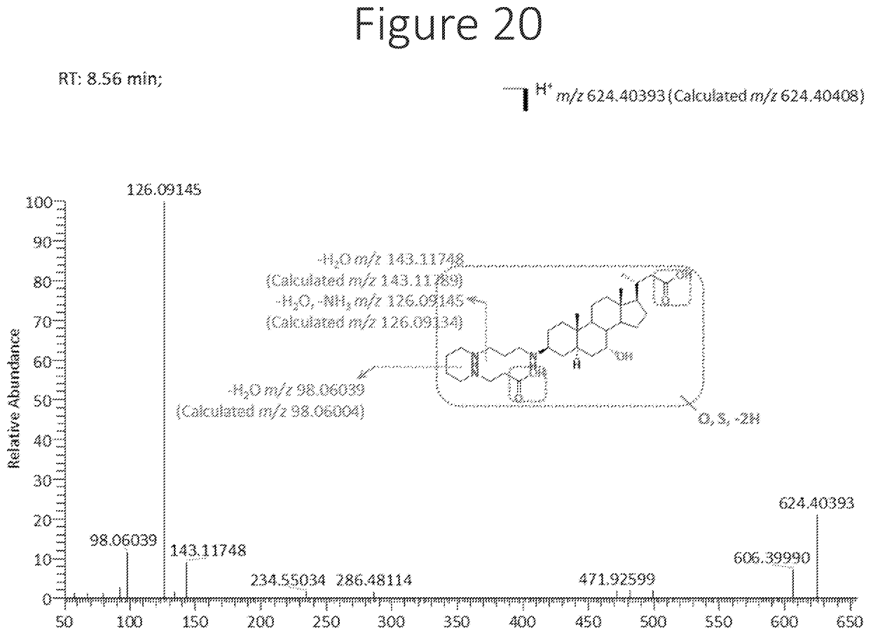

In particular, the XIC chromotagraphs from rat bile samples are shown in FIG. 15, FIG. 16 shows the MS/MS fragmentation spectrum of ENT-03S. FIG. 17 shows the MS/MS fragmentation spectrum of an ENT-03S derivative designated as M1-RB. FIG. 18 shows the MS/MS fragmentation spectrum of an ENT-03S derivative designated as M2-RB. FIG. 19 shows the MS/MS fragmentation spectrum of an ENT-03S derivative designated as M3-RB. FIG. 20 shows the MS/MS fragmentation spectrum of an ENT-03S derivative designated as M4-RB.

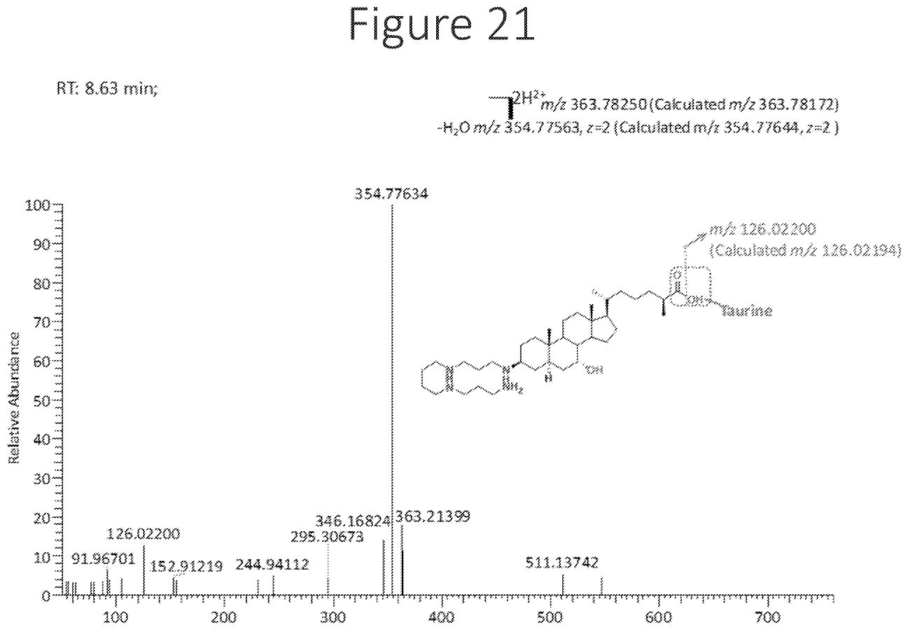

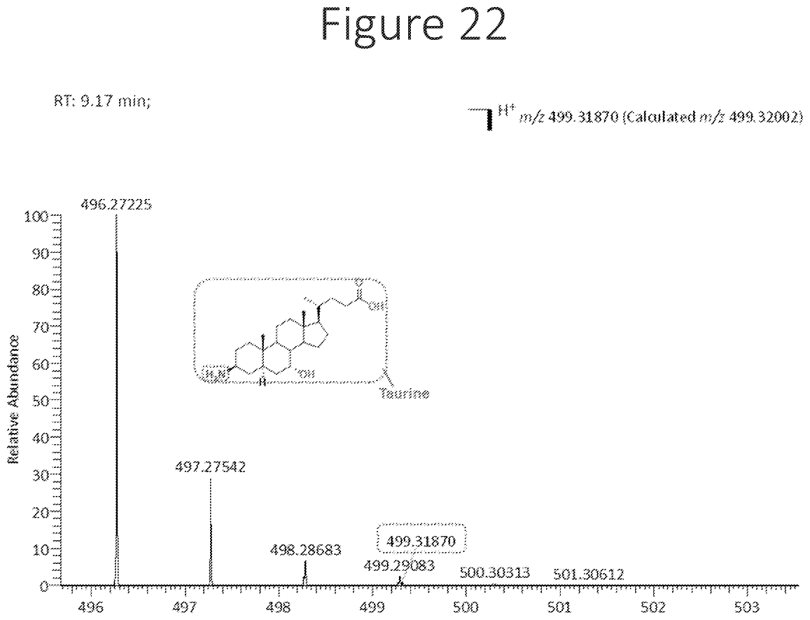

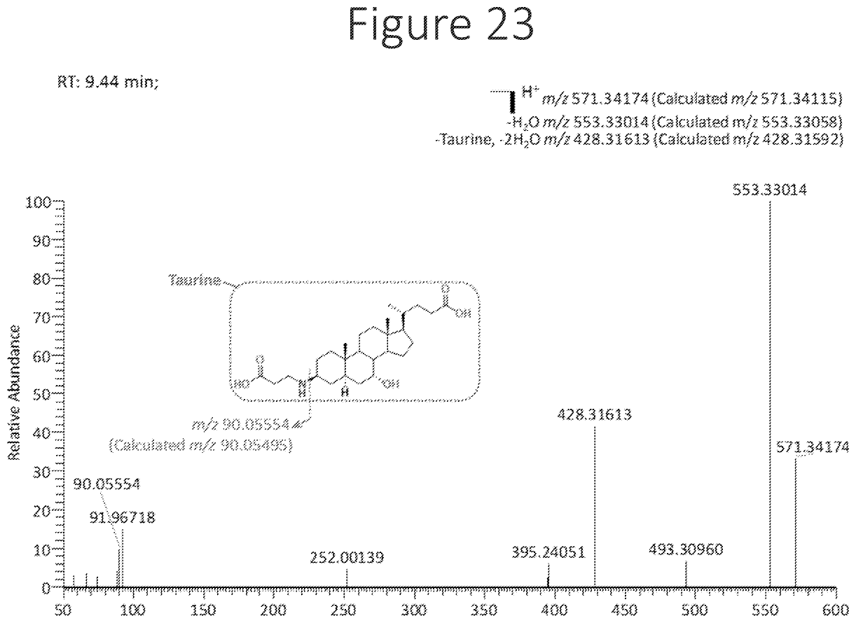

FIG. 21 shows the MS/MS fragmentation spectrum of an ENT-03S derivative designated as MS-RB. FIG. 22 shows the full MS spectrum of an ENT-03S derivative designated as M6-RB. FIG. 23 shows the MS/MS fragmentation spectrum of an ENT-03S derivative designated as M8-RB.

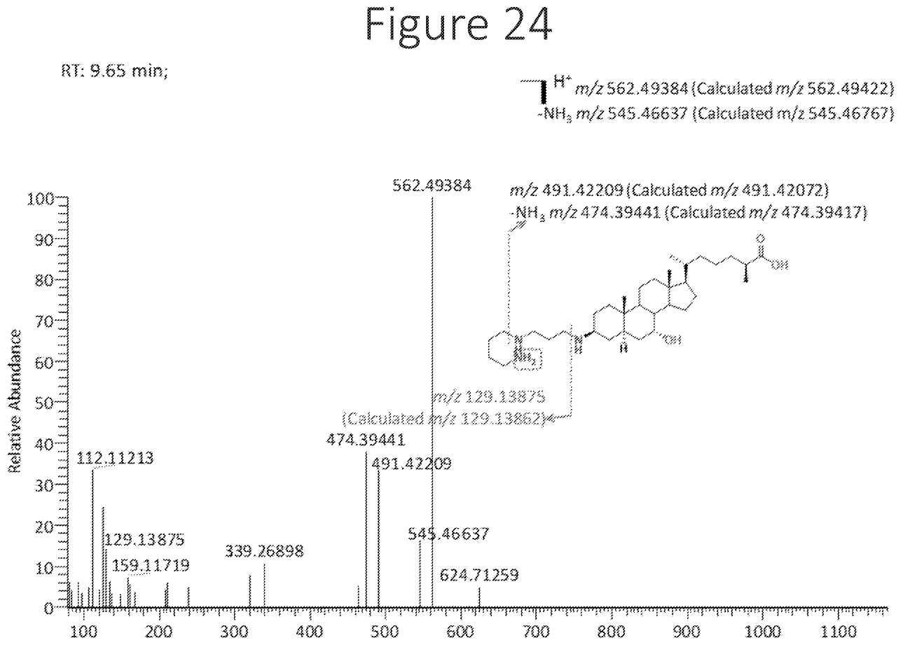

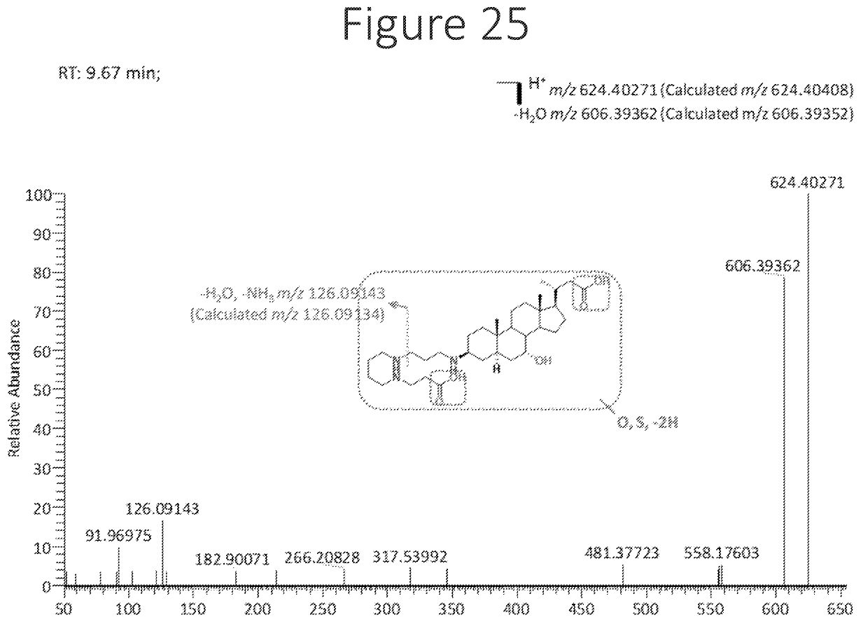

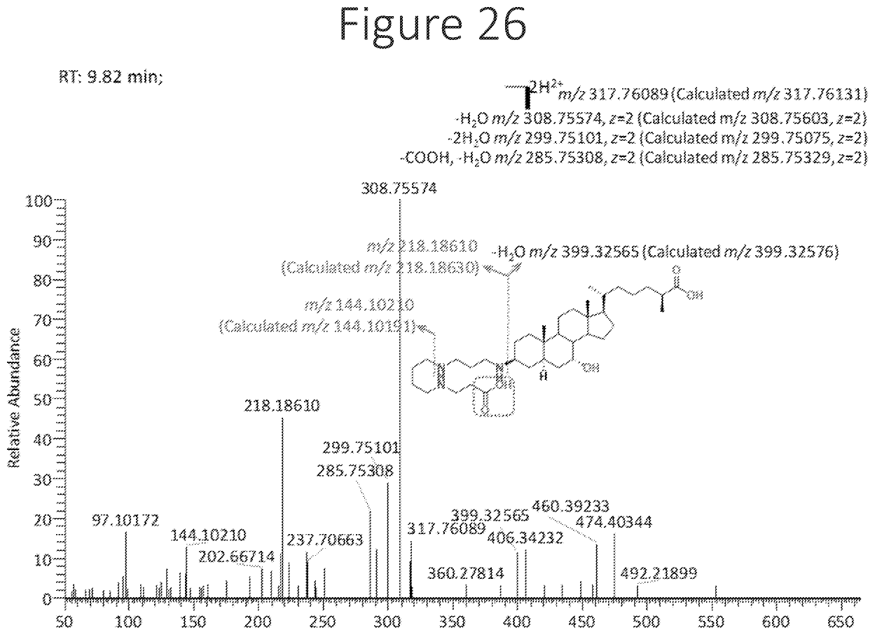

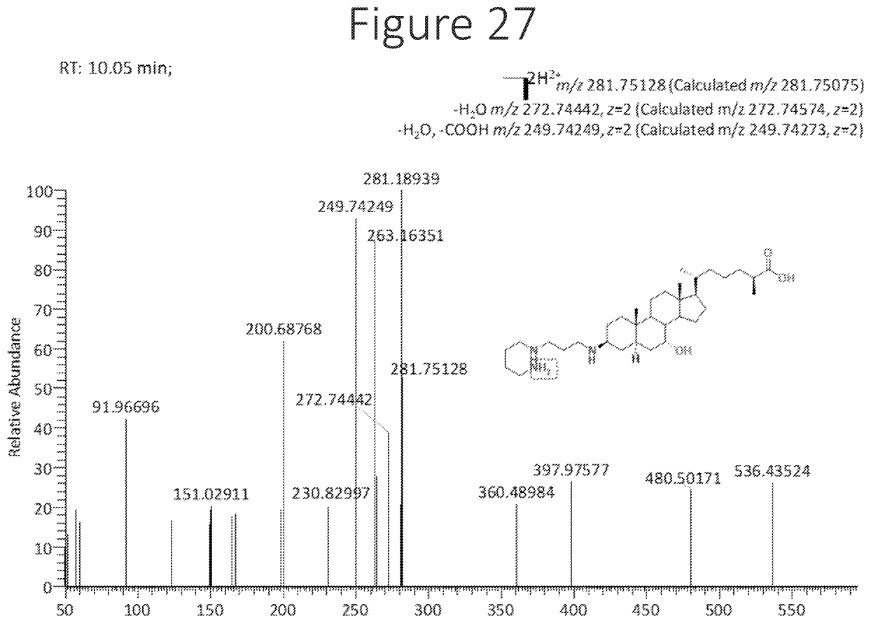

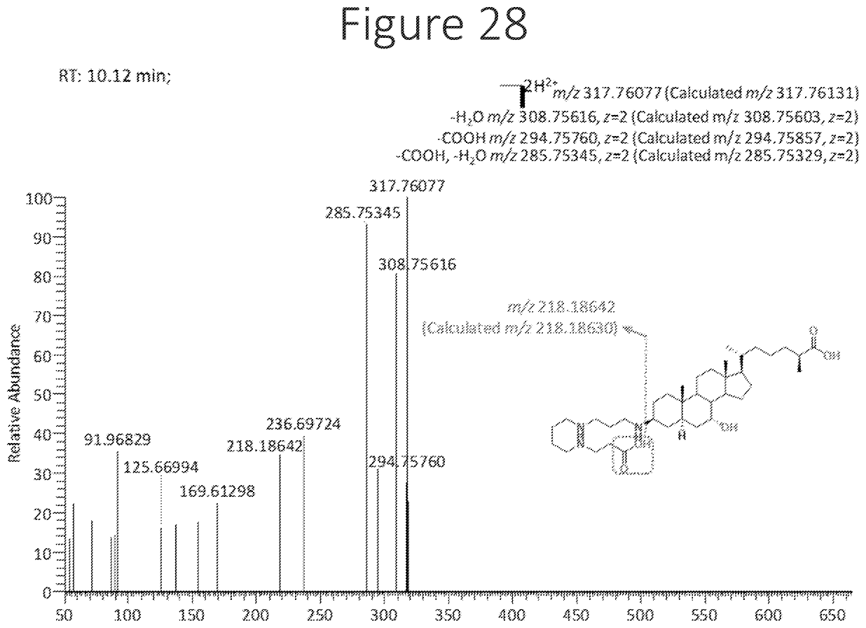

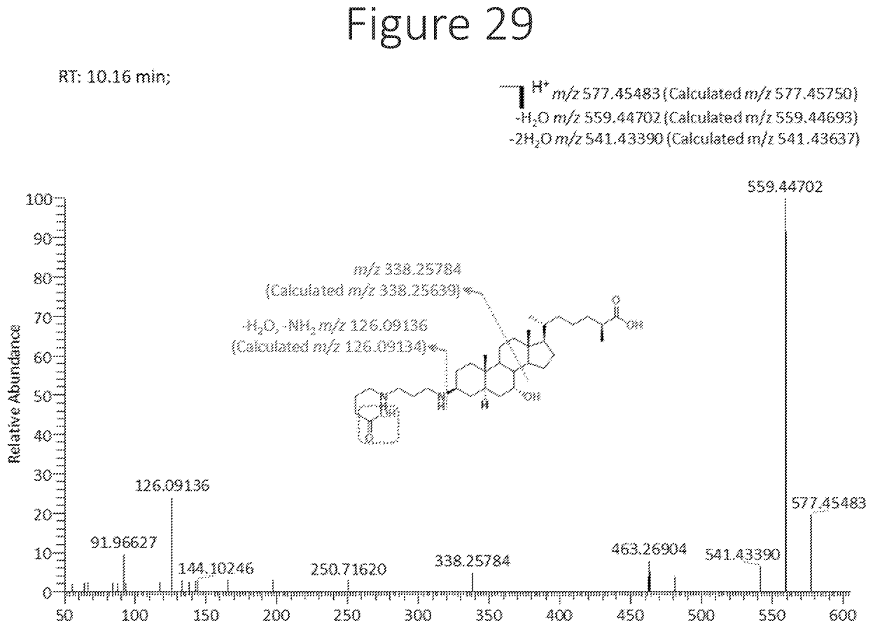

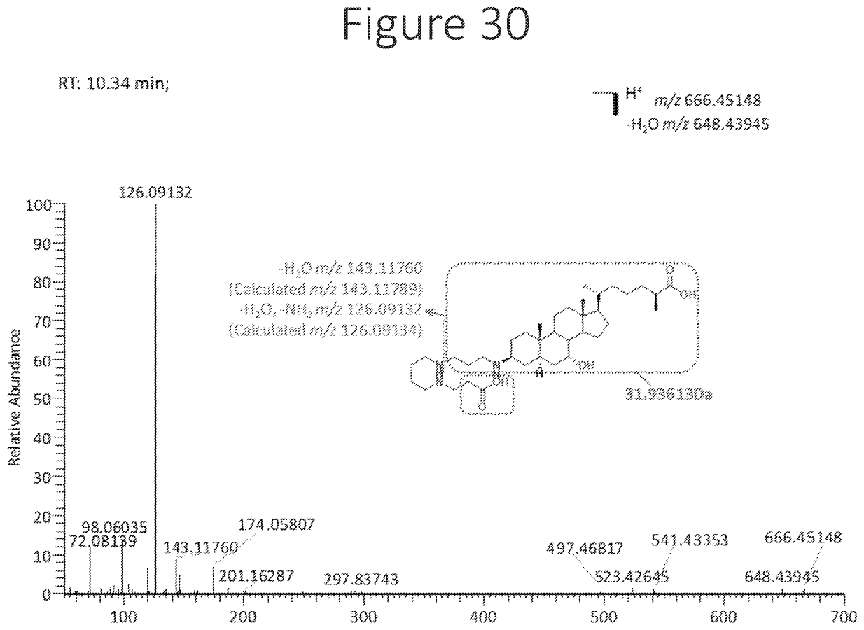

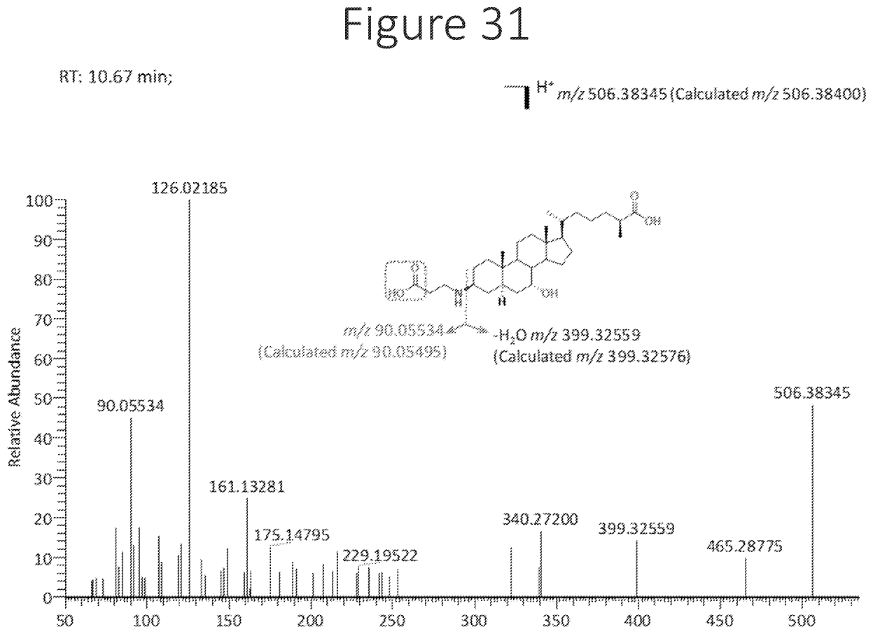

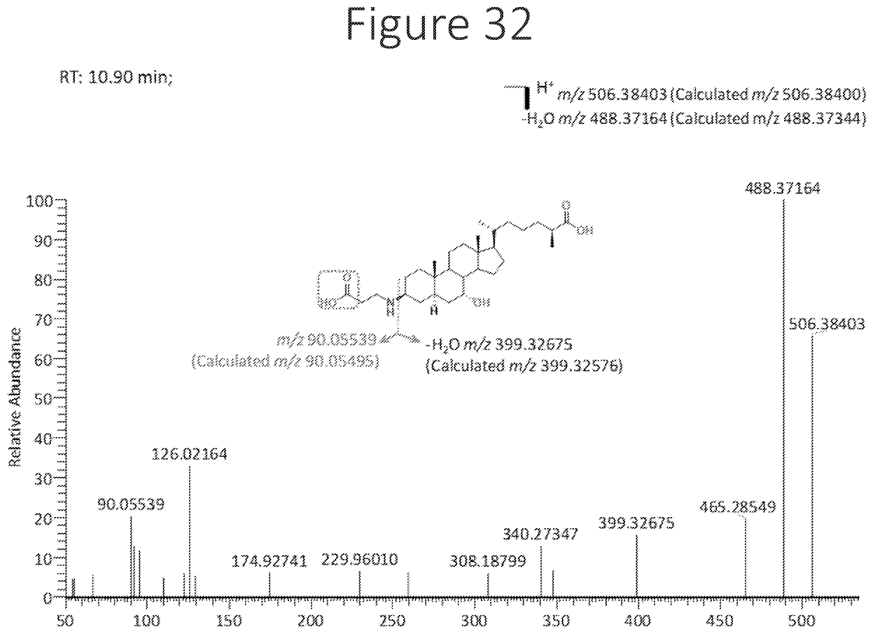

FIG. 24 shows the MS/MS fragmentation spectrum of an ENT-03S derivative designated as M9-RB. FIG. 25 shows the MS/MS fragmentation spectrum of an ENT-03S derivative designated as M10-RB. FIG. 26 shows the MS/MS fragmentation spectra of an ENT-03S derivative designated as ENT-25S (M11-RB). FIG. 27 shows the MS/MS fragmentation spectrum of an ENT-03S derivative designated as M12-RB. FIG. 28 shows the MS/MS fragmentation spectrum of an ENT-03S derivative designated as M13-RB. FIG. 29 shows the MS/MS fragmentation spectrum of an ENT-03S derivative designated as M14-RB. FIG. 30 shows the MS/MS fragmentation spectra of an ENT-03S derivative designated as M15-RB. FIG. 31 shows the MS/MS fragmentation spectrum of an ENT-03S derivative designated as M16-RB. FIG. 32 shows the MS/MS fragmentation spectrum of an ENT-03S derivative designated as M17-RB.

### Example 3

The purpose of this example was to further characterize the novel aminosterol designated as ENT-19.

The novel aminosterol 3-((aminopropyl)amino)butanoic-7uc-hydroxy-25[R,S]-cholestenoic acid i-CI Salt, designated as ENT-19, has the following chemical structure:

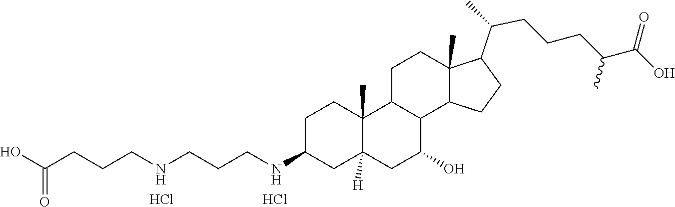 [SMILES: CC(CCC[C@@H](C)C1CCC2C3C(CC[C@@]21C)[C@@]1(C)CC[C@H](NCCCNCCCC(=O)O)C[C@@H]1C[C@H]3O)C(=O)O ; Cl ; Cl]

The compound has a molecular weight of 649.78 (576.86), and a chemical formula of C₃₄H₆₀N₂O₅-2HCl.

ENT-19 was found to be greater than 95% pure as determined by HPCL (95.27%), and the compound conforms to structure as verified by LC-MS (Shimadzu LCMS2020). Testing parameters are shown below in Table 15.

TABLE 15
Identity by LC-MS

| Instrument | Shimadzu LCMS2020 | Ionization | ES+ |
|---|---|---|---|
| Mobile Phase | A: 0.1% TFA in Water B: 0.1% TFA in Acetonitrile | | |
| Column | Luna C8 (4.6 × | Gradient | 10-90% B/20 min |
| | 150 mm, 5 μm) | | |
| Flow Rate | 1 mL/min | | |

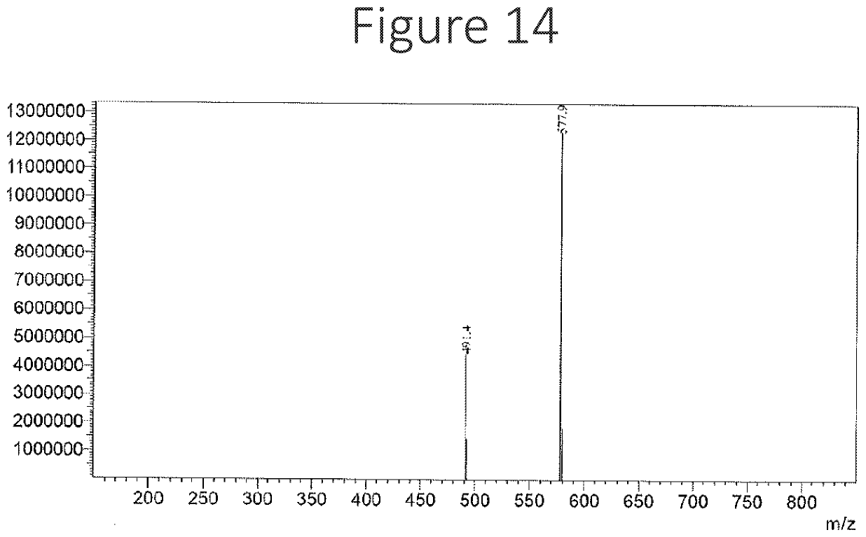

A chromatogram of the compound is shown in FIG. 14. Characterization of Peaks 1-5 is shown below in Table 16

TABLE 16

| | | | | Peak Area |
|---|---|---|---|---|
| Peak | Retention Time (min) | Area | Height | Percentage |
| 1 | 12.385 | 50032 | 9073 | 1.002 |
| 2 | 12.555 | 4757714 | 681512 | 95.266 |
| 3 | 12.915 | 31229 | 5452 | 0.625 |
| 4 | 13.104 | 99850 | 14337 | 1.999 |
| 5 | 13.993 | 55321 | 14468 | 1.108 |

### Example 4: Preparation of an Aminosterol Subcutaneous Formulation

The purpose of this example was to describe preparation of a subcutaneous formulation of the aminosterol ENT-03S. The structure of ENT-03S is shown below:

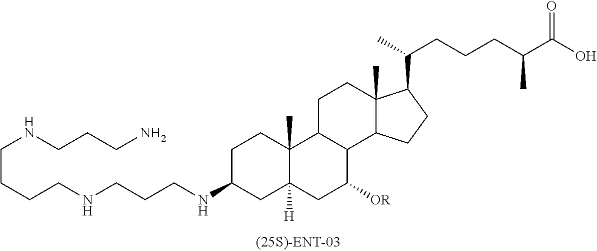

Preparation of a 10 ml formulation sample comprised the following steps: First, 2.51 grams of 2-HP β cyclodextrin as a powder and 0.5 grams of ENT-03S (free base sodium salt) as a powder were added to a vessel and the powders mixed thoroughly. 7.5 ml of distilled water was added to the vessel, the mixture stirred or shaken, resulting in a clear solution with small amounts of undissolved solids. The solution was warmed at 40° C. until all particulates had solubilized. The solution was adjusted to pH 7.3 by addition of IM H3PO₄ and left at room temperature for 24 hours, during which the pH rose to about 7 6. An additional IM H3P04 was then added to bring the pH to 7.4-7.5. The volume of the solution was brought to 10 ml with distilled water. The solution was sterile filtered through a 0.2 μm cellulose acetate membrane, followed by storage at room temperature.

The final composition consisted of the following:
ENT-03S 50 mg/ml (active)
2-hydroxypropyl β-cyclodextrin 25% (w/v)
Phosphate, about 80 mM
pH 7.4-7.6.

This example demonstrates the successful preparation of an aminosterol cyclodextrin formulation suitable for subcutaneous administration.

### Example 5: Evaluation of the Subcutaneous Aminosterol Cyclodextrin Formulation of Example 4

The purpose of this example was to evaluate the aminosterol cyclodextrin formulation prepared in Example 4.

An insulin syringe was loaded with 1 ml of the subcutaneous formulation of ENT-03S prepared in Example 4. The contents of the syringe were injected subcutaneously in the abdominal area of a human subject creating a visible swelling. A mild stinging sensation was noted, which disappeared within a few minutes. Over the course of an hour, the swelling disappeared.

This example demonstrates that the aminosterol cyclodextrin formulation can be administered subcutaneously, with minimal or no adverse effects, such as pain upon administration.

### Example 6: Pharmacokinetic Evaluation of the Aminosterol Cyclodextrin Formulation of Example 4

The purpose of this example was to evaluate the pharmacokinetic properties of the aminosterol cyclodextrin formulation prepared as in Example 1.

In this example, ENT-03S was formulated with unmodified beta-cyclodextrin at a concentration of 25 mg/ml ENT-03, and 20% beta-cyclodextrin, pH 7.5, buffered with phosphate.

Male Sprague-Dawley rats were administered the ENT-03-cyclodextrin formulation subcutaneous at a dose of 10 mg/kg. Blood samples were drawn from the tail vein at various times, the concentrations of ENT-03 determined, and PK parameters determined.

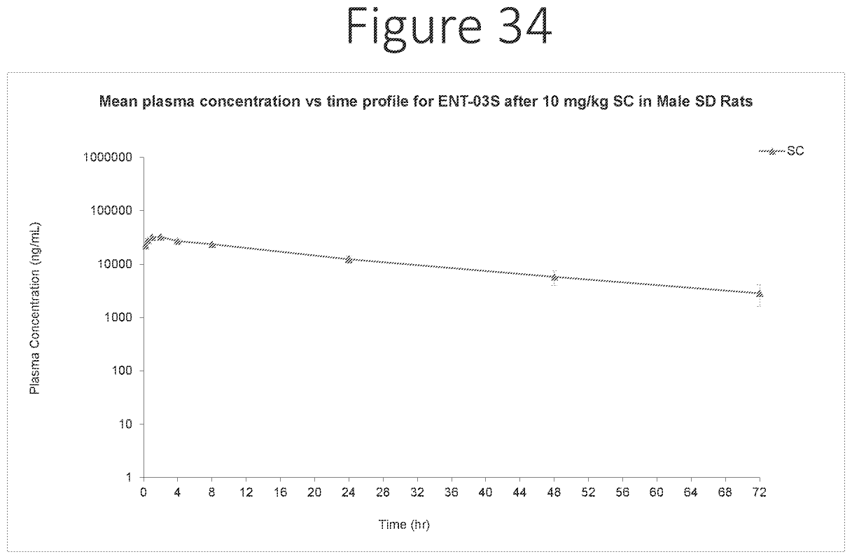

As shown in Table 17 and FIG. 34, ENT-03 rapidly diffuses from the site of subcutaneous administration into the bloodstream, reaching a peak concentration at 2 hours and progressively decreasing over the course of 72 hours.

TABLE 17

| Time | Concentration (ng/ml) | | | Mean | SD | CV |
|---|---|---|---|---|---|---|
| (h) | Rat 1 | Rat 2 | Rat 3 | (ng/mL) | (ng/mL) | (%) |
| 0.25 | 23100 | 22000 | 20800 | 21967 | 1150 | 5.24 |
| 0.5 | 25700 | 27100 | 30200 | 27667 | 2303 | 8.32 |
| 1 | 30700 | 31700 | 33000 | 31800 | 1153 | 3.63 |
| 2 | 31300 | 32700 | 33300 | 32433 | 1026 | 3.16 |
| 4 | 25700 | 29600 | 25400 | 26900 | 2343 | 8.71 |
| 8 | 22700 | 25000 | 23000 | 23567 | 1250 | 5.31 |
| 24 | 10300 | 14100 | 12600 | 12333 | 1914 | 15.5 |
| 48 | 4080 | 7700 | 5620 | 5767 | 1823 | 31.6 |
| 72 | 1850 | 4200 | 2440 | 2830 | 1223 | 43.2 |

The T_{1/2}, Tₘₐₓ, Cₘₐₓ, AUCₗₐₛₜ, AUC_{inf}, and additional pharmacokinetic parameters are shown below in Table 18.

TABLE 18
Summary of ENT-03S SC Plasma pharmacokinetic parameters
SC Dose 10 mg/kg

| PK parameters | Unit | Rat 1 | Rat 2 | Rat 3 | Mean | SD | CV(%) |
|---|---|---|---|---|---|---|---|
| T_{1/2} | h | 19.4 | 27.5 | 20.3 | 22.4 | 4.4 | 19.8 |
| Tₘₐₓ | h | 2.00 | 2.00 | 2.00 | 2.00 | 0.00 | 0.000 |
| Cₘₐₓ | ng/ml | 31300 | 32700 | 33300 | 32433 | 1026 | 3.16 |
| AUCₗₐₛₜ | h*ng/ml | 715608 | 944488 | 811185 | 823760 | 114957 | 14.0 |
| AUC_{lnf} | h*ng/ml | 767327 | 1110949 | 882526 | 920267 | 174892 | 19.0 |
| AUC_% Extrap_obs | % | 6.74 | 15.0 | 8.08 | 9.9 | 4.4 | 44.5 |
| MRT_{lnf}_obs | h | 24.3 | 36.3 | 27.2 | 29.3 | 6.3 | 21.4 |
| AUCₗₐₛₜ/D | h*mg/ml | 71561 | 94449 | 81119 | 82376 | 11496 | 14.0 |
| F | % | NA | NA | NA | NA | NA | NA |

### Example 7: Comparison with Non-Cyclodextrin Aminosterol Formulation

The benefits provided by the aminosterol-cyclodextrin formulation disclosed herein can be appreciated by comparing the pharmacokinetics of an aqueous solution of ENT-03S in the absence of cyclodextrin at a concentration of ENT-03S of 2 mg/ml at pH 74.

The dose chosen was the maximum ENT-03S concentration that could be delivered to the rat without causing visible local tissue necrosis.

Injection of 0. 1l of this formulation into a human subject caused severe pain within seconds that persisted for several hours, associated with local swelling, redness, and warmth that lasted for several days.

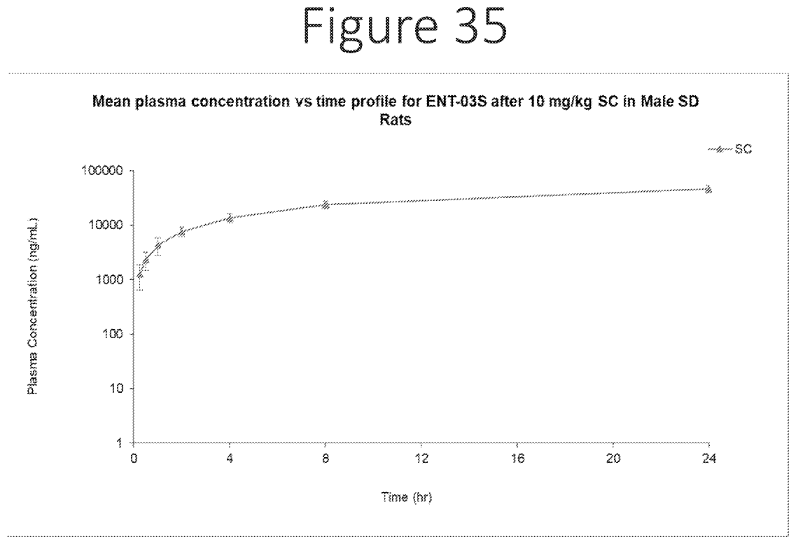

The experiment was conducted in male Sprague-Dawley rats following the same protocol as above. The formulation was administered subcutaneously at a dose of 10 mg/kg. The data obtained are shown below in Tables 19 and 20 and FIG. 35.

TABLE 19

| Time | Concentration (ng/ml) | | | Mean | SD | CV |
|---|---|---|---|---|---|---|
| (h) | Rat 1 | Rat 2 | Rat 3 | (ng/ml) | (ng/ml) | (%) |
| 0.25 | 1150 | 702 | 1930 | 1261 | 621 | 49.3 |
| 0.5 | 2300 | 1490 | 3220 | 2337 | 866 | 37.0 |
| 1 | 4910 | 2590 | 5570 | 4357 | 1565 | 35.9 |
| 2 | 8380 | 5900 | 8620 | 7633 | 1506 | 19.7 |
| 4 | 14100 | 10800 | 16100 | 13667 | 2676 | 19.6 |
| 8 | 26900 | 19600 | 24800 | 23767 | 3758 | 15.8 |
| 24 | 43300 | 40000 | 54300 | 45867 | 7488 | 16.3 |

TABLE 20

| PK parameters | Unit | Rat 1 | Rat 2 | Rat 3 | Mean | SD | CV(%) |
|---|---|---|---|---|---|---|---|
| T_{1/2} | h | NA | NA | NA | NA | NA | NA |
| Tₘₐₓ | h | 24.0 | 24.0 | 24.0 | 24.0 | 0.0 | 0.000 |
| Cₘₐₓ | ng/ml | 43300 | 40000 | 54300 | 45867 | 7488 | 16.3 |
| AUCₗₐₛₜ | h*ng/ml | 675103 | 559927 | 749498 | 661509 | 95514 | 14.4 |
| AUC_{lnf} | hang/mL | NA | NA | NA | NA | NA | NA |
| AUC_ %Extrap_obs | % | NA | NA | NA | NA | NA | NA |
| MRT_{lnf} __obs | h | NA | NA | NA | NA | NA | NA |
| AUCₗₐₛₜ/D | h*mg/mL | 67510 | 55993 | 74950 | 66151 | 9551 | 14.4 |
| F | % | NA | NA | NA | NA | NA | NA |

The slow entry of the aminosterol compound from the site of injection into the bloodstream is evident from the pharmacokinetic properties of the ENT-03S solution. See also FIG. 35. Maximal concentrations are not reached over 72 hours. The slow entry of ENT-03 into the circulation is likely due to both to binding to polyanionic proteoglycans present within subcutaneous layer of skin and local tissue damage.

### Example 8: Preparation of an Exemplary ENT-03 Cyclodextrin Formulation

The purpose of this example was to prepare an exemplary ENT-03 cyclodextrin formulation.

The resultant formulation consisted of:
50 mg/ml ENT-03 (active)
2HPβCD25% (w/v)
Sodium phosphate 40 mM
pH 7.4-5
Final volume: 12 ml

In the preparation process, 840 mg of ENT-03S HCl (70%) was added to a vessel. Next, 8 ml of water was added to the ENT-03S powder and the powder was dissolved completely.

Next, 1800 mg of 2H CD (sigma #778966) was added to the ENT-03S solution and dissolved completely. Next, 0.960 ml 0 5M Na2HPO4 was added (pH was about 4.5) (about 480 μmoles). 1M NaOH was titrated in (about 1 ml/l mmole to bring final pH to 7.4-7.5). Final volume was brought to 12 ml with water. Finally, the composition was sterile filtered through a 0.2 μm surfactant free cellulose acetate membrane.

This example demonstrates successful preparation of an ENT-03 cyclodextrin formulation suitable for administration intravenously, subcutaneously, or intramuscularly.

### Example 9: Preparation of an exemplary ENT-03 cyclodextrin formulation

The purpose of this example was to prepare an exemplary ENT-03 cyclodextrin formulation. The formulation procedure described in this example is for Subcutaneous ENT-03S Na+ Batch (e.g. for 402.5 mL Batch Size).

The final formulation consisted of the following components:

TABLE 21

| Component | Amount | |
|---|---|---|
| ENT-03S Na Powder (~82.68% | 23 g (active Free | |
| purity) | Base, 20.125 g) | |
| (2-Hydroxypropyl)-β-cyclodextrin | 100.7 | g |
| Phosphoric acid | ~30.84 | mmol |

| | |
|---|---|
| Milli Q Water (Particle free) | Up to 402.5 mL |

In a first step, 100.7 g of (2-Hydroxypropyl)-β-cyclodextrin were added into a sterile IL glass bottle, followed by the addition of 300 mL of accurately measured USP purified water into the container The solution was mixed well using a magnetic stir plate until it becomes a clear solution.

Next, the (2-Hydroxypropyl)-β-cyclodextrin solution was transferred into a HDPE container that contains 23 g of ENT-03 Na+ powder (API). The solution was mixed well by hand and then immediately transferred to a 37° C. water bath on a magnetic stir plate. The contents were then mixed further, and the solids were dissolved while warming until dissolution was completed. (pH1 is >9). Undissolved particles may remain at this stage. The pH was then adjusted to 7.5 by titrating the solution with IM H3PO4.

Next, the pH was adjusted to 7.3 (±0.1) by slowly adding more IM H3PO4 while the solution is still in the 37° C. water bath. The mixture is continued to be stirred to mix. The final volume (QS) was adjusted to 402.5 mL by adding ~102.5 mL of USP purified water. The solution was then mixed well using the magnetic stir plate until all particles are dissolved and the solution was clear while it was still at 37° C.

The solution was transferred to room temperature (RT) and allowed to sit at RT for −24 hrs. After 24 hrs, the pH was measured and adjusted to pH 7 3. The solution was then filtered through a 0.2 μm filter (PES) into a new sterile container (or the solution can be filtered into a final sterile B3DS container (bag) that is used for the Microcell filling machine). The formulated DIP solution is stored at RT and vials are filled within 24 hours of preparation.

While certain embodiments have been illustrated and described, it should be understood that changes and modifications can be made therein in accordance with ordinary skill in the art without departing from the technology in its broader aspects as defined in the following claims.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. An aminosterol compound, or a pharmaceutically acceptable salt, solvate, prodrug, enantiomer, or derivative thereof, having a structure selected from the group consisting of:

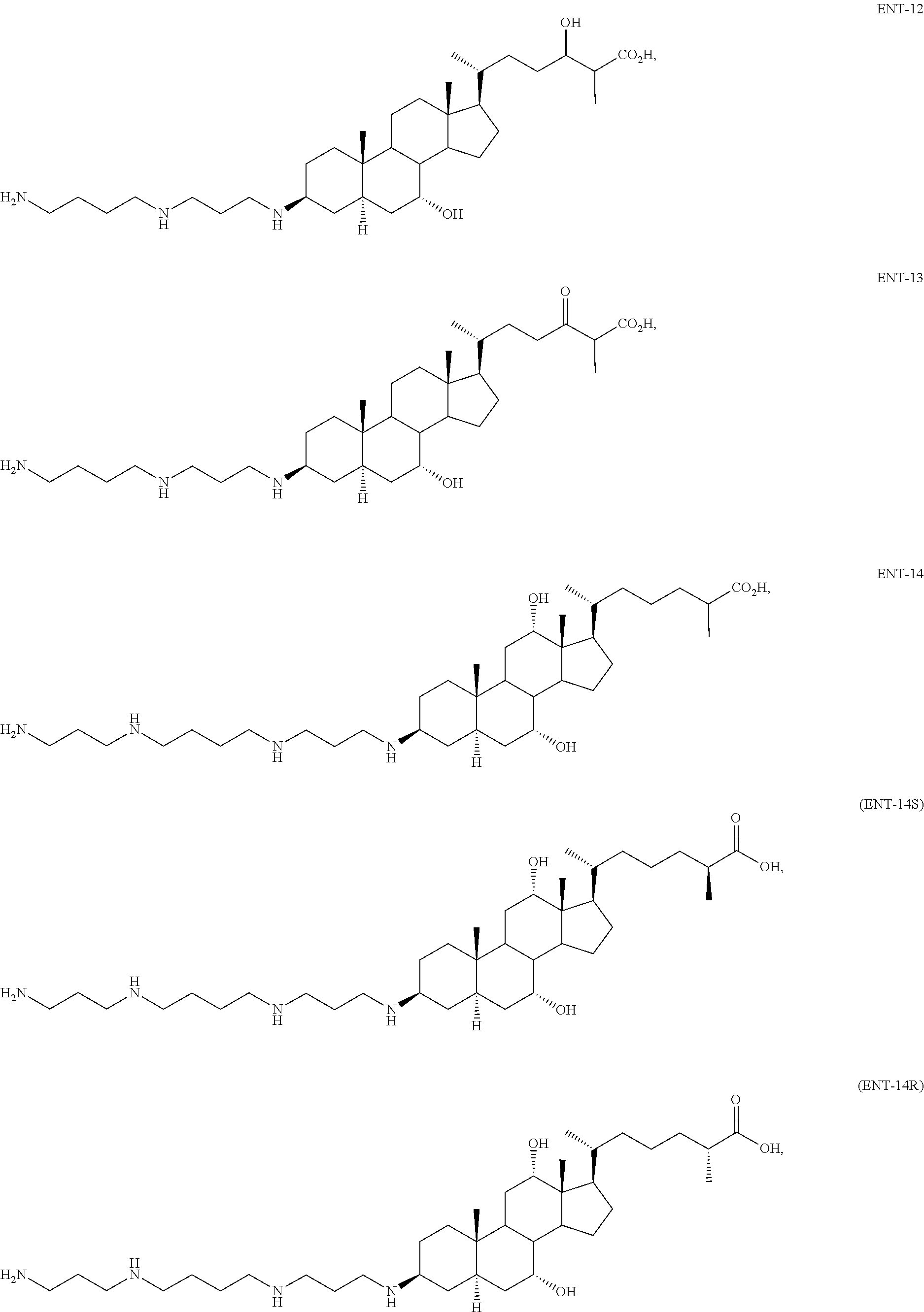

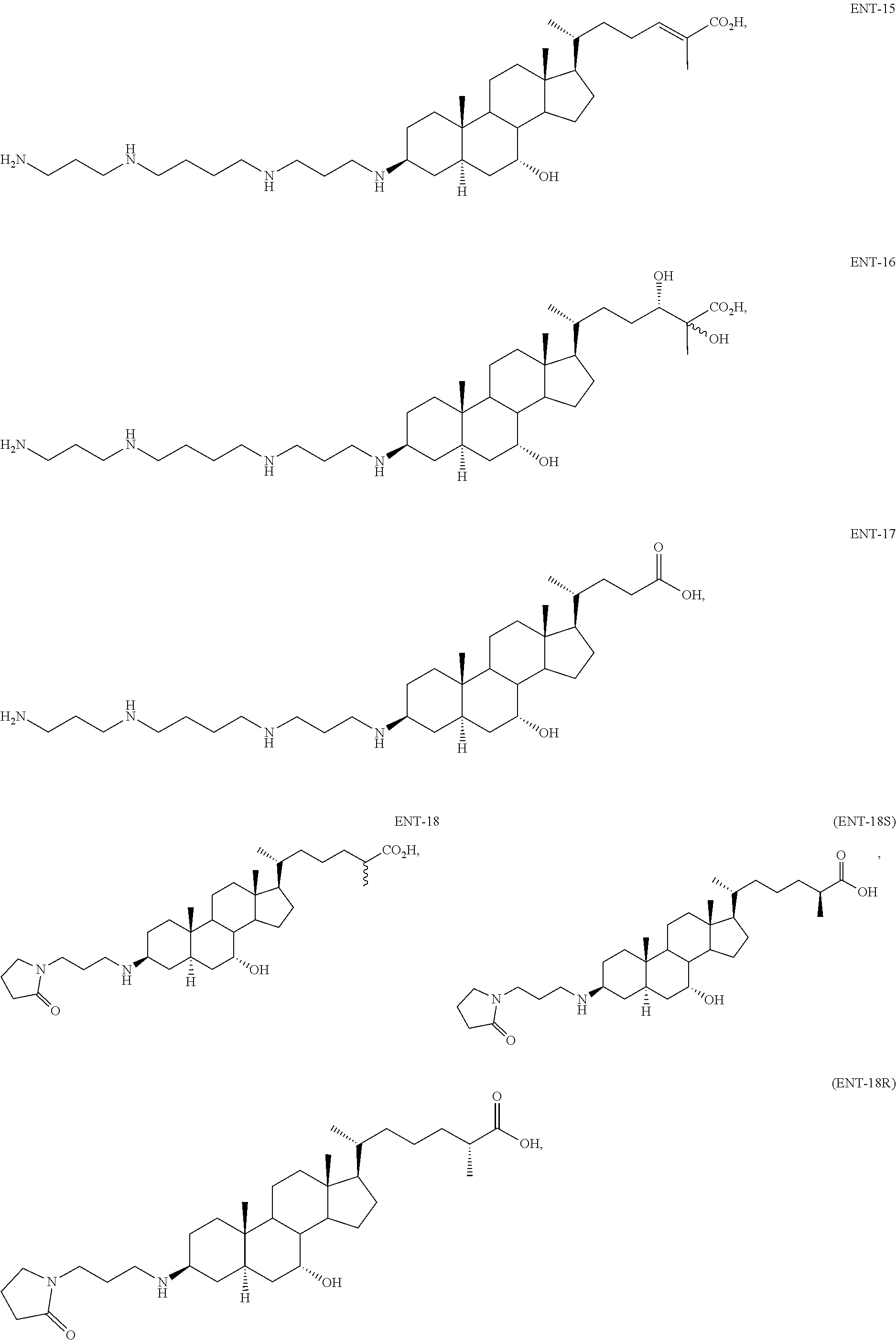

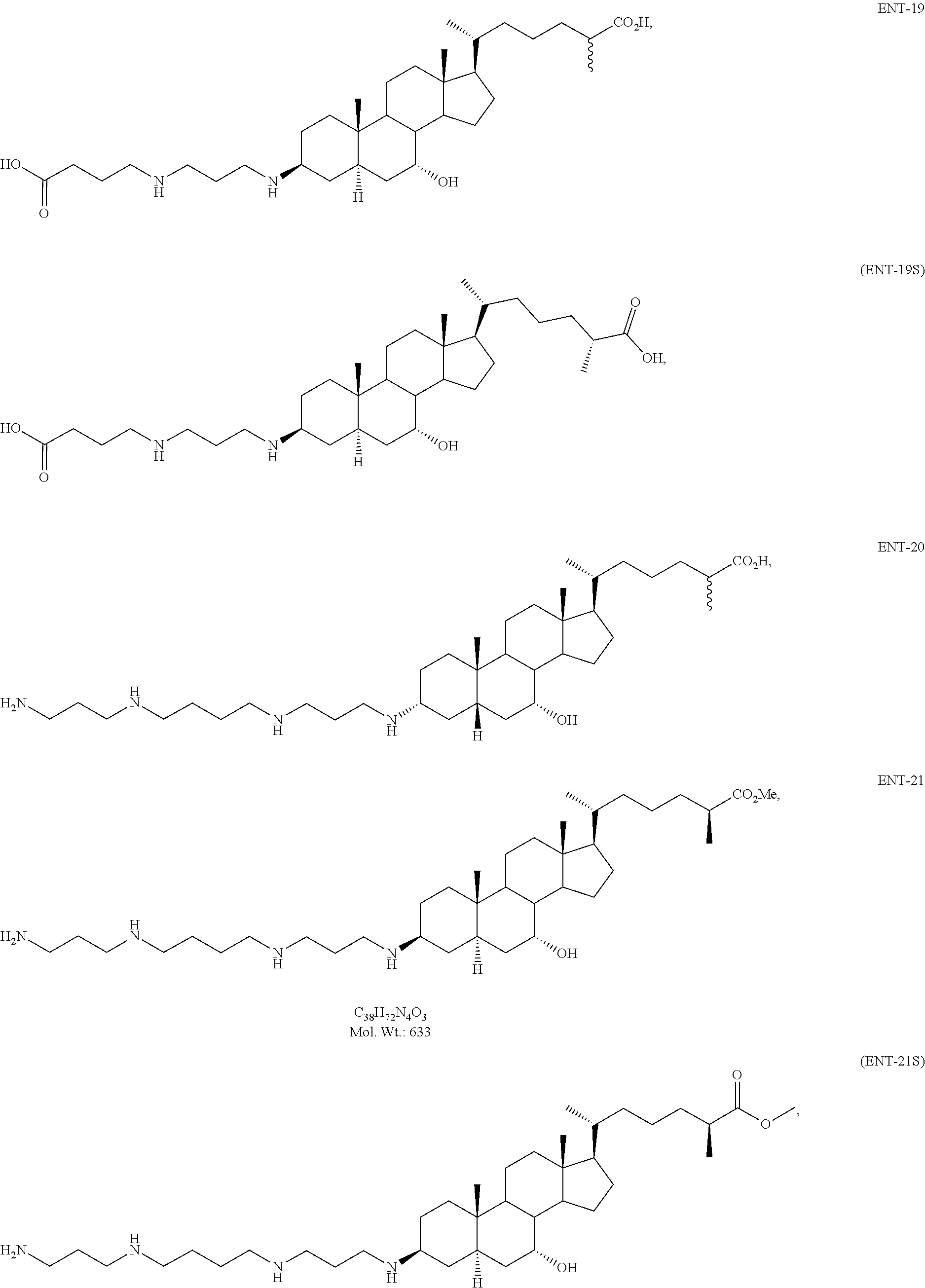

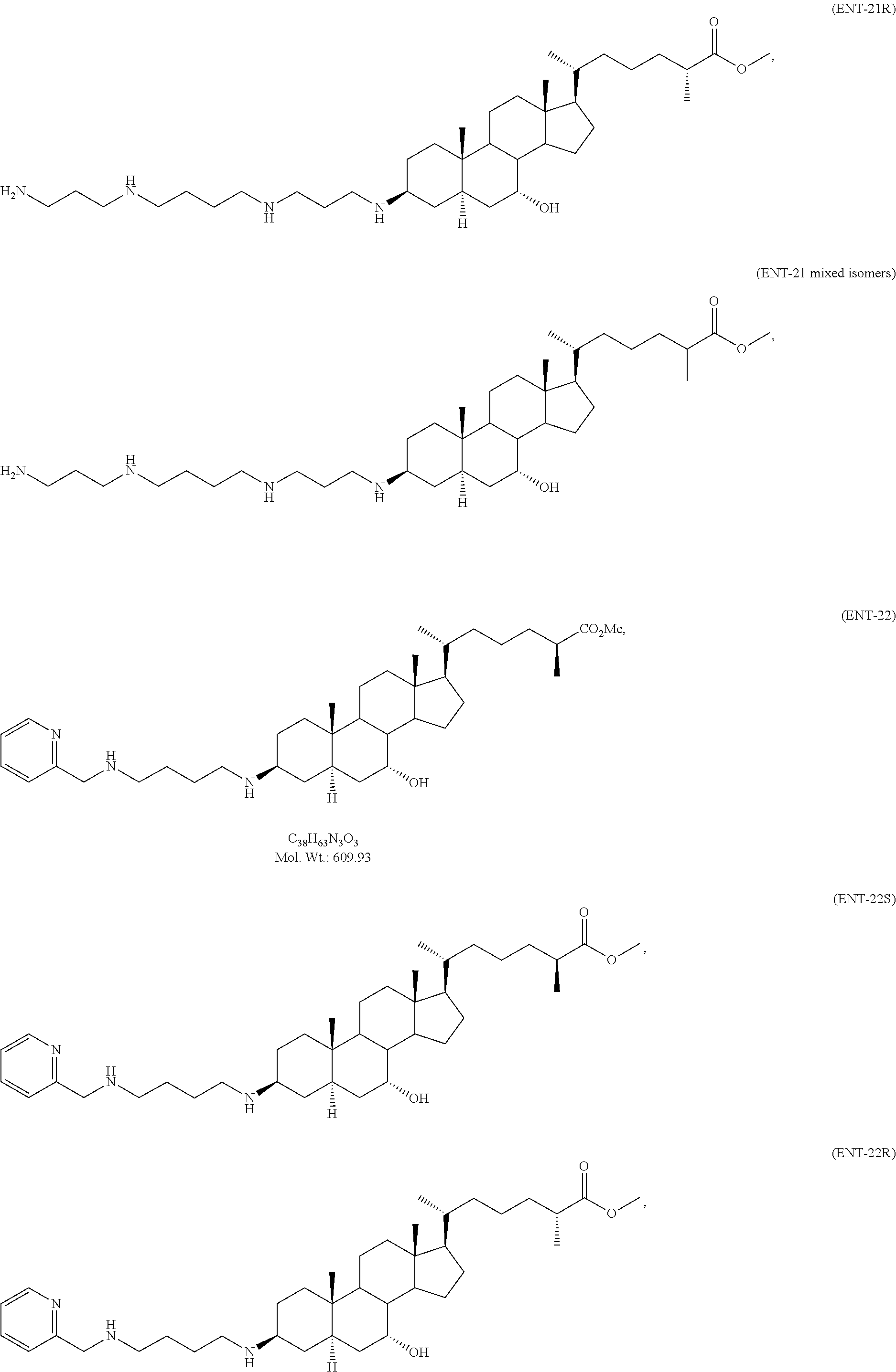

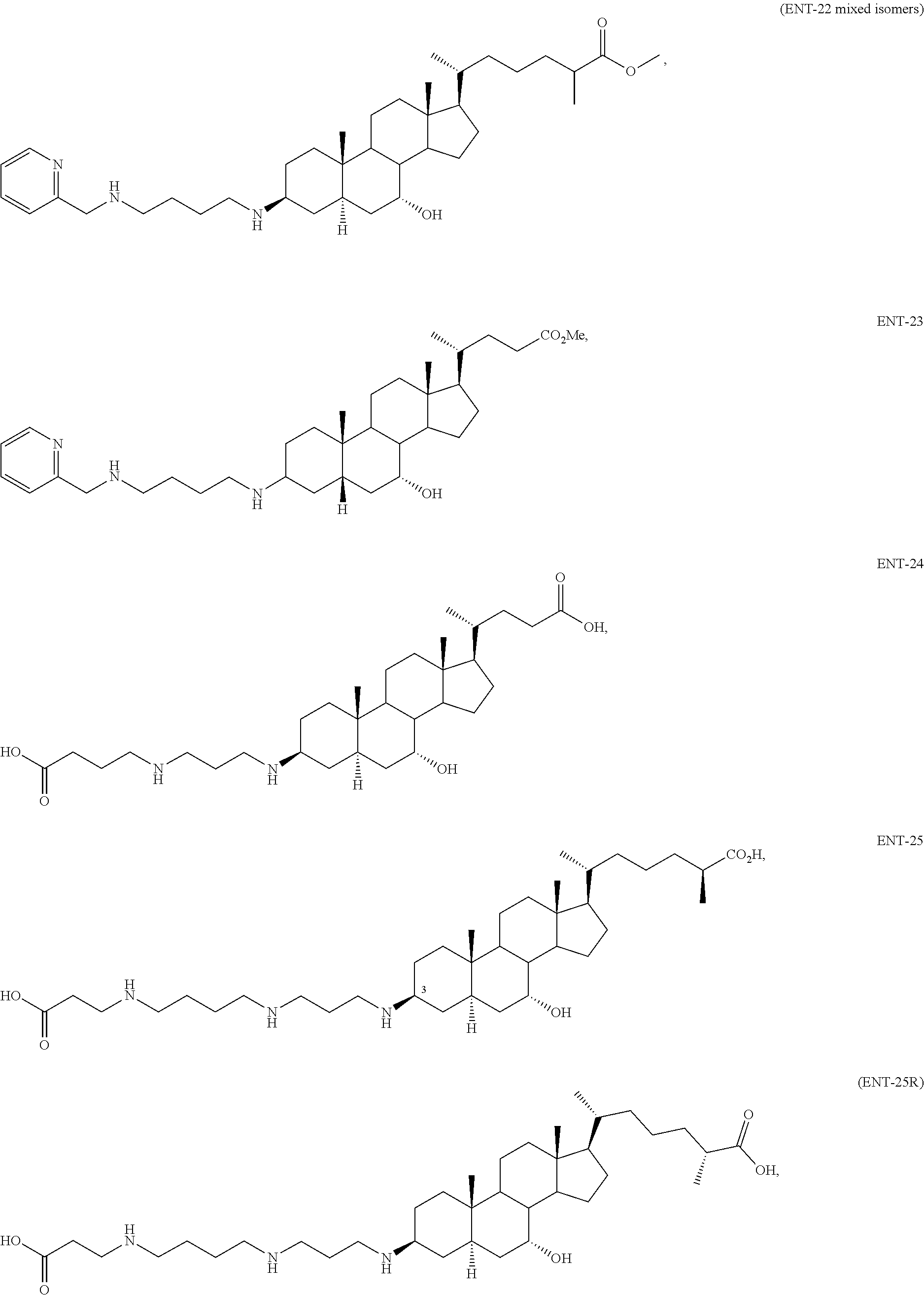 [SMILES: COC(=O)C(C)CCC[C@@H](C)[C@H]1CCC2C3C(CC[C@@]21C)[C@@]1(C)CC[C@H](NCCCCNCc2ccccn2)C[C@@H]1C[C@H]3O ; COC(=O)CC[C@@H](C)[C@H]1CCC2C3C(CC[C@@]21C)[C@@]1(C)CCC(NCCCCNCc2ccccn2)C[C@H]1C[C@H]3O ; C[C@H](CCC(=O)O)[C@H]1CCC2C3C(CC[C@@]21C)[C@@]1(C)CC[C@H](NCCCNCCCC(=O)O)C[C@@H]1C[C@H]3O ; C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; C[C@H](CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O]

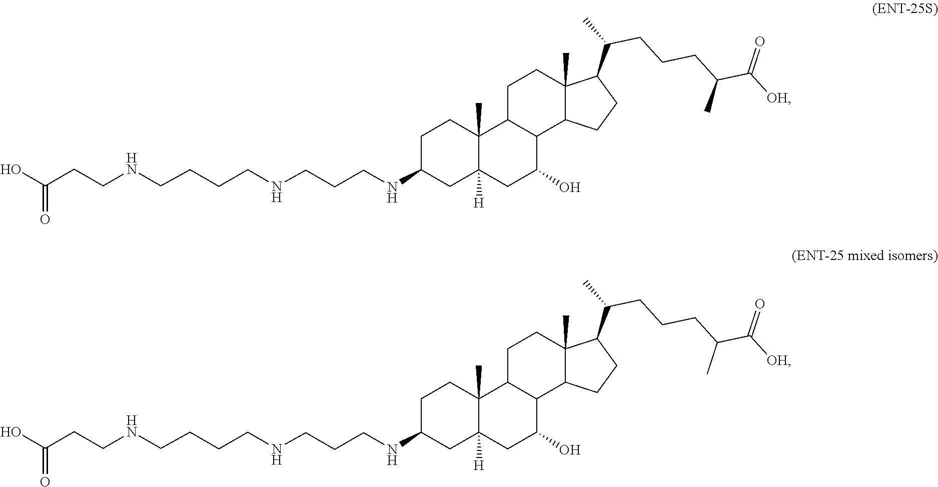 [SMILES: C[C@H](CCC[C@H](C)C(=O)O)[C@H]1CCC2C3C(CC[C@@]21C)[C@@]1(C)CC[C@H](NCCCNCCCCNCCC(=O)O)C[C@@H]1C[C@H]3O ; CC(CCC[C@@H](C)[C@H]1CCC2C3C(CC[C@@]21C)[C@@]1(C)CC[C@H](NCCCNCCCCNCCC(=O)O)C[C@@H]1C[C@H]3O)C(=O)O]

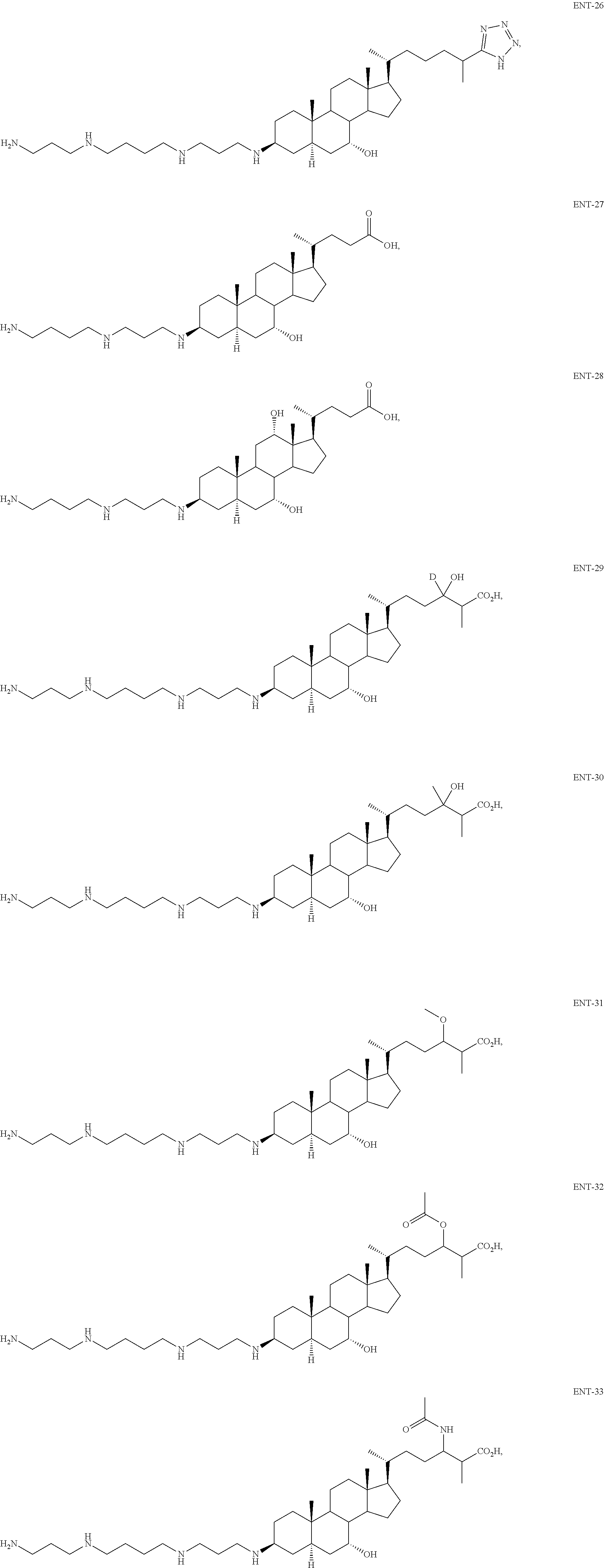

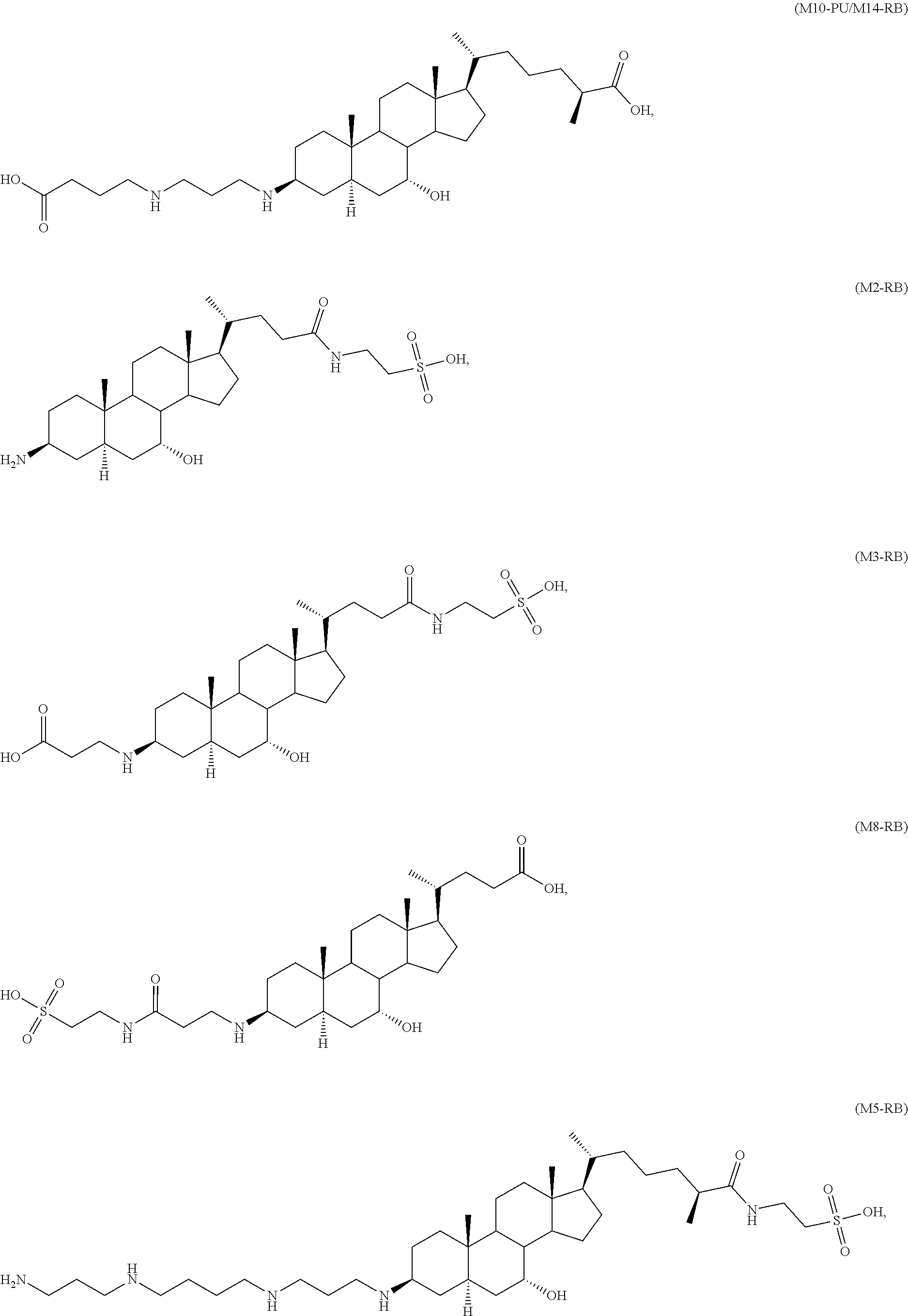 [SMILES: C[C@H](CCC[C@H](C)C(=O)O)[C@H]1CCC2C3C(CC[C@@]21C)[C@@]1(C)CC[C@H](NCCCNCCCC(=O)O)C[C@@H]1C[C@H]3O ; C[C@H](CCC(=O)NCCS(=O)(=O)O)[C@H]1CCC2C3C(CC[C@@]21C)[C@@]1(C)CC[C@H](N)C[C@@H]1C[C@H]3O ; C[C@H](CCC(=O)NCCS(=O)(=O)O)[C@H]1CCC2C3C(CC[C@@]21C)[C@@]1(C)CC[C@H](NCCC(=O)O)C[C@@H]1C[C@H]3O ; C[C@H](CCC(=O)O)[C@H]1CCC2C3C(CC[C@@]21C)[C@@]1(C)CC[C@H](NCCC(=O)NCCS(=O)(=O)O)C[C@@H]1C[C@H]3O ; C[C@H](CCC[C@H](C)C(=O)NCCS(=O)(=O)O)[C@H]1CCC2C3C(CC[C@@]21C)[C@@]1(C)CC[C@H](NCCCNCCCCNCCCN)C[C@@H]1C[C@H]3O]

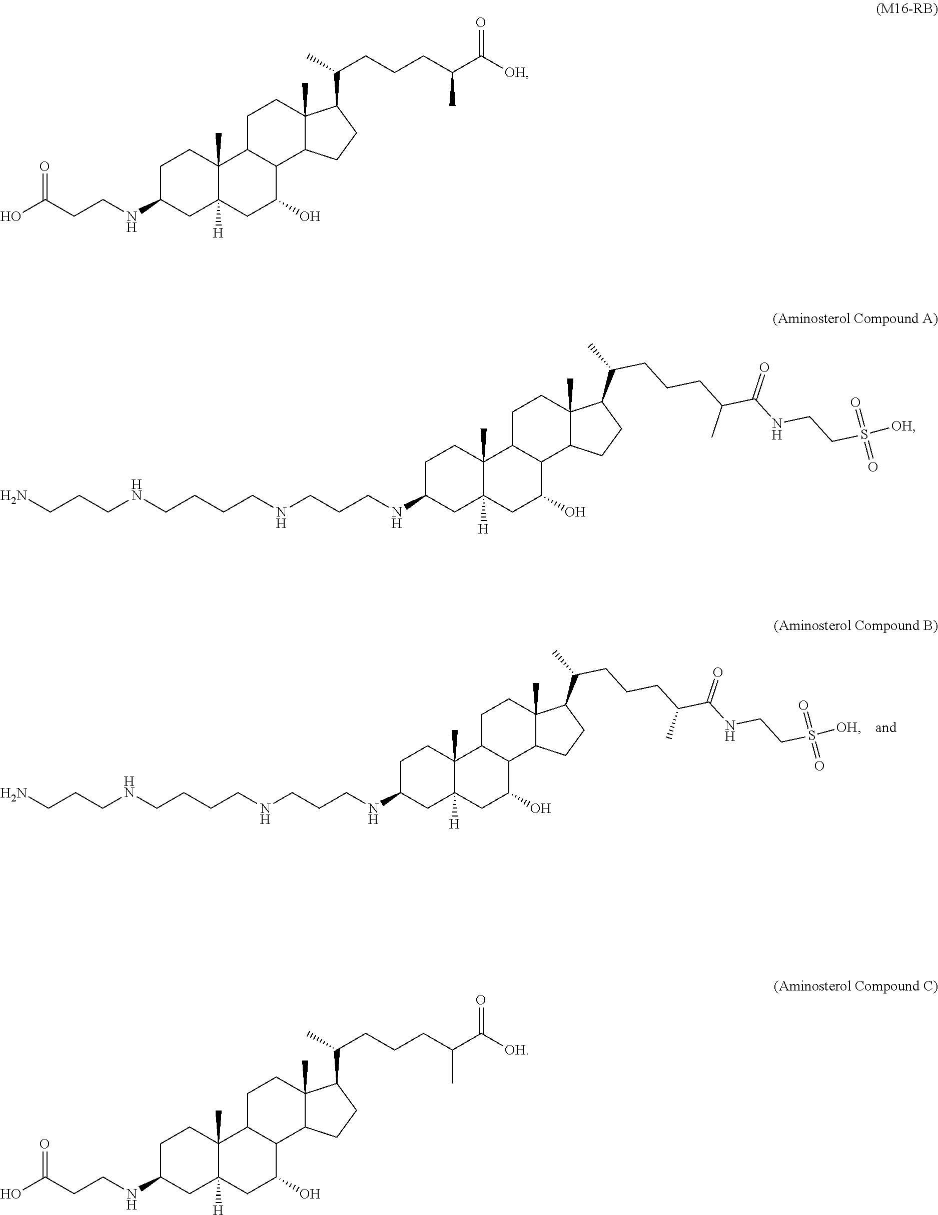

2. An aminosterol compound of Formula (I): 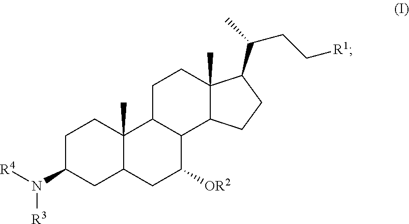
wherein:
R¹ is an optionally substituted C₁-C₈ alkyl, optionally substituted 2 to 8-membered heteroalkyl, optionally substituted heterocyclyl, or optionally substituted C₃-C₈ cycloalkyl;
R² is H or —C(O)R⁵, wherein R⁵ is an optionally substituted aryl, optionally substituted heteroaryl, optionally substituted C₁-C₆ alkyl, optionally substituted heterocyclyl, or optionally substituted C₃-C₈ cycloalkyl; and
R³ and R⁴ are each independently H or an optionally substituted 2 to 14-membered heteroalkyl;
or a pharmaceutically acceptable salt, solvate, prodrug, or derivative thereof;
wherein the compound is not:
(24R)-3β-({3-[(3-Aminopropyl)amino]propyl}amino)-7α-hydroxycholestan-24-yl hydrogen sulfate (squalamine);
(3R,6R)-6-((3S,5R,7R,8R,9S,10S,13R,14S,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptan-3-yl hydrogen sulfate (aminosterol 1436);
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid (ENT-03);
(2R,6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid (C₂₅ (R) ENT-03);
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic-2,3,3-d3 acid (ENT-03 d3);
(6R)-6-((3S,5R,7R,10S,13R,17R)-3-((3-((4-((3-aminopropyl)amino)butyl)amino)propyl)amino)-7-(benzoyloxy)-10,13-dimethylhexadecahydro-1H-cyclopenta[a]phenanthren-17-yl)-2-methylheptanoic acid;
(6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
(6R)-6-((3R,5S,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid;
(6R)-6-((3R,5S,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylheptanoic acid;
(6R)-6-((3R,5S,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methylheptanoic acid;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic-2-d acid;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic-2-d acid;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylheptanoic-2-d acid;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methylheptanoic-2-d acid;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-(methyl-d3)heptanoic acid;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-(methyl-d3)heptanoic acid;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-(methyl-d3)heptanoic acid;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-(methyl-d3)heptanoic acid;
(6R,E)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylhept-2-enoic acid;
(6R,E)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylhept-2-enoic acid;
(6R,E)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methylhept-2-enoic acid;
(6R,E)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methylhept-2-enoic acid;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-3-hydroxy-2-methylheptanoic acid;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-3-hydroxy-2-methylheptanoic acid;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-3-hydroxy-2-methylheptanoic acid;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-3-hydroxy-2-methylheptanoic acid;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)heptan-3-one;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)heptan-3-one;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)heptan-3-one;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)heptan-3-one;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methyl-3-oxoheptanoic acid;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoic acid;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoic acid;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoic acid;
methyl(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methyl-3-oxoheptanoate;
methyl(6R)-6-((3 S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoate;
methyl(6R)-6-((3 S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoate;
methyl(6R)-6-((3 S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoate;
isopropyl(6R)-6-((3 S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methyl-3-oxoheptanoate;
isopropyl(6R)-6-((3 S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methyl-3-oxoheptanoate;
isopropyl(6R)-6-((3 S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2-methyl-3-oxoheptanoate;
isopropyl(6R)-6-((3 S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2-methyl-3-oxoheptanoate;
(6R)-6-((3 S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2,3-dihydroxy-2-methylheptanoic acid;
(6R)-6-((3 S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2,3-dihydroxy-2-methylheptanoic acid;
(6R)-6-((3S,5R,7R,10S,13R,17R)-3-((3-((4-aminobutyl)amino)propyl)amino)-7,12-dihydroxy-10,13-dimethylhexadecahydro-1H-cyclopenta[a]phenanthren-17-yl)-2,3-dihydroxy-2-methylheptanoic acid;
(6R)-6-((3S,5R,7R,10S,13R,17R)-3-((3-((4-((3-aminopropyl)amino)butyl)amino)propyl)amino)-7,12-dihydroxy-10,13-dimethylhexadecahydro-1H-cyclopenta[a]phenanthren-17-yl)-2,3-dihydroxy-2-methylheptanoic acid;
(6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptane-2,3-diol;
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptane-2,3-diol;
(3S,5R,7R,10S,13R,17R)-3-((3-((4-aminobutyl)amino)propyl)amino)-17-((2R)-5,6-dihydroxy-6-methylheptan-2-yl)-10,13-dimethylhexadecahydro-1H-cyclopenta[a]phenanthrene-7,12-diol;
(3S,5R,7R,10S,13R,17R)-3-((3-((4-((3-aminopropyl)amino)butyl)amino)propyl)amino)-17-((2R)-5, 6-dihydroxy-6-methylheptan-2-yl)-10,13-dimethylhexadecahydro-1H-cyclopenta[a]phenanthrene-7,12-diol;
(6R)-6-((3S,5R,7R,10S,13R,17R)-3-((3-((4-ammoniobutyl)ammonio)propyl)ammonio)-7,12-dihydroxy-10,13-dimethylhexadecahydro-1H-cyclopenta[a]phenanthren-17-yl)-2-methylheptanoate (ENT-05);
(6R)-6-((3S,5R,7R,10S,13R,17R)-3-((3-((4-ammoniobutyl)ammonio)propyl)ammonio)-7-hydroxy-10,13-dimethylhexadecahydro-1H-cyclopenta[a]phenanthren-17-yl)-2-methylheptanoate (ENT-06);
(6R)-6-((3S,5R,7R,10S,13R,17R)-3-((3-((4-((3-ammoniopropyl)ammonio)butyl)ammonio)propyl)ammonio)-7-hydroxy-10,13-dimethylhexadecahydro-1H-cyclopenta[a]phenanthren-17-yl)-3-hydroxy-2-methylheptanoate-2,3-d2;
(2S,6R)-6-((3 S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-methylheptanoic acid (C₂₅ (S) ENT-03);
(2R,6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
(2S,6R)-6-((3R,5S,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid;
(2R,6R)-6-((3 S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid (C₂₅ (R) ENT-06);
(2S,6R)-6-((3 S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptanoic acid (C₂₅ (S) ENT-06);
(6R)-6-((3 S,5R,7R,10S,13R)-3-((3-((4-aminobutyl)amino)propyl)amino)-7-hydroxy-10,13-dimethylhexadecahydro-1H-cyclopenta[a]phenanthren-17-yl)-2-(methyl-d3)heptan-3-yl-1,1,1,2-d4 hydrogen sulfate (D7-squalamine);
(3R,6R)-6-((3S,5R,7R,10S,13R,17R)-3-((3-((4-aminobutyl)amino)propyl)amino)-7-hydroxy-10,13-dimethylhexadecahydro-1H-cyclopenta[a]phenanthren-17-yl)-2-methylheptan-3-yl-3-d hydrogen sulfate (24-D₁-squalamine); or
(6R)-6-((3S,5R,7R,10S,13R,17R)-3-((3-((4-((3-aminopropyl)amino)butyl)amino)propyl)amino)-7-hydroxy-10,13-dimethylhexadecahydro-1H-cyclopenta[a]phenanthren-17-yl)-2-(methyl-d3)heptan-3-yl-1,1,1,2-d4 hydrogen sulfate (D7-1436).

3. The aminosterol compound of claim 2, wherein R¹ is selected from

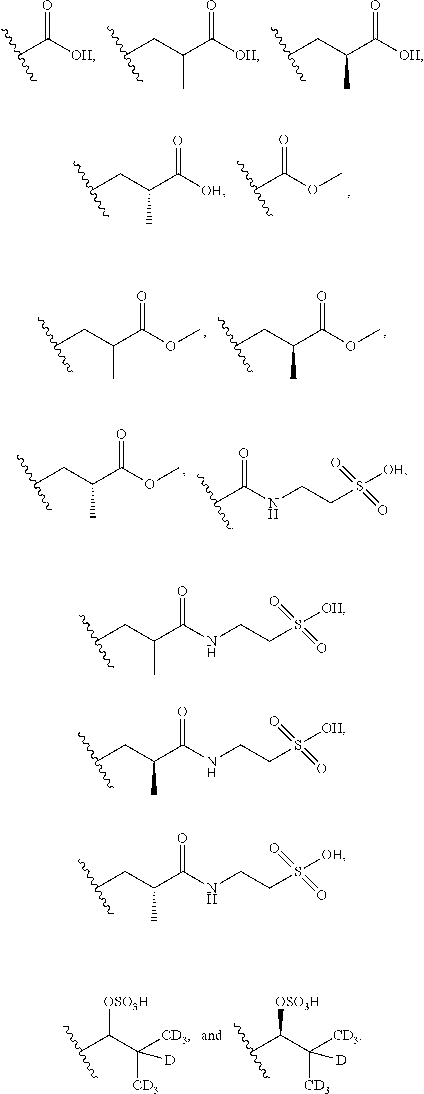

4. The aminosterol compound of claim 2, wherein R² is H.

5. The aminosterol compound of claim 2, wherein R³ and R⁴ are independently selected from H,

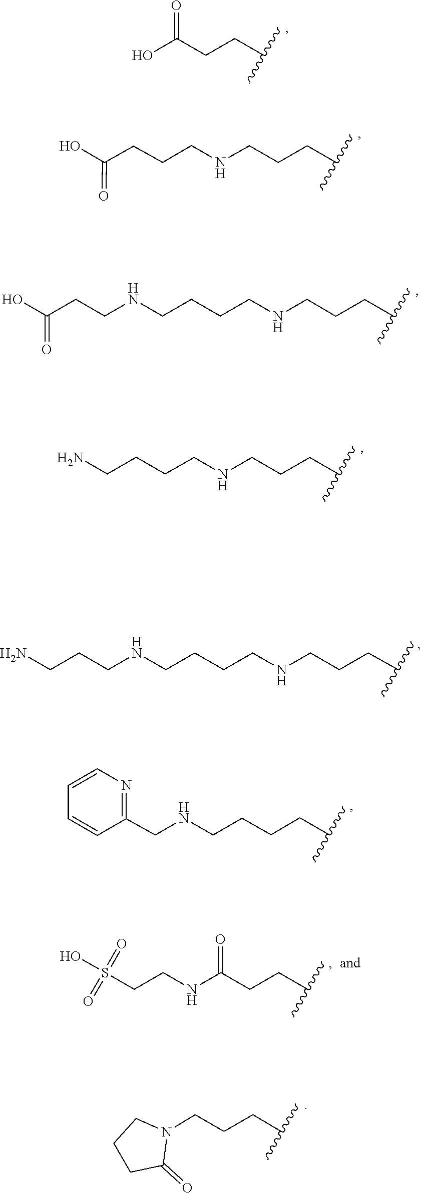

6. The aminosterol compound of claim 2, wherein R³ is H and R⁴ is independently selected from

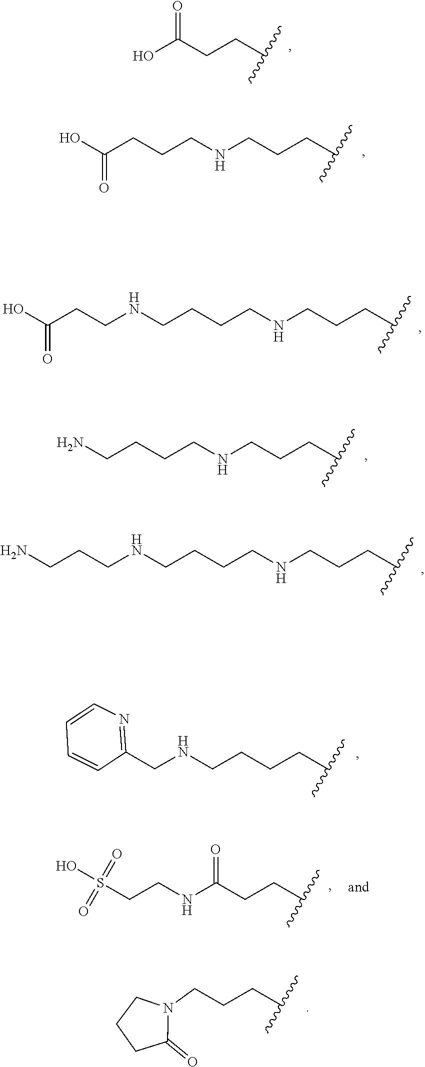

7. The aminosterol compound of claim 1, which is a hydrochloride salt or a sodium salt.

8. (canceled)

9. The aminosterol compound of claim 1, wherein:
(a) the diastereomeric excess (de), with respect to the C₂₅ carbon is about 80% to about 90%, about 90% to about 95%, about 95% to about 99%, about 99% to about 99.9%, or about 100% and/or
(b) at least one hydrogen of the aminosterol compound is replaced with deuterium.

10. (canceled)

11. A composition comprising an aminosterol compound according to claim 1, and at least one pharmaceutically acceptable carrier or excipient.

12. The composition of claim 11, comprising:
(a) one or more of the following: an aqueous carrier, a buffer, a sugar; and/or a polyol compound; and optionally
(b) wherein the composition further comprises at least one additional active agent.

13. The composition of claim 11, wherein the composition is formulated:
(a) for administration selected from the group consisting of oral, pulmonary, rectal, colonic, parenteral, intracisternal, intravaginal, intraperitoneal, intravenous, subcutaneous, intramuscular, nebulization, inhalation, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, and mixed immediate release and controlled release formulations;
(d) for oral administration;
(e) as an oral tablet or capsule;
(f) for intranasal administration; or
(g) any combination of (a), (b), (c), (d), (e), and/or (f).

14. An aqueous aminosterol composition comprising:
(a) an aminosterol, or a pharmaceutically acceptable salt, solvate, prodrug, enantiomer, or derivative thereof, and
(b) a cyclodextrin,
wherein the formulation is buffered to a pH of about 4 to 9, and
wherein the cyclodextrin is reversibly bound to a molecule of the aminosterol, or a pharmaceutically acceptable salt, solvate, prodrug, or derivative thereof, to form a cyclodextrin-aminosterol complex.

15. The aqueous aminosterol composition of claim 14, wherein the cyclodextrin is:
(a) an unmodified beta cyclodextrin or an unmodified gamma cyclodextrin:
(b) 2hydroxypropyl-β-Cyclodextrin (2HP-βCD); or
(c) Sulfobutylether-β-Cyclodextrin (SBE-β-CD).

16. (canceled)

17. The aqueous aminosterol composition of claim 14, wherein the pH:
(a) is from about 5 to about 8, or about 7 to about 8; or
(b) is from about 7.3 to about 7.5.

18. The aqueous aminosterol composition of claim 14, wherein the buffer is selected from the group consisting of an acetate buffer, phosphate buffer, citrate buffer, histidine buffer, tris(hydroxymethyl)aminomethane (TRIS), sodium phosphate, citric acid, acetic acid, KH₂PO₄, CHES, Borate, tromethamine, gluconic acid, lactic acid, tartaric acid, aspartic acid, glutamic acid, citric acid cycle intermediates, [tris(hydroxymethyl)methylamino]propanesulfonic acid (TAPS), 2-(bis(2-hydroxyethyl)amino)acetic acid (Bicine), N-[tris(hydroxymethyl)methyl]glycine (Tricine), 3-[N-tris(hydroxymethyl)methylamino]-2-hydroxypropanesulfonic acid (TABSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), piperazine-N,N′-bis(2-ethanesulfonic acid) (PIPES), dimethylarsenic acid (Cacadylate), and 2-(N-morpholino)ethanesulfonic acid (MES).

19. (canceled)

20. The aqueous aminosterol composition of claim 14, wherein:
(1) the cyclodextrin is present in an amount of:
(a) from about 10% up to about 40%, about 15% up to about 30%, about 18% to about 25%, or about 20% up to about 25%; or
(b) about 20% up to about 25% and/or
(2) the aminosterol is present in an amount of:
(a) from about 20 mg/ml up to about 75 mg/ml;
(b) from about 30 mg/ml up to about 65 mg/ml;
(c) from about 40 mg/ml up to about 55 mg/ml; or
(d) about 50 mg/ml; and/or
(3) the ratio of the concentrations (in mg/ml) of the aminosterol to the cyclodextrin is:
(a) about 0.1 to about 0.75:
(b) about 0.15 to about 0.5: or
(c) about 0.20.

21. (canceled)

22. (canceled)

23. (canceled)

24. The aqueous aminosterol composition of claim 14, wherein the aminosterol is selected from the group consisting of:

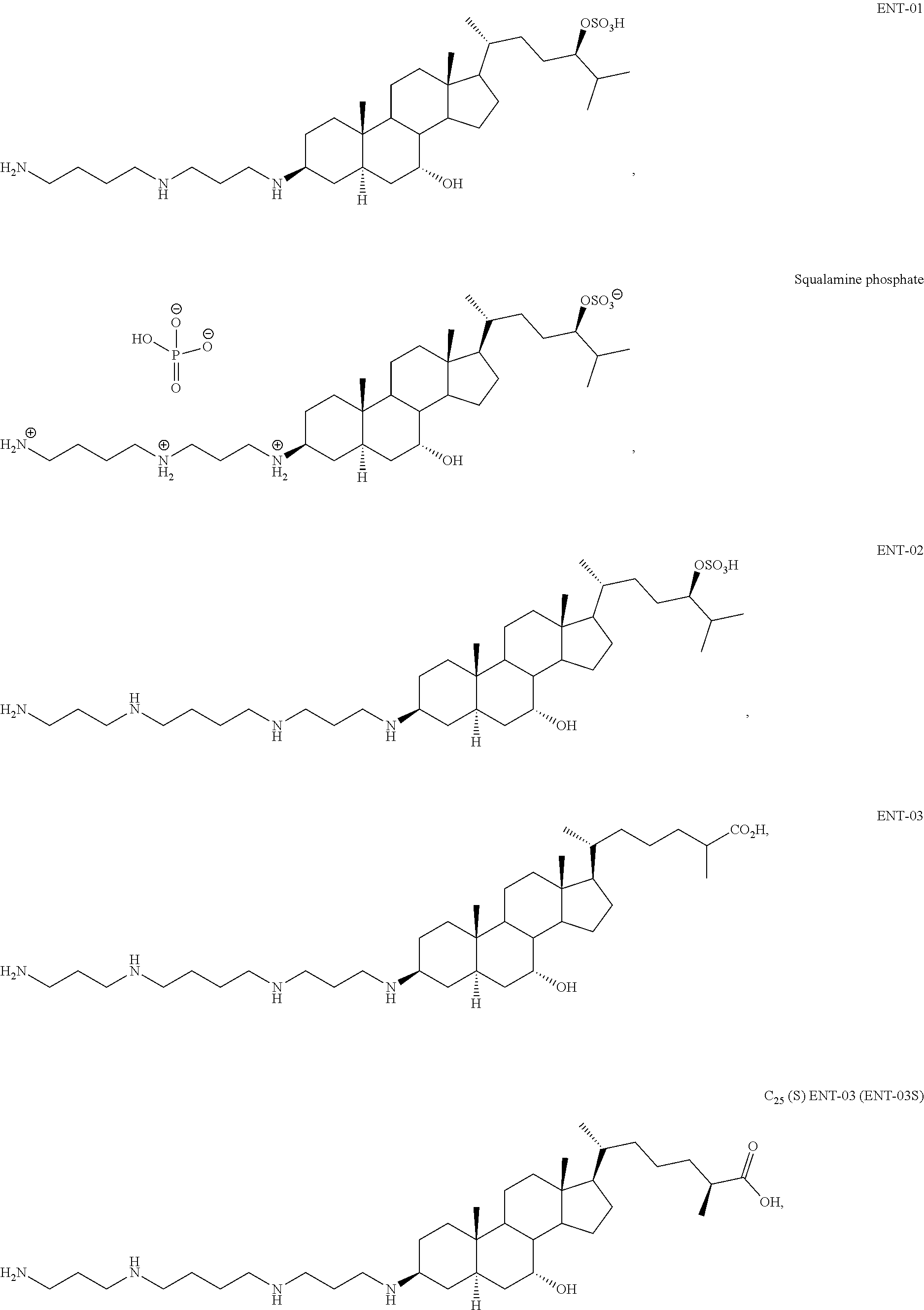

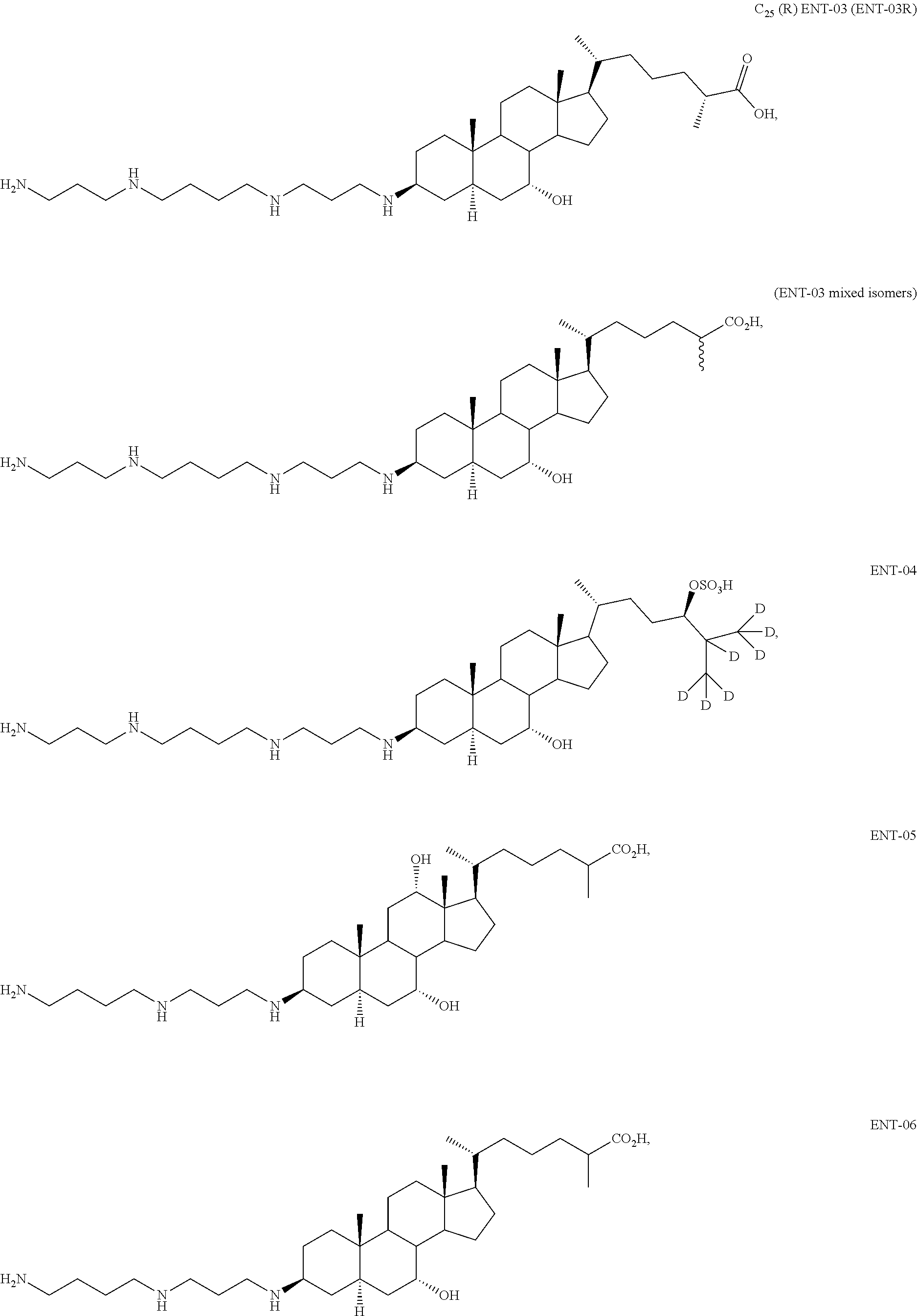

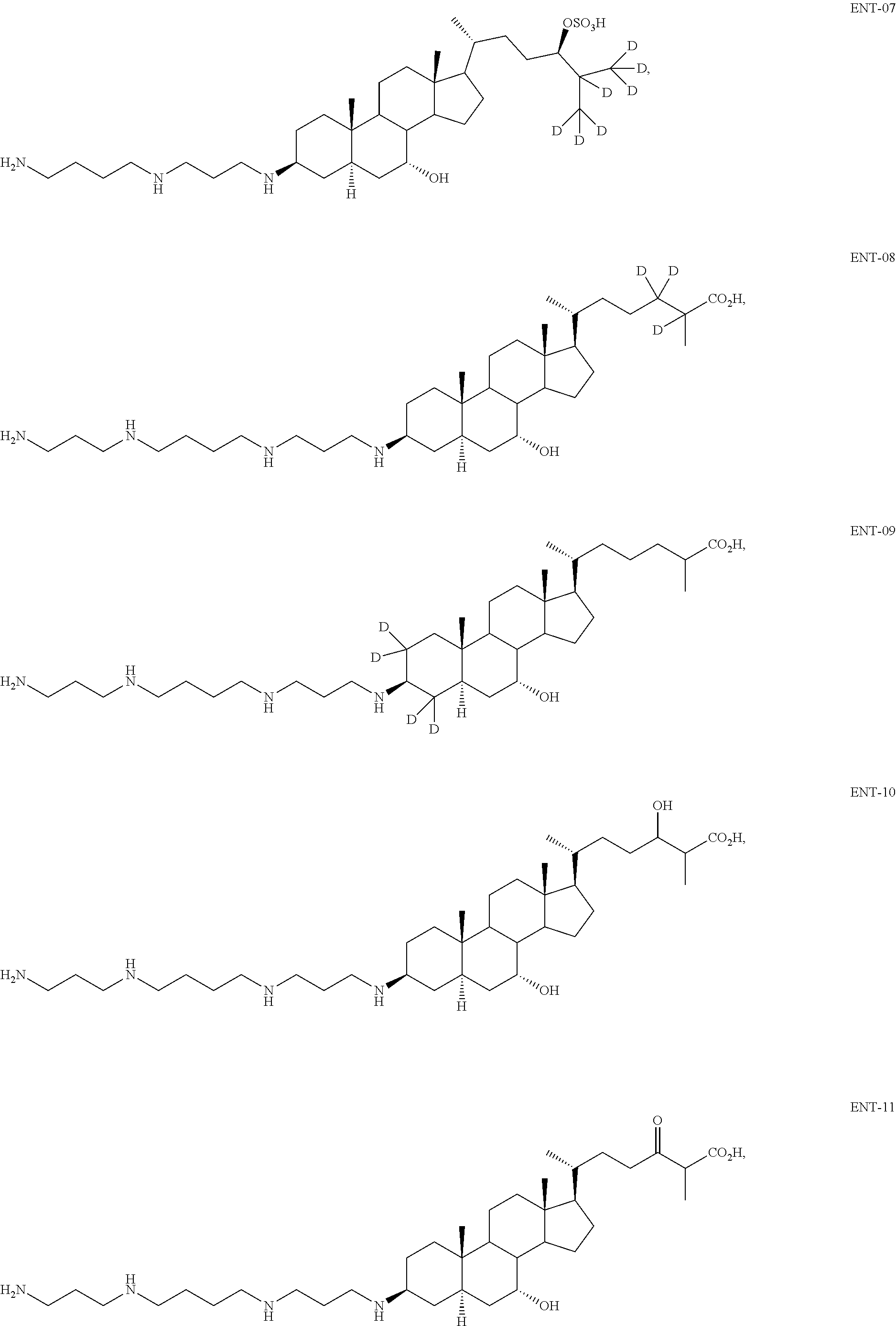

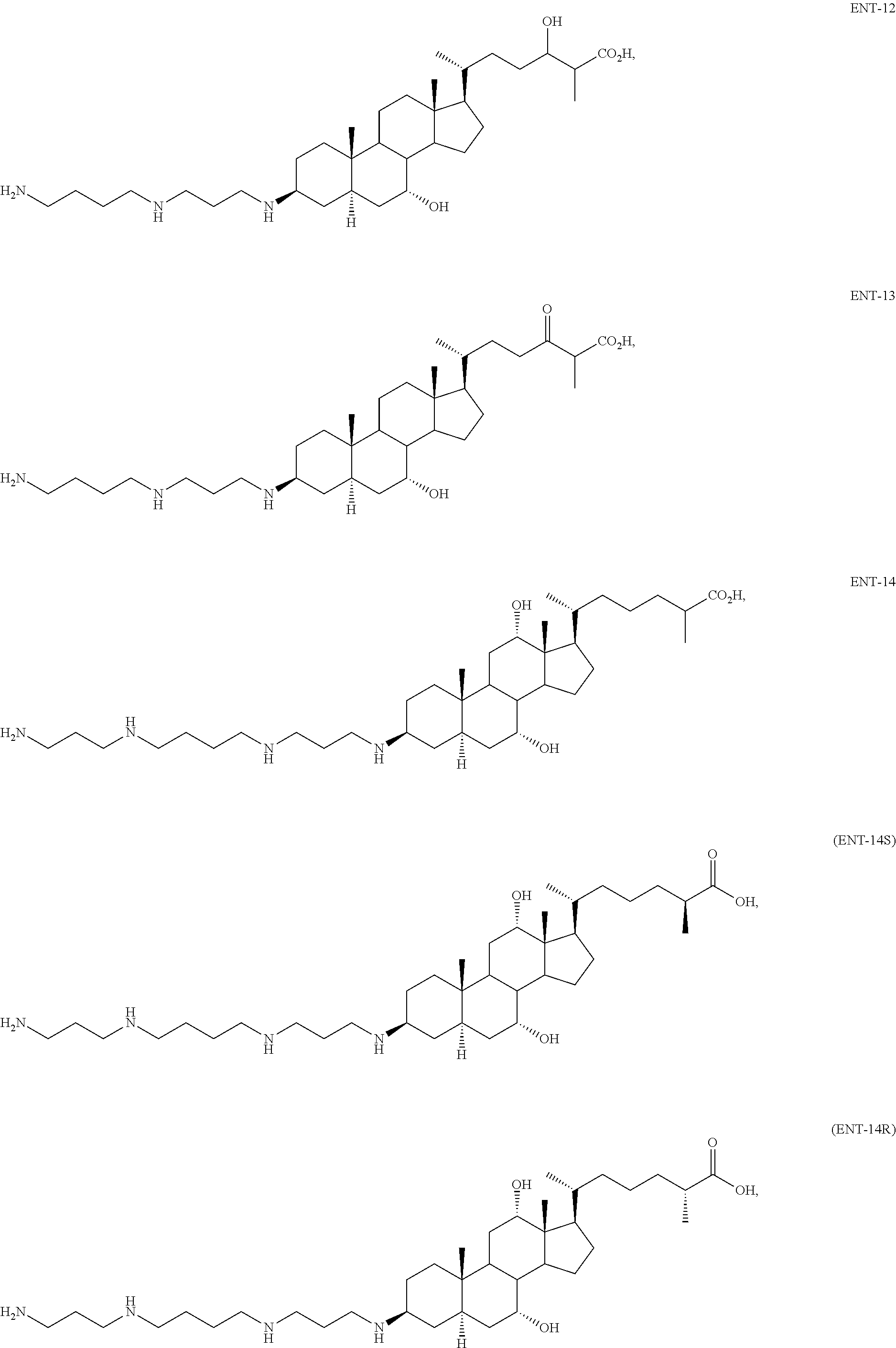

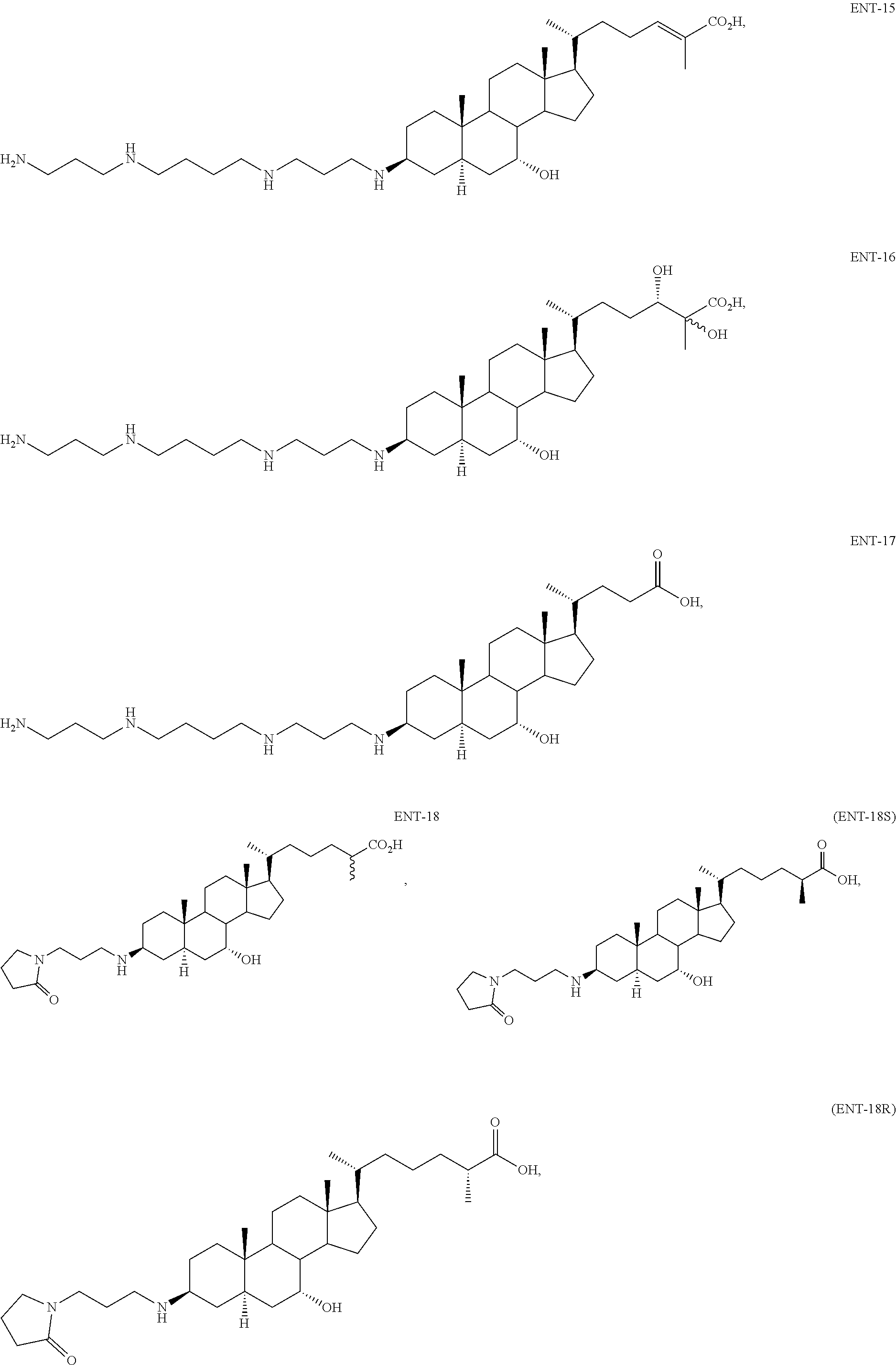

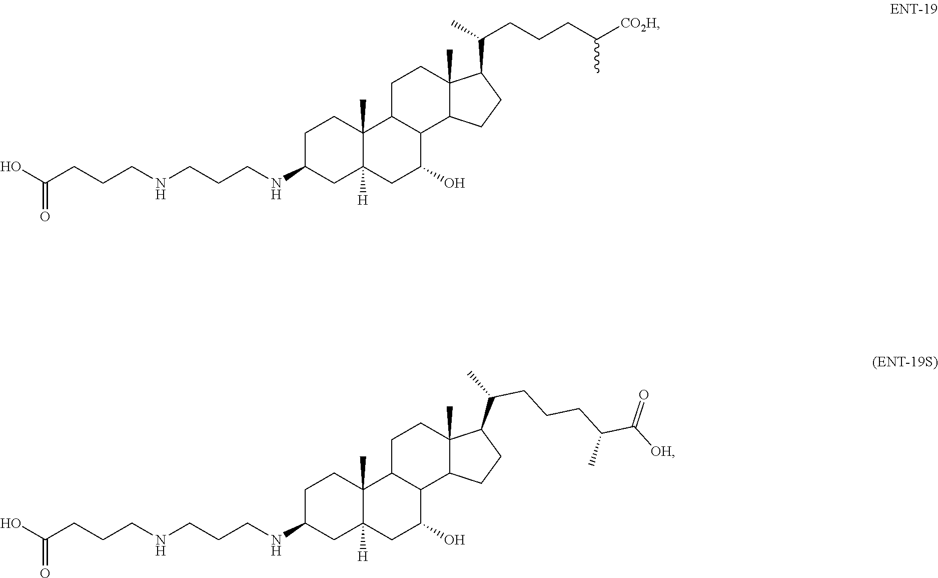 [SMILES: CC(CCC[C@@H](C)[C@H]1CCC2C3C(CC[C@@]21C)[C@@]1(C)CC[C@H](NCCCNCCCC(=O)O)C[C@@H]1C[C@H]3O)C(=O)O ; C[C@H](CCC[C@@H](C)[C@H]1CCC2C3C(CC[C@@]21C)[C@@]1(C)CC[C@H](NCCCNCCCC(=O)O)C[C@@H]1C[C@H]3O)C(=O)O]

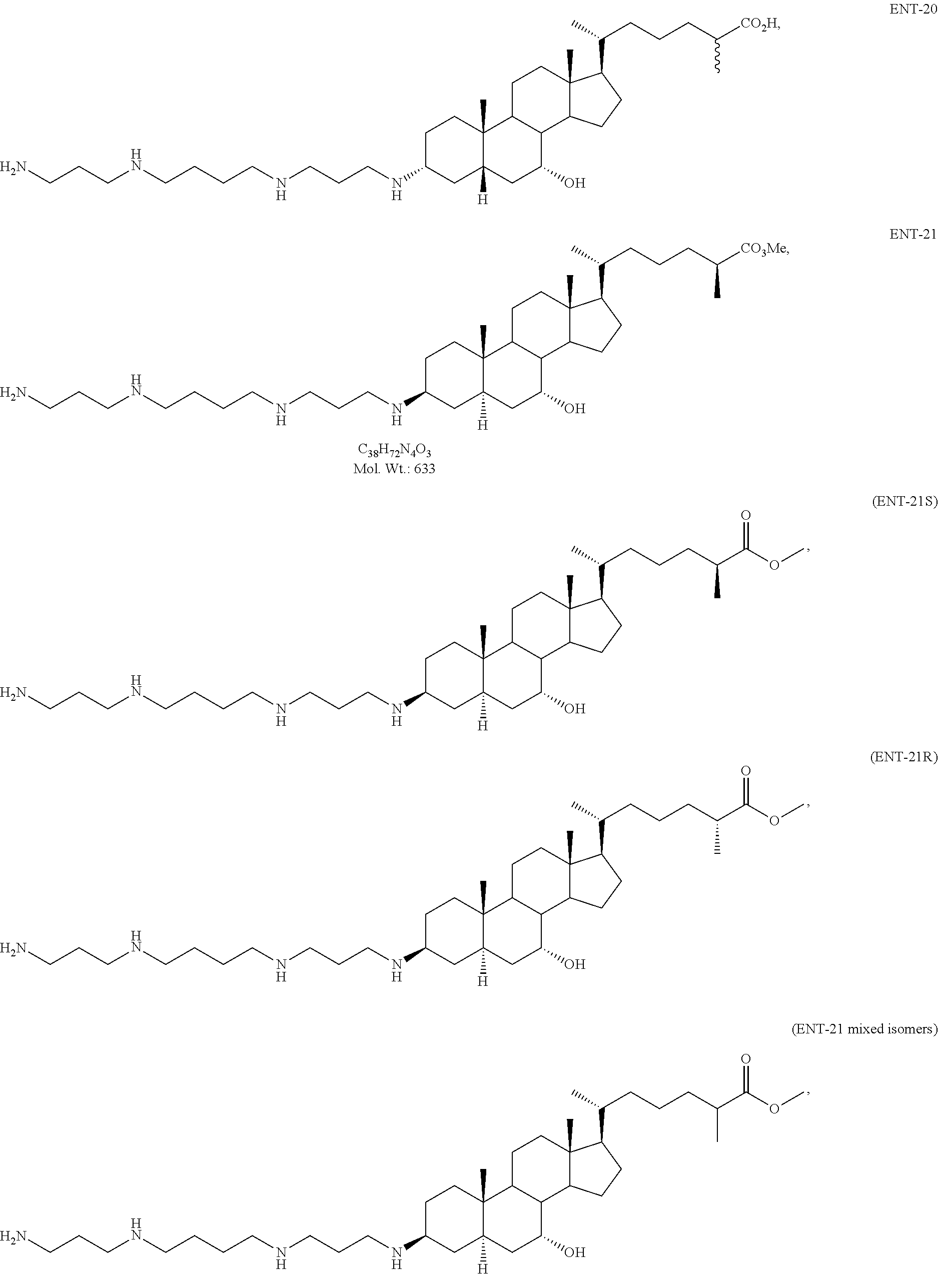

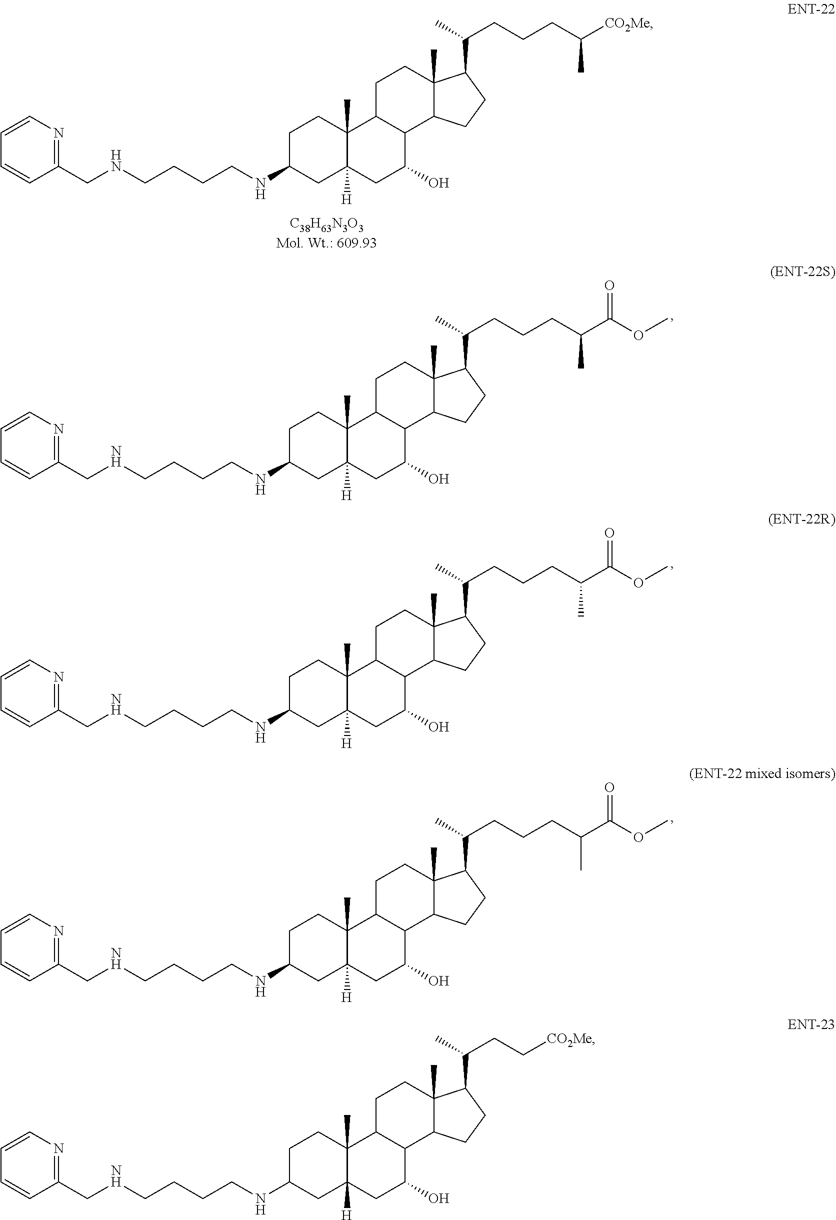

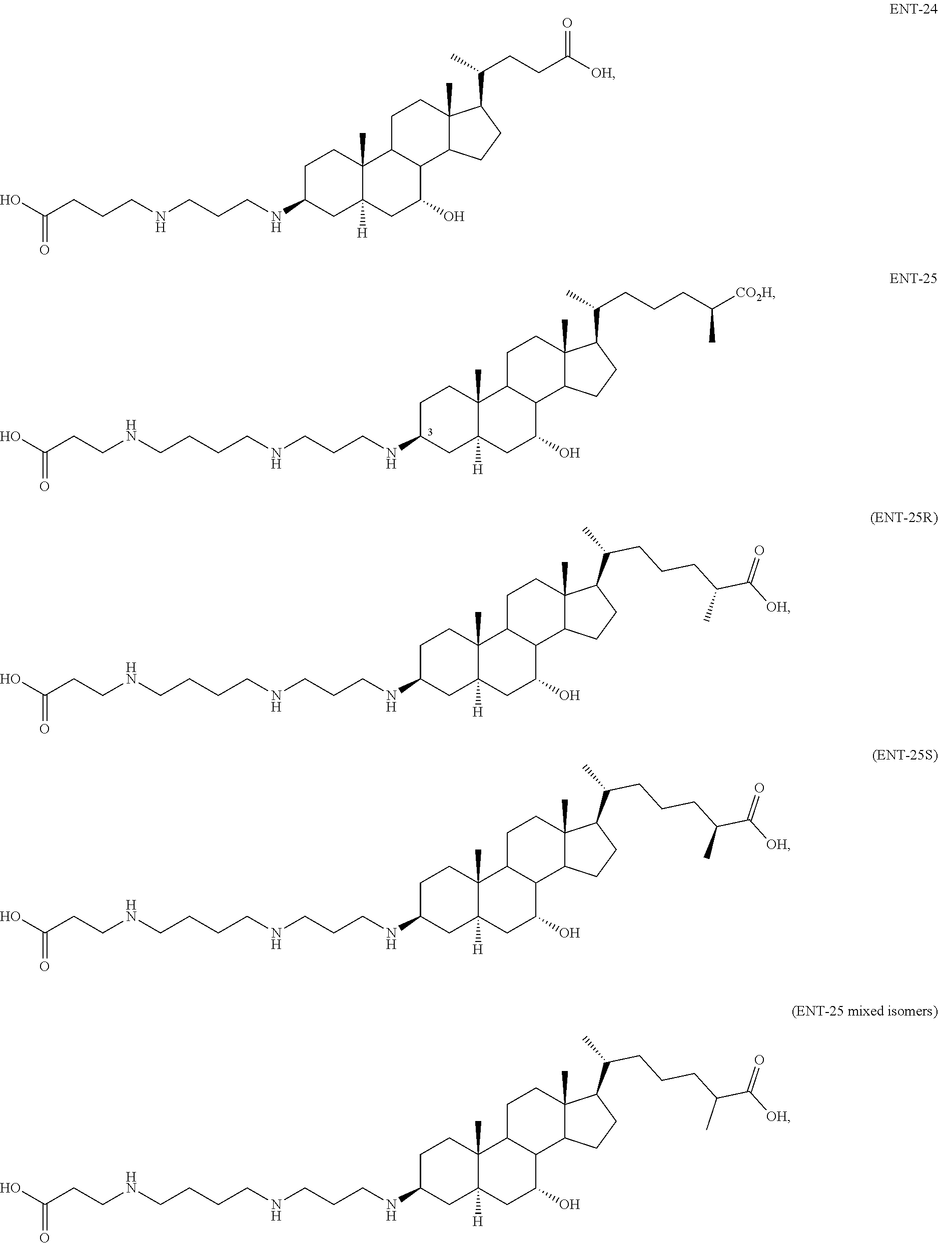

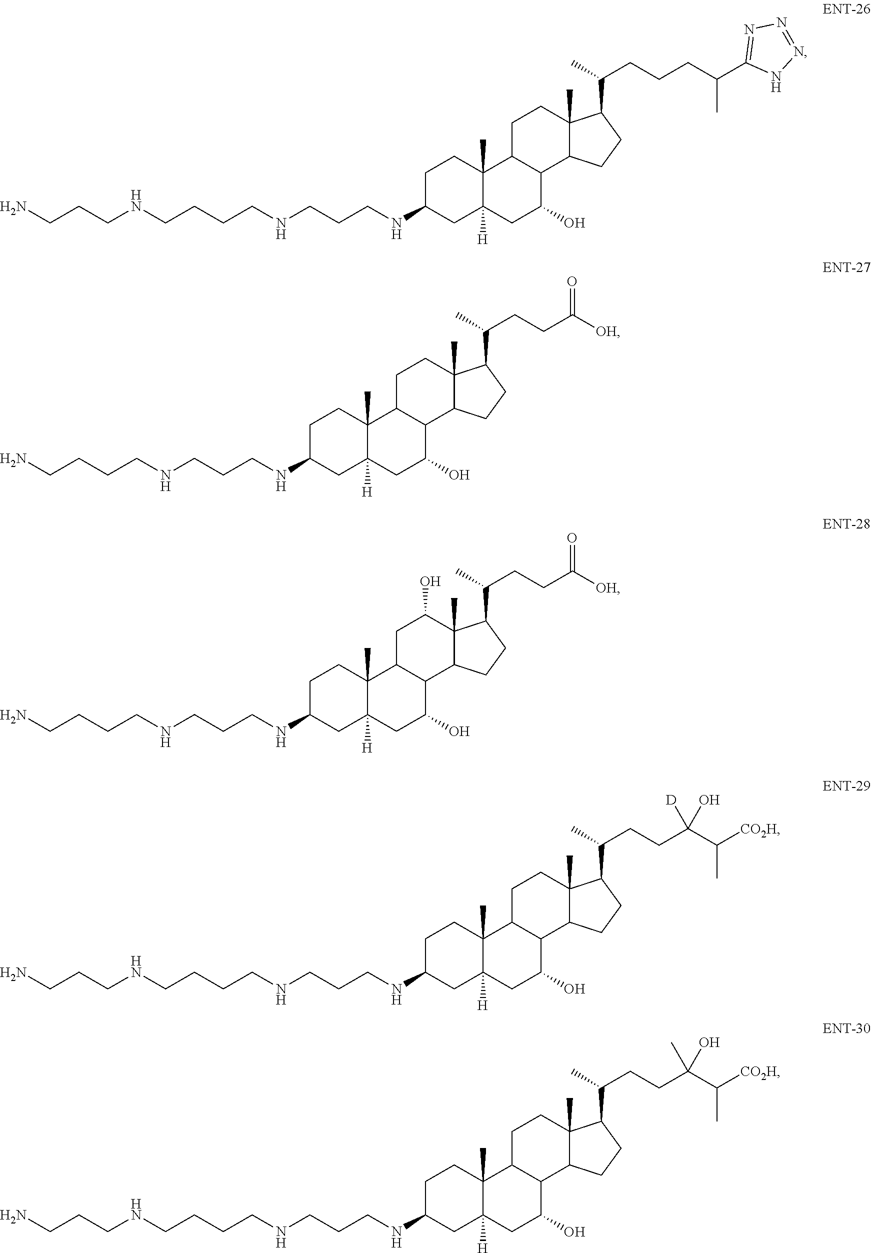 [SMILES: CC(CCC[C@@H](C)[C@H]1CCC2C3C(CC[C@@]21C)[C@@]1(C)CC[C@H](NCCCNCCCCNCCCN)C[C@@H]1C[C@H]3O)c1nnn[nH]1 ; C[C@H](CCC(=O)O)[C@H]1CCC2C3C(CC[C@@]21C)[C@@]1(C)CC[C@H](NCCCNCCCCN)C[C@@H]1C[C@H]3O ; C[C@H](CCC(=O)O)[C@H]1CCC2C3C(C[C@H](O)[C@@]21C)[C@@]1(C)CC[C@H](NCCCNCCCCN)C[C@@H]1C[C@H]3O ; [2H]C(O)(CC[C@@H](C)[C@H]1CCC2C3C(CC[C@@]21C)[C@@]1(C)CC[C@H](NCCCNCCCCNCCCN)C[C@@H]1C[C@H]3O)C(C)C(=O)O ; CC(C(=O)O)C(C)(O)CC[C@@H](C)[C@H]1CCC2C3C(CC[C@@]21C)[C@@]1(C)CC[C@H](NCCCNCCCCNCCCN)C[C@@H]1C[C@H]3O]

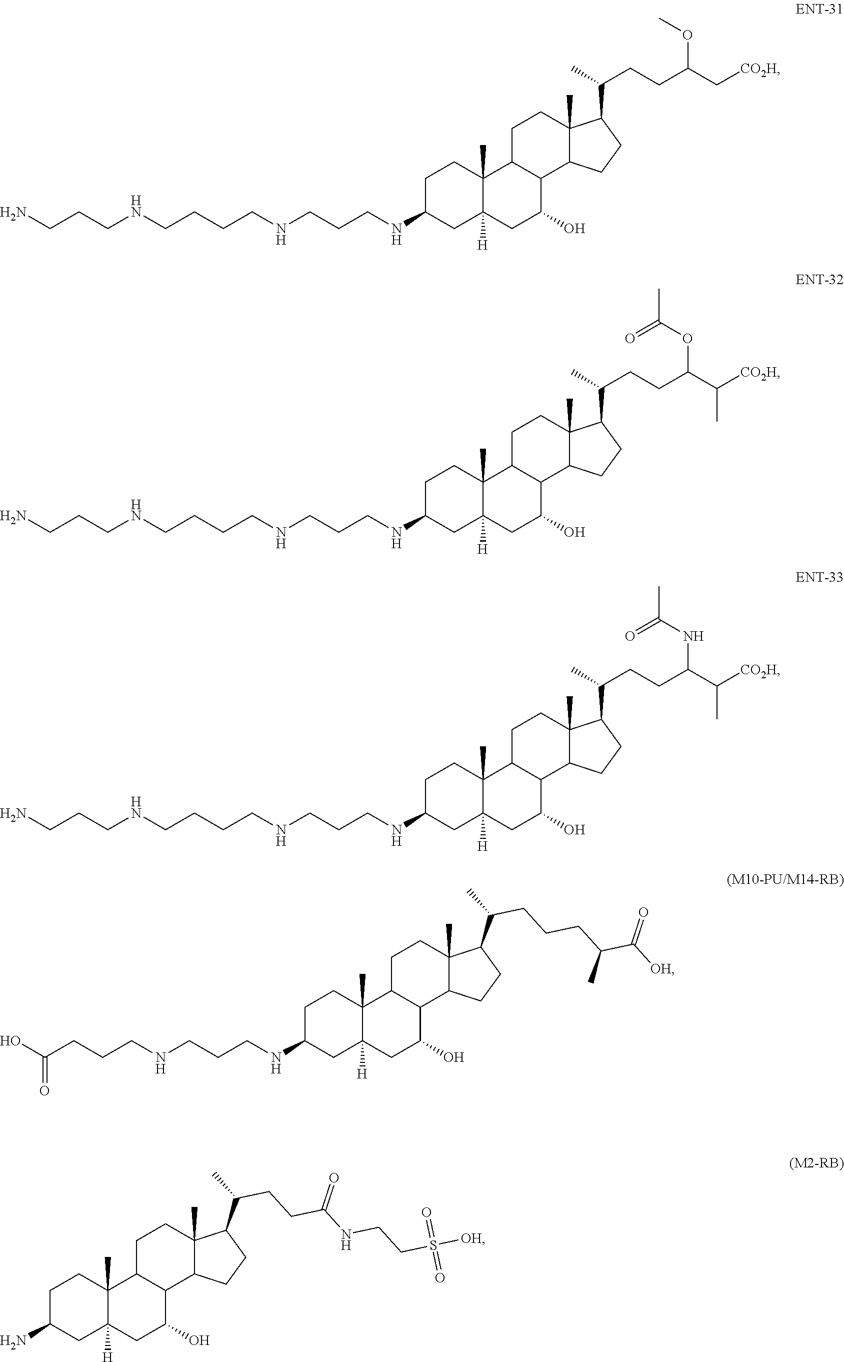

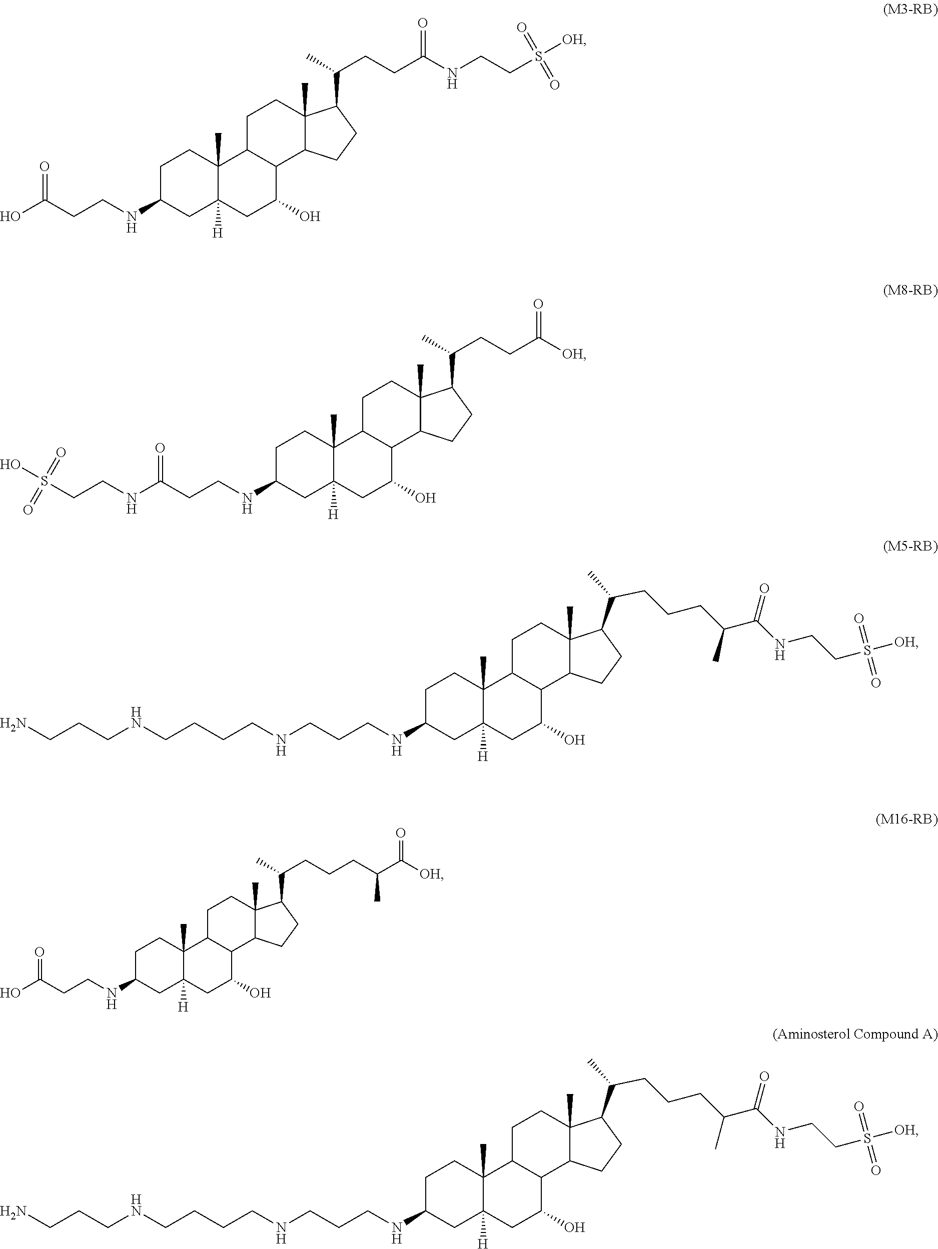

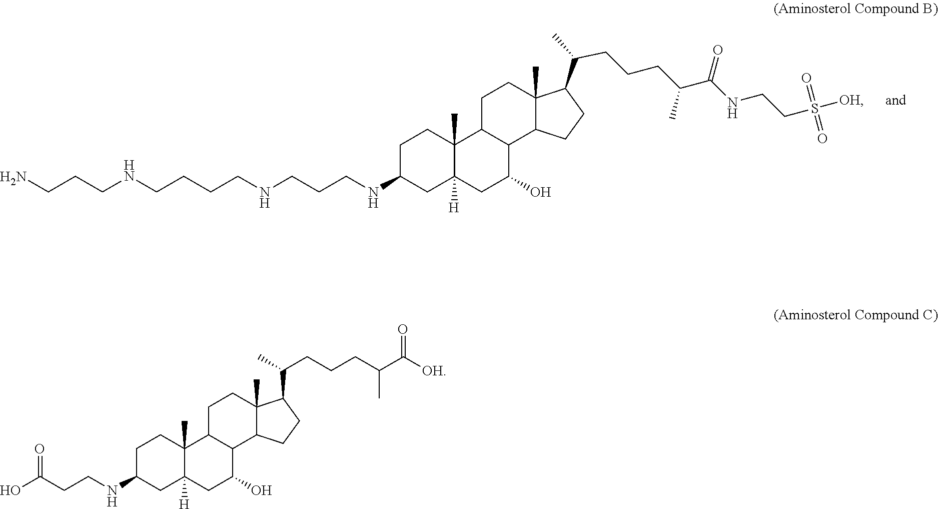 [SMILES: C[C@H](CCC[C@@H](C)[C@H]1CCC2C3C(CC[C@@]21C)[C@@]1(C)CC[C@H](NCCCNCCCCNCCCN)C[C@@H]1C[C@H]3O)C(=O)NCCS(=O)(=O)O ; CC(CCC[C@@H](C)[C@H]1CCC2C3C(CC[C@@]21C)[C@@]1(C)CC[C@H](NCCC(=O)O)C[C@@H]1C[C@H]3O)C(=O)O]

25. The aqueous aminosterol composition of claim 14, wherein:
(a) the aminosterol is ENT-03S, and optionally wherein the composition comprises 50 mg/ml of ENT-03S and about 20 to about 25% 2HP-3-CD, buffered to pH 7.3-7.5 with phosphate;
(b) the aminosterol is ENT-03R, and optionally wherein the composition comprises 50 mg/ml of ENT-03R and about 20 to about 25% 2HP-3-CD, buffered to pH 7.3-7.5 with phosphate;
(c) the aminosterol is racemic ENT-03, and optionally wherein the composition comprises 50 mg/ml of racemic ENT-03 and about 20 to about 25% 2HP-3-CD, buffered to pH 7.3-7.5 with phosphate;
(d) the aminosterol is a racemic aminosterol selected from the group consisting of racemic ENT-01, racemic squalamine phosphate, racemic ENT-02, racemic ENT-04, racemic ENT-05, racemic ENT-06, racemic ENT-07, racemic ENT-08, racemic ENT-09, racemic ENT-10, racemic ENT-11, racemic ENT-12, racemic ENT-13, racemic ENT-14, racemic ENT-15, racemic ENT-16, racemic ENT-17, racemic ENT-18, racemic ENT-19, racemic ENT-20, racemic ENT-21, racemic ENT-22, racemic ENT-23, racemic ENT-24, racemic ENT-25, racemic ENT-26, racemic ENT-27, racemic ENT-28, racemic ENT-29, racemic ENT-30, racemic ENT-31, racemic ENT-32, racemic ENT-33, racemic M10-PU/M14-RB, racemic M2-RB, racemic M3-RB, racemic M8-RB, racemic M5-RB, racemic M16-RB, racemic Aminosterol Compound A, racemic Aminosterol Compound B, and racemic Aminosterol Compound C, and optionally wherein the composition comprises 50 mg/ml of a racemic aminosterol and about 20 to about 25% 2HP-β-CD, buffered to pH 7.3-7.5 with phosphate.

26. A method of treating a subject in need having a condition susceptible to treatment with an aminosterol, comprising administering to the subject a therapeutically effective amount of an aminosterol compound, or a pharmaceutically acceptable salt, solvate, prodrug, enantiomer, or derivative thereof, or an aminosterol composition, according to claim 14.

27. The method of claim 26, wherein the condition is correlated with abnormal αS pathology, dopaminergic dysfunction, and/or protein misfolding, and optionally wherein the misfolded protein is α-synuclein, tau, Huntingtin, TDP-43 and/or amyloid-β.

28. A method of treating, preventing, and/or slowing the onset or progression of a condition or disorder, or a related symptom, correlated with abnormal αS pathology, dopaminergic dysfunction, and/or protein misfolding in a subject in need, comprising administering a therapeutically effective amount of an aminosterol compound, or a pharmaceutically acceptable salt, solvate, prodrug, enantiomer, or derivative thereof, or an aminosterol composition, according to claim 14.

29. (canceled)

30. (canceled)

31. A method of;
(a) suppressing, preventing and/or slowing the onset or progression of appetite or weight gain, and/or one or more related symptoms, in a subject in need thereof, comprising administering to the subject a therapeutically effective amount of an aminosterol compound, or a pharmaceutically acceptable salt, solvate, prodrug, enantiomer, or derivative thereof, or an aminosterol composition, according to claim 14,
(b) improving or resolving impaired reproductive function, impaired fertility and/or impaired reproductive behavior in a subject in need thereof, comprising administering to the subject a therapeutically effective amount of an aminosterol compound, or a pharmaceutically acceptable salt, solvate, prodrug, enantiomer, or derivative thereof, or an aminosterol composition, according to claim 14: or
(c) treating, preventing, and/or improving diabetes, which can be either type 1 or type 2 diabetes, in a subject in need thereof, comprising administering to the subject a therapeutically effective amount of an aminosterol compound, or a pharmaceutically acceptable salt, solvate, prodrug, enantiomer, or derivative thereof, or an aminosterol composition, according to claim 14.

32-40. (canceled)
